(19) United States
(12) Patent Application Publication
(10) Pub. No.: US 2009/0023728 A1
(43) Pub. Date: Jan. 22, 2009

(54) 1,2,3,4-Tetrahydroisoquinoline Derivatives, Preparations Thereof and Uses Thereof

(75) Inventors: Justin Ripper, Queensland (AU); Ian Jenkins, Queensland (AU); Phuc Van Le, Queensland (AU); Ron Quinn, Queensland (AU); Christian Janssen, Capricorn (SG)

Correspondence Address: ASTRA ZENECA PHARMACEUTICALS LP;GLOBAL INTELLECTUAL PROPERTY, 1800 CONCORD PIKE, WILMINGTON, DE, 19850-5437, US

(73) Assignee: ASTRAZENECA AB, Sodertalje (SE)

(21) Appl. No.: 10/596,546

(22) PCT Filed: Dec. 20, 2004
(86) PCT No.: PCT/SE04/01939

§ 371 Date: Jun. 16, 2006

(30) Foreign Application Priority Data
Dec. 22, 2003 (SE) 0303542-5

(51) Int. Cl. A61K 31/4725; C07D 405/06; C07D 409/06; C07D 401/06; A61K 31/5377; A61P 1/00; A61P 25/00; C07D 413/14; C07D 491/056

(52) U.S. Cl. 514/235.2; 546/148; 544/128; 546/115; 514/307

(57) ABSTRACT

Compounds of general formula (I) wherein D, E, R¹, R², R³, R⁴, R⁵, R⁶ and R⁷ are as defined in the specification, as well as salts, enantiomers thereof and pharmaceutical compositions including the compounds are prepared. They are useful in therapy, in particular in the management of pain.

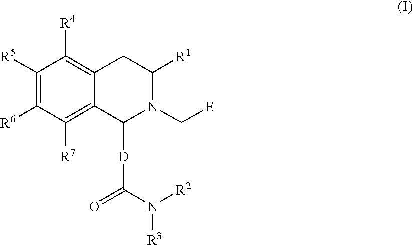

### BACKGROUND OF THE INVENTION

1. Field of the Invention

The present invention is directed to novel compounds, processes for their preparation, their uses and pharmaceutical compositions comprising the novel compounds. The novel compounds are useful in therapy, and in particular for the treatment of pain, anxiety and functional gastrointestinal disorders.

2. Discussion of Relevant Art

The δ receptor has been identified as having a role in many bodily functions such as circulatory and pain systems. Ligands for the δ receptor may therefore find potential use as analgesics, and/or as antihypertensive agents. Ligands for the 3 receptor have also been shown to possess immunomodulatory activities.

The identification of at least three different populations of opioid receptors (μ, δ and κ) is now well established and all three are apparent in both central and peripheral nervous systems of many species including man. Analgesia has been observed in various animal models when one or more of these receptors has been activated.

With few exceptions, currently available selective opioid 3 ligands are peptidic in nature and are unsuitable for administration by systemic routes. One example of a non-peptidic δ-agonist is SNC80 (Bilsky E. J. et al., Journal of Pharmacology and Experimental Therapeutics, 273(1), pp. 359-366 (1995)).

Many δ agonist compounds that have been identified in the prior art have many disadvantages in that they suffer from poor pharmacokinetics and are not analgesic when administered by systemic routes. Also, it has been documented that many of these δ agonist compounds show significant convulsive effects when administered systemically.

Therefore, there is still a need for improved δ-agonists.

### DESCRIPTION OF THE EMBODIMENTS

Thus, the problem underlying the present invention was to find new analgesics having improved analgesic effects, but also with an improved side-effect profile over current μ agonists, as well as having improved systemic efficacy.

We have now found certain compounds that exhibit surprisingly improved properties, i.e. improved δ agonist potency, in vivo potency, pharmacokinetic, bioavailability, in vitro stability and/or lower toxicity.

Accordingly, it is an objective of certain embodiments of the present invention to provide improved δ receptor ligands.

Unless specified otherwise within this specification, the nomenclature used in this specification generally follows the examples and rules stated in Nomenclature of Organic Chemistry, Sections A, B, C, D, E, F, and H, Pergamon Press, Oxford, 1979, which is incorporated by references herein for its exemplary chemical structure names and rules on naming chemical structures. Optionally, a name of a compound may be generated using a chemical naming program: ACD/ChemSketch, Version 5.09/September 2001, Advanced Chemistry Development, Inc., Toronto, Canada.

The term “Cₘ₋ₙ” or “Cₘ₋ₙ group” used alone or as a prefix, refers to any group having m to n carbon atoms.

The term “hydrocarbon” used alone or as a suffix or prefix, refers to any structure comprising only carbon and hydrogen atoms up to 14 carbon atoms.

The term “hydrocarbon radical” or “hydrocarbyl” used alone or as a suffix or prefix, refers to any structure as a result of removing one or more hydrogens from a hydrocarbon.

The term “alkyl” used alone or as a suffix or prefix, refers to a saturated monovalent straight or branched chain hydrocarbon radical comprising 1 to about 12 carbon atoms. Illustrative examples of alkyls include, but are not limited to, C₁₋₆alkyl groups, such as methyl, ethyl, propyl, isopropyl, 2-methyl-1-propyl, 2-methyl-2-propyl, 2-methyl-1-butyl, 3-methyl-1-butyl, 2-methyl-3-butyl, 2,2-dimethyl-1-propyl, 2-methyl-1-pentyl, 3-methyl-1-pentyl, 4-methyl-1-pentyl, 2-methyl-2-pentyl, 3-methyl-2-pentyl, 4-methyl-2-pentyl, 2,2-dimethyl-1-butyl, 3,3-dimethyl-1-butyl, 2-ethyl-1-butyl, butyl, isobutyl, t-butyl, pentyl, isopentyl, neopentyl, and hexyl, and longer alkyl groups, such as heptyl, and octyl. An alkyl can be unsubstituted or substituted with one or two suitable substituents.

The term “alkylene” used alone or as suffix or prefix, refers to a saturated divalent straight or branched chain hydrocarbon radicals comprising 1 to about 12 carbon atoms, which serves to links two structures together.

The term “alkenyl” used alone or as suffix or prefix, refers to a monovalent straight or branched chain hydrocarbon radical having at least one carbon-carbon double bond and comprising at least 2 up to about 12 carbon atoms. The double bond of an alkenyl can be unconjugated or conjugated to another unsaturated group. Suitable alkenyl groups include, but are not limited to C₂₋₆alkenyl groups, such as vinyl, allyl, butenyl, pentenyl, hexenyl, butadienyl, pentadienyl, hexadienyl, 2-ethylhexenyl, 2-propyl-2-butenyl, 4-(2-methyl-3-butene)-pentenyl. An alkenyl can be unsubstituted or substituted with one or two suitable substituents.

The term “alkynyl” used alone or as suffix or prefix, refers to a monovalent straight or branched chain hydrocarbon radical having at least one carbon-carbon triple bond and comprising at least 2 up to about 12 carbon atoms. The triple bond of an alkynyl group can be unconjugated or conjugated to another unsaturated group. Suitable alkynyl groups include, but are not limited to, C₂₋₆alkynyl groups, such as ethynyl, propynyl, butynyl, pentynyl, hexynyl, methylpropynyl, 4-methyl-1-butynyl, 4-propyl-2-pentynyl, and 4-butyl-2-hexynyl. An alkynyl can be unsubstituted or substituted with one or two suitable substituents.

The term “cycloalkyl,” used alone or as suffix or prefix, refers to a saturated monovalent ring-containing hydrocarbon radical comprising at least 3 up to about 12 carbon atoms. Examples of cycloalkyls include, but are not limited to, C₃₋₇cycloalkyl groups, such as cyclopropyl, cyclobutyl, cyclopentyl, cyclohexyl, and cycloheptyl, and saturated cyclic and bicyclic terpenes. A cycloalkyl can be unsubstituted or substituted by one or two suitable substituents. Preferably, the cycloalkyl is a monocyclic ring or bicyclic ring.

The term “cycloalkenyl” used alone or as suffix or prefix, refers to a monovalent ring-containing hydrocarbon radical having at least one carbon-carbon double bond and comprising at least 3 up to about 12 carbon atoms.

The term “cycloalkynyl” used alone or as suffix or prefix, refers to a monovalent ring-containing hydrocarbon radical having at least one carbon-carbon triple bond and comprising about 7 up to about 12 carbon atoms.

The term “aryl” used alone or as suffix or prefix, refers to a monovalent hydrocarbon radical having one or more polyunsaturated carbon rings having aromatic character, (e.g., 4n+2 delocalized electrons) and comprising 5 up to about 14 carbon atoms.

The term “arylene” used alone or as suffix or prefix, refers to a divalent hydrocarbon radical having one or more polyunsaturated carbon rings having aromatic character, (e.g., 4n+2 delocalized electrons) and comprising 5 up to about 14 carbon atoms, which serves to links two structures together.

The term “heterocycle” used alone or as a suffix or prefix, refers to a ring-containing structure or molecule having one or more multivalent heteroatoms, independently selected from N, O and S, as a part of the ring structure and including at least 3 and up to about 20 atoms in the ring(s). Heterocycle may be saturated or unsaturated, containing one or more double bonds, and heterocycle may contain more than one ring. When a heterocycle contains more than one ring, the rings may be fused or unfused. Fused rings generally refer to at least two rings share two atoms therebetween. Heterocycle may have aromatic character or may not have aromatic character.

The term “heteroalkyl” used alone or as a suffix or prefix, refers to a radical formed as a result of replacing one or more carbon atom of an alkyl with one or more heteroatoms selected from N, O and S.

The term “heteroaromatic” used alone or as a suffix or prefix, refers to a ring-containing structure or molecule having one or more multivalent heteroatoms, independently selected from N, O and S, as a part of the ring structure and including at least 3 and up to about 20 atoms in the ring(s), wherein the ring-containing structure or molecule has an aromatic character (e.g., 4n+2 delocalized electrons).

The term “heterocyclic group,” “heterocyclic moiety,” “heterocyclic,” or “heterocyclo” used alone or as a suffix or prefix, refers to a radical derived from a heterocycle by removing one or more hydrogens therefrom.

The term “heterocyclyl” used alone or as a suffix or prefix, refers a monovalent radical derived from a heterocycle by removing one hydrogen therefrom.

The term “heterocyclylene” used alone or as a suffix or prefix, refers to a divalent radical derived from a heterocycle by removing two hydrogens therefrom, which serves to links two structures together.

The term “heteroaryl” used alone or as a suffix or prefix, refers to a heterocyclyl having aromatic character.

The term “heterocylcoalkyl” used alone or as a suffix or prefix, refers to a monocyclic or polycyclic ring comprising carbon and hydrogen atoms and at least one heteroatom, preferably, 1 to 3 heteroatoms selected from nitrogen, oxygen, and sulfur, and having no unsaturation. Examples of heterocycloalkyl groups include pyrrolidinyl, pyrrolidino, piperidinyl, piperidino, piperazinyl, piperazino, morpholinyl, morpholino, thiomorpholinyl, thiomorpholino, and pyranyl. A heterocycloalkyl group can be unsubstituted or substituted with one or two suitable substituents. Preferably, the heterocycloalkyl group is a monocyclic or bicyclic ring, more preferably, a monocyclic ring, wherein the ring comprises from 3 to 6 carbon atoms and form 1 to 3 heteroatoms, referred to herein as C₃₋₆heterocycloalkyl.

The term “heteroarylene” used alone or as a suffix or prefix, refers to a heterocyclylene having aromatic character.

The term “heterocycloalkylene” used alone or as a suffix or prefix, refers to a heterocyclylene that does not have aromatic character.

The term “six-membered” used as prefix refers to a group having a ring that contains six ring atoms.

The term “five-membered” used as prefix refers to a group having a ring that contains five ring atoms.

A five-membered ring heteroaryl is a heteroaryl with a ring having five ring atoms wherein 1, 2 or 3 ring atoms are independently selected from N, O and S.

Exemplary five-membered ring heteroaryls are thienyl, furyl, pyrrolyl, imidazolyl, thiazolyl, oxazolyl, pyrazolyl, isothiazolyl, isoxazolyl, 1,2,3-triazolyl, tetrazolyl, 1,2,3-thiadiazolyl, 1,2,3-oxadiazolyl, 1,2,4-triazolyl, 1,2,4-thiadiazolyl, 1,2,4-oxadiazolyl, 1,3,4-triazolyl, 1,3,4-thiadiazolyl, and 1,3,4-oxadiazolyl.

A six-membered ring heteroaryl is a heteroaryl with a ring having six ring atoms wherein 1, 2 or 3 ring atoms are independently selected from N, O and S.

Exemplary six-membered ring heteroaryls are pyridyl, pyrazinyl, pyrimidinyl, triazinyl and pyridazinyl.

The term “substituted” used as a prefix refers to a structure, molecule or group, wherein one or more hydrogens are replaced with one or more C₁₋₁₂hydrocarbon groups, or one or more chemical groups containing one or more heteroatoms selected from N, O, S, F, Cl, Br, I, and P. Exemplary chemical groups containing one or more heteroatoms include heterocyclyl, —NO₂, —OR, —Cl, —Br, —I, —F, —CF₃, —C(═O)R, —C(═O)OH, —NH₂, —SH, —NHR, —NR₂, —SR, —SO₃H, —SO₂R, —S(═O)R, —CN, —OH, —C(═O)OR, —C(═O)NR₂, —NRC(═O)R, oxo (═O), imino (═NR), thio (═S), and oximino (═N—OR), wherein each “R” is a C₁₋₁₂hydrocarbyl. For example, substituted phenyl may refer to nitrophenyl, pyridylphenyl, methoxyphenyl, chlorophenyl, aminophenyl, etc., wherein the nitro, pyridyl, methoxy, chloro, and amino groups may replace any suitable hydrogen on the phenyl ring.

The term “substituted” used as a suffix of a first structure, molecule or group, followed by one or more names of chemical groups refers to a second structure, molecule or group, which is a result of replacing one or more hydrogens of the first structure, molecule or group with the one or more named chemical groups. For example, a “phenyl substituted by nitro” refers to nitrophenyl.

The term “optionally substituted” refers to both groups, structures, or molecules that are substituted and those that are not substituted.

Heterocycle includes, for example, monocyclic heterocycles such as: aziridine, oxirane, thiirane, azetidine, oxetane, thietane, pyrrolidine, pyrroline, imidazolidine, pyrazolidine, pyrazoline, dioxolane, sulfolane 2,3-dihydrofuran, 2,5-dihydrofuran, tetrahydrofuran, thiophane, piperidine, 1,2,3,6-tetrahydro-pyridine, piperazine, morpholine, thiomorpholine, pyran, thiopyran, 2,3-dihydropyran, tetrahydropyran, 1,4-dihydropyridine, 1,4-dioxane, 1,3-dioxane, dioxane, homopiperidine, 2,3,4,7-tetrahydro-1H-azepine homopiperazine, 1,3-dioxepane, 4,7-dihydro-1,3-dioxepin, and hexamethylene oxide.

In addition, heterocycle includes aromatic heterocycles, for example, pyridine, pyrazine, pyrimidine, pyridazine, thiophene, furan, furazan, pyrrole, imidazole, thiazole, oxazole, pyrazole, isothiazole, isoxazole, 1,2,3-triazole, tetrazole, 1,2,3-thiadiazole, 1,2,3-oxadiazole, 1,2,4-triazole, 1,2,4-thiadiazole, 1,2,4-oxadiazole, 1,3,4-triazole, 1,3,4-thiadiazole, and 1,3,4-oxadiazole.

Additionally, heterocycle encompass polycyclic heterocycles, for example, indole, indoline, isoindoline, quinoline, tetrahydroquinoline, isoquinoline, tetrahydroisoquinoline, 1,4-benzodioxan, coumarin, dihydrocoumarin, benzofuran, 2,3-dihydrobenzofuran, isobenzofuran, chromene, chroman, isochroman, xanthene, phenoxathiin, thianthrene, indolizine, isoindole, indazole, purine, phthalazine, naphthyridine, quinoxaline, quinazoline, cinnoline, pteridine, phenanthridine, perimidine, phenanthroline, phenazine, phenothiazine, phenoxazine, 1,2-benzisoxazole, benzothiophene, benzoxazole, benzthiazole, benzimidazole, benztriazole, thioxanthine, carbazole, carboline, acridine, pyrolizidine, and quinolizidine.

In addition to the polycyclic heterocycles described above, heterocycle includes polycyclic heterocycles wherein the ring fusion between two or more rings includes more than one bond common to both rings and more than two atoms common to both rings. Examples of such bridged heterocycles include quinuclidine, diazabicyclo[2.2.1]heptane and 7-oxabicyclo[2.2.1]heptane.

Heterocyclyl includes, for example, monocyclic heterocyclyls, such as: aziridinyl, oxiranyl, thiiranyl, azetidinyl, oxetanyl, thietanyl, pyrrolidinyl, pyrrolinyl, imidazolidinyl, pyrazolidinyl, pyrazolinyl, dioxolanyl, sulfonyl, 2,3-dihydrofuranyl, 2,5-dihydrofuranyl, tetrahydrofuranyl, thiophenyl, piperidinyl, 1,2,3,6-tetrahydropyridinyl, piperazinyl, morpholinyl, thiomorpholinyl, pyranyl, thiopyranyl, 2,3-dihydropyranyl, tetrahydropyranyl, 1,4-dihydropyridinyl, 1,4-dioxanyl, 1,3-dioxanyl, dioxanyl, homopiperidinyl, 2,3,4,7-tetrahydro-1H-azepinyl, homopiperazinyl, 1,3-dioxepanyl, 4,7-dihydro-1,3-dioxepinyl, and hexamethylene oxidyl.

In addition, heterocyclyl includes aromatic heterocyclyls or heteroaryl, for example, pyridinyl, pyrazinyl, pyrimidinyl, pyridazinyl, thienyl, furyl, furazanyl, pyrrolyl, imidazolyl, thiazolyl, oxazolyl, pyrazolyl, isothiazolyl, isoxazolyl, 1,2,3-triazolyl, tetrazolyl, 1,2,3-thiadiazolyl, 1,2,3-oxadiazolyl, 1,2,4-triazolyl, 1,2,4-thiadiazolyl, 1,2,4-oxadiazolyl, 1,3,4-triazolyl, 1,3,4-thiadiazolyl, and 1,3,4 oxadiazolyl.

Additionally, heterocyclyl encompasses polycyclic heterocyclyls (including both aromatic or non-aromatic), for example, indolyl, indolinyl, isoindolinyl, quinolinyl, tetrahydroquinolinyl, isoquinolinyl, tetrahydroisoquinolinyl, 1,4-benzodioxanyl, coumarinyl, dihydrocumarinyl, benzofuranyl, 2,3-dihydrobenzofuranyl, isobenzofuranyl, chromenyl, chromanyl, isochromanyl, xanthenyl, phenoxathiinyl, thianthrenyl, indolizinyl, isoindolyl, indazolyl, purinyl, phthalazinyl naphthyridinyl, quinoxalinyl quinazolinyl, cinnolinyl, pteridinyl, phenanthridinyl, perimidinyl, phenanthrolinyl, phenazinyl, phenothiazinyl, phenoxazinyl 1,2-benzisoxazolyl, benzothiophenyl, benzoxazolyl, benzthiazolyl, benzimidazolyl, benztriazolyl, thioxanthinyl, carbazolyl, carbolinyl, acridinyl, pyrolizidinyl, and quinolizidinyl.

In addition to the polycyclic heterocyclyls described above, heterocyclyl includes polycyclic heterocyclyls wherein the ring fusion between two or more rings includes more than one bond common to both rings and more than two atoms common to both rings. Examples of such bridged heterocycles include quinuclidinyl, diazabicyclo[2.2.1]heptyl; and 7-oxabicyclo[2.2.1]heptyl.

The term “alkoxy” used alone or as a suffix or prefix, refers to radicals of the general formula —O—R, wherein R is selected from a hydrocarbon radical. Exemplary alkoxy includes methoxy, ethoxy, propoxy, isopropoxy, butoxy, t-butoxy, isobutoxy, cyclopropylmethoxy, allyloxy, and propargyloxy.

The term “amine” or “amino” used alone or as a suffix or prefix, refers to radicals of the general formula —NRR′, wherein R and R′ are independently selected from hydrogen or a hydrocarbon radical.

“Acyl” used alone, as a prefix or suffix, means C(═O)—R, wherein R is an optionally substituted hydrocarbyl, hydrogen, amino or alkoxy. Acyl groups include, for example, acetyl, propionyl, benzoyl, phenyl acetyl, carboethoxy, and dimethylcarbamoyl.

Halogen includes fluorine, chlorine, bromine and iodine.

“Halogenated,” used as a prefix of a group, means one or more hydrogens on the group is replaced with one or more halogens.

“RT” or “rt” means room temperature.

A first ring group being “fused” with a second ring group means the first ring and the second ring share at least two atoms therebetween.

“Link,” “linked,” or “linking,” unless otherwise specified, means covalently linked or bonded.

In one aspect, the invention provides a compound of formula I, a pharmaceutically acceptable salt thereof, diastereomers thereof, enantiomers thereof, and mixtures thereof:

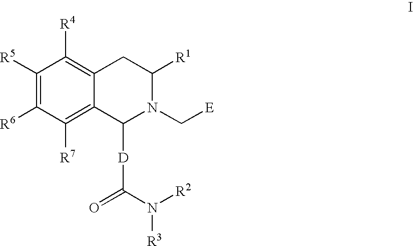

wherein

R¹ is selected from —H and C₁₋₆alkyl;

R² and R³ are independently selected from —H and C₁₋₆alkyl;

R⁴, R⁵, R⁶ and R⁷ are independently selected from —H, —OH, halogen, —NO₂, C₁₋₆alkyl, C₆₋₁₀aryl, C₁₋₆alkoxy, C₃₋₆cycloalkoxy, C₃₋₆heterocyclyl-oxy, C₃₋₆heterocyclyl-C₁₋₄alkoxy, C₆₋₁₀aryl-oxy, C₆₋₁₀aryl-C₁₋₄alkoxy, C₁₋₆alkyl-S(═O)₂—O—, C₆₋₁₀aryl-S(═O)₂—O—, C₁₋₆alkyl-NH—S(═O)₂—O—, and (C₁₋₆alkyl)₂N—S(═O)₂—O—; or any two adjacent groups selected from R⁴, R⁵, R⁶ and R⁷ form a portion of a 5 or 6-membered ring that fused with the benzene ring of formula I, wherein said C₁₋₆alkyl, C₆₋₁₀aryl, C₁₋₆alkoxy, C₃₋₆cycloalkoxy, C₃₋₆heterocyclyl-oxy, C₃₋₆heterocyclyl-C₁₋₄alkoxy, C₆₋₁₀aryl-oxy, C₆₋₁₀aryl-C₁₋₄alkoxy, C₁₋₆alkyl-S(═O)₂—O—, C₆₋₁₀aryl-S(═O)₂—O—, C₁₋₆alkyl-NH—S(═O)₂—O—, and (C₁₋₆alkyl)₂N—S(═O)₂—O— are optionally substituted with one or more groups selected from halogen, C₁₋₃alkoxy, —OH, —NO₂, C₁₋₃alkyl, —NH₂, and —CO₂—C₁₋₃alkyl;

E is a 5-membered heterocyclyl optionally substituted with one or more groups selected from halogen, C₁₋₆alkyl, —C(═O)—O—C₁₋₆alkyl, C₆₋₁₀aryl, C₆₋₁₀aryl-C₁₋₄alkyl, and C₆₋₁₀aryl-S(═O)₂—; and

D is a divalent group comprising a benzene ring.

In one embodiment, the compounds of the present invention are represented by formula I, wherein

R¹ is selected from —H and C₁₋₃alkyl;

R² and R³ are independently C₁₋₃alkyl;

R⁴, R⁵, R⁶ and R⁷ are independently selected from —H, —OH, halogen, —NO₂, C₁₋₆alkyl, phenyl, C₁₋₆alkoxy, C₃₋₆cycloalkoxy, tetrahydropyranyloxy, pyridinyloxy, morpholinyloxy, tetrahydropyranyl-C₁₋₄alkoxy, pyridinyl-C₁₋₄alkoxy, morpholinyl-C₁₋₆alkoxy, phenoxy, benzyloxy, C₁₋₆alkyl-S(—O)₂—O—, phenyl-S(—O)₂—O—, C₁₋₃alkyl-NH—S(═O)₂—O—, and (C₁₋₃alkyl)₂N—S(═O)₂—O—; or any two adjacent groups selected from R⁴, R⁵, R⁶ and R⁷ form a divalent group selected from —O—CH₂—O— and —O—CH₂—CH₂—O—, wherein said C₁₋₆alkyl, phenyl, C₁₋₆alkoxy, C₃₋₆cycloalkoxy, tetrahydropyranyloxy, pyridinyloxy, morpholinyloxy, tetrahydropyranyl-C₁₋₄alkoxy, pyridinyl-C₁₋₄alkoxy, morpholinyl-C₁₋₄alkoxy, phenoxy, benzyloxy, C₁₋₆alkyl-S(═O)₂—O—, phenyl-S(═O)₂—O—, C₁₋₃alkyl-NH—S(═O)₂—O—, and (C₁₋₃alkyl)₂N—S(═O)₂—O— are optionally substituted with one or more groups selected from halogen, methoxy, —OH, —NO₂, and C₁₋₃alkyl;

E is selected from furyl, thienyl, imidazolyl, pyrazolyl, and thiazolyl, wherein said furyl, thienyl, imidazolyl, pyrazolyl, and thiazolyl are optionally substituted with one or more groups selected from halogen, C₁₋₆alkyl, —C(═O)—O—C₁₋₃alkyl, phenyl, benzyl, and benzenesulfonyl; and

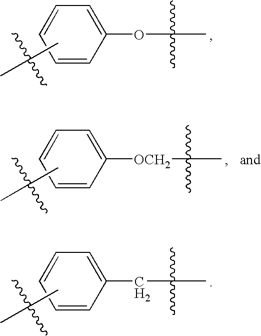

D is selected from phenylene, pyridylene,

In another embodiment, the compounds of the present invention are represented by formula I, wherein

R¹ is selected from —H and methyl;

R² and R³ are selected from ethyl and isopropyl;

R⁴, R⁵ and R⁶ are independently selected from —H, —OH, halogen, —NO₂, C₁₋₆alkyl, phenyl, C₁₋₆alkoxy, C₃₋₆cycloalkoxy, tetrahydropyranyloxy, pyridinyloxy, morpholinyloxy, tetrahydropyranyl-C₁₋₄alkoxy, pyridinyl-C₁₋₄alkoxy, morpholinyl-C₁₋₄alkoxy, phenoxy, benzyloxy, C₁₋₆alkyl-S(═O)₂—O—, phenyl-S(═O)₂—O—, C₁₋₃alkyl-NH—S(═O)₂—O—, and (C₁₋₃alkyl)₂N—S(═O)₂—O—; or any two adjacent groups selected from R⁴, R⁵ and R⁶ form —O—CH₂—O—, wherein said phenoxy, benzyloxy, and phenyl-S(═O)₂—O— are optionally substituted with one or more groups selected from halogen and methoxy;

R⁷ is selected from —H and C₁₋₃alkoxy;

E is selected from furyl, thienyl, imidazolyl, pyrazolyl, and thiazolyl, wherein said furyl, thienyl, imidazolyl, pyrazolyl, and thiazolyl are optionally substituted with one or more groups selected from halogen, C₁₋₄alkyl, —C(—O)—O—C₁₋₃alkyl, phenyl, benzyl, and benzenesulfonyl; and

D is selected from para-phenylene, para-benzylene,

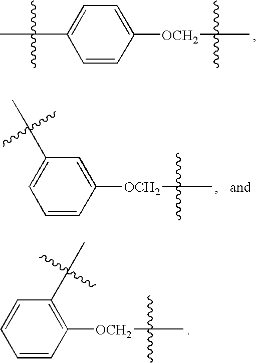

In a flier embodiment, the compounds of the present invention are represented by formula I, wherein

R¹ is selected from —H and methyl;

R² and R³ are ethyl;

R⁴ is selected from —H, NO₂ and methoxy; R⁵ is selected from —H, —Br, —F, —OH, methoxy, methylsulfonyloxy, N,N-dimethylsulfamyloxy; and R⁶ is selected from —H, —OH, —NO₂, methoxy, ethoxy, isopropyloxy, neopentyloxy, cyclobutyloxy, 4-tetrahydro-2H-pyranyloxy, 2-(4-morpholino)ethoxy, benzyloxy, phenoxy, 4-fluorophenoxy, 3-methoxyphenoxy, 4-methoxyphenoxy, 3-pyridinyloxy, methanesulfonyloxy, benzenesulfonyloxy, dimethylsulfamyloxy; or any two adjacent groups selected from R⁴, R⁵ and R⁶ form —O—CH₂—O—;

R⁷ is selected from —H and methoxy;

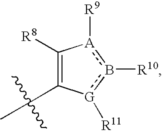

E is wherein A and B are independently selected from C, N and S, and G is selected from C, N, O and S with a proviso that at least one of A, B and G is C, at most one of A, B and G is S and one of the bonds between A and B, and between B and G is a double bond;

wherein R⁸ is selected from —H, —Cl, methyl, —CO₂Me and phenyl; R⁹ is selected from —H and methyl; R¹⁰ is selected from —H, methyl, n-butyl and phenyl; R¹¹ is selected from —H, methyl, benzyl and benzenesulfonyl.

D is selected from para-phenylene, para-benzylene,

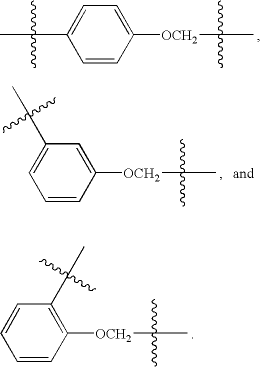

It will be understood that when compounds of the present invention contain one or more chiral centers, the compounds of the invention may exist in, and be isolated as, enantiomeric or diastereomeric forms, or as a racemic mixture. The present invention includes any possible enantiomers, diastereomers, racemates or mixtures thereof, of a compound of Formula I. The optically active forms of the compound of the invention may be prepared, for example, by chiral chromatographic separation of a racemate, by synthesis from optically active starting materials or by asymmetric synthesis based on the procedures described thereafter.

It will also be appreciated that certain compounds of the present invention may exist as geometrical isomers, for example E and Z isomers of alkenes. The present invention includes any geometrical isomer of a compound of Formula I. It will further be understood that the present invention encompasses tautomers of the compounds of the formula I.

It will also be understood that certain compounds of the present invention may exist in solvated, for example hydrated, as well as unsolvated forms. It will further be understood that the present invention encompasses all such solvated forms of the compounds of the formula I.

Within the scope of the invention are also salts of the compounds of the formula I. Generally, pharmaceutically acceptable salts of compounds of the present invention may be obtained using standard procedures well known in the art, for example by reacting a sufficiently basic compound, for example an alkyl amine with a suitable acid, for example, HCl or acetic acid, to afford a physiologically acceptable anion. It may also be possible to make a corresponding alkali metal (such as sodium, potassium, or lithium) or an alkaline earth metal (such as a calcium) salt by treating a compound of the present invention having a suitably acidic proton, such as a carboxylic acid or a phenol with one equivalent of an alkali metal or alkaline earth metal hydroxide or alkoxide (such as the ethoxide or methoxide), or a suitably basic organic amine (such as choline or meglumine) in an aqueous medium, followed by conventional purification techniques.

In one embodiment, the compound of formula I above may be converted to a pharmaceutically acceptable salt or solvate thereof, particularly, an acid addition salt such as a hydrochloride, hydrobromide, phosphate, acetate, fumarate, maleate, tartrate, citrate, methanesulphonate or p-toluenesulphonate.

The novel compounds of the present invention are useful in therapy, especially for the treatment of various pain conditions such as chronic pain, neuropathic pain, acute pain, cancer pain, pain caused by rheumatoid arthritis, migraine, visceral pain etc. This list should however not be interpreted as exhaustive.

Compounds of the invention are useful as immunomodulators, especially for autoimmune diseases, such as arthritis, for skin grafts, organ transplants and similar surgical needs, for collagen diseases, various allergies, for use as anti-tumour agents and anti viral agents.

Compounds of the invention are useful in disease states where degeneration or dysfunction of opioid receptors is present or implicated in that paradigm. This may involve the use of isotopically labelled versions of the compounds of the invention in diagnostic techniques and imaging applications such as positron emission tomography (PET).

Compounds of the invention are useful for the treatment of diarrhea, depression, anxiety and stress-related disorders such as post-traumatic stress disorders, panic disorder, generalized anxiety disorder, social phobia, and obsessive compulsive disorder, urinary incontinence, premature ejaculation, various mental illnesses, cough, lung oedema, various gastrointestinal disorders, e.g. constipation, functional gastrointestinal disorders such as Irritable Bowel Syndrome and Functional Dyspepsia, Parkinson's disease and other motor disorders, traumatic brain injury, stroke, cardioprotection following miocardial infarction, spinal injury and drug addiction, including the treatment of alcohol, nicotine, opioid and other drug abuse and for disorders of the sympathetic nervous system for example hypertension.

Compounds of the invention are useful as an analgesic agent for use during general anaesthesia and monitored anaesthesia care. Combinations of agents with different properties are often used to achieve a balance of effects needed to maintain the anaesthetic state (e.g. amnesia, analgesia, muscle relaxation and sedation). Included in this combination are inhaled anesthetics, hypnotics, anxiolytics, neuromuscular blockers and opioids.

Also within the scope of the invention is the use of any of the compounds according to the formula I above, for the manufacture of a medicament for the treatment of any of the conditions discussed above.

A further aspect of the invention is a method for the treatment of a subject suffering from any of the conditions discussed above, whereby an effective amount of a compound according to the formula I above, is administered to a patient in need of such treatment.

Thus, the invention provides a compound of formula L or pharmaceutically acceptable salt or solvate thereof, as hereinbefore defined for use in therapy.

In a further aspect the present invention provides the use of a compound of formula I, or a pharmaceutically acceptable salt or solvate thereof, as hereinbefore defined in the manufacture of a medicament for use in therapy.

In the context of the present specification, the term “therapy” also includes “prophylaxis” unless there are specific indications to the contrary. The term “therapeutic” and “therapeutically” should be contrued accordingly. The term “therapy” within the context of the present invention further encompasses to administer an effective amount of a compound of the present invention, to mitigate either a pre-existing disease state, acute or chronic, or a recurring condition. This definition also encompasses prophylactic therapies for prevention of recurring conditions and continued therapy for chronic disorders.

The compounds of the present invention are useful in therapy, especially for the therapy of various pain conditions including, but not limited to: acute pain, chronic pain, neuropathic pain, acute pain, back pain, cancer pain, and visceral pain.

In use for therapy in a warm-blooded animal such as a human, the compound of the invention may be administered in the form of a conventional pharmaceutical composition by any route including orally, intramuscularly, subcutaneously, topically, intranasally, intraperitoneally, intrathoracially, intravenously, epidurally, intrathecally, intracerebroventricularly and by injection into the joints.

In one embodiment of the invention, the route of administration may be orally, intravenously or intramuscularly.

The dosage will depend on the route of administration, the severity of the disease, age and weight of the patient and other factors normally considered by the attending physician, when determining the individual regimen and dosage level at the most appropriate for a particular patient.

For preparing pharmaceutical compositions from the compounds of this invention, inert, pharmaceutically acceptable carriers can be either solid and liquid. Solid form preparations include powders, tablets, dispersible granules, capsules, cachets, and suppositories.

A solid carrier can be one or more substances, which may also act as diluents, flavoring agents, solubilizers, lubricants, suspending agents, binders, or table disintegrating agents; it can also be an encapsulating material.

In powders, the carrier is a finely divided solid, which is in a mixture with the finely divided compound of the invention, or the active component. In tablets, the active component is mixed with the carrier having the necessary binding properties in suitable proportions and compacted in the shape and size desired.

For preparing suppository compositions, a low-melting wax such as a mixture of fatty acid glycerides and cocoa butter is first melted and the active ingredient is dispersed therein by, for example, stirring. The molten homogeneous mixture in then poured into convenient sized moulds and allowed to cool and solidify.

Suitable carriers are magnesium carbonate, magnesium stearate, talc, lactose, sugar, pectin, dextrin, starch, tragacanth, methyl cellulose, sodium carboxymethyl cellulose, a low-melting wax, cocoa butter, and the like.

The term composition is also intended to include the formulation of the active component with encapsulating material as a carrier providing a capsule in which the active component (with or without other carriers) is surrounded by a carrier which is thus in association with it. Similarly, cachets are included.

Tablets, powders, cachets, and capsules can be used as solid dosage forms suitable for oral administration.

Liquid form compositions include solutions, suspensions, and emulsions. For example, sterile water or water propylene glycol solutions of the active compounds may be liquid preparations suitable for parenteral administration. Liquid compositions can also be formulated in solution in aqueous polyethylene glycol solution.

Aqueous solutions for oral administration can be prepared by dissolving the active component in water and adding suitable colorants, flavoring agents, stabilizers, and thickening agents as desired. Aqueous suspensions for oral use can be made by dispersing the finely divided active component in water together with a viscous material such as natural synthetic gums, resins, methyl cellulose, sodium carboxymethyl cellulose, and other suspending agents known to the pharmaceutical formulation art.

Depending on the mode of administration, the pharmaceutical composition will preferably include from 0.05% to 99% w (percent by weight), more preferably from 0.10 to 50% w, of the compound of the invention, all percentages by weight being based on total composition.

A therapeutically effective amount for the practice of the present invention may be determined, by the use of known criteria including the age, weight and response of the individual patient, and interpreted within the context of the disease which is being treated or which is being prevented, by one of ordinary skills in the art.

Within the scope of the invention is the use of any compound of formula I as defined above for the manufacture of a medicament.

Also within the scope of the invention is the use of any compound of formula I for the manufacture of a medicament for the therapy of pain.

Additionally provided is the use of any compound according to Formula I for the manufacture of a medicament for the therapy of various pain conditions including, but not limited to: acute pain, chronic pain, neuropathic pain, acute pain, back pain, cancer pain, and visceral pain.

A further aspect of the invention is a method for therapy of a subject suffering from any of the conditions discussed above, whereby an effective amount of a compound according to the formula I above, is administered to a patient in need of such therapy.

Additionally, there is provided a pharmaceutical composition comprising a compound of Formula I, or a pharmaceutically acceptable salt thereof, in association with a pharmaceutically acceptable carrier.

Particularly, there is provided a pharmaceutical composition comprising a compound of Formula I, or a pharmaceutically acceptable salt thereof, in association with a pharmaceutically acceptable carrier for therapy, more particularly for therapy of pain.

Further, there is provided a pharmaceutical composition comprising a compound of Formula I, or a pharmaceutically acceptable salt thereof, in association with a pharmaceutically acceptable carrier use in any of the conditions discussed above.

In a further aspect, the present invention provides a method of preparing the compounds of the present invention.

In one embodiment, the invention provides a process for preparing a compound of formula II,

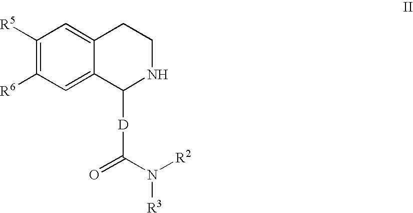

comprising of the step of reacting a compound of formula III with a compound of formula IV in the presence of HNR²R³:

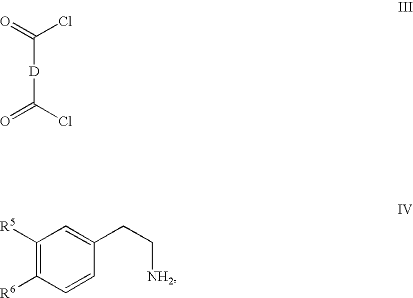

wherein

D, R¹, R⁵ and R⁶ are as defined above.

In another embodiment, the invention provides a process for preparing a compound of formula V,

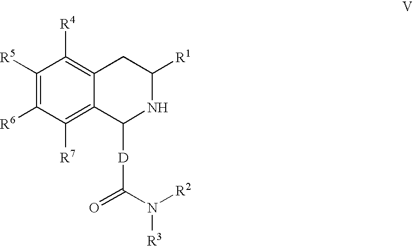

comprising of the step of reacting a compound of formula VI with a compound of formula VII in the presence of an acid catalyst such as trifluoroacetic acid or formic acid:

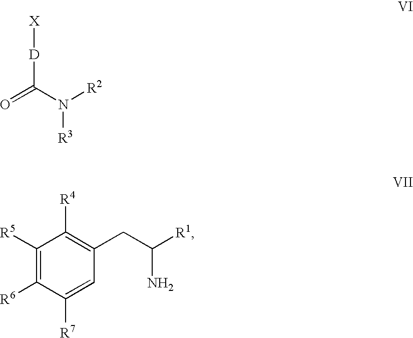

wherein

X is selected from CH(OEt)₂, ═CHOMe and —CHO; and

D, R¹, R², R³, R⁴, R⁵, R⁶ and R⁷ are as defined above.

In another embodiment, the present invention provides a process for preparing a compound of formula I,

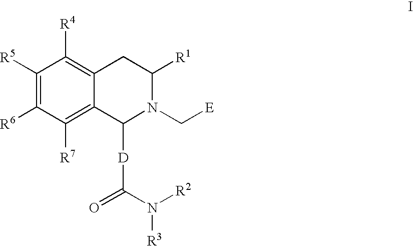

comprising: reacting a compound of formula VIII with E-CHO:

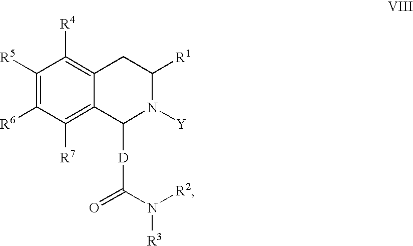

wherein

Y is selected from —H and —C(═O)—O-t-butyl; and

D, E, R¹, R², R³, R⁴, R⁵, R⁶ and R⁷ are as defined above.

In a further embodiment, the present invention provides a process for preparing a compound of formula IX,

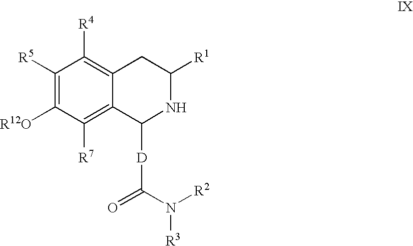

comprising: reacting a compound of formula X with R¹²—OH or R¹²—B(OH)₂:

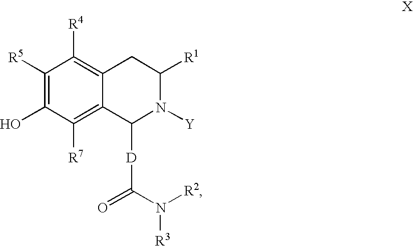

### Wherein

wherein

Y is selected from —H and C(═O)—O-t-butyl;

R¹² is selected from C₁₋₆alkyl, C₃₋₆cycloalkyl, C₁₋₁₀aryl-C₁₋₄alkyl, C₃heterocyclyl-C₁₋₄alkyl, C₆₋₁₀aryl, and C₃₋₆heteroaryl wherein said C₆₋₁₀aryl, C₃₋₆heterocyclyl and C₃₋₆heteroaryl are optionally substituted with one or more groups selected from halogen, C₁₋₃alkoxy, —OH, —NO₂, C₁₋₃alkyl, —NH₂ and —O₂—C₁₋₃alkyl; and

D, R¹, R², R³, R⁴, R⁵, and R⁷ are as defined above.

In a further embodiment, the present invention provides a process for preparing a compound of formula XI,

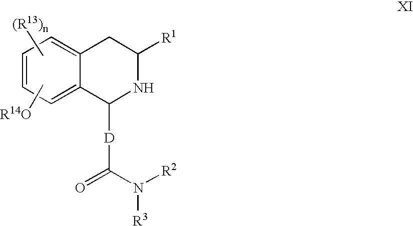

comprising:

reacting a compound of formula XI with NsCl, NsBr, or (CF₃CO)₂O to protect the ═NH group of formula XI;

reacting the protected compound with R¹⁴—Y¹ followed by deprotecting the ═NH group:

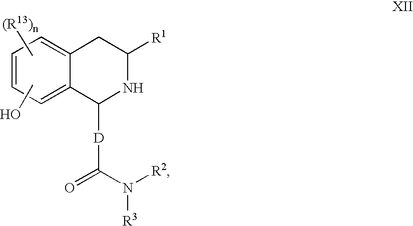

wherein

n is 0, 1, 2 or 3;

each R¹³ is independently selected from R⁴, R⁵, R⁶ and R⁷ as defined above;

Y¹ is halogen;

R¹⁴ is selected from C₁₋₆alkyl-S(═O)₂—, C₁₋₁₀aryl-S(═O)₂—, C₁₋₆alkyl-NH—S(═O)—, and (C₁₋₆alkyl)₂N—S(═O)₂—; and

D, R¹, R², R³, and R⁴ are as defined above.

More particularly, the compounds of the present invention and intermediates used for the preparation thereof can be prepared according to the synthetic routes as exemplified in Schemes 1-20, wherein, unless otherwise defined, R¹⁻¹¹, D and E are defined as above.

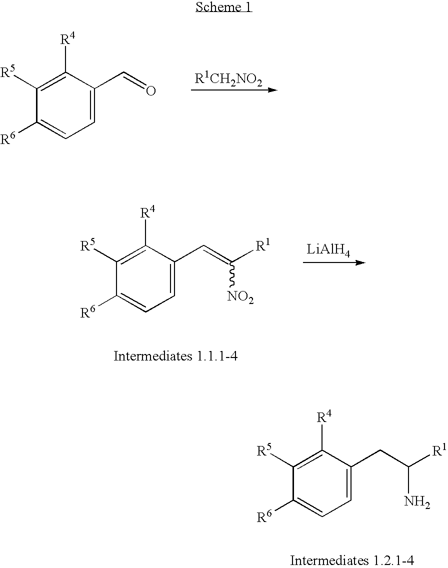

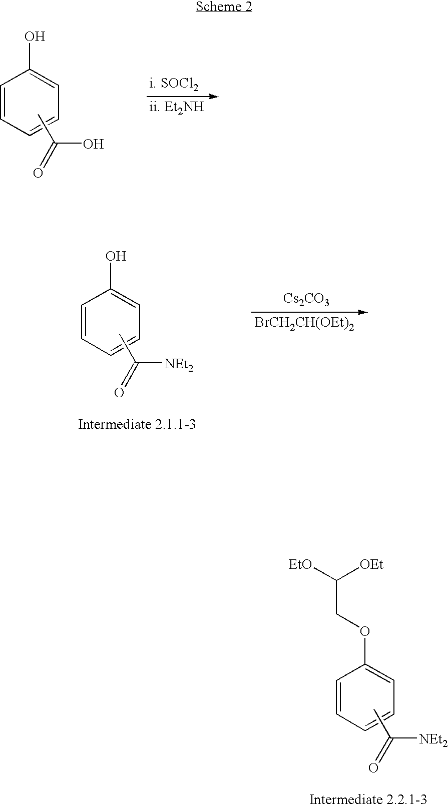

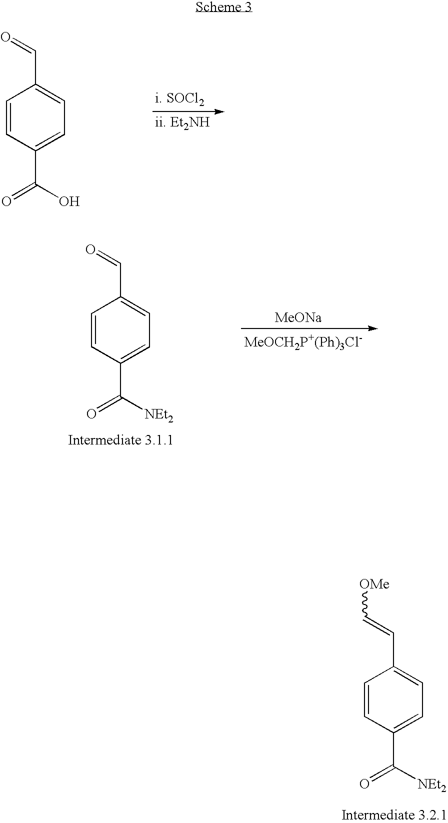

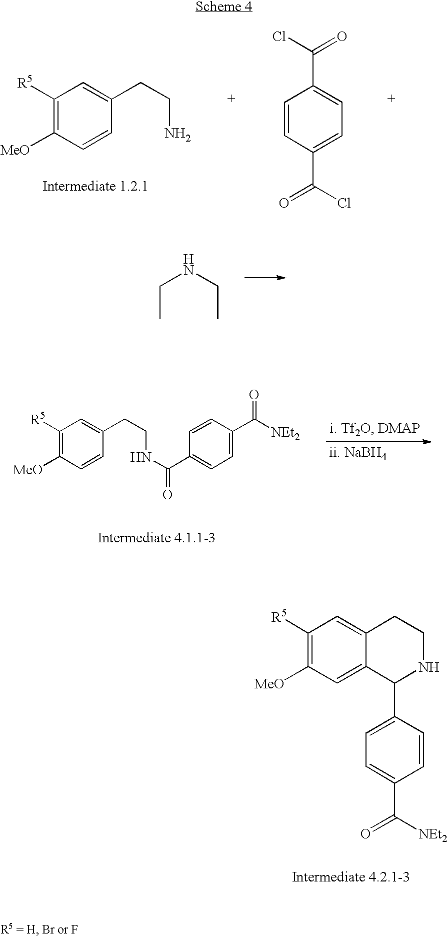

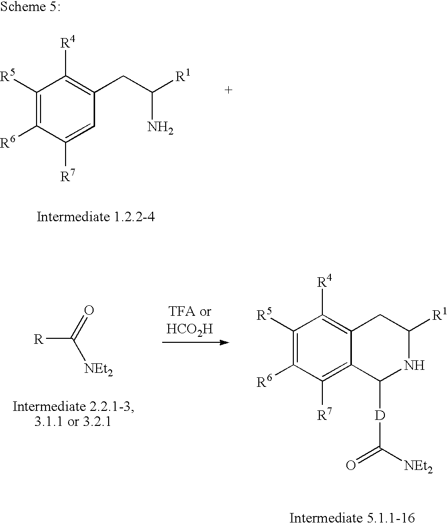

wherein R⁴=H, OMe, or R⁴+R⁵=methylenedioxy,
R⁵=H, OMe, or R⁵+R⁶=methylenedioxy, or R⁴+R⁵=methylenedioxy;
R⁶=H, OMe, or R⁵+R⁶=methylenedioxy;

### R⁷=H or OMe;

R=phenylene-OCH₂CH(OEt)₂, phenylene-CH═CHOMe or phenylene-CHO,
D=methyleneoxyphenyl, benzylene or phenylene.

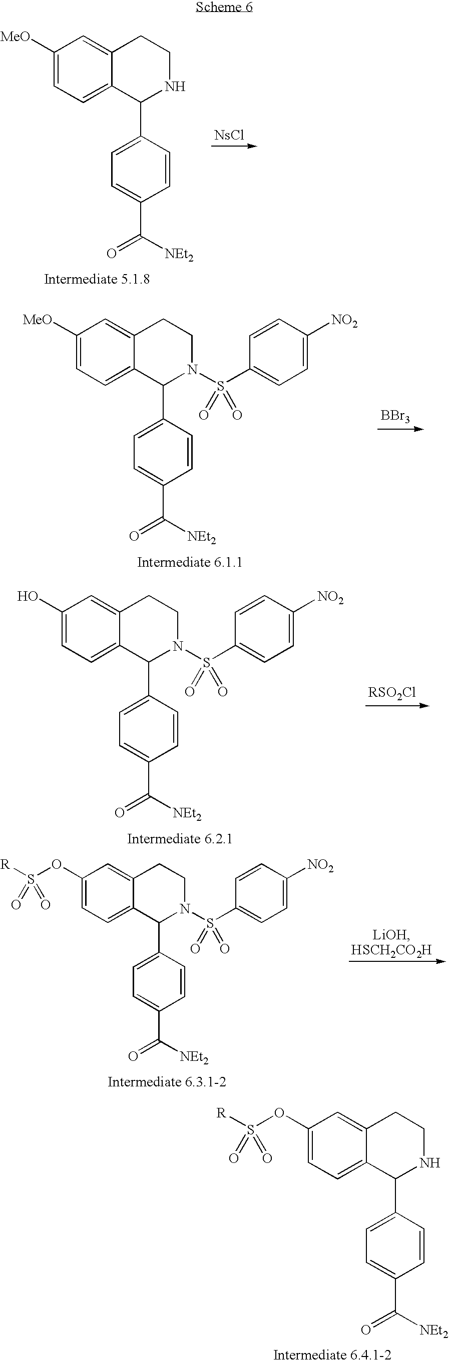

wherein R=Me or Me₂N.

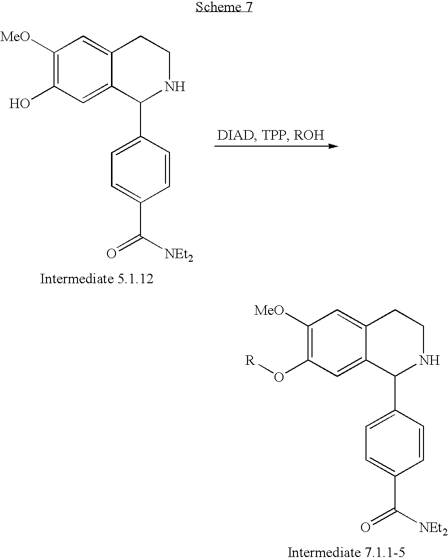

wherein R=Et, i-Pr, 2-(4-morpholino)ethyl, neopentyl or cyclobutyl.

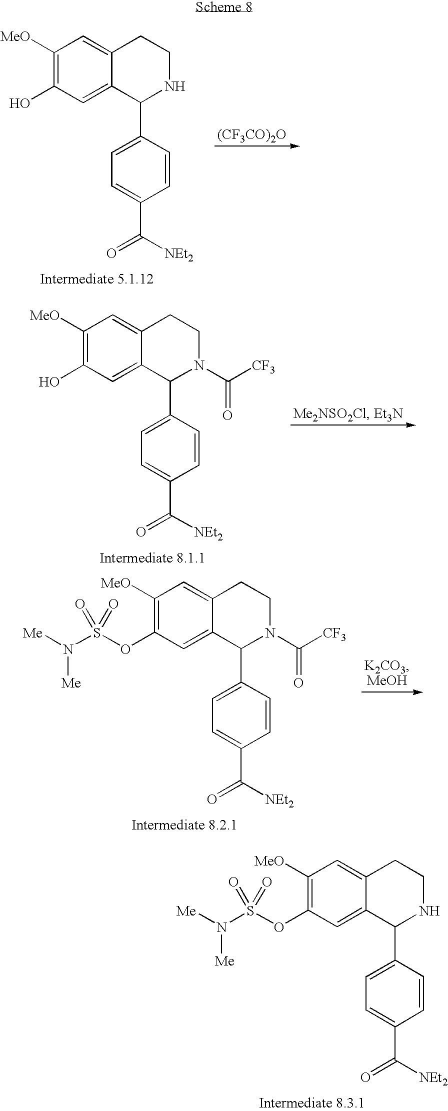

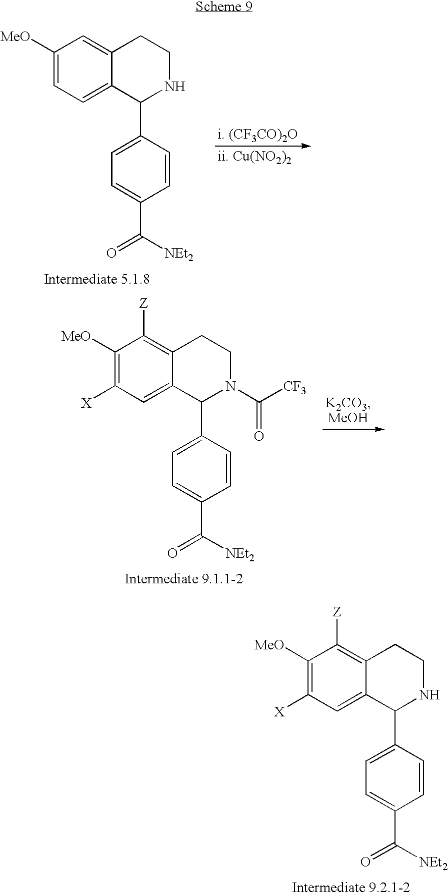

wherein X=H or NO₂, Z=H or NO₂.

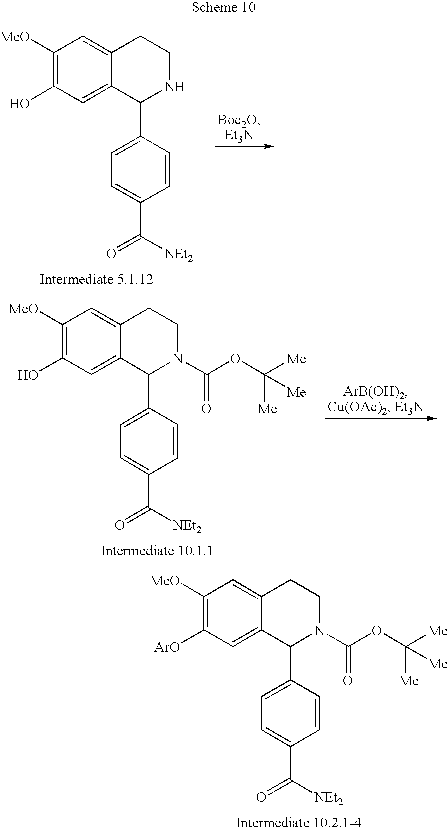

wherein Ar=phenyl, 4-fluorophenyl, 4-methoxyphenyl or 3-pyridinyl.

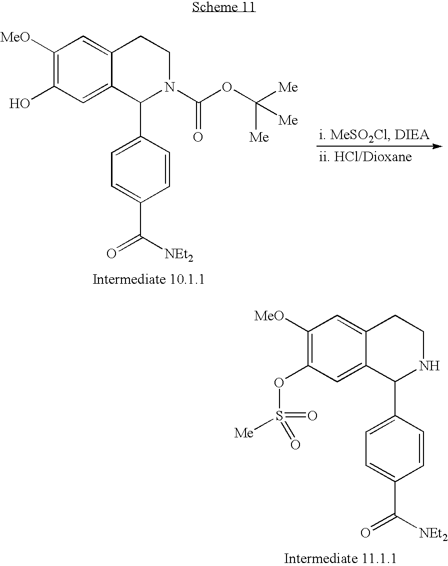

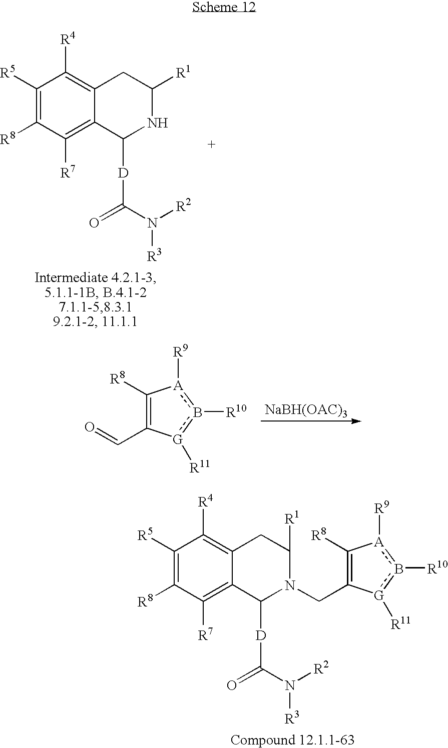

wherein

### A=C, N or S;

### B=C, N or S;

### G=C, N, O or S;

D=para-phenyl, para-benzyl, ortho-methyleneoxyphenyl, meta-methyleneoxyphenyl or para-methyleneoxyphenyl;

### R⁷=H or OMe;

R⁶=H, OH, NO₂, OMe, OEt, OiPr, neopentyloxy, cyclobutyloxy, 2-(4-morpholino)ethoxy, methanesulfonyloxy or R⁵+R⁶=methylenedioxy;
R⁵=H, Br, F, OH, OMe, methanesulfonyloxy, dimethylsulfamyloxy, R⁵+R⁶=methylenedioxy or R⁴+R⁵=methylenedioxy,
R⁴=H, OMe or R⁴+R⁵=methylenedioxy,

### R¹=H, Me;

### R⁸=H, Cl, Me, CO₂Me or Phenyl;

### R⁹=H or Me;

R¹⁰=H, Me, Et, n-Bu or Phenyl;
R¹¹=H, Me, benzyl or benzenesulfonyl.

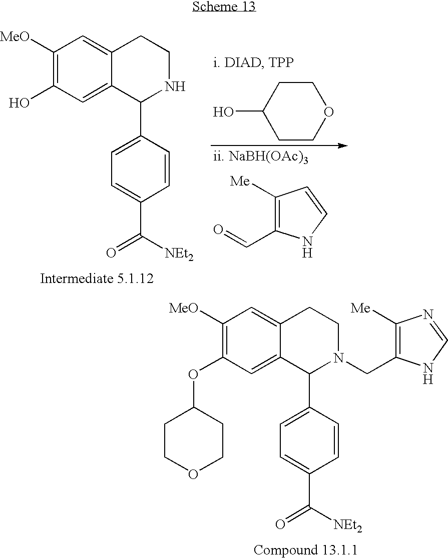

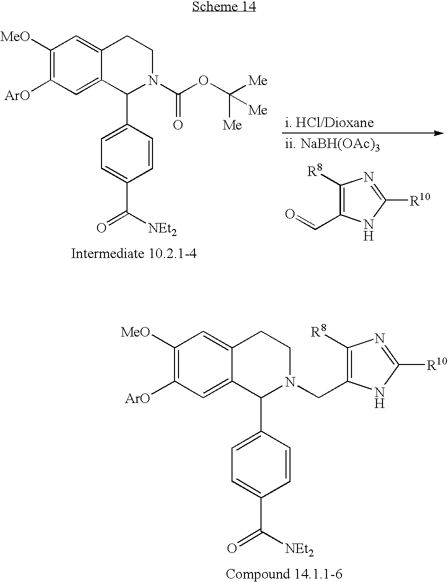

Wherein:

Ar=Ph, 4-fluorophenyl, 4-methoxyphenyl or 3-pyridinyl,

R⁸=H or Me,

R¹⁰=H or Phenyl.

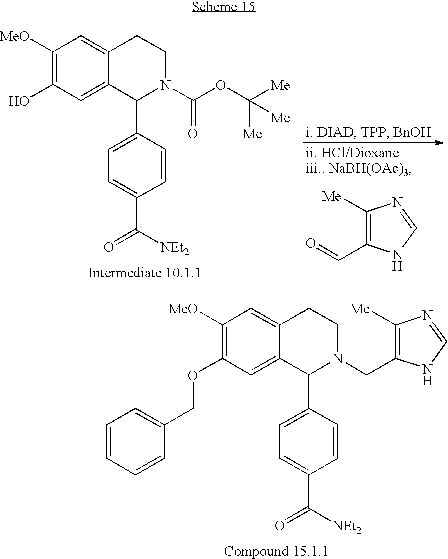

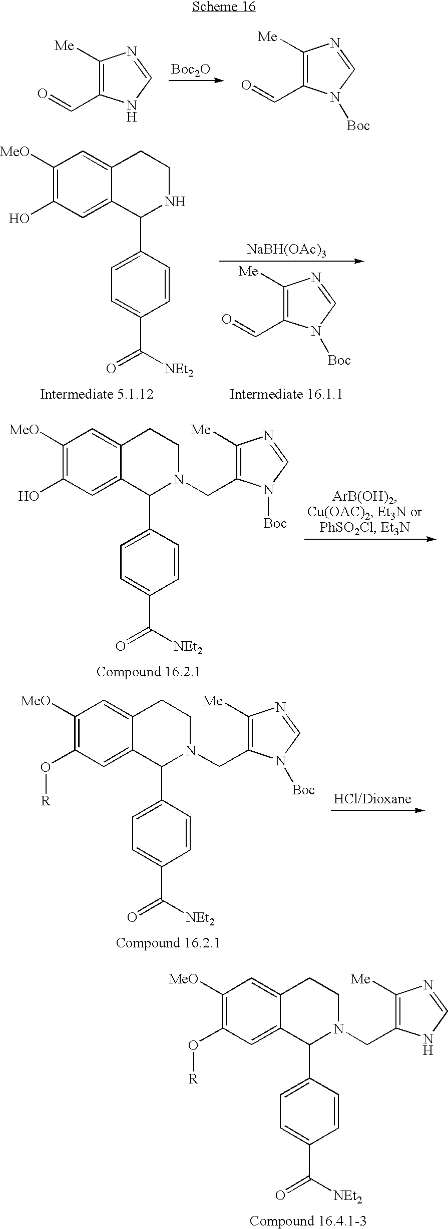

Wherein Ar=3-methoxyphenyl or 4-methoxyphenyl,
R=3-methoxyphenyl, 4-methoxyphenyl or phenylsulfonyl.

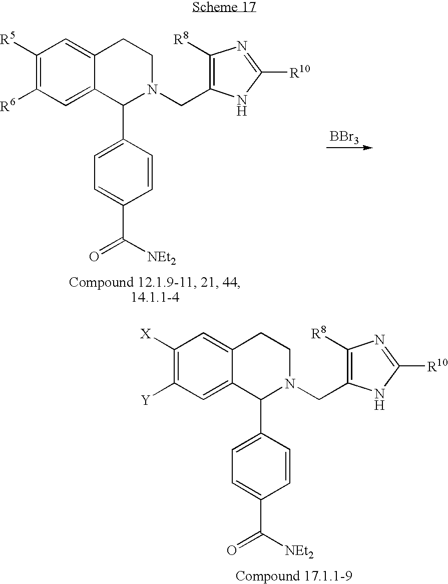

### Wherein

R⁶=H, OMe, phenoxy or 4-fluorophenoxy,

### R⁵=H, OMe,

### R⁸=H or Me,

R¹⁰=H or phenyl.
At least one of X and Y is OH; the other one of X and Y is H, OH, OMe, phenoxy or 4-fluorophenoxy.

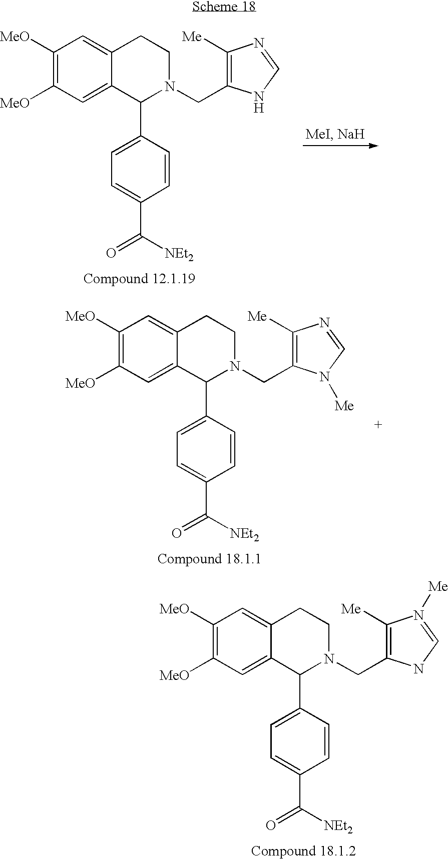

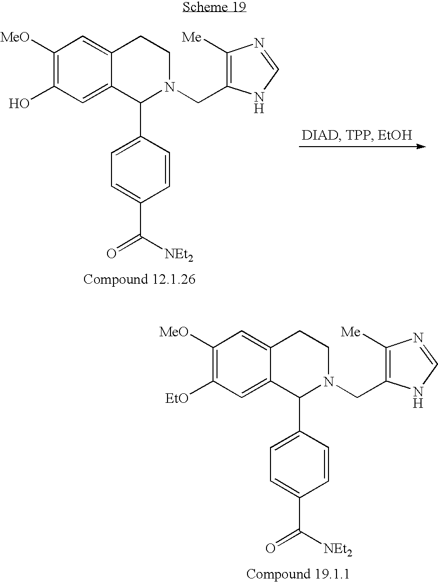

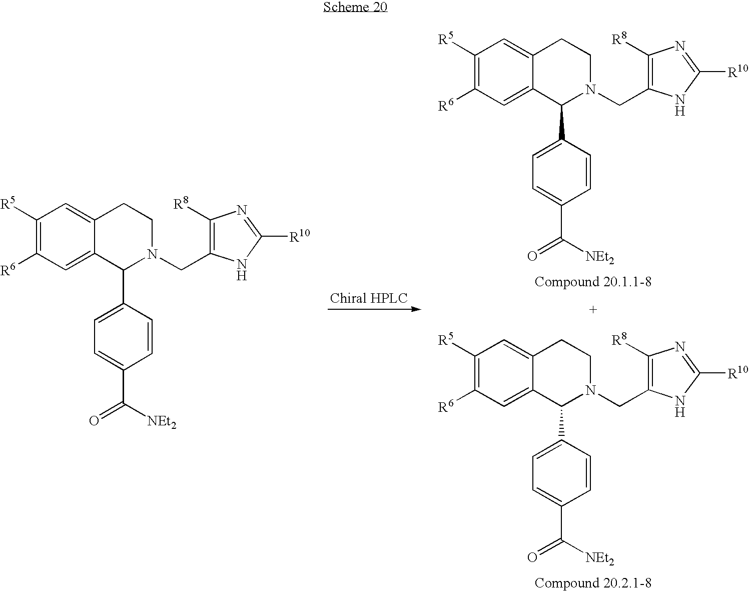

wherein R⁶=H, OMe, OiPr, phenoxy or 2-(4-morpholino)ethoxy,

### R⁵=OH or OMe,

### R⁸=H or Me,

### R¹⁰=H or Phenyl.

### Biological Evaluation

The compounds of the invention are found to be active towards δ receptors in warm-blooded animal, e.g., human. Particularly the compounds of the invention are found to be effective δ receptor ligands. In vitro assays, infra, demonstrate these surprising activities, especially with regard to agonists potency and efficacy as demonstrated in the rat brain functional assay and/or the human δ receptor functional assay (low). This feature may be related to in vivo activity and may not be linearly correlated with binding affinity. In these in vitro assays, a compound is tested for their activity toward δ receptors and IC₅₀ is obtained to determine the selective activity for a particular compound towards δ receptors. In the current context, IC₅₀ generally refers to the concentration of the compound at which 50% displacement of a standard radioactive δ receptor ligand has been observed.

The activities of the compound towards κ and μ receptors are also measured in a similar assay.

### In Vitro Model

### Cell Culture

Human 293S cells expressing cloned human κ, δ and μ receptors and neomycin resistance are grown in suspension at 37° C. and 5% CO₂ in shaker flasks containing calcium-free DMEM10% FBS, 5% BCS, 0.1% Pluronic F-68, and 600 μg/ml geneticin.

Rat brains are weighed and rinsed in ice-cold PBS (containing 2.5 mM EDTA, pH 7.4). The brains are homogenized with a polytron for 30 sec (rat) in ice-cold lysis buffer (50 mM Tris, pH 7.0, 2.5 mM EDTA, with phenylmethylsulfonyl fluoride added just prior use to 0.5 MmM from a 0.5M stock in DMSO:ethanol).

### Membrane Preparation

Cells are pelleted and resuspended in lysis buffer (50 mM Tris, pH 7.0, 2.5 mM EDTA, with PMSF added just prior to use to 0.1 mM from a 0.1 mM stock in ethanol), incubated on ice for 15 min, then homogenized with a polytron for 30 sec. The suspension is spun at 1000 g (max) for 10 min at 4° C. The supernatant is saved on ice and the pellets resuspended and spun as before. The supernatants from both spins are combined and spun at 46,000 g (max) for 30 min. The pellets are resuspended in cold Tris buffer (50 mM Tris/Cl, pH 7.0) and spun again. The final pellets are resuspended in membrane buffer (50 mM Tris, 0.32 M sucrose, pH 7.0). Aliquots (1 ml) in polypropylene tubes are frozen in dry ice/ethanol and stored at −70° C. until use. The protein concentrations are determined by a modified Lowry assay with sodium dodecyl sulfate.

### Binding Assays

Membranes are thawed at 37° C., cooled on ice, passed 3 times through a 25-gauge needle, and diluted into binding buffer (50 mM Tris, 3 mM MgCl₂, 1 mg/ml BSA (Sigma A-7888), pH 7.4, which is stored at 4° C. after filtration through a 0.22 m filter, and to which has been freshly added 5 μg/ml aprotinin, 10 μM bestatin, 10 μM diprotin A, no DTT). Aliquots of 100 μl are added to iced 12×75 mm polypropylene tubes containing 100 μl of the appropriate radioligand and 100 μl of test compound at various concentrations. Total (TB) and nonspecific (NS) binding are determined in the absence and presence of 10 μM naloxone respectively. The tubes are vortexed and incubated at 25° C. for 60-75 min, after which time the contents are rapidly vacuum-filtered and washed with about 12 ml/tube iced wash buffer (50 mM Tris, pH 7.0, 3 mM MgCl₂) through GF/B filters (Whatman) presoaked for at least 2 h in 0.1% polyethyleneimine. The radioactivity (dpm) retained on the filters is measured with a beta counter after soaking the filters for at least 12 h in minivials containing 6-7 ml scintillation fluid. If the assay is set up in 96-place deep well plates, the filtration is over 96-place PEI-soaked unifilters, which are washed with 3×1 ml wash buffer, and dried in an oven at 55° C. for 2 h. The filter plates are counted in a TopCount (Packard) after adding 50 μl MS-20 scintillation fluid/well.

### Functional Assays

The agonist activity of the compounds is measured by determining the degree to which the compounds receptor complex activates the binding of GTP to G-proteins to which the receptors are coupled. In the GTP binding assay, GTP[γ]³⁵S is combined with test compounds and membranes from HEK-293S cells expressing the cloned human opioid receptors or from homogenised rat and mouse brain. Agonists stimulate

GTP[γ]³⁵S binding in these membranes. The EC₅₀ and Eₘₐₓ values of compounds are determined from dose-response curves. Right shifts of the dose response curve by the delta antagonist naltrindole are performed to verify that agonist activity is mediated through delta receptors. For human δ receptor functional assays, EC₅₀ (low) is measured when the human δ receptors used in the assay were expressed at lower levels in comparison with those used in determining EC₅₀ (high). The Eₘₐₓ values were determined in relation to the standard δ agonist SNC80, i.e., higher than 100% is a compound that have better efficacy than SNC80.

### Procedure for Rat Brain GTP

Rat brain membranes are thawed at 37° C., passed 3 times through a 25-gauge blunt-end needle and diluted in the GTPγS binding (50 mM Hepes, 20 mM NaOH, 100 mM NaCl, 1 mM EDTA, 5 mM MgCl₂, pH 7.4, Add fresh: 1 mM DTT, 0.1% BSA). 120 μM GDP final is added membranes dilutions. The EC50 and Emax of compounds are evaluated from 10-point dose-response curves done in 300 μl with the appropriate amount of membrane protein (20 μg/well) and 100000-130000 dpm of GTPγ³⁵S per well (0.11-0.14 nM). The basal and maximal stimulated binding are determined in absence and presence of 3 μM SNC-80

### Data Analysis

The specific binding (SB) was calculated as TB-NS, and the SB in the presence of various test compounds was expressed as percentage of control SB. Values of IC₅₀ and Hill coefficient (n_{H}) for ligands in displacing specifically bound radioligand were calculated from logit plots or curve fitting programs such as Ligand, GraphPad Prism, SigmaPlot, or ReceptorFit. Values of Kᵢ were calculated from the Cheng-Prussoff equation. Mean±S.E.M. values of IC₅₀, Kᵢ and n_{H} were reported for ligands tested in at least three displacement curves.

Based on the above testing protocols, we find that the compounds of the present invention and some of the intermediates used in the preparation thereof are active toward human δ receptors. Generally, the IC₅₀ towards human δ receptor for most compounds of the present invention is less than 1000 nM.

### Receptor Saturation Experiments

Radioligand K_{δ} values are determined by performing the binding assays on cell membranes with the appropriate radioligands at concentrations ranging from 0.2 to 5 times the estimated K_{δ} (up to 10 times if amounts of radioligand required are feasible). The specific radioligand binding is expressed as pmole/mg membrane protein. Values of K_{δ} and Bₘₐₓ from individual experiments are obtained from nonlinear fits of specifically bound (B) vs. nM free (F) radioligand from individual according to a one-site model.

### Determination of Mechano-Allodynia Using Von Frey Testing

Testing is performed between 08:00 and 16:00 h using the method described by Chaplan et al. (1994). Rats are placed in Plexiglas cages on top of a wire mesh bottom which allows access to the paw, and are left to habituate for 10-15 min. The area tested is the mid-plantar left hind paw, avoiding the less sensitive foot pads. The paw is touched with a series of 8 Von Frey hairs with logarithmically incremental stiffness (0.41, 0.69, 1.20, 2.04, 3.63, 5.50, 8.51, and 15.14 grams; Stoelting, Ill., USA). The von Frey hair is applied from underneath the mesh floor perpendicular to the plantar surface with sufficient force to cause a slight buckling against the paw, and held for approximately 6-8 seconds. A positive response is noted if the paw is sharply withdrawn. Flinching immediately upon removal of the hair is also considered a positive response. Ambulation is considered an ambiguous response, and in such cases the stimulus is repeated.

### Testing Protocol

The animals are tested on postoperative day 1 for the FCA-treated group. The 50% withdrawal threshold is determined using the up-down method of Dixon (1980). Testing is started with the 2.04 g hair, in the middle of the series. Stimuli are always presented in a consecutive way, whether ascending or descending. In the absence of a paw withdrawal response to the initially selected hair, a stronger stimulus is presented; in the event of paw withdrawal, the next weaker stimulus is chosen. Optimal threshold calculation by this method requires 6 responses in the immediate vicinity of the 50% threshold, and counting of these 6 responses begins when the first change in response occurs, e.g. the threshold is first crossed. In cases where thresholds fall outside the range of stimuli, values of 15.14 (normal sensitivity) or 0.41 (maximally allodynic) are respectively assigned. The resulting pattern of positive and negative responses is tabulated using the convention, X=no withdrawal; O=withdrawal, and the 50% withdrawal threshold is interpolated using the formula:

50%gthreshold=10^{(Xf+kδ)}/10,000

where Xf=value of the last von Frey hair used (log units); k=tabular value (from Chaplan et al. (1994)) for the pattern of positive/negative responses; and δ=mean difference between stimuli (log units). Here δ=0.224.

Von Frey thresholds are converted to percent of maximum possible effect (% MPE), according to Chaplan et al. 1994. The following equation is used to compute % MPE:

$$\% M P E = \frac{\begin{matrix}Drug treated threshold ( g ) - \\ allodynia threshold ( g )\end{matrix}}{Control threshold ( g ) - allodynia threshold ( g )} \times 100$$

### Administration of Test Substance

Rats are injected (subcutaneously, intraperitoneally, intravenously or orally) with a test substance prior to von Frey testing, the time between administration of test compound and the von Frey test varies depending upon the nature of the test compound.

### Writhing Test

Acetic acid will bring abdominal contractions when administered intraperitoneally in mice. These will then extend their body in a typical pattern. When analgesic drugs are administered, this described movement is less frequently observed and the drug selected as a potential good candidate.

A complete and typical Writhing reflex is considered only when the following elements are present: the animal is not in movement; the lower back is slightly depressed; the plantar aspect of both paws is observable. In this assay, compounds of the present invention demonstrate significant inhibition of writhing responses after oral dosing of 1-100 μmol/kg.

### (i) Solutions Preparation

Acetic acid (AcOH): 120 μL of Acetic Acid is added to 19.88 ml of distilled water in order to obtain a final volume of 20 ml with a final concentration of 0.6% AcOH. The solution is then mixed (vortex) and ready for injection.
Compound (drug): Each compound is prepared and dissolved in the most suitable vehicle according to standard procedures.

### (ii) Solutions Administration

The compound (drug) is administered orally, intraperitoneally (i.p.), subcutaneously (s.c.) or intravenously (i.v.)) at 10 ml/kg (considering the average mice body weight) 20, 30 or 40 minutes (according to the class of compound and its characteristics) prior to testing. When the compound is delivered centrally: Intraventricularly (i.c.v.) or intrathecally (i.t.) a volume of 5 μL is administered.

The AcOH is administered intraperitoneally (i.p.) in two sites at 10 ml/kg (considering the average mice body weight) immediately prior to testing.

(iii) Testing

The animal (mouse) is observed for a period of 20 minutes and the number of occasions (Writhing reflex) noted and compiled at the end of the experiment. Mice are kept in individual “shoe box” cages with contact bedding. A total of 4 mice are usually observed at the same time: one control and three doses of drug.

For the anxiety and anxiety-like indications, efficacy has been established in the geller-seifter conflict test in the rat.

For the functional gastrointestina disorder indication, efficacy can be established in the assay described by Coutinho S V et al, in American Journal of Physiology—Gastrointestinal & Liver Physiology. 282(2):G307-16, 2002 February, in the rat.

### Additional In Vivo Testing Protocols

### Subjects and Housing

Naïve male Sprague Dawley rats (175-200 g) are housed in groups of 5 in a temperature controlled room (22° C., 40-70% humidity, 12-h light/dark). Experiments are performed during the light phase of the cycle. Animals have food and water ad libitum and are sacrificed immediately after data acquisition.

### Sample

Compound (Drug) testing includes groups of rats that do not receive any treatment and others that are treated with E. coli lipopolysaccharide (LPS). For the LPS-treated experiment, four groups are injected with LPS, one of the four groups is then vehicle-treated whilst the other three groups are injected with the drug and its vehicle. A second set of experiments are conducted involving five groups of rats; all of which receive no LPS treatment. The naïve group receives no compound (drug) or vehicle; the other four groups are treated with vehicle with or without drug. These are performed to determine anxiolytic or sedative effects of drugs which can contribute to a reduction in USV.

### Administration of LPS

Rats are allowed to habituate in the experimental laboratory for 15-20 min prior to treatment. Inflammation is induced by administration of LPS (endotoxin of gram-negative E. coli bacteria serotype 0111:B4, Sigma). LPS (2.4 μg) is injected intracerebro-ventricularly (i.c.v.), in a volume of 101, using standard stereotaxic surgical techniques under isoflurane anaesthesia. The skin between the ears is pushed rostrally and a longitudinal incision of about 1 cm is made to expose the skull surface. The puncture site is determined by the coordinates: 0.8 mm posterior to the bregma, 1.5 mm lateral (left) to the lambda (sagittal suture), and 5 mm below the surface of the skull (vertical) in the lateral ventricle. LPS is injected via a sterile stainless steel needle (26-G ⅜) of 5 mm long attached to a 100-μl Hamilton syringe by polyethylene tubing (PE20; 10-15 cm). A 4 mm stopper made from a cut needle (20-G) is placed over and secured to the 26-G needle by silicone glue to create the desired 5 mm depth.

Following the injection of LPS, the needle remains in place for an additional 10 s to allow diffusion of the compound, then is removed. The incision is closed, and the rat is returned to its original cage and allowed to rest for a minimum of 3.5 h prior to testing.

### Experimental Setup for Air-Puff Stimulation

The rats remains in the experimental laboratory following LPS injection and compound (drug) administration. At the time of testing all rats are removed and placed outside the laboratory. One rat at a time is brought into the testing laboratory and placed in a clear box (9×9×18 cm) which is then placed in a sound-attenuating ventilated cubicle measuring 62 (w)×35 (d)×46 (h) cm (BRS/LVE, Div. Tech-Serv Inc). The delivery of air-puffs, through an air output nozzle of 0.32 cm, is controlled by a system (AirStim, San Diego Intruments) capable of delivering puffs of air of fixed duration (0.2 s) and fixed intensity with a frequency of 1 puff per 10 s. A maximum of 10 puffs are administered, or until vocalisation starts, which ever comes first. The first air puff marks the start of recording.

### Experimental Setup for and Ultrasound Recording

The vocalisations are recorded for 10 minutes using microphones (G.R.A.S. sound and vibrations, Vedbaek, Denmark) placed inside each cubicle and controlled by LMS (LMS CADA-X 3.5B, Data Acquisition Monitor, Troy, Mich.) software. The frequencies between 0 and 32000 Hz are recorded, saved and analysed by the same software (LMS CADA-X 3.5B, Time Data Processing Monitor and UPA (User Programming and Analysis)).

### Compounds (Drugs)

All compounds (drugs) are pH-adjusted between 6.5 and 7.5 and administered at a volume of 4 ml/kg. Following compound (drug) administration, animals are returned to their original cages until time of testing.

### Analysis

The recording is run through a series of statistical and Fourier analyses to filter (between 20-24 kHz) and to calculate the parameters of interest. The data are expressed as the mean±SEM. Statistical significance is assessed using T-test for comparison between naive and LPS-treated rats, and one way ANOVA followed by Dunnett's multiple comparison test (post-hoc) for drug effectiveness. A difference between groups is considered significant with a minimum p value of ≦0.05. Experiments are repeated a minimum of two times.

### Examples

The invention will further be described in more detail by the following Examples which describe methods whereby compounds of the present invention may be prepared, purified, analyzed and biologically tested, and which are not to be construed as limiting the invention.

### INTERMEDIATE 1.1.1: 2-FLUORO-1-METHOXY-4-[(E)-2-NITROVINYL]BENZENE

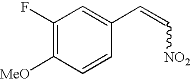

3-Fluoro-4-methoxybenzaldehyde (1.70 g, 11.0 mmol) and ammonium acetate (0.94 g, 12.2 mmol) were dried in vacuum for 3 h and then dissolved in nitromethane (12 mL, 222.0 mmol). The mixture was stirred under nitrogen and refluxed at 96° C. for 90 min. The nitromethane was removed in vacuo and the solid residue taken up in EtOAc (30 mL) and washed with 3 M HCl (3×15 mL), sat. NaHCO₃ (15 mL), brine (15 mL) and water (15 mL). The organic layer was evaporated in vacuo and the residue (2.14 g) was purified by chromatography on SiO₂ column (hexane:DCM 70:30) to yield INTERMEDIATE 1.1.1 as a green solid (1.04 g, 48%). ¹H NMR (500 MHz, CDCl₃): δ 3.97 (s, 3H), 7.04 (t, J 8.5 Hz, 1H), 7.31 (br t, J 10 Hz, 2H), 7.50 (d, J 13.5 Hz, 1H), 7.93 (d, J 13.5 Hz, 1H); ¹³C NMR (125 MHz, CDCl₃): δ 56.59, 113.84, 115.91 (d, J 18.8 Hz), 123.22 (d, J 6.4 Hz), 127.32, 136.35, 138.17, 151.41 (d, J 11 Hz), 152.71 (d, J 248 Hz); INTERMEDIATE 1.1.1 did not ionise under normal LRESIMS conditions.

### INTERMEDIATE 1.1.2: 4-[(L)-2-NITROVINYL]-1,3-BENZODIOXOLE

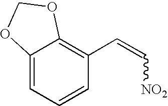

2,3-Methylenedioxybenzaldehyde (1.20 g, 7.99 mmol) and ammonium acetate (0.62 g, 7.99 mmol) were dried in a vacuum for 3 h and then dissolved in nitromethane (3.76 mL, 69.4 mmol). The mixture was stirred under nitrogen and refluxed at 96° C. for 90 min. The nitromethane was removed in vacuo and the solid residue taken up in EtOAc (30 mL) and washed with 3 M HCl (3×15 mL), sat. NaHCO₃ (15 ml), brine (15 mL) and water (15 mL). The organic layer was evaporated in vacuo and the dark brown residue (1.65 g) purified by flash chromatography on SiO₂ column (hexane:DCM 70:30) with a total of 42 fractions collected. Fractions 4 to 9 were combined and concentrated to dryness yielding pure INTERMEDIATE 1.1.2 (181.2 mg) as a yellow solid. Fractions 1 to 3 and 10 to 13 were combined and recrystallised (hexane:DCM 50:50) to afford INTERMEDIATE 1.1.2 as a green solid (443 mg). Combined yield: 624.2 mg, 40%. ¹H NMR (500 MHz, CDCl₃): δ 6.14 (s, 2H), 6.95 (m, 3H), 7.84 (d, J 13.6 Hz, 1H), 7.93 (d, J 14.2 Hz, 1H); ¹³C NMR (125 MHz, CDCl₃): δ 102.21, 111.77, 113.23, 122.66, 124.25, 134.07, 139.55, 147.48, 148.42; INTERMEDIATE 1.1.2 did not ionise under normal LRESIMS conditions.

### INTERMEDIATE 1.1.3: 1,2,3-TRIMETHOXY-4-[(E)-2-NITROVINYL]BENZENE

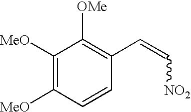

2,3,4-Trimethoxybenzaldehyde (1.26 g, 6.4 mmol) and ammonium acetate (0.53 g, 6.9 mmol) were dried in vacuum for 3 h and then dissolved in nitromethane (4 mL, 73.9 mmol). The mixture was stirred under nitrogen and refluxed at 96° C. for 90 min. The nitromethane was removed in vacuo and the solid residue taken up in EtOAc (30 mL) and washed with 3 M HCl (3×15 mL), sat. NaHCO₃ (15 mL), brine (15 mL) and water (15 mL). The organic layer was evaporated in vacuo and the dark yellow residue (1.5452 g) was purified by flash chromatography on SiO₂ column (hexane:DCM 60:40) to yield INTERMEDIATE 1.1.3 as a yellow oil (1.22 g, 79%). ¹H NMR (500 MHz, CDCl₃) δ 3.88 (s, 3H), 3.94 (s, 3H), 4.00 (s, 3H), 6.73 (d, J 9 Hz, 2H), 7.21 (d, J 8.6 Hz, 1H), 7.77 (d, J 13.6 Hz, 1H), 8.09 (d, J 14 Hz, 1H); ¹³C NMR (125 MHz, CDCl₃): δ 56.42, 61.14, 61.40, 107.98, 117.29, 126.78, 135.52, 136.80, 142.70, 154.50, 157.61; (+) LRESIMS m/z 240.08 [M+H]⁺, 262.06 [N+Na]⁺.

### INTERMEDIATE 1.1.4: 1,2-DIMETHOXY-4-[(1E)-2-NITROPROP-1-ENYL]BENZENE

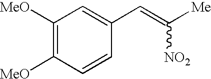

3,4-Dimethoxybenzaldehyde (0.57 g, 3.5 mmol) and ammonium acetate (0.28 g, 3.7 mmol) were dried in vacuum for 3 h and then dissolved in nitroethane (4 mL, 57.2 mmol). The mixture was stirred under nitrogen and refluxed at 90° C. for 15 h. The nitroethane was removed in vacuo and the solid residue taken up in EtOAc (15 mL) and washed with 3 M HCl (2×10 mL), sat. NaHCO₃ (3×10 mL), brine (10 mL) and water (10 mL). The organic layer was evaporated in vacuo to afford INTERMEDIATE 1.1.4 as a yellow solid (736 mg, 95%). ¹H NMR (500 MHz, CDCl₃): δ 2.45 (s, 3H), 3.89 (s, 3H), 3.91 (s, 3H), 6.93 (m, 1H), 7.06 (m, 1H), 8.02 (s, 1H); ¹³C NMR (125 MHz, CDCl₃): δ 14.32, 56.21, 111.55, 113.41, 124.26, 125.22, 133.97, 146.13, 149.36, 151.06; (+) LRESIMS m/z 224.07 [M+H]⁺, 246.04 [M+Na]⁺.

### INTERMEDIATE 1.2.1: 2-(3-FLUORO-4-METHOXYPHENYL)ETHANAMINE

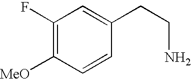

To a refluxing suspension of LiAlH₄ (1 M solution in THF, 20 mL, 759 mg, 20 mmol), Intermediate 1.1.1 (989.8 mg, 5.02 mmol) in anhydrous THF (10 mL) was added and stirred for 1 h. After hydrolysis with water (3 mL), the solvent was removed in vacuo. The residue was dissolved in 2 N HCl (35 mL) and extracted with EtOAc (2×20 mL). The combined organic layers were extracted with 2 N HCl (2×10 mL) and the combined aqueous layers were treated with tartaric acid (5.4 g, 36 mmol). Concentrated NH₄OH was used to adjust the solution to pH>10. The aqueous layer was then extracted with CHCl₃ (3×30 mL). The combined organic layers were washed with water (20 mL) and evaporated to yield INTERMEDIATE 1.2.1 as a light-yellow oil (754 mg, 89%). ¹H NMR (500 MHz, CDCl₃): δ 2.37 (br s, 2H), 2.66, (t, J 7 Hz, 2H), 2.91 (t, J 7 Hz, 2H), 3.84 (s, 3H), 6.86-6.91 (m, 3H); ¹³C NMR (125 MHz, CDCl₃): δ 38.91, 43.43, 56.60, 113.89, 116.60 (d, J 17.6 Hz), 124.57, 133.03, 146.24 (d, J 10.5 Hz), 152.60 (d, J 244 Hz); (+) LRESIMS m/z 170.11.

### INTERMEDIATE 1.2.2: 2-(1,3-BENZODIOXOL-4-YL)ETHANAMINE

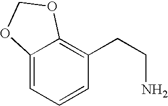

To a refluxing suspension of LiAlH₄ in anhydrous THF (1 M solution, 20 mL, 759 mg, 20 mmol), INTERMEDIATE 1.1.2 (624.2 mg, 3.23 mmol) in anhydrous THF (10 mL) was added dropwise and stirred for 1 h. After hydrolysis with water (2 mL), the solvent was removed in vacuo. The residue was dissolved in 2 N HCl (30 mL) and extracted with EtOAc (2×10 mL). The combined organic layers were extracted with 2 N HCl (2×10 mL) and the combined aqueous layers were treated with tartaric acid (3.4467 g, 22.96 mmol). Concentrated NH₄OH was used to adjust the solution to pH>10. The aqueous layer was then extracted with CHCl₃ (3×40 mL). The combined organic layers were washed with water (2×10 mL) and evaporated to yield INTERMEDIATE 1.2.2 as an orange oil in quantitative yield. ¹H NMR (500 MHz, CDCl₃): δ 2.76 (t, J 7.2 Hz, 2H), 2.99 (t, J 7.2 Hz, 2H), 3.51 (brs, 2H), 5.88 (s, 2H), 6.65-6.70 (m, 2H), 6.74 (m, 1H); ¹³C NMR (125 z, CDCl₃): δ 33.3, 41.6, 100.7, 107.1, 121.1, 121.8, 123.1, 146.0, 147.4; (+) LRESIMS m/z 166.13 [M+H]⁺.

### INTERMEDIATE 1.2.3: 2-(2,3,4-TRIMETHOXYPHENYL)ETHANAMINE

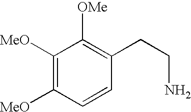

INTERMEDIATE 1.1.3 (1.11 g, 4.6 mmol) dissolved in anhydrous THF (10 mL) was added drop wise to a refluxing suspension of LiAlH₄ (1 M solution in THF, 29 mL, 1.1001 g, 29.0 mmol) and refluxed for 1 h. After hydrolysis with water (6 mL), the solvent was removed in vacuo. The residue was dissolved in 2 N HCl (30 mL) and washed with EtOAc (50 mL). The organic layer was extracted with 2 N HCl (12 mL) and the combined aqueous layers were treated with tartaric acid (4.95 g, 33.0 mmol). Concentrated NH₄OH was used to adjust the solution to pH>10, and the aqueous layer then extracted with CHCl₃ (3×30 mL). The combined organic layers were washed with water (2×20 mL) and evaporated to yield INTERMEDIATE 1.2.3 as a dark orange oil (828 mg, 84%). ¹H NMR (500 MHz, CDCl₃): δ 2.68 (t, J 7 Hz, 2H), 2.88 (t, J 7 Hz, 2H), 3.83 (s, 3H), 3.85 (s, 3H), 3.86 (s, 3H), 6.60 (d, J 8 Hz, 1H), 6.81 (d, J 8 Hz, 1H); ¹³C NMR (125 MHz, CDCl₃): δ 34.39, 43.22, 56.23, 60.89, 61.11, 107.51, 124.53, 125.92, 142.62, 152.34, 152.53; (+) LRESIMS m/z 212.11 [M+H]⁺.

### INTERMEDIATE 1.2.4: 1-(3,4-DIMETHOXYPHENYL)PROPAN-2-AMINE

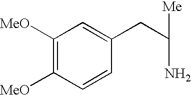

INTERMEDIATE 1.1.4 (706 mg, 3.16 mmol) in anhydrous THF (5 mL) was added drop wise to a refluxing suspension of LiAlH₄ (1 M solution in THF, 25 mL, 948 mg, 25.0 mmol) and refluxed for 1 h. After hydrolysis with water (10 mL), the solvent was removed in vacuo. The residue was dissolved in 2 N HCl (20 mL) and washed with EtOAc (20 mL). The organic layer was extracted with 2 N HCl (10 mL) and the combined aqueous layers were treated with tartaric acid (3.374 g, 22.5 mmol). Concentrated NH₄OH was used to adjust the solution to pH>10, and the aqueous layer then extracted with CHCl₃ (3×20 mL). The combined organic layers were washed with water (2×15 mL) and evaporated to yield INTERMEDIATE 1.2.4 as an orange oil (540 mg, 87%). ¹H NMR (500 MHz, CDCl₃): δ 1.13 (d, J 6.5 Hz, 3H), 2.06 (br s, 2H), 2.48 (dd, J 7.8, 13.2 Hz, 1H), 2.69 (dd, J 5.2, 13.2 Hz, 1H), 3.15 (m, 1H), 3.85 (s, 3H), 3.86 (s, 3H), 6.71 (s, 1H), 6.73 (m, 1H), 6.80 (d, J 8 Hz, 1H); ¹³C NMR (125 MHz, CDCl₃): δ 23.46, 46.12, 48.78, 56.09, 56.16, 111.61, 112.77, 121.43, 132.37, 147.81, 149.14; (+) LRESIMS m/z 196.14 [M+H]⁺.

### INTERMEDIATE 2.1.1 N,N-DIETHYL-4-HYDROXYBENZAMIDE

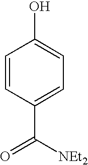

A mixture of 4-hydroxybenzoic acid (1.00 g, 7.2 mmol) and thionyl chloride (5 mL) was heated at reflux for 0.5 h. Excess thionyl chloride was removed in vacuo and the residue was taken up in dichloromethane (20 mL). Diethylamine (5 mL) was then added to the reaction mixture dropwise and the solution was left stirring for 1 h. 1M HCl (50 mL) was added and the mixture was extracted with dichloromethane (2×50 mL). The organic phase was washed with saturated sodium hydrogen carbonate, dried (MgSO₄), filtered and the solvent removed in vacuo. Diethylamine was added to the residue and the mixture was heated at reflux for 18 h afterwhich the excess diethylamine was removed in vacuo and the residue purified by flash chromatography (acetonitrile/dichloromethane, 2/8) to give INTERMEDIATE 2.1.1 (0.96 g, 69%) as a white solid. ¹H NMR (500 MHz, CDCl₃) δ 1.18 (br s, 6H), 3.35 (br s, 2H), 3.50 (br s, 2H), 6.70 (d, J 8.5 Hz, 2H), 7.15 (d, J 8.5 Hz, 2H); ¹³C NMR (125 MHz, CDCl₃): δ 12.83, 13.86, 38.85, 43.83, 115.54, 126.60, 128.03, 158.60, 172.67; (+) LRESIMS m/z 192 [M−H]⁺ (100).

### INTERMEDIATE 2.1.2: N,N-DIETHYL-3-HYDROXYBENZAMIDE

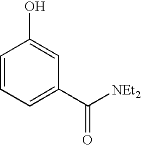

A mixture of 3-hydroxybenzoic acid (5.00 g, 36.2 mmol) and thionyl chloride (20 mL) was heated at reflux for 1 h. After cooling excess thionyl chloride was removed in vacuo and the residue was taken up in dichloromethane (50 mL). The dichloromethane solution was then added dropwise to a solution of diethylamine (20 mL) in dichloromethane (100 mL), cooled to 0° C. and the resulting solution left to stir for 1 h. The excess diethylamine and dichloromethane was removed in vacuo and the residue taken up in neat diethylamine (20 mL) and heated at reflux for 18 h. The excess diethylamine was removed once more in vacuo and the residue dissolved in dichloromethane (300 mL). The organic phase was washed with 1M hydrochloric acid (100 mL), water (100 mL), dried (MgSO₄), filtered and the solvent removed in vacuo. The residue was purified by squat chromatography (ethyl acetate/dichloromethane, 4/6) to give INTERMEDIATE 2.1.2 (6.50 g, 93%) as a white solid. ¹H NMR (500 MHz, CDCl₃) δ 1.09 (br s, 3H), 1.24 (br s, 31), 3.26 (br s, 2H), 3.54 (br s, 2H), 6.77 (d, J 7.5 Hz, 1H), 6.79 (d, J 8.5 Hz, 1H), 6.88 (s, 1H), 7.15 (t, J 8 Hz, 1H); ¹³C NMR (125 MHz, CDCl₃): δ 12.75, 14.05, 39.63, 43.62, 114.04, 116.96, 117.21, 129.46, 136.87, 157.12, 172.20; (+) LRESIMS m/z 192 [M−H]⁺ (100).

### INTERMEDIATE 2.1.3: N,N-DIETHYL-2-HYDROXYBENZAMIDE

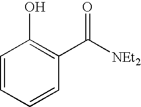

A mixture of 2-hydroxybenzoic acid (5.00 g, 36.2 mmol) and thionyl chloride (20 mL) was heated at reflux for 1 h. After cooling excess thionyl chloride was removed in vacuo and the residue was taken up in dichloromethane (50 mL). The dichloromethane solution was then added dropwise to a solution of diethylamine (20 mL) in dichloromethane (100 mL), cooled to 0° C. and the resulting solution left to stir for 1 h. The excess diethylamine and dichloromethane was removed in vacuo and the residue taken up in neat diethylamine (20 mL) and heated at reflux for 18 h. Excess diethylamine was removed once more in vacuo and the residue dissolved in dichloromethane (300 mL). The organic phase was washed with 1M hydrochloric acid (100 mL), water (100 mL), dried (MgSO₄), filtered and the solvent removed in vacuo. The residue was purified by squat chromatography (ethyl acetate/dichloromethane, 3/7) to give INTERMEDIATE 2.1.3 (6.11 g, 87%) as a white solid. ¹H NMR (500 MHz, CDCl₃) δ 1.27 (t, J 7 Hz, 6H), 3.52 (q, J 7 Hz, 4H), 6.85 (t, J 7.5 Hz, 1H), 7.00 (d, J 8.5 Hz, 1H), 7.26 (d, J 7 Hz, 1H), 7.30 (t, J 8 Hz, 1H), 9.40 (br s, 1H); ¹³C NMR (125 MHz, CDCl₃): δ 13.30, 42.08, 117.84, 118.42, 127.20, 132.06, 158.24, 171.34; (+) LRESIMS m/z 192 [M−H]⁺ (100).

### INTERMEDIATE 2.2.1: 4-(2,2-DIETHOXYMETHOXY)-N,N-DIETHYLBENZAMIDE

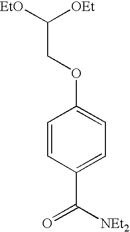

To a mixture of INTERMEDIATE 2.1.1 (3.00 g, 15.5 mmole) and cesium carbonate (11.00 g, 34 mmole) in N,N-dimethylformamide (20 mL) was added bromoacetaldehyde diethyl acetal (4.7 mL, 31 mmole) and the resulting mixture was heated at reflux under nitrogen for 1 h. The reaction mixture was cooled, water (100 mL) added and extracted with ethyl acetate (3×100 mL). The combined organic extracts were washed with water (2×100 mL), dried (MgSO₄), filtered and the solvent removed in vacuo. The residue was purified by squat chromatography (ethyl acetate/hexane, 4/6) to give INTERMEDIATE 2.2.1 (4.46 g, 93%) as a clear liquid. ¹H NMR (500 MHz, CDCl₃) δ1.74 (br s, 6H), 1.25 (t, J 7 Hz, 6H), 3.41 (br s, 4H), 3.64 (m, 2H), 3.77 (m, 2H), 4.03 (d, J 5.5 Hz, 2H), 4.83 (t, J 5.5 Hz, 1H), 6.92 (d, J 8.5 Hz, 2H), 7.33 (d, J 8.5 Hz, 2H); ¹³C NMR (125 MHz, CDCl₃): δ 13.50, 15.27, 62.65, 68.63, 100.42, 114.37, 128.12, 129.74, 159.30, 171.13; (+) LRESIMS m/z 310 [M+H]⁺ (100).

### INTERMEDIATE 2.2.2: 3-(2,2-DIETHOXYETHOXY)-N,N-DIETHYLBENZAMIDE

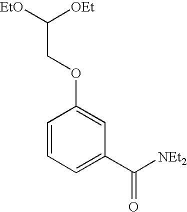

To a mixture of INTERMEDIATE 2.1.2 (3.00 g, 15.5 mmole) and cesium carbonate (11.00 g, 34 mmole) in N,N-dimethylformamide (20 mL) was added bromoacetaldehyde diethyl acetal (4.7 mL, 31 mmole) and the resulting mixture was heated at 80° C. for 1.8 h under nitrogen. The reaction mixture was cooled, water (100 mL) added and the mixture extracted with ethyl acetate (3×100 mL). The combined organic extracts were washed with water (3×100 mL), dried (MgSO₄), filtered and the solvent removed in vacuo. The residue was purified by squat chromatography (ethyl acetate/hexane, 4/6) to give INTERMEDIATE 2.2.2 (4.70 g, 98%) as a clear liquid. ¹H NMR (500 MHz, CDCl₃) δ 1.09 (br s, 6H), 1.21 (t, J 7 Hz, 6H), 3.23 (br s, 2H), 3.49 (br s, 2H), 3.60 (m, 2H), 3.73 (m, 2H), 3.99 (d, J 5.5 Hz, 2H), 4.80 (t, J 5.5 Hz, 1H), 6.90 (m, 3H), 7.26 (t, J 7.5 Hz, 1H); ¹³C NMR (125 MHz, CDCl₃): δ 12.78, 14.07, 15.20, 39.13, 43.12, 62.56, 68.64, 100.38, 112.47, 115.49, 118.67, 129.45, 138.47, 158.50, 170.77; (+) LRESIMS m/z 310 [M+H]⁺ (100).

### INTERMEDIATE 2.2.3: 2-(2,2-DIETHOXYETHOXY)-N,N-DIETHYLBENZAMIDE

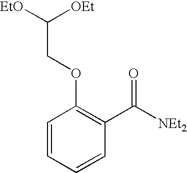

To a mixture of INTERMEDIATE 2.1.3 (3.00 g, 15.5 mmole) and cesium carbonate (11.00 g, 34 mmole) in N,N-dimethylformamide (20 mL) was added bromoacetaldehyde diethyl acetal (4.7 mL, 31 mmole) and the resulting mixture was heated at 80° C. for 18 h under nitrogen. The reaction mixture was cooled, water (100 mL) added and the mixture extracted with ethyl acetate (3×100 mL). The combined organic extracts were washed with water (3×100 mL), dried (MgSO₄), filtered and the solvent removed in vacuo. The residue was purified by squat chromatography (ethyl acetate/hexane, 4/6) to give INTERMEDIATE 2.2.3 (4.50 g, 94%) as a clear liquid. ¹H NMR (500 MHz, CDCl₃) δ 0.98 (t, J 7 Hz, 3H), 1.17 (m, 9H), 3.11 (br p, J 7.5 Hz, 2H), 3.36 (br m, 1H), 3.55 (br p, J 7.5 Hz, 2H), 3.66 (br m, 3H), 3.95 (d, J 5 Hz, 2H), 4.72 (t, J 5 Hz, 1H), 6.84 (d, J 8 Hz, 1H), 6.92 (t, J 7.5 Hz, 1H), 7.14 (d, J 7.5 Hz, 1H), 7.25 (t, J 8 Hz, 1H); ¹³C NMR (125 MHz, CDCl₃): δ 12.66, 13.75, 15.09, 38.69, 42.60, 62.64, 62.69, 69.09, 100.51, 111.89, 120.95, 127.06, 127.34, 129.66, 153.90, 168.36; (+) LRESIMS m/z 310 [M+H]⁺ (100).

### INTERMEDIATE 3.1.1: N,N-DIETHYL-4-FORMYLBENZAMIDE

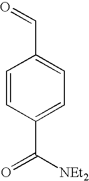

A mixture of 4-carboxybenzaldehyde (2.00 g, 13.3 mmole) and thionyl chloride (5 mL) was heated at reflux util the reaction mixture clarified. The reaction mixture was allowed to cool to room temperature and the excess thionyl chloride removed in vacuo. The residue was dissolved in dichloromethane (50 mL) and added slowly to a solution of diethylamine (5 mL) in dichloromethane (50 mL) while cooling the reaction mixture with an ice/water bath. After complete addition the reaction mixture was allowed to warm to room temperature over 1 h and 1 M hydrochloric acid (50 mL) was added and the mixture filtered through a 1PS filter paper washing the aqueous phase with dichloromethane (50 mL). The solvent was removed from the combined organic phases and the residue purified by flash chromatography (ethyl acetate/hexanes, 50/50) to give INTERMEDIATE 3.1.1 (2.38 g, 87%) as a viscous yellow oil; ¹H NMR (500 MHz, CDCl₃): δ 1.07, 1.22 (2 br s, 6H), 3.18, 3.52 (2 br s, 4H), 7.49 (d, J 8 Hz, 2H), 7.88 (d, J 8 Hz, 2H), 10.00 (s, 1H); ¹³C NMR (125 MHz, CDCl₃): δ 12.96, 14.67, 39.46, 43.32, 127.01, 129.96, 136.65, 143.09, 169.96, 191.61.

### INTERMEDIATE 3.2.1: N,N-DIETHYL-4-[(E)-2-METHOXYVINYL]BENZAMIDE

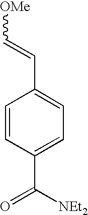

8.57 g (0.025 mmol, 1.25 eq) Methoxymethyltriphenylphosphonium chloride was dissolved in 70 mL methanol and 5.74 mL of a 25% solution of sodium methoxide in methanol was added dropwise at room temperature. The mixture was stirred for 2 h during which a white precipitate was formed. Evaporation to dryness followed by co-evaporation with benzene (2×) yielded the corresponding Wittig reagent. 4.20 g (0.020 mmol) INTERMEDIATE 3.1.1 dissolved in 70 mL THF was added. The red reaction mixture was refluxed for 6 h. After cooling to room temperature a 1:1 mixture of silica and sea sand (30 g) was added and the volatiles were evaporated. Column chromatography of the residue (250 g, pentane/EA 2:1) yielded 2.36 g (0.010 mmol, 50%) INTERMEDIATE 3.2.1 as a 1:1 mixture of E/Z isomers. R_{f}: 0.31 (pentane/EA 2:1), ¹H NMR (500 MHz, CDCl₃): δ 1.03, 1.21 (2 brs, 6H), 2.74-2.80, 2.87-2.98, 3.00-3.08, 3.18-3.24, 3.45-3.55 (5 m, 8H), 3.63, 3.87 (2 s, 6H), 5.08 (s, 0.5H), 6.23 (s, 0.5H), 6.63 (s, 0.5H), 7.26-7.35 (m, 5H).

### INTERMEDIATE 4.1.1: N,N-DIETHYL-N′-[2-(3-FLUORO-4-METHOXYPHENYL)ETHYL]TEREPHTHALAMIDE

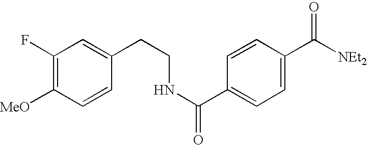

To an ice-cooled solution of INTERMEDIATE 1.2.1 (510 mg, 3.01 mmol) and diethylamine (220 mg, 3.01 mmol) in DCM (20 mL), terephthaloyl chloride (556 mg, 2.74 mmol) in DCM (10 mL) was added dropwise. After the addition was completed the reaction was stirred at room temperature for 17 h. The reaction mixture was washed with 1 M HCl (10 mL) and water (10 mL). After removal of the organic solvent, the residue was purified by repeated flash chromatography on SiO₂ column (EtOAc:DCM 50:50) yielding INTERMEDIATE 4.1.1 as a light-yellow oil (247 mg, 24%). ¹H NMR (500 MHz, CDCl₃): δ 1.00 (br s, CH₃), 1.18 (br s, CH₃), 2.77 (br s, 1H), 3.13 (br s, 31), 3.50 (m, 4H), 3.77 (s, OCH₃), 6.84 (m, 4H), 7.20 (d, J 6 Hz, 1H), 7.33 (br s, 1H), 7.68 (d, J 6 Hz, 1H); ¹³C NMR (125 MHz, CDCl₃): δ 13.03, 14.32, 34.78, 39.64, 41.49, 43.53, 56.47, 113.85, 116.51 (d, J 17.6 Hz), 124.55, 126.61, 127.53, 135.60, 138.14, 139.73, 146.28 (d, J 10.5 Hz), 152.46 (d, J 244 Hz), 171.80, 171.83; (+) LRESIMS m/z 373.20 [M+H]⁺.

### INTERMEDIATE 4.1.2: N¹,N¹-DIETHYL-N⁴-[2-(4-METHOXYPHENYL)ETHYL]TEREPHTHALAMIDE

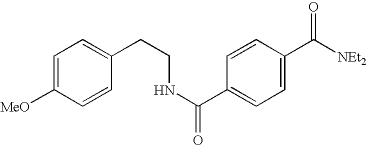

To an ice cooled solution of diethylamine (2.5 mL, 24.5 mmole) and 4-methoxyphenethylamine (3.7 g, 24.5 mmole) in dichloromethane (100 mL) was added a solution of terephthaloyl chloride (2 g, 9.8 mmole) in dichloromethane (50 mL) dropwise while stirring under nitrogen. After complete addition the mixture was stirred for 4 h after which saturated sodium hydrogen carbonate (100 mL) was added and the phases separated. The aqueous phase was then extracted with ethyl acetate (3×100 mL), the combined organic phases dried (MgSO₄), filtered and the solvent removed in vacuo. The residue was purified by flash chromatography (ethyl acetate:dichloromethane, 40:60) to give the product (1.63 g, 47%) as a white solid; ¹H NMR (500 MHz, CDCl₃): δ 1.10, 1.26 (2 br s, 6H), 2.88 (t, J 7 Hz, 2H), 3.22, 3.53 (2 br s, 4H), 3.67 (m, 2H), 3.80 (s, 3H), 6.45 (br s, 1H), 6.87 (d, J 8 Hz, 2H), 7.15 (d, J 8 Hz, 2H), 7.35, 7.71 (2 d, J 7.5 Hz, 4H); ¹³C NMR (125 MHz, CDCl₃): δ 12.86, 14.17, 34.68, 41.38, 39.41, 43.18, 55.25, 114.13, 126.41, 127.13, 129.71, 130.80, 135.42, 139.69, 158.35, 166.89, 170.47; (+) LRESIMS m/z 355 [M+H]⁺ (100).

### INTERMEDIATE 4.1.3: N′-[2-(3-BROMO-4-METHOXYPHENYL)ETHYL]-N,N-DIETHYLTEREPHTHALAMIDE

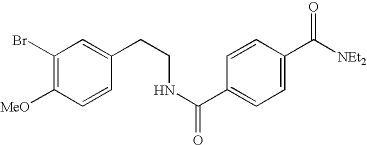

To an ice-cooled solution of 1.35 mL (2.2 eq, 8.13 mmol) 3-bromo-4-methoxyphenethylamine and 0.84 mL (2.2 eq, 8.13 mmol) diethylamine in 40 mL dichloromethane 1.5 g (7.39 mmol) terephthaloyl chloride in 40 mL dichloromethane was added dropwise. After complete addition the reaction was stirred at room temperature for 18 h. To the crude mixture silica (10 g), sea sand (10 g), and methanol (50 mL) were added and the volatiles were removed in vacuo. Column chromatography (ethyl acetate/dichloromethane 3:1) yielded 920 mg (1.56 mmol, 21%) of the desired product. ¹H NMR (500 MHz, CDCl₃): δ 1.12, 1.28 (2 brs, 6H), 2.89 (t, J 7.0 Hz, 2H), 3.24, 3.58 (2 brs, 4H), 3.70 (dd, J 7.0, 12.5 Hz, 2H), 3.91 (s, 3H), 6.16 (s, 1H), 6.88, 7.15 (2 d, J 8.5 Hz, 2H), 7.42 (s, 1H), 7.45, 7.74 (2 d, J 8.5 Hz, 4H). (+) LRESIMS m/z 433, 445 [M+H]⁺.

### INTERMEDIATE 4.2.1: N,N-DIETHYL-4-(6-FLUORO-7-METHOXY-1,2,3,4-TETRAHYDROISOQUINOLIN-1-YL)BENZAMIDE

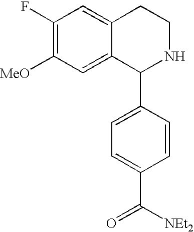

A solution of trifluoromethanesulfonic anhydride (455 mg, 1.61 mmol) in DCM (2 mL) was added dropwise to an ice-cooled solution of INTERMEDIATE 4.1.1 (200 mg, 0.54 mmol) and DMAP (240 mg, 1.61 mmol) in DCM (2 mL). The mixture was allowed to warm to RT overnight. Saturated NaHCO₃ solution (10 mL) was added and the phases separated. The aqueous phase was extracted with DCM (2×10 mL) and the combined organic layers concentrated in vacuo. The residue was dissolved in MeOH (5 mL) and NaBH₄ (41 mg, 1.07 mmol) added. After 30 min stirring 1 M NaOH (10 mL) was added and the mixture extracted with DCM (3×10 mL). The combined organic layers concentrated to dryness in vacuo and the residue purified by SiO₂ flash chromatography (EtOAc:CHCl₃:MeOH 30:63:7) to give INTERMEDIATE 4.2.1 as a yellow oil (122 mg, 64%). ¹H NMR (500 MHz, CDCl₃): δ 1.07 (br s, 3H), 1.20 (br s, 3H), 2.68 (dt, J 4.8, 16 Hz, 1H), 2.86 (m, 1H), 2.99 (m, 1H), 3.16 (m, 1H), 3.23 (br s, 2H), 3.50 (br s, 2H), 3.61 (s, 3H), 5.02 (s, 1H), 6.28 (d, J 9 Hz, 1H), 6.81 (d, J 11.5 Hz, 1H), 7.24 (d, J 8 Hz, 1H), 7.31 (d, J 8 Hz, 1H); ¹³C NMR (125 MHz, CDCl₃): δ 13.10, 14.38, 29.08, 39.54, 41.87, 43.51, 56.52, 61.47, 113.37, 116.21 (d, J 17.5 Hz), 126.79, 128.55, 129.11, 133.43, 136.67, 145.79, 145.83, 151.33 (d, J 244 Hz), 171.29; (+) LRESIMS m/z 357.20 [M+H]⁺.

### INTERMEDIATE 4.2.2: N,N-DIETHYL-4-(7-METHOXY-1,2,3,4-TETRAHYDROISOQUINOLIN-1-YL)BENZAMIDE

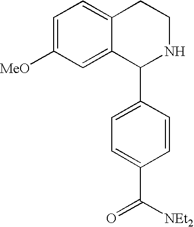

To a solution of INTERMEDIATE 4.1.2 (1.00 g, 2.8 mmole) and 4-(dimethylamino)pyridine (1.03 g, 8.5 mmole) in dichloromethane (80 mL) was added a solution of trifluoromethanesulfonic anhydride (1.5 mL, 8.9 mmole) in dichloromethane (7 mL) dropwise at 0° C. under nitrogen. After complete addition the reaction mixture was allowed to warm to room temperature over 18 h. Saturated sodium hydrogen carbonate (100 mL) was added and the mixture extracted with dichloromethane (2×50 mL). The organic phase was dried (MgSO₄), filtered and the solvent removed in vacuo. The residue was dissolved in methanol (40 mL) and sodium borohydride (0.17 g, 4.5 mmole) added. After 0.5 h, 1M sodium hydroxide (100 mL) was added and the mixture extracted with dichloromethane (3×50 mL). The combined organics were dried (MgSO₄), filtered and the solvent removed in vacuo. The crude was purified by flash chromatography (ethyl acetate/10% methanol in chloroform, 3/7) to give INTERMEDIATE 4.2.2 as an off-white solid (0.63 g, 66%). ¹H NMR (500 MHz, CDCl₃): δ 1.10, 1.25 (2 brs, 6H), 2.90, 3.00, 3.22, 3.53 (4 m, 9H), 3.64 (s, 3H), 5.15 (s, 1H), 6.28 (d, J 2 Hz, 1H), 6.75 (dd, J 2, 8 Hz, 1H), 7.07 (d, J 8 Hz, 1H), 7.30, 7.33 (m, 4H). ¹³C NMR (125 MHz, CDCl₃): δ 12.8, 14.2, 28.2, 39.3, 43.3, 41.8, 55.2, 61.3, 112.9, 113.1, 126.6, 129.2, 126.8, 130.0, 136.6, 137.4, 144.3, 157.7, 171.0). (+) LRESIMS m/z 339 [M+H]⁺.

### INTERMEDIATE 4.2.3: 4-(6-BROMO-7-METHOXY-1,2,3,4-TETRAHYDROISOQUINOLINE-1-YL)-N,N-DIETHYLBENZAMIDE

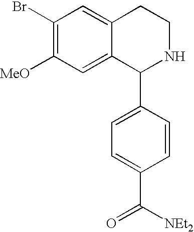

To an ice-cooled solution of 840 mg (1.94 mmol) INTERMEDIATE 4.1.3 and 0.71 g (3.0 eq, 5.82 mmol) 4-dimethylaminopyridine in 50 mL dichloromethane was added 1.63 mL (5.0 eq, 9.69 mmol) triflic anhydride in 5 mL dichloromethane dropwise over the course of 15 min. The reaction was slowly allowed to warm to room temperature and stirred for 18 h. Saturated aqueous sodium bicarbonate solution was added and after phase separation, the aqueous phase was extracted with dichloromethane. The combined organic phases were washed with brine, dried, and evaporated. The crude product was dissolved in 40 mL methanol and 294 mg (4.0 eq, 7.76 mmol) sodium borohydride was added portionwise. The mixture was stirred for 30 min at room temperature. After addition of 80 mL 1 M aqueous sodium hydroxide solution the mixture was extracted with dichloromethane. The combined organic phases were washed with brine, dried, and evaporated in vacuo. Flash chromatography (40 g, dichloromethane/methanol 30:1) yielded 465 mg (1.11 mmol, 57%) of the desired product. ¹H NMR (500 MHz, CDCl₃): δ 1.12, 1.26 (2 brs, 6H), 2.77, 2.95, 3.06, 3.24 (4 m, 6H), 3.58 (brs, 2H), 5.10 (s, 1H), 6.29 (s, 1H), 7.30, 7.37 (2 m, 5H). (+) LRESIMS m/z 417, 419[M+H]⁺.

### INTERMEDIATE 5.1.1: 4-[(6,7-DIMETHOXY-1,2,3,4-TETRAHYDROISOQUINOLIN-1-YL)METHOXY]-N,N-DIETHYLBENZAMIDE

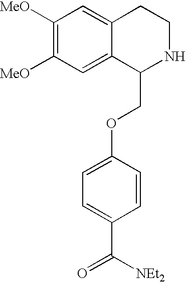

A solution of 3,4-dimethoxyphenethylamine (1.00 g, 5.6 mmole) and INTERMEDIATE 2.2.1 (1.15 g, 3.7 mmole) in formic acid (6 mL) was stirred at 80° C. for 3 h. The reaction mixture was then cooled to room temperature, ice/water (50 mL) added and the mixture basified by addition of concentrated ammonia solution. Chloroform (150 mL) was added and the mixture filtered through a Whatman 1PS filter paper. The solvent was removed from the organic phase in vacuo and the residue purified by flash chromatography (methanol/chloroform, 5/95) to give INTERMEDIATE 5.1.1 (1.16 g, 78%) as a viscous oil. ¹H NMR (500 MHz, CDCl₃) δ 1.16 (br s, 6H), 2.79 (m, 2H), 3.05 (m, 1H), 3.21 (m, 1H), 3.99 (br s, 4H), 3.84 (s, 3H), 3.85 (s, 31), 4.19 (m, 2H), 4.38 (m, 1H), 6.62 (s, 1H), 6.68 (s, 1H), 6.94 (d, J 9 Hz, 2H), 7.33 (d, J 9 Hz, 2H); (+) LRESIMS m/z 399 [M+H]⁺.

### INTERMEDIATE 5.1.2: 3-[(6,7-DIMETHOXY-1,2,3,4-TETRAHYDROISOQUINOLIN-1-YL)METHOXY]-N,N-DIETHYLBENZAMIDE

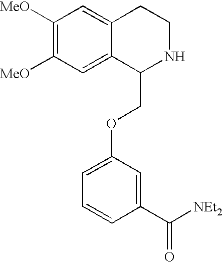

A solution of 3,4-dimethoxyphenethylamine (1.03 g, 5.7 mmole) and INTERMEDIATE 2.2.2 (1.18 g, 3.8 mmole) in formic acid (6 mL) was stirred at room temperature for 48 h. Ice/water (50 mL) was then added and the mixture basified by addition of concentrated ammonia solution. Chloroform (150 mL) was added and the mixture filtered through a Whatman 1PS filter paper. The solvent was removed from the organic phase in vacuo and the residue purified by flash chromatography (methanol/chloroform, 2.5/97.5) to give INTERMEDIATE 5.1.2 (1.16 g, 78%) as a viscous oil. ¹H NMR (500 MHz, CDCl₃) δ 1.09 (br s, 3H), 1.22 (br s, 3H), 2.79 (br s, 2H), 3.04 (m, 1H), 3.23 (br m, 1H), 3.52 (br s, 2H), 3.84 (s, 3H), 3.85 (s, 3H), 4.18 (m, 2H), 4.38 (m, 1H), 6.62 (s, 1H), 6.68 (s, 1H), 6.94 (m, 3H), 7.29 (t, J 6.5 Hz, 1H); ¹³C NMR (125 MHz, CDCl₃): δ 12.80, 14.15, 28.98, 39.17, 39.77, 43.21, 54.56, 55.79, 55.83, 56.01, 56.04, 70.75, 109.55, 112.03, 112.38, 115.64, 118.68, 125.86, 127.98, 129.54, 138.60, 147.32, 147.99, 158.72, 170.81; (+) LRESIMS m/z 399 [M+H]⁺ (100).

### INTERMEDIATE 5.1.3: 2-[(6,7-DIMETHOXY-1,2,3,4-TETRAHYDROISOQUINOLIN-1-YL)METHOXY]-N,N-DIETHYLBENZAMIDE

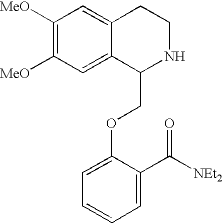

A solution of 3,4-dimethoxyphenethylamine (1.11 g, 6.2 mmole) and INTERMEDIATE 2.2.3 (1.27 g, 4.1 mmole) in formic acid (6 mL) was stirred at room temperature for 48 h. Ice/water (50 mL) was then added and the mixture basified by addition of concentrated ammonia solution. Chloroform (150 mL) was added and the mixture filtered through a Whatman 1PS filter paper. The solvent was removed from the organic phase in vacuo and the residue purified by flash chromatography (methanol/chloroform, 2.5/97.5) to give INTERMEDIATE 5.1.2 (1.34 g, 82%) as a viscous oil. ¹H NMR (500 MHz, CDCl₃) δ 0.99, 1.22 (2 br s, 6H), 2.80, 3.01, 3.16 (br m, 8H), 3.84 (s, 3H), 3.85 (s, 3H), 4.27 (br m, 2H), 4.35 (br m, 1H), 6.61 (s, 1H), 6.43 (s, 1H), 6.95 (d, J 8 Hz, 1H), 7.00 (t, J 7.5 Hz, 1H), 7.20 (d, J 6 Hz, 1H), 7.32 (t, J 8 Hz, 1H). (+) LRESIMS m/z 399 [M+H]⁺.

### INTERMEDIATE 5.1.4: N,N-DIETHYL-3-[(6-METHOXY-1,2,3,4-TETRAHYDROISOQUINOLIN-1-YL)METHOXY]BENZAMIDE

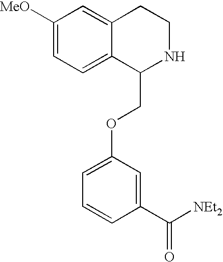

A solution of 3-methoxyphenethylamine (0.07 g, 0.46 mmole) and INTERMEDIATE 2.2.2 (0.1 g, 0.3 mmole) in formic acid (1 mL) was stirred at 100° C. for 3 h. Ice/water (20 mL) was then added and the mixture basified by addition of concentrated ammonia solution. Chloroform (60 mL) was added and the mixture filtered through a Whatman 1PS filter paper. The solvent was removed from the organic phase in vacuo and the residue purified by flash chromatography (methanol/chloroform, 2.5/97.5) to give INTERMEDIATE 5.1.4 (0.08 g, 70%) as a viscous oil. ¹H NMR (500 MHz, CDCl₃): δ 1.08, 1.21 (2 br s, 6H), 2.76 (br s, 1H), 2.83 (m, 2H), 3.02 (m, 1H), 3.20 (br m, 3H), 3.50 (br s, 2H), 3.76 (s, 3H), 4.10 (dd, J 3.5, 9 Hz, 1H), 4.18 (dd, J 3.5, 9 Hz, 1H), 4.37 (dd, J 3.5, 9 Hz, 1H), 6.66 (s, 1H), 6.72 (dd, J 2.5, 9 Hz, 1H), 6.93 (m, 2H), 7.08 (d, J 8 Hz, 1H), 7.27 (t, J 8 Hz, 1H). ¹³C NMR (125 MHz, CDCl₃): δ 12.78, 14.13, 29.83, 39.06, 39.66, 43.16, 54.35, 55.08, 70.72, 112.13, 112.38, 113.91, 115.46, 118.57, 126.33, 127.33, 129.47, 137.13, 138.52, 158.14, 158.67, 170.75. (+) LRESIMS m/z 369 [M+H]⁺.

### INTERMEDIATE 5.1.5: 4-[(6,7-DIMETHOXY-1,2,3,4-TETRAHYDROISOQUINOLIN-1-YL)METHYL]-N,N-DIETHYLBENZAMIDE

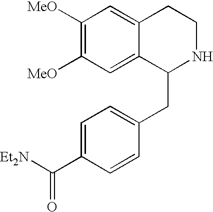

3.43 mL (0.020 mol, 2 eq) 3,4-dimethoxyphenethylamine was dissolved in 20 mL formic acid under ice cooling and the resulting solution was added to 2.36 g (0.010 mol) vinyl ether INTERMEDIATE 3.2.1. The mixture was stirred under reflux for 2 h. After cooling to room temperature the solution was poured onto crushed ice and DCM was added. After adjusting the aqueous layer to pH 10 it was extracted with DCM. The organic phase was washed with water, brine, and dried. Flash chromatography (2×90 g, chloroform/methanol 50:1 to 10:1) yielded 0.35 g (0.93 mmol, 9%) of the product. ¹H NMR (500 MHz, CDCl₃): δ 1.14, 1.23 (2 brs, 6H), 2.68-3.02 (m, 4H), 3.20-3.24 (m, 2H), 3.28, 3.55 (2 brs, 4H), 3.82, 3.86 (2 s, 6H), 4.19-4.23 (m, 1H), 6.60, 6.63 (2 s, 2H), 7.29-7.36 (m, 4H). (+) LRESIMS m/z 383 [M+H]⁺.

### INTERMEDIATE 5.1.6: N,N-DIETHYL-2-[(6-METHOXY-1,2,3,4-TETRAHYDROISOQUINOLIN-1-YL)METHOXY]BENZAMIDE

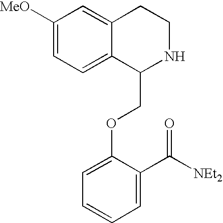

A solution of 3-methoxyphenethylamine (0.74 g, 4.9 mmole) and INTERMEDIATE 2.2.3 (1.00 g, 3.3 mmole) in formic acid (5 mL) was stirred at room temperature for 2 days. Ice/water (50 mL) was then added and the mixture basified by addition of concentrated ammonia solution. Chloroform (150 mL) was added and the mixture filtered through a Whatman 1PS filter paper. The solvent was removed from the organic phase in vacuo and the residue purified by flash chromatography (methanol/chloroform, 2.5/97.5) to give INTERMEDIATE 5.1.6 (1.09 g, 89%) as a viscous oil. ¹H NMR (500 MHz, CDCl₃) δ 0.91, 1.01 (2 br s, 31), 1.20 (br s, 3H), 2.76 (br s, 3H), 2.95 (m, 21), 3.01, 3.17 (2 br s, 2H), 3.31 (br s, 1H), 3.72 (br s, 1H), 3.73 (s, 3H), 4.24 (br m, 3H), 6.62 (s, 1H), 6.69 (d, J 8.5 Hz, 1H), 6.90 (d, J 8 Hz, 1H), 6.94 (t, J 7.5 Hz, 1H), 7.04 (d, J 8 Hz, 1H), 7.17 (d, J 6.5 Hz, 1H), 7.26 (t, J 7.5 Hz, 1H); ¹³C NMR (125 MHz, CDCl₃): δ 12.65, 13.74, 29.74. 38.66, 40.22, 42.51, 54.26, 54.88, 70.90, 111.86, 112.26, 113.68, 120.92, 126.97, 127.07, 129.65, 137.07, 153.91, 157.90, 168.32; (+) LRESIMS m/z 369 [M+H]⁺ (100).

### INTERMEDIATE 5.1.7: 4-(6,7-DIMETHOXY-1,2,3,4-TETRAHYDROISOQUINOLIN-1-YL)-N,N-DIETHYLBENZAMIDE

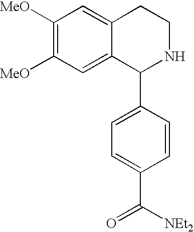

1.87 g (9.11 mmol) of INTERMEDIATE 3.1.1 and 2.00 mL (1.3 eq, 11.8 mmol) of 3,4-dimethoxyphenethylamine were dissolved in 30 mL TFA and stirred under reflux for 18 h. The reaction mixture was concentrated in vacuo and redissolved in DCM. The organic phase was washed with saturated aqueous sodium bicarbonate solution, water, and brine, dried, and concentrated in vacuo. The resulting syrup was purified by flash chromatography (90 g, chloroform/methanol 9:1) to yield 3.21 g (8.71 mmol, 96%) of a reddish foam. ¹H NMR (500 MHz, CDCl₃): δ 1.15, 1.25 (2 brs, 6H), 2.77, 2.96, 3.07, 3.26 (4 ddd, 4H), 3.28, 3.59 (2 brs, 4H), 3.66, 3.89 (2 s, 6H), 5.11 (s, 1H), 6.25 (s, 1H), 6.65 (s, 1H), 7.31, 7.35 (2 d, J 8 Hz, 4H). ¹³C NMR (125 MHz, CDCl₃): δ 13.2, 14.5, 29.3, 39.5, 43.6, 42.0, 56.1, 56.2, 61.2, 111.2, 111.8, 126.8, 129.2, 127.8, 129.3, 136.7, 145.7, 147.5, 148.2, 171.4. (+) LRESIMS m/z 369 [M+H]⁺.

### INTERMEDIATE 5.1.8: N,N-DIETHYL-4-(6-METHOXY-1,2,3,4-TETRAHYDROISOQUINOLIN-1-YL)BENZAMIDE

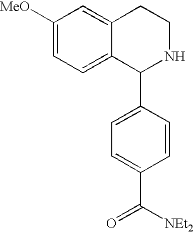

INTERMEDIATE 3.1.1 (3.30 g, 16.1 mmol) and 3-methoxyphenethylamine (3.27 mL, 22.5 mmol) were dissolved in TFA (50 mL) and stirred under reflux for 18 h. The reaction mixture was concentrated in vacuo and redissolved in DCM. The organic phase was washed with saturated aqueous sodium bicarbonate solution, water, and brine, dried, and concentrated in vacuo. The resulting syrup was purified by flash chromatography to yield a reddish foam (4.07 g, 12.03 mmol, 75%). ¹H NMR (500 MHz, CDCl₃): δ 1.04, 1.16 (2 brs, 6H), 2.86-3.30 (m, 4H), 3.46 (brs, 4H), 3.70, 3.73 (2 s, 6H), 5.43 (s, 1H), 6.58-6.66 (m, 3H), 7.24, 7.30 (2 d, J 8 Hz, 4H). (+) LRESIMS m/z 339 [M+H]⁺.

### INTERMEDIATE 5.1.9: 3-(6,7-DIMETHOXY-1,2,3,4-TETRAHYDROISOQUINOLIN-1-YL-N,N-DIETHYLBENZAMIDE

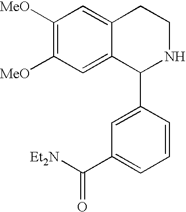

N,N-diethyl-3-formylbenzamide (200 mg, 0.97 mmol) and 3,4-dimethoxyphenethylamine (0.25 mL, 1.50 mmol) were dissolved in formic acid (1.5 mL) and stirred under reflux for 18 h. The reaction mixture was concentrated in vacuo and redissolved in DCM. The organic phase was washed with saturated aqueous sodium bicarbonate solution, water, and brine, dried, and concentrated in vacuo. The resulting syrup was purified by flash chromatography to yield a white foam (0.25 g, 0.68 mmol, 70%). ¹H NMR (500 MHz, CDCl₃): δ 1.03, 1.21 (2 brs, 6H), 2.74, 2.90, 3.04 (3 m, 3H), 3.18 (m, 5H), 3.63, 3.87 (2 s, 6H), 5.08 (s, 1H), 6.23 (s, 1H), 6.63 (s, 1H), 7.26-7.38 (m, 4H). (+) LRESIMS m/z 369 [M+H]⁺.

### INTERMEDIATE 5.1.10: 4-(5,8-DIMETHOXY-1,2,3,4-TETRAHYDROISOQUINOLIN-1-YL)-N,N-DIETHYLBENZAMIDE

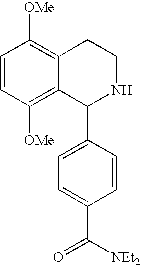

INTERMEDIATE 3.1.1 (100 mg, 0.49 mmol) and 2,5-dimethoxyphenethylamine (0.09 mL, 0.54 mmol) were dissolved in methanol (2 mL) and stirred at room temperature for 18 h. The solvent was removed in vacuo and the residue was redissolved in TFA (1.5 mL). The reaction mixture was refluxed for 3 d and afterwards concentrated in vacuo and redissolved in DCM. The organic phase was washed with saturated aqueous sodium bicarbonate solution, water, and brine, dried, and concentrated in vacuo. The resulting syrup was purified by flash chromatography to yield a foam (68 mg, 0.185 mmol, 38%). ¹H NMR (500 MHz, CDCl₃): δ 1.12, 1.24 (2 brs, 6H), 2.82-3.20 (m, 4H), 3.28, 3.55 (2 brs, 4H), 3.53, 3.84 (2 s, 6H), 5.59 (s, 1H), 6.67, 6.78 (2 d, J 9 Hz, 2H), 7.26, 7.32 (2 d, J 8 Hz, 4H). ¹³C NMR (125 MHz, —CDCl₃): δ 14.2, 15.5, 22.2, 37.1, 39.7, 43.8, 54.5, 55.8, 55.9, 108.3, 109.1, 124.0, 124.9, 136.7, 126.6, 129.1, 150.5, 151.4, 171.3. (+) LRESIMS m/z 369 [M+H]⁺.

### INTERMEDIATE 5.1.11: 4-[(6,7-DIHYDROXY-1,2,3,4-TETRAHYDROISOQUINOLIN-1-YL)METHOXY]-N,N-DIETHYLBENZAMIDE

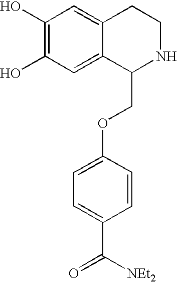

A solution of 3,4-dihydroxyphenethylamine hydrochloride (0.19 g, 1.0 mmole) and INTERMEDIATE 2.2.1 (0.20 g, 0.7 mmole) in formic acid (2 mL) was stirred at room temperature for 48 h. Ice/water (20 mL) was added and the mixture basified by addition of concentrated ammonia solution. Chloroform (50 mL) was added and the mixture filtered through a Whatman 1PS filter paper. The solvent was removed from the organic phase in vacuo and the residue purified by flash chromatography (methanol/chloroform, 1/9) to give INTERMEDIATE 5.1.11 (0.15 g, 61%) as a viscous oil. ¹H NMR (500 MHz, CDCl₃): δ 1.16 (br s, 6H), 2.58 (br s, 2H), 2.94 (br s, 1H), 3.12 (br s, 1H), 3.31, 3.48 (2 br s, 4H), 4.00 (br s, 2H), 4.15 (br s, 1H), 6.05 (br s, 2H), 6.42 (s, 2H), 6.81 (d, J 8 Hz, 2H), 7.26 (d, J 8 Hz, 2H). ¹³C NMR (125 MHz, CDCl₃): δ 12.80, 14.03, 27.70, 39.57, 39.60, 43.75, 54.02, 69.77, 112.87, 114.41, 115.60, 123.46, 126.07, 128.08, 129.29, 143.65, 144.61, 159.26, 171.56. (+) LRESIMS m/z 371 [M+H]⁺.

### INTERMEDIATE 5.1.12: N,N-DIETHYL-4-(7-HYDROXY-6-METHOXY-1,2,3,4-TETRAHYDROISOQUINOLIN-1-YL)BENZAMIDE

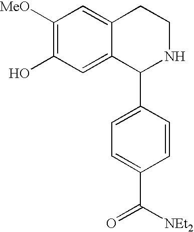

To a cooled mixture of 3-methoxytyramine hydrochloride (210 mg, 1.03 mmol) and INTERMEDIATE 3.1.1 (180 mg, 0.87 mmol) was slowly added trifluoroacetic acid (3.2 mL). The solution was heated to reflux for 20 hr afterwhich excess trifluoroacetic acid was removed in vacuo and the residue redissolved in dichloromethane (20 mL). The solution was basified with saturated aqueous sodium bicarbonate to pH=10. Dichloromethane fraction was separated, washed with saturated aqueous sodium chloride (5×5 mL), dried over MgSO₄ and concentrated. The product was purified by flash chromatography using silica column and dichloromethane/methanol (95:5) as solvent to give 120 mg (0.3389 mmol, 33%) of INTERMEDIATE 5.1.12 as light yellow solid. ¹H NMR (500 MHz, CDCl₃): δ 1.12 (br s, 3H), 1.23 (brs, 3H), 2.75 (m, 1H), 2.93 (m, 1H), 3.04 (m, 1H), 3.20 (m, 1H), 3.27 (br s, 2H), 3.54 (br s, 2H), 3.86 (s, 3H), 5.01 (s, 1H), 6.28 (s, 1H), 6.61 (s, 1H), 7.26-7.32 (m, 4H). ¹³C NMR (125 MHz, CDCl₃): δ 13.14, 14.45, 29.30, 39.50, 42.17, 43.53, 56.12, 61.23, 111.20, 114.19, 126.72, 126.92, 129.18, 130.11, 136.49, 144.06, 145.92, 171.44. (+) LRESIMS m/z 355 [M+H]⁺.

### INTERMEDIATE 5.1.13: N,N-DIETHYL-4-(5,6,7,8-TETRAHYDRO[1,3]DIOXOLO[4,5-G]ISOQUINOLIN-5-YL)BENZAMIDE

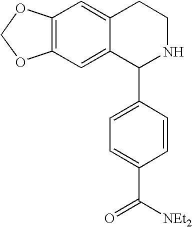

1.00 g (4.96 mmol) 3,4-methylenedioxyphenethylamine hydrochloride and 1.20 g (1.2 eq, 5.95 mmol) INTERMEDIATE 3.1.1 were dissolved in 7 mL TFA and the solution was stirred under reflux for 18 h. After evaporation of the volatiles the residue was taken up in DCM, washed with 1 M aqueous sodium hydroxide solution, water, and brine and dried. Flash chromatography (40 g, DCM/MeOH 30:1) yielded 330 mg (0.94 mmol, 19%) of a reddish foam. ¹H NMR (500 MHz, CDCl₃): δ 1.13, 1.25 (2 brs, 6H), 2.68, 2.95, 3.06, 3.22 (4 m, 4H), 3.28, 3.58 (brs, 2H), 5.10 (s, 1H), 6.29 (s, 1H), 7.30, 7.37 (2 m, 5H). (+) LRESIMS m/z 417, 419[M+H]⁺.

### INTERMEDIATE 5.1.14: 4-(6,7-DIMETHOXY-3-METHYL-1,2,3,4-TETRAHYDROISOQUINOLIN-1-YL)-N,N-DIETHYLBENZAMIDE

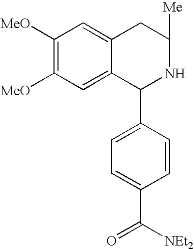

INTERMEDIATE 1.2.4 (517 mg, 2.64 mmol) was dissolved in TFA (7 mL) at 0° C. and the resulting solution added to INTERMEDIATE 3.1.1 (575.5 mg, 2.80 mmol) and refluxed for 17 h at 98° C. The TFA was then removed in vacuo and water (10 mL) added. Concentrated NH₄OH was added until pH 11. DCM (2×30 mL) was used to extract the aqueous phase. The organic layer was washed with brine (2×10 mL) and the solvent removed in vacuo yielding a dark green tar as the residue. Repeated chromatography on SiO₂ column (EtOAc:MeOH 93:7) afforded INTERMEDIATE 5.1.14 as an oil (291 mg, 29%). ¹H NMR (500 MHz, CDCl₃): δ 1.02 (br s, 3H), 1.16 (br s, 3H), 1.18 (s, 3H), 2.62 (m, 2H), 3.10 (m, 1H), 3.19 (br s, 2H), 3.46 (m, 2H, 3.51 (s, 3H), 3.77 (s, 3H), 5.02 (s, 1H), 6.08 (s, 1H), 6.53 (s, 1H), 7.27-7.30 (m, 4H); ¹³C NMR (125 MHz, CDCl₃): δ 13.09, 14.36, 22.63, 37.96, 39.54, 43.49, 50.07, 56.03, 56.07, 63.38, 110.90, 111.63, 126.82, 128.10, 129.22, 130.17, 136.63, 146.10, 147.31, 147.88, 171.35; (+) LRESIMS m/z 383.24 [M+H]⁺.

### INTERMEDIATE 5.1.15: N,N-DIETHYL-4-(6,7,8,9-TETRAHYDRO[1,3]DIOXOLO[4,5-F]ISOQUINOLIN-6-YL)BENZAMIDE

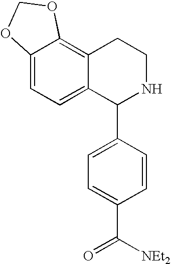

INTERMEDIATE 1.2.2 (670 mg, 4.06 mmol) was dissolved in TFA (7 mL) at 0° C. and the resulting solution added to the aldehyde INTERMEDIATE 3.1.1 (842 mg, 4.10 mmol) and refluxed for 15 h at 98° C. The TFA was removed in vacuo and water (10 mL) added. Concentrated NH₄OH was added until pH 11. EtOAc (2×30 mL) was used to extract the aqueous phase. The organic layer was washed with brine (2×10 mL) and concentrated to dryness in vacuo. The residue was purified by flash chromatography on SiO₂ column EtOAc:MeOH 95:5) to afford INTERMEDIATE 5.1.15 as an oil in quantitative yield. ¹H NMR (500 MHz, CDCl₃): δ 1.11 (br s, 3H), 1.22 (br s, 3H), 1.91 (s, 3H), 3.00 (m, 1H), 3.18-3.24 (m, 4H), 3.39 (m, 1H), 3.51 (br s, 2H), 5.51 (s, 1H), 6.01 (br s, 2H), 6.27 (d, J 7.5 Hz, 1H), 6.63 (d, J 8.5 Hz, 1H), 7.29-7.39 (m, 4H); ¹³C NMR (125 MHz, CDCl₃): δ 12.93, 14.29, 19.80, 39.69, 39.97, 43.66, 59.74, 101.95, 107.67, 115.12, 121.66, 125.13, 126.81, 130.63, 138.04, 138.23, 145.33, 147.01, 171.03; (+) LRESIMS m/z 353.18 [4+H]⁺.

### INTERMEDIATE 5.1.16: N,N-DIETHYL-4-(5,6,7-TRIMETHOXY-1,2,3,4-TETRAHYDROISOQUINOLIN-1-YL)BENZAMIDE

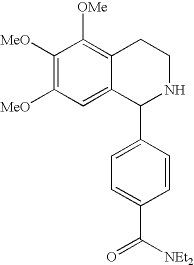

INTERMEDIATE 1.2.3 (828 mg, 3.92 mmol) was dissolved in TFA (7 mL) at 0° C. and the resulting solution added to the aldehyde INTERMEDIATE 3.1.1 (804.4 mg, 3.92 mmol) and refluxed for 15 h at 98° C. The TFA was removed in vacuo and water (10 mL) added. Concentrated NH₄OH was added until pH 11. EtOAc (2×30 mL) was used to extract the aqueous phase. The organic layer was washed with brine (2×10 mL) and concentrated to dryness in vacuo. The residue was purified by flash chromatography on SiO₂ column (EtOAc:MeOH 95:5) to afford INTERMEDIATE 5.1.16 as an oil in quantitative yield. ¹H NMR (500 MHz, CDCl₃): δ 1.11 (br s, 3H), 1.25 (br s, 3H), 1.99 (s, 3H), 2.98-3.14 (m, 2H), 3.20-3.25 (m, 3H), 3.35 (m, 1H), 3.54 (m, 2H), 3.64 (s, 3H), 3.88 (s, 3H), 3.93 (s, 3H), 5.53 (s, 1H), 6.06 (s, 1H), 7.35-7.38 (m, 4H); ¹³C NMR (125 MHz, CDCl₃): δ 13.01, 14.40, 22.75, 39.69, 39.88, 43.59, 56.31, 59.16, 60.83, 61.07, 106.84, 119.72, 126.28, 127.05, 130.57, 137.64, 138.50, 142.13, 151.11, 152.94, 171.35; (+) LRESIMS m/z 399.19 [M+H]⁺.

### INTERMEDIATE 6.1.1: N,N-DIETHYL-4-{6-METHOXY-2-[(4-NITROPHENYL)SULFONYL]-1,2,3,4-TETRAHYDROISOQUINOLIN-1-YL}BENZAMIDE

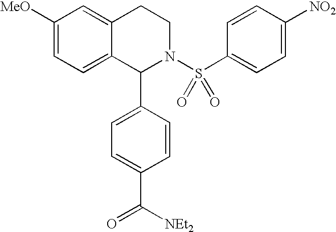

2.17 g (6.39 mmol) INTERMEDIATE 5.1.8 and 2.63 mL (3.0 eq, 0.020 mmol) triethylamine were dissolved in 200 mL DCM, cooled to 0 C, and 1.70 g (1.15 eq, 7.64 mmol) nosyl chloride was added. The mixture was allowed to warm to room temperature and stirred for 18 h. The reaction mixture was poured onto crushed ice and extracted with DCM. The combined organic phases were washed with 1 M hydrochloric acid, 1 M sodium hydroxide solution, water, and brine. Drying and evaporation yielded the crude product, which was purified by flash chromatography (90 g, DCM/methanol 70:1). 2.09 g (3.99 mmol, 62%) of a light yellow foam was isolated. The spectra indicate the presence of two rotamers of which the major one is described. ¹H NMR (CDCl₃, 500 MHz): δ 1.13 (brs, 31, 1.24 (brs, 3H), 2.62 (m, 2H, 3.12 (m, 1H), 3.26 (brs, 21, 3.39 (m, 1H), 3.54 (brs, 2H), 3.76 (s, 31), 6.22 (s, 1H), 6.51 (d, J 2.0 Hz, 1H), 6.73 (dd, J 2.0, 9.0 Hz, 1H), 6.92 (d, J 9.0 Hz, 1H), 7.28 (m, 4H), 7.88 (d, J 9.0 Hz, 2H), 8.20 (d, J 9.0 Hz, 2H). (+) LRESIMS m/z 524 [M+1]⁺.

### INTERMEDIATE 6.2.1: N,N-DIETHYL-4-{6-HYDROXY-2-[(4-NITROPHENYL)SULFONYL]-1,2,3,4-TETRAHYDROISOQUINOLIN-1-YL}BENZAMIDE

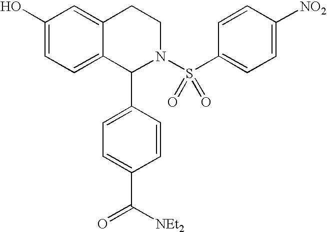

2.05 g (3.91 mmol) INTERMEDIATE 6.1.1 was dissolved in 100 mL DCM and cooled to −78 C. At this temperature, a solution of 0.57 mL (5.87 mmol, 1.5 eq) boron tribromide in 20 mL DCM was added dropwise. The solution was allowed to warm to room temperature and was stirred for another 60 min. TLC (DCM/MeOH 30:1) indicated mainly the presence of starting material. The reaction mixture was cooled to −78 C and another 1.14 mL (2 eq, neat) boron tribromide was added. The reaction mixture was allowed to warm to room temperature and stirred for 60 min. The mixture was poured onto crushed ice and extracted with DCM twice. The combined organic layers were washed with saturated aqueous sodium bicarbonate solution, water, and brine. After drying and evaporating the residue was purified by flash chromatography (DCM/MeOH 70:1, 90 g). 1.99 g (3.91 mmol, quant.) of a light yellow foam was isolated. The spectra indicate the presence of two rotamers of which the major one is described. ¹H NMR (CDCl₃, 500 MHz): δ 1.15 (brs, 3H), 1.27 (brs, 3H), 2.48 (m, 2H), 3.29 (brs, 2H), 3.32 (m, 1H), 3.57 (brs, 21), 3.79 (m, 1H), 5.32 (s, 1H), 6.39 (d, 2.0 Hz), 6.42 (dd, 2.0, 9.0 Hz, 1H), 6.73 (d, 9.0 Hz, 1H), 7.31 (m, 4H), 7.86 (d, 9.0 Hz, 2H), 8.18 (d, 9.0 Hz, 2H). (+) LRESIMS m/z 510 [M+1]⁺.

### INTERMEDIATE 6.3.1: 1-{4-[(DIETHYLAMINO)CARBONYL]PHENYL}-2-[(4-NITROPHENYL)SULFONYL]-1,2,3,4-TETRAHYDROISOQUINOLIN-6-YL METHANESULFONATE

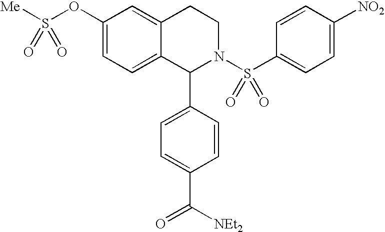

150 mg (0.294 mmol) Intermediate 6.2.1 and 153 ul (3 eq, 0.87 mmol) Hüig's base were dissolved in 5 mL dichloromethane. The solution was cooled to 0° C. and 45 ul (2 eq, 0.60 mmol) methanesulfonyl chloride was added. The reaction was allowed to warm to room temperature and stirred for 3 h after which TLC (DCM/MeOH 100:1) indicated complete consumption of the starting material. Crushed ice and more DCM were added. After separation of the layers the organic phase was washed with water and brine, dried, and evaporated. Flash chromatography (DCM to DCM/MeOH 100:1) gave 152 mg (0.259 mmol, 88%) of the product, which was directly used for the preparation of INTERMEDIATE 6.4.1.

### INTERMEDIATE 6.3.2: 1-{4-[(DIETHYLAMINO)CARBONYL]PHENYL}-2-[(4-NITROPHENYL)SULFONYL]-1,2,3,4-TETRAHYDROISOQUINOLIN-6-YL DIMETHYLSULFAMATE

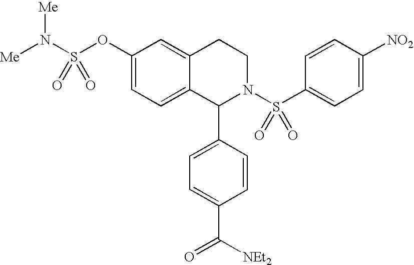

150 mg (0.29 mmol) INTERMEDIATE 6.2.1 and 153 ul (3 eq, 0.87 mmol) Hünig's base were dissolved in dichloromethane. The solution was cooled to 0° C. and 63 ul (2 eq, 0.58 mmol) dimethylsulfamoyl chloride was added. The reaction was allowed to warm to room temperature and stirred for 18 h. DCM and water were added and after phase separation the organic layer was washed with 1 M hydrochloric acid, sat. sodium bicarbonate solution, water and brine, dried, and evaporated. Flash chromatography (DCM to DCM/MeOH 30:1) of the residue yielded 116 mg (0.19 mmol, 66%) of the product, which contained traces of the starting material and which was directly used for the preparation of INTERMEDIATE 6.4.2.

### INTERMEDIATE 6.4.1: 1-{4-[(DIETHYLAMINO)CARBONYL]PHENYL}-1,2,3,4-TETRAHYDROISOQUINOLIN-6-YL METHANESULFONATE

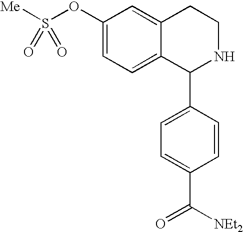

152 mg (0.259 mmol, 88%) INTERMEDIATE 6.3.1 was dissolved in 10 mL DMF and 43 mg (4 eq, 1.04 mmol) lithium hydroxide and 44 mg (1.5 eq, 0.389 mmol) mercaptoacetic acid as its sodium salt were added. The mixture was stirred at room temperature for 18 h. TLC indicated the presence of starting material next to the formation of a new spot that stained iodine (DCM/MeOH 30:1). The same amount of reagents was added and the mixture was stirred for another 3 h TLC and MS both indicated presence of starting material. Another 3 eq of both of the reagents were added and the reaction was stirred for another 18 h. TLC could still detect the starting material and another 5 eq of the mercaptoacetic acid was added. After stirring at room temperature for 3 h the volatiles were removed in vacuo. The residue was taken up in water and extracted twice with DCM. The combined organic layers were washed with water (3×) and brine, dried, and evaporated. Flash chromatography (DCM/MeOH 50:1 to 85:15) yielded 54 mg (0.134 mmol, 52%) of a clear gum. ¹H NMR (CDCl₃, 500 MHz): δ 1.12 (brs, 3H), 1.24 (brs, 3H), 2.86 (m, 2H), 3.10 (m, 1H), 3.14 (s, 3H), 3.27 (m, 3H), 3.58 (brs, 2H), 5.10 (s, 1H), 6.77 (d, J 9.0 Hz, 1H), 6.95 (dd, J 2.0, 9.0 Hz, 1H), 7.09 (d, 2.0 Hz, 1H), 7.30 (m, 4H). (+) LRESIMS m/z 403 [M+1]⁺.

### INTERMEDIATE 6.4.2: 1-{4-[(DIETHYLAMINO)CARBONYL]PHENYL}-1,2,3,4-TETRAHYDROISOQUINOLIN-6-YL DIMETHYLSULFAMATE

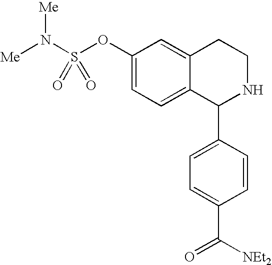

116 mg (0.19 mmol) INTERMEDIATE 6.3.2 was dissolved in 10 mL DMF and 32 mg (4 eq, 0.76 mmol) lithium hydroxide and 33 mg (1.5 eq, 0.29 mmol) mercaptoacetic acid, sodium salt were added. The mixture was stirred at room temperature for 18 h. TLC indicated the presence of starting material next to the formation of a new spot that stained iodine (DCM/MeOH 30:1). The same amount of reagents was added and the mixture was stirred for another 3 h. TLC and MS both indicated presence of starting material. Another 3 eq of both of the reagents was added and the reaction was stirred for another 18 h. Another 5 eq of the mercaptoacetic acid was added. After stirring at room temperature for 3 h the volatiles were removed in vacuo. The residue was taken up in water and extracted twice with DCM. The combined organic layers were washed with water (3×) and brine, dried and evaporated. Flash chromatography (DCM/MeOH 50:1 to 85:15) yielded 24 mg (0.056 mmol, 29%) of a clear gum. ¹H NMR (CDCl₃, 500 MHz): δ 1.13 (brs, 3H), 1.25 (brs, 3H), 2.88 (m, 1H), 2.99 (s, 6H), 3.26 (m, 2H), 3.28 (m, 3H), 3.59 (brs, 2H), 5.11 (s, 1H), 6.76 (d, J 9.0 Hz, 1H), 6.97 (dd, J 2.0, 9.0 Hz, 1H), 7.11 (d, 9.0 Hz, 1H), 7.34 (m, 4H). (+) LRESIMS m/z 432 [M+1]⁺.

### INTERMEDIATE 7.1.1: 4-(7-ETHOXY-6-METHOXY-1,2,3,4-TETRAHYDROISOQUINOLIN-1-YL)-N,N-DIETHYLBENZAMIDE

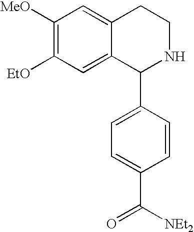

To a solution of triphenylphosphine (2 eq, 0.112 mmol, 29.3 mg) in anhydrous dichloromethane (0.3 mL) at 0° C. was added diisopropylazodicarboxylate (DIAD, 2 eq, 0.112 mmol, 22.6 mL, 22 uL). After stirring at 0° C. for 5 min a solution of ethanol (2 eq, 0.112 mmol, 5.1 mg, 6.5 uL) and INTERMEDIATE 5.1.12 (1 eq, 0.056 mmol, 20 mg) in anhydrous dichloromethane (1 mL) was added. The reaction mixture was allowed to stir at RT for 6 h then ethanol/water was added and extracted to EtOAc, dried over MgSO₄ and concentrated to dryness. The product was purified by flash chromatography, using silica column and DCM/MeOH (100:5) to give 13 mg (0.034 mmol, 61%) of INTERMEDIATE 7.1.1 as oil. ¹H NMR (500 MHz, CDCl₃) δ 1.15 (br s, 3H), 1.28 (br s, 3H), 1.35 (t, J 7 Hz, 3H), 1.45 (m, 1H), 2.85-3.15-3.28 (m, 4H), 3.30 (br s, 2H), 3.55 (br s, 2H), 3.90 (s, 3H), 3.95 (q, J 7 Hz, 2H), 5.20 (s, 1H), 6.25 (s, 1H), 6.68 (s, 1H), 7.35 (s, 4H). ¹³C NMR (125 MHz, CDCl₃) δ 12.35, 13.40, 15.50, 28.10, 40.00, 42.05, 43.10, 56.05, 60.10, 64.50, 112.30, 113.00, 127.50, 129.50, 130.00, 137.80, 143.90, 147.00, 149.00, 171.10. (+) LRESIMS m/z 383 (M+H)⁺.

### INTERMEDIATE 7.1.2: N,N-DIETHYL-4-(7-ISOPROPOXY-6-METHOXY-1,2,3,4-TETRAHYDROISOQUINOLIN-1-YL)BENZAMIDE

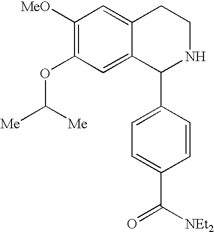

To a solution of triphenylphosphine (133 mg, 0.5085 mmol) in anhydrous dichloromethane (1 mL) at 0° C. was added diisopropylazodicarboxylate (102 mg, 0.1 mL, 0.584 mmol). After stirring at 0° C. for 5 min a solution of isopropanol (39 uL, 30.6 mg, 0.5102 mmol) and INTERMEDIATE 5.1.12 (70 mg, 0.1977 mmol) in anhydrous dichloromethane (1 mL) was added. The reaction mixture was allowed to stir at RT for 6 hr then water was added and extracted to EtOAc, dried over MgSO₄ and concentrated to dryness. The product was purified by flash chromatography, using silica column and DCM/MeOH (100:5) to give 48 mg (0.121 mmol, 61%) of INTERMEDIATE 7.1.2 as a white solid. ¹H NMR (500 MHz, CDCl₃) δ 1.10 (br s, 3H), 1.18 (d, J 7 Hz, 3H), 1.23 (d, J 7 Hz, 3H), 2.25 (br s, 3H), 2.44 (br s, 1H), 2.77-2.91 (m, 2H), 3.12-3.20 (m, 2H), 3.23 (br s, 2H), 3.58 (br s, 2H), 3.85 (s, 3H), 3.85 (s, 3H), 4.25 (q, J 7 Hz, 1H), 5.06 (s, 1H), 6.26 (s, 1H), 6.26 (s, 1H), 6.64 (s, 1H), 7.28-7.34 (m, 4H). ¹³C NMR (125 MHz, CDCl₃) δ 13.14, 14.41, 22.06, 22.15, 29.32, 39.51, 42.16, 43.48, 56.18, 61.25, 71.72, 112.48, 116.34, 126.69, 128.41, 129.24, 136.62, 145.50, 145.84, 149.63, 171.40. (+) LRESIMS m/z 397 (M+H)⁺.

### INTERMEDIATE 7.1.3: N,N-DIETHYL-4-[6-METHOXY-7-(2-MORPHOLIN-4-YLETHOXY)-1,2,3,4-TETRAHYDROISOQUINOLIN-1-YL]BENZAMIDE

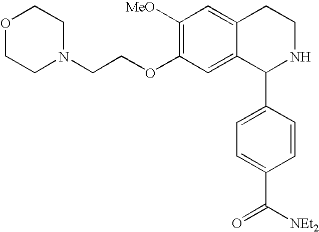

To a solution of triphenylphosphine (133 mg, 0.5085 mmol) in anhydrous dichloromethane (1 mL) at 0° C. was added diisopropylazodicarboxylate (100 mg, 100 uL, 0.5084 mmol). After stirring at 0° C. for 5 min a solution of N-morpholinoethanol (0.071 mL, 76 mg, 0.580 mmol) and INTERMEDIATE 5.1.12 (70 mg, 0.1977 mmol) in anhydrous dichloromethane (1 mL) was added. The reaction mixture was allowed to stir at RT for 20 hr then water was added and extracted to EtOAc, dried over MgSO₄ and concentrated to dryness. The product was purified by flash chromatography, using silica column and DCM/MeOH (100:5) to give 52 mg (0.111 mmol, 56%) of INTERMEDIATE 7.1.3 as light yellow oil. ¹H NMR (500 MHz, CDCl₃) δ 1.13 (br s, 3H), 1.25 (br s, 3H), 2.52 (br s, 4H), 2.73 (m, 2H), 2.8-3.1-3.2 (m, 4H), 3.30 (br s, 2H), 3.50 (br s, 2H), 3.69 (br s, 4H), 3.86 (s, 3H), 3.91 (m, 2H), 5.14 (s, 1H), 6.29 (s, 1H), 6.66 (s, 1H), 7.28-7.36 (m, 4H). ¹³C NMR (125 MHz, CDCl₃) δ 13.11, 14.47, 28.72, 39.57, 41.62, 43.55, 54.24, 56.18, 57.71, 60.83, 67.09, 112.31, 113.81, 126.84, 128.21, 129.53, 136.98, 144.50, 146.82, 148.96, 171.25. (+) LRESIMS m/z 468 (M+H)⁺.

### INTERMEDIATE 7.1.4: N,N-DIETHYL-4-[6-METHOXY-7-(NEOPENTYLOXY)-1,2,3,4-TETRAHYDROISOQUINOLIN-1-YL]-BENZAMIDE

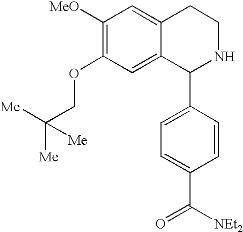

To a solution of INTERMEDIATE 5.1.12 (70.8 mg, 0.2 mmol), triphenylphosphine (62.8 mg, 0.24 mmol, 1.2 eq), neopentyl alcohol (21.12 mg, 0.24 mmol, 1.2 eq) in anhydrous toluene (0.14 mL) was added diisopropyldiazodicarboxylate (DIAD, 48 mg, 0.24 mmol, 1.2 eq). The reaction mixture was sealed for microwave reaction. Microwave was set to 100° C. for 0.5 hr. After cooling down to room temperature, the reaction vessel was removed from microwave and evaporating solvent to dryness. Product was purified by flash chromatography to afford 41 mg (0.0966 mmol, 74%) and recovered 25 mg (0.0706 mmol) of INTERMEDIATE 5.1.12. ¹H NMR (500 MHz, CD₂Cl₂): δ 0.99 (s, 9H), 1.15 (br s, 3H), 1.23 (br s, 3H), 1.95 (m, 1H), 2.74 (m, 1H), 2.88 (m, 1H), 3.03 (m, 1H), 3.19 (m, 1H), 3.29 (br s, 2H), 3.40 (d, J 10 Hz, 1H), 3.46 (d, J 10 Hz, 1H), 3.53 (br s, 2H), 3.86 (s, 3H), 5.06 (s, 1H), 6.20 (s, 1H), 6.70 (s, 1H), 7.30-7.34 (m, 4H). ¹³C NMR (125 MHz, CD₂Cl₂): δ 12.90, 14.20, 26.54 (3C), 29.51, 32.15, 39.45, 41.93, 43.44, 56.52, 61.23, 79.50, 113.30, 113.83, 126.42 (2C), 128.35, 129.08 (2C), 130.22, 136.65, 146.56, 147.84, 148.80, 171.06. (+) LRESIMS m/z 425 [M+H]⁺.

### INTERMEDIATE 7.1.5: 4-[7-(CYCLOBUTYLOXY)-6-METHOXY-1,2,3,4-TETRAHYDROISOQUINOLIN-1-YL]-N,N-DIETHYLBENZAMIDE

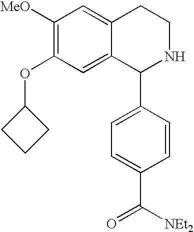

To a solution of INTERMEDIATE 5.1.12 (70.8 mg, 0.2 mmol), triphenylphosphine (62.8 mg, 0.24 mmol, 1.2 eq), cyclobutyl alcohol (21.6 mg, 0.30 mmol, 1.5 eq) in anhydrous toluene (0.14 mL) was added diisopropyldiazodicarboxylate (DIAD, 48 mg, 0.24 mmol, 1.2 eq). The reaction mixture was sealed for microwave reaction. Microwave was set to 100° C. for 0.5 hr. After cooling down to room temperature, the reaction vessel was removed from microwave and evaporating solvent to dryness. Product was purified by flash chromatography to afford 38 mg (0.093 mmol, 71%) of INTERMEDIATE 7.1.5 as colorless oil and recovered 25 mg (0.0706 mmol) of INTERMEDIATE 5.1.12. ¹H NMR (500 mHz, CD₂Cl₂): δ 1.13 (br s, 3H), 1.23 (br, 3H), 1.54 (m, 1H), 1.75 (m, 1H), 1.95 (m, 1H), 2.03 (br m, 2), 2.29 (br s, 1H), 2.74 (m, 1H), 2.94 (m, 1H), 3.06 (m, 1H), 3.23 (m, 1H), 3.24 (br s, 2H), 3.53 (br s, 2H), 3.84 (s, 3H), 5.04 (s, 1H), 6.10 (s, 1H), 6.67 (s, 1H), 7.39 (br s, 4H). ¹³C NMR (125 MHz, CD₂Cl₂): δ 13.20, 14.22, 29.49, 30.62, 30.75, 39.90, 42.57, 43.48, 55.98, 61.55, 72.11, 112.29, 113.49, 126.41, 128.63, 129.08, 130.25, 136.75, 145.05, 146.52, 148.18, 171.06. (+) LRESIMS m/z 409 [M+H]⁺.

### INTERMEDIATE 8.1.1: N,N-DIETHYL-4-[7-HYDROXY-6-METHOXY-2-(TRIFLUOROACETYL-1,2,3,4-TETRAHYDROISOQUINOLIN-1-YL]BENZAMIDE

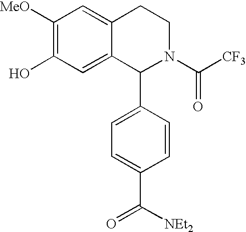

0.54 g (1.56 mmol) INTERMEDIATE 5.1.12 was refluxed in 25 mL TFAA for 18 h. After cooling down to room temperature the solution was evaporated in vacuo and purified by flash chromatography (DCM/methanol 100:1) to yield 0.67 g (1.49 mmol, 96%) of the desired product. ¹H NMR (500 MHz, CDCl₃): δ 1.17, 1.29 (2 brs, 6H), 2.79, 3.05, 3.42 (3 m, 3H), 3.31, 3.59 (2 brs, 4H), 3.97 (s, 3H, 3.96 (m, 1H), 6.61, 6.69, 6.74 (3 s, 3H), 7.31 (m, 4H).

### INTERMEDIATE 8.2.1: 1-{4-[(DIETHYLAMINO)CARBONYL]PHENYL}-6-METHOXY-2-(TRIFLUOROACETYL)-1,2,3,4-TETRAHYDROISOQUINOLIN-7-YL DIMETHYLSULFAMATE

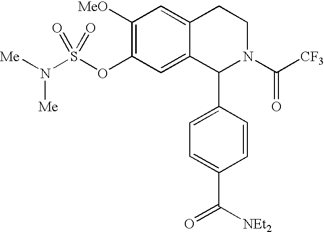

To a solution of INTERMEDIATE 8.1.1 (150 mg, 0.33 mmol) and triethylamine (71 ul, 0.50 mmol, 1.5 eq) in DCM (5 mL) was added dimethylsulfamoyl chloride (50 ul, 0.47 mmol, 1.4 eq) at 0 C. The solution was stirred at room temperature for 4 h. TLC (DCM/methanol 100:1) indicated the presence of starting material. Another 10 eq of both reagents was added and stirring continued for another 18 h. DCM and water were added and the organic phase was washed with brine, dried, and evaporated Flash chromatography yielded a white foam (210 mg, 0.33 mmol, quant.). ¹H NMR (500 MHz, CDCl₃): δ 1.05, 1.19 (2 brs, 6H), 2.78 (s, 3H), 2.90 (s, 3H), 2.91 (m, 1H), 3.01-3.15 (m, 2H), 3.21 (brs, 2H), 3.38 (m, 1H), 3.70 (brs, 2H), 3.88 (s, 3H), 6.73 (s, 1H), 6.80 (s, 1H), 7.00 (s, 1H), 7.27 (m, 4H).

### INTERMEDIATE 8.3.1: 1-{4-[(DIETHYLAMINO)CARBONYL]PHENYL}-6-METHOXY-1,2,3,4-TETRAHYDROISOQUINOLIN-7-YL DIMETHYLSULFAMATE

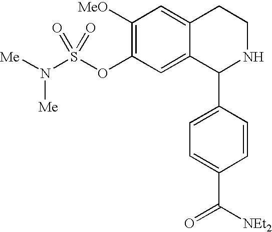

INTERMEDIATE 8.2.1 (210 mg, 0.33 mmol) was dissolved in methanol (2 mL) and water (2 mL) and potassium carbonate (100 mg, 0.72 mmol) added. The reaction was stirred for 6 h. Silica gel was added and the volatiles were removed in vacuo. Flash chromatography of the residue yielded the desired product (106 mg, 0.23 mmol, 70%). ¹H NMR (500 MHz, CDCl₃): 1.09, 1.23 (2 brs, 6H), 2.79, 2.85 (2 s, 6H), 3.00 (m, 2H), 3.21 (m, 4H), 3.52 (brs, 2H), 3.86 (s, 3H), 5.15 (s, 1H), 6.68 (s, 1H), 6.72 (s, 1H), 7.28 (m, 4H). (+) LRESIMS m/z 462 [M+H]⁺.

### INTERMEDIATE 9.1.1: N,N-DIETHYL-4-[6-METHOXY-5-NITRO-2-(TRIFLUOROACETYL)-1,2,3,4-TETRAHYDROISOQUINOLIN-1-YL]BENZAMIDE

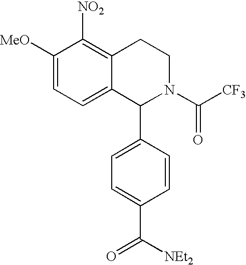

A solution of INTERMEDIATE 5.1.8 (11.09 g, 32.8 mmole) in trifluoroacetic anhydride (100 mL) was heated at reflux until all the amine dissolved (˜1 h). The reaction mixture was cooled to 0° C. and copper(II) nitrate (3.72 g, 19.8 mmole) added in one portion. The resulting reaction mixture was allowed to warm to room temperature over 3 h after which the excess trifluoroacetic anhydride was removed in vacuo and ice water (100 mL) added to the residue. The aqueous phase was extracted with dichloromethane (3×100 mL) and the combined organic extracts were washed with water (100 mL), dried (MgSO₄) and filtered. The solvent was removed in vacuo and the residue purified by flash chromatography (ethyl acetate/hexane, 40/60) to give INTERMEDIATE 9.1.1 (4.51 g, 29%) as an off-white solid; ¹H NMR (500 MHz, CDCl₃): δ 1.13, 1.23 (2 br s, 6H), 2.81 (dd, J 2, 17.5 Hz, 1H), 3.05 (m, 1H), 3.26 (br s, 2H), 3.43 (m, 1H), 3.54 (br s, 2H), 3.92 (s, 3H), 4.00 (dd, J 5, 14 Hz, 1H), 6.83 (s, 1H), 6.99 (d, J 9 Hz, 1H), 7.15 (d, J 9 Hz, 1H), 7.23 (d, J 8.5 Hz, 2H), 7.34 (d, J 8.5 Hz, 2H); (+) LRESIMS m/z 480 [M+H]⁺ (100).

### INTERMEDIATE 9.1.2: N,N-DIETHYL-4-[6-METHOXY-7-NITRO-2-(TRIFLUOROACETYL)-1,2,3,4-TETRAHYDROISOQUINOLIN-1-YL]BENZAMIDE

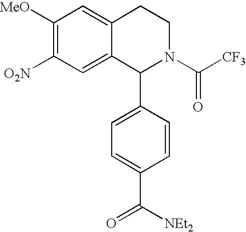

A solution of INTERMEDIATE 5.1.8 (11.09 g, 32.8 mmole) in trifluoroacetic anhydride (100 mL) was heated at reflux until all the amine dissolved (˜1 h). The reaction mixture was cooled to 0° C. and copper(II) nitrate (3.72 g, 19.8 mmole) added in one portion. The resulting reaction mixture was allowed to warm to room temperature over 3 h after which the excess trifluoroacetic anhydride was removed in vacuo and ice water (100 mL) added to the residue. The aqueous phase was extracted with dichloromethane (3×100 mL) and the combined organic extracts were washed with water (100 mL), dried (MgSO₄) and filtered. The solvent was removed in vacuo and the residue purified by flash chromatography (ethyl acetate/hexane, 60/40) to give INTERMEDIATE 9.1.2 (4.61 g, 29%) as an off-white foam. ¹H NMR (500 MHz, CDCl₃): δ 1.13, 1.24 (2 br s, 6H), 2.97 (dd, J 2, 17 Hz, 1H), 3.15 (m, 1H), 3.27 (br s, 2H), 3.47 (m, 1H), 3.54 (br s, 2H), 3.99 (s, 4H), 6.84 (s, 1H), 6.94 (s, 1H), 7.22 (d, J 8 Hz, 2H), 7.34 (d, J 8 Hz, 2H), 7.64 (s, 1H); (+) LRESIMS m/z 480 [M+H]⁺.

### INTERMEDIATE 9.2.1: N,N-DIETHYL-4-(6-METHOXY-5-NITRO-1,2,3,4-TETRAHYDROISOQUINOLIN-1-YL)BENZAMIDE

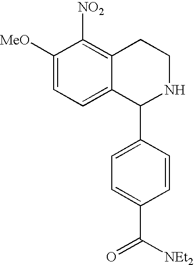

A solution of INTERMEDIATE 9.1.1 (1.97 g, 4.10 mmole) and potassium carbonate (ca. 2 g) in methanol (27 mL), tetrahydrofuran (10 mL) and water (3 mL) was heated at reflux for 4 h. The reaction mixture was cooled to room temperature and water (100 mL) and chloroform (150 mL) added and the mixture filtered through a Whatman 1PS filter paper. The solvent was removed from the organic phase in vacuo and the residue purified by flash chromatography (acetone/hexane, 4/6) to give INTERMEDIATE 9.2.1 (0.38 g, 24%) as a yellow solid; ¹H NMR (500 MHz, CDCl₃): δ 1.10, 1.22 (2 br s, 6H), 2.61 (br s, 1H), 2.69 (dt, J 4, 17 Hz, 1H), 2.92 (m, 1H), 3.05 (m, 1H), 3.24 (m, 3H), 3.52 (br s, 2H), 3.82 (s, 3H), 5.06 (s, 1H), 6.75 (d, J 8.5 Hz, 1H), 6.79 (d, J 8.5 Hz, 1H), 7.28 (d, J 8 Hz, 2H), 7.33 (d, J 8 Hz, 2H); ¹³C NMR (125 MHz, CDCl₃): δ 12.74, 14.07, 24.48, 39.17, 41.01, 43.17, 56.22, 60.89, 110.15, 126.52, 128.37, 128.88, 130.55, 130.88, 136.73, 141.05, 144.43, 149.07, 170.80; (+) LRESIMS m/z 384 [M+H]⁺.

### INTERMEDIATE 9.2.2: N,N-DIETHYL-4-(6-METHOXY-7-NITRO-1,2,3,4-TETRAHYDROISOQUINOLIN-1-YL)BENZAMIDE

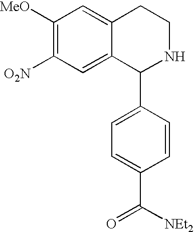

To a solution of INTERMEDIATE 9.1.2 (1.07 g, 2.2 mmole) in methanol (30 mL), tetrahydrofuran (30 mL) and water (15 mL) was added lithium hydroxide monohydrate (0.0.92 g, 22.0 mmole) and the resulting reaction mixture was stirred at room temperature for 18 h. The volatiles were removed in vacuo, water (60 mL), chloroform (50 mL) added and the mixture filtered through a 1PS filter paper. The solvent was removed from the organic phase and the residue purified by flash chromatography (ethyl acetate/chloroform/methanol, 50/45/5) to give INTERMEDIATE 9.2.2 (0.48 g, 56%) as a yellow solid; ¹H NMR (500 MHz, CDCl₃): δ 1.08, 1.19 (2 br s, 6H), 2.32 (br s, 1H), 2.83, 3.03, 3.22, 3.50 (m, 8H), 3.89 (s, 3H), 5.02 (s, 1H), 6.83 (s, 1H), 7.23 (s, 1H), 7.25 (d, J 8 Hz, 2H), 7.31 (d, J 8 Hz, 2H); ¹³C NMR (125 MHz, CDCl₃): δ 12.68, 14.03, 29.91, 39.10, 43.15, 41.35, 56.32, 60.63, 113.52, 125.04, 126.55, 128.64, 130.14, 136.67, 137.37, 142.91, 144.23, 151.20, 170.68. (+) LRESIMS m/z 384 [M+H]⁺ (100).

### INTERMEDIATE 10.1.1: TERT-BUTYL 1-{4-[(DIETHYLAMINO)CARBONYL]PHENYL}-7-HYDROXY-6-METHOXY-3,4-DIHYDROISOQUINOLINE-2(1H)-CARBOXYLATE

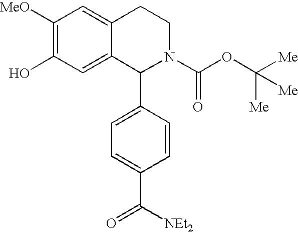

To a solution of INTERMEDIATE 5.1.12 (420 mg, 1.186 mmol) in anhydrous methanol (15 mL) was added di-tert-butyl dicarbonate (250 mg) and followed by triethyl amine (150 uL). The reaction mixture was stirred at room temperature for 2.5 hr, then quenched with water (5 mL) and extracted with ethyl acetate (3×30 mL). The extracted ethyl acetate was washed with 0.1% HCl, brine and dried over MgSO₄, then was concentrated to give INTERMEDIATE 10.1.1 (532 mg, 1.171 mmol, 99%) as white solid. ¹H NMR (500 MHz, CDCl₃) δ 1.16 (br s, 6H), 1.49 (s, 9H), 2.68 (m, 2H), 2.91 (br m, 2H), 3.89 (s, 3H), 6.02 (br s, 1H), 6.59 (br s, 1H), 6.65 (s, 1H), 7.24-7.28 (m, 4H). ¹³C NMR (125 MHz, CDCl₃) δ 13.16, 14.23, 28.76, 38.06, 43.49, 56.18, 57.00, 66.80, 80.37, 111.00, 114.40, 126.43, 126.87, 127.89, 128.49, 136.25, 144.31, 144.41, 146.13, 154.90, 171.54. (+) LRESIMS m/z 455 (M+H)⁺.

### INTERMEDIATE 10.2.1: TERT-BUTYL 1-{4-[(DIETHYLAMINO)CARBONYL]PHENYL}-6-METHOXY-7-PHENOXY-3,4-DIHYDROISOQUINOLINE-2(1H)-CARBOXYLATE

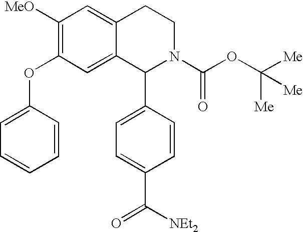

To a solution of INTERMEDIATE 10.1.1 (114 mg, 0.251 mmol) in anhydrous dichloromethane (3 mL) were added phenylboronic acid (3 eq, 0.753 mmol, 91 mg) and copper (II) acetate (3 eq, 0.753 mmol, 136 mg) and followed by triethyl amine (4 eq, 1.004 mmol, 101.4 mg, 138 uL). The reaction mixture was stirred at RT for 24 hr then filtered through a celite layer. Product was purified by flash chromatography to give INTERMEDIATE 10.2.1 (60 mg, 0.113 mmol, 65%) as colourless oil, and the starting material was recovered (36 mg, 0.079 mmol). ¹H NMR (500 MHz, CDCl₃) δ 1.18 (br s, 6H), 1.52 (s, 9H), 2.75-3.18 (br m, 4H), 2.29 (br s, 2H), 2.58 (br s, 2H), 3.85 (s, 3H), 3.90 (s, 1H), 6.70 (br s, 1H), 6.80 (s, 1H), 6.90-7.30 (m, 9H). ¹³C NMR (125 MHz, CDCl₃) δ 13.10, 15.10, 28.42, 28.76, 38.00, 39.50, 43.10, 56.00, 57.90, 80.10 (3C), 113.10, 116.95, 121.39, 122.55, 126.47 (2C), 127.85, 128.43, 129.70 (2C), 132.08, 136.44, 143.33, 144.12, 150.82, 158.29, 171.31. (+) LRESIMS m/z 531 (M+H)⁺.

### INTERMEDIATE 10.2.2: TERT-BUTYL 1-{4-[(DIETHYLAMINO)CARBONYL]PHENYL}-7-(4-FLUOROPHENOXY)-6-METHOXY-3,4-DIHYDROISOQUINOLINE-2(1H)-CARBOXYLATE

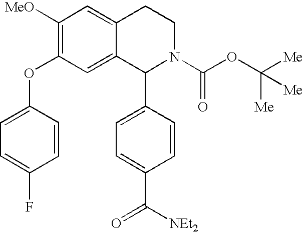

To a solution of INTERMEDIATE 10.1.1 (114 mg, 0.251 mmol) in anhydrous dichloromethane (3 mL) were added 4-fluorophenylboronic acid (3 eq, 0.0753 mmol, 105 mg), copper(II) acetate (3 eq, 0.753 mmol, 136 mg) and triethyl amine (4 eq, 1.004 mmol, 101.4 mg, 138 uL). The reaction mixture was stirred at room temperature overnight then concentrated. Product was purified by flash chromatography using FlashTube™ 2008 with 5% MeOH/DCM to give 21 mg (0.038 mmol, 17%) of INTERMEDIATE 10.2.2 and starting material was recovered 10 mg (0.028 mmol). ¹H NMR (500 MHz, CDCl₃) δ 1.18 (br s, 6H), 1.46 (s, 9H), 2.68-3.18 (br m, 4H), 3.30 (br s, 2H), 3.50 (br s, 2H), 3.89 (s, 3H), 4.10 (s, 1H), 6.58 (s, 1H), 6.80 (s, 1H), 6.65-7.30 (m, 8H). ¹³C NMR (125 MHz, CDCl₃) δ 13.00, 14.10, 28.00, 28.10, 39.00, 39.50, 40.45, 56.10, 57.20, 80.10, 111.03, 113.10, 116.12 (d, J 23 Hz), 118.30 (d, J 8 Hz), 120.86, 127.85, 128.10, 132.07, 136.26, 136.47, 144.36, 146.18, 154.14, 154.85, 158.42 (d, J 240 Hz), 171.39. (+) LRESIMS m/z 549 (M+H)⁺.

### INTERMEDIATE 10.2.3: TERT-BUTYL 1-{4-[(DIETHYLAMINO)CARBONYL]PHENYL}-6-METHOXY-7-(4-METHOXYPHENOXY)-3,4-DIHYDROISOQUINOLINE-2(1H)-CARBOXYLATE

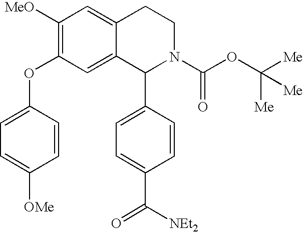

To a solution of INTERMEDIATE 10.1.1 (50 mg, 0.110 mmol) in anhydrous dichloromethane (2.5 mL) was added copper(II) acetate (40 mg, 0.189 mmol, 1.7 eq), 4-methoxyphenyl boronic acid (33 mg, 0.220 mmol, 2 eq), molecular sieves (40 mg, 4 A) and triethylamine (24.2 mg, 0.22 mmol, 2 eq). The reaction mixture was stirred at room temperature for overnight and then filtered through a silica layer and wash the silica with a solution of methanol/dichloromethane (1:99, 20 mL). After evaporation of solvent, the residue was purified by flash chromatography on silica column to afford INTERMEDIATE 10.2.3 (38 mg, 0.068 mmol, 61%). ¹H NMR (500 MHz, CDCl₃): δ 1.10 (br s, 3H), 1.20 (br s, 3H), 1.50 (s, 6H), 1.53 (s, 3H), 2.71 (br m, 1H), 2.82 (br m, 1H), 2.82 (nr m, 1H), 3.15 (br m, 1H), 3.28 (br m, 1H), 3.29 (br s, 2H), 3.52 (br s, 2H), 3.83 (s, 6H), 6.59 (s, 1H), 6.60 (s, 1H), 6.80-7.30 (m, 8H). (+) LRESIMS m/z 561 [M+H]⁺.

### INTERMEDIATE 10.2.4: TERT-BUTYL 1-{4-[(DIETHYLAMINO)CARBONYL]PHENYL}-6-METHOXY-7-(PYRIDIN-3-YLOXY)-3,4-DIHYDROISOQUINOLINE-2(1H)-CARBOXYLATE

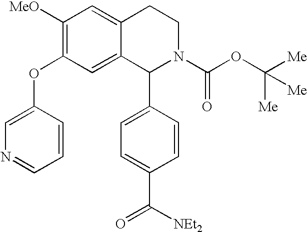

To a solution of INTERMEDIATE 10.1.1 (50 mg, 0.110 mmol) in anhydrous dichloromethane (2.5 mL) were added copper(II) acetate (40 mg, 0.189 mmol, 1.7 eq), 3-pyridyl boronic acid (27 mg, 0.220 mmol, 2 eq), molecular sieves (40 mg, 4 A) and triethylamine (24.2 mg, 0.22 mmol, 2 eq). The reaction mixture was stirred at room temperature for overnight and then filtered through a silica layer and wash the silica with a solution of methanol/dichloromethane (1:99, 20 mL). After evaporation of solvent, the residue was purified by flash chromatography on silica column to afford INTERMEDIATE 10.2.4 (52 mg, 0.097 mmol, 89%). ¹H NMR (500 MHz, CDCl₃): δ 1.10 (br s, 3H), 1.21 (br, 3H), 2.70 (br m, 1H), 2.95 (br m, 1H), 3.15 (br m, 1H), 3.28 (br m, 2H), 3.55 (br m, 2H), 3.85 (s, 3H), 6.60 (s, 1H), 6.63 (s, 1H), 6.78-7.31 (br m, 8H). (+) LRESIMS m/z 532 [M+H]⁺.

### INTERMEDIATE 11.1.1: 1-{4-[(DIETHYLAMINO)CARBONYL]PHENYL}-6-METHOXY-1,2,3,4-TETRAHYDROISOQUINOLIN-7-YL METHANESULFONATE

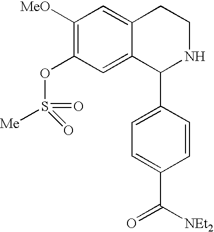

Methylsulfonyl chloride (43 ul, 0.55 mmol) was added to a solution of INTERMEDIATE 10.1.1 (0.13 g, 0.28 mmol) and Hünig's base (145 ul, 0.80 mmol) in DCM (5 mL) at 0 C. The mixture was allowed to warm to room temperature and stirred for 18 h. The reaction mixture was poured onto crushed ice and extracted with DCM. The organic phase was washed with sat. aqueous sodium bicarbonate solution, water, and brine, dried, and evaporated. The crude product was dissolved in 4 M hydrochloric acid in 1,4-dioxane (2.5 mL) and stirred for 1 h. Water was added. The mixture was basified and extracted with DCM (3×). The combined organic layers were washed with water and brine, dried, and evaporated. Flash chromatography of the residue yielded the compound as a white foam (38 mg, 0.09 mmol, 31%). ¹H NMR (500 MHz, CDCl₃): 1.12, 1.23 (2 brm, 6H), 2.81 (m, 1H), 3.05 (m, 2H), 3.15 (s, 3H), 3.24 (m, 3H), 3.58 (brs, 2H), 3.70 (s, 3H), 5.18 (s, 1H), 6.68 (s, 1H), 6.78 (s, 1H), 7.35 (m, 4H). (+) LRESIMS m/z). 433 (100).

### COMPOUND 12.1.1: N,N-DIETHYL-2-{[2-(2-FURYLMETHYL)-6,7-DIMETHOXY-1,2,3,4-TETRAHYDROISOQUINOLIN-1-YL]METHOXY}BENZAMIDE

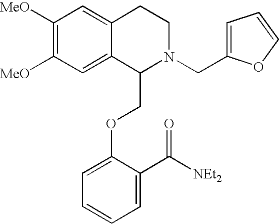

The compound was prepared on an Argonaut Quest 210 synthesiser. To a solution of INTERMEDIATE 5.1.3 (50 mg, 0.125 mmol) in 1,2-dichloroethane (0.5 mL) 2-furaldehyde (12 ul, 0.14 mmol) was added. After agitating the solution for 15 min, sodium triacetoxyborohydride (40 mg, 0.19 mmol) was added to each reaction vessel and the agitation continued over night. 1 M aqueous sodium hydroxide solution (1 mL) was added, and after phase separation the organic phase was dried with sodium sulphate. The aqueous layer was extracted another two times with dichloromethane, which was passed through the pad of sodium sulphate, and the combined organic layers were concentrated in vacuo. The crude product was purified by flash chromatography to give the desired product (18 mg, 0.038 mmol, 30%). ¹H NMR (500 MHz, CDCl₃): δ0.90 (brs, 3H), 1.18 (m, 3H), 2.53-3.18 (m, 6H), 3.38-3.55 (m, 2H), 3.78 (s, 61), 4.00-4.32 (m, 5H), 6.19, 6.22 (2 s, 2H), 6.50, 6.63 (2 s, 2H), 6.80 (d, J 6 Hz, 1H), 6.90 (t, J 6 Hz, 1H), 7.11 (d, J 6 Hz, 1H), 7.20 (m, 2H) (+) LRESIMS m/z 479 [M+H]⁺.

### COMPOUND 12.1.2: 2-{[6,7-DIMETHOXY-2-(THIEN-3-YLMETHYL)-1,2,3,4-TETRAHYDROISOQUINOLIN-1-YL]METHOXY}-N,N-DIETHYLBENZAMIDE

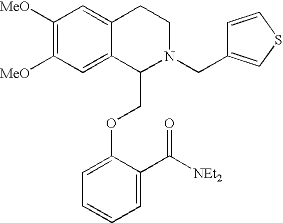

The compound was prepared on an Argonaut Quest 210 synthesiser. To a solution of INTERMEDIATE 5.1.3 (50 mg, 0.125 mmol) in 1,2-dichloroethane (0.5 mL) 3-thiophencarbaldehyde (13 ul, 0.14 mmol) was added. After agitating the solution for 15 min, sodium triacetoxyborohydride (40 mg, 0.19 mmol) was added to each reaction vessel and the agitation continued over night. 1 M aqueous sodium hydroxide solution (1 mL) was added, and after phase separation the organic phase was dried with sodium sulphate. The aqueous layer was extracted another two times with dichloromethane, which was passed through the pad of sodium sulphate, and the combined organic layers were concentrated in vacuo. The crude product was purified by flash chromatography to give the desired product (15 mg, 0.030 mmol, 24%). ¹H NMR (500 MHz, CDCl₃): δ0.92 (brs, 3H), 1.22 (m, 3H), 2.53-3.18 (m, 6H), 3.38-3.0 (m, 2H), 3.78, 3.79 (2 s, 6H), 3.92 (m, 1H), 3.99-4.23 (m, 4H), 6.50, 6.62 (2 s, 2H), 6.78 (d, J 6 Hz, 1H), 6.90 (t, J 6 Hz, 1H), 7.05 (brs, 1H), 7.12, 7.20 (m, 4H). (+) LRESIMS m/z 495 [M+H]⁺.

### COMPOUND 12.1.3: N,N-DIETHYL-3-{[2-(2-FURYLMETHYL)-6,7-DIMETHOXY-1,2,3,4-TETRAHYDROISOQUINOLIN-1-YL]METHOXY}BENZAMIDE

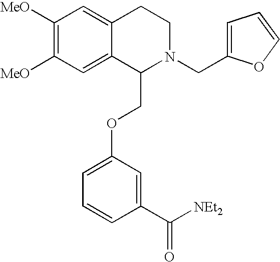

The compound was prepared on an Argonaut Quest 210 synthesiser. To a solution of INTERMEDIATE 5.1.2 (50 mg, 0.125 mmol) in 1,2-dichloroethane (0.5 mL) 2-furaldehyde (12 ul, 0.14 mmol) was added. After agitating the solution for 15 min. sodium triacetoxyborohydride (40 mg, 0.19 mmol) was added to each reaction vessel and the agitation continued over night. 1 M aqueous sodium hydroxide solution (1 mL) was added, and after phase separation the organic phase was dried with sodium sulphate. The aqueous layer was extracted another two times with dichloromethane, which was passed through the pad of sodium sulphate, and the combined organic layers were concentrated in vacuo. The crude product was purified by flash chromatography to give the desired product (28 mg, 0.058 mmol, 47%). ¹H NMR (500 MHz, CDCl₃): δ1.12, 1.23 (2 brs, 6H), 2.62 (m, 1H), 2.83-2.99 (m, 2H), 3.25 (brm, 3H), 3.58 (brs, 2H), 3.82, 3.85 (2 s, 6H), 3.88-4.38 (m, 5H), 6.32, 6.39 (2 s, 2H), 6.61, 6.72 (2 s, 2H), 6.90-6.94 (m, 3H), 7.28 (t, J 6.0 Hz, 1H), 7.42 (s, 1H). (+) LRESIMS m/z 479 [M+H]⁺.

### COMPOUND 12.1.4: 3-{[6,7-DIMETHOXY-2-(THIEN-3-YLMETHYL)-1,2,3,4-TETRAHYDROISOQUINOLIN-1-YL]METHOXY}-N,N-DIETHYLBENZAMIDE

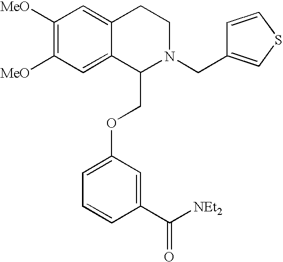

The compound was prepared on an Argonaut Quest 210 synthesiser. To a solution of INTERMEDIATE 5.1.2 (50 mg, 0.125 mmol) in 1,2-dichloroethane (0.5 mL) 3-thiophencarbaldehyde (13 ul, 0.14 mmol) was added. After agitating the solution for 15 min, sodium triacetoxyborohydride (40 mg, 0.19 mmol) was added to each reaction vessel and the agitation continued over night. 1 M aqueous sodium hydroxide solution (1 mL) was added, and after phase separation the organic phase was dried with sodium sulphate. The aqueous layer was extracted another two times with dichloromethane, which was passed through the pad of sodium sulphate, and the combined organic layers were concentrated in vacuo. The crude product was purified by flash chromatography to give the desired product (32 mg, 0.065 mmol, 52%). ¹H NMR (500 MHz, CDCl₃): δ1.12, 1.24 (2 brs, 6H), 2.60 (m, 1H), 2.90-2.99 (m, 2H), 3.25 (brm, 3H), 3.58 (brs, 2H), 3.83, 3.85 (2 s, 6H), 3.93 (s, 2H), 4.03, 4.15, 4.37 (3 m, 3H), 6.61, 6.72 (2 s, 2H), 6.90-6.94 (m, 3H), 7.19, 7.22 (2 s, 2H), 7.30-7.33 (m, 2H). (+) LRESIMS m/z 495 [M+H]⁺.

### COMPOUND 12.1.5: N,N-DIETHYL-4-{[2-(2-FURYLMETHYL)-6,7-DIMETHOXY-1,2,3,4-TETRAHYDROISOQUINOLIN-1-YL]METHOXY}BENZAMIDE

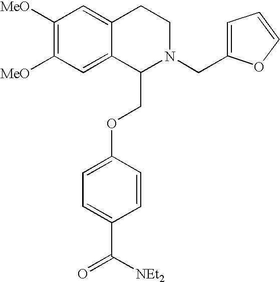

The compound was prepared on an Argonaut Quest 210 synthesiser. To a solution of INTERMEDIATE 5.1.1 (50 mg, 0.125 mmol) in 1,2-dichloroethane (0.5 mL) 2-furaldehyde (12 ul, 0.14 mmol) was added. After agitating the solution for 15 min. sodium triacetoxyborohydride (40 mg, 0.19 mmol) was added to each reaction vessel and the agitation continued over night. 1 M aqueous sodium hydroxide solution (1 mL) was added, and after phase separation the organic phase was dried with sodium sulphate. The aqueous layer was extracted another two times with dichloromethane, which was passed through the pad of sodium sulphate, and the combined organic layers were concentrated in vacuo. The crude product was purified by flash chromatography to give the desired product (26 mg, 0.054 mmol, 44%). ¹H NMR (500 MHz, CDCl₃): δ1.08 (brs, 6H), 2.58 (m, 1H), 2.80, 2.89 (2 m, 2H), 3.19 (m, 1H), 3.37 (brs, 4H), 3.77, 3.79 (2 s, 6H), 3.85 (m, 2H), 3.98, 4.22 (2 m, 2H), 4.07 (m, 1H), 6.20, 6.28 (2 s, 2H), 6.52, 6.72 (2 s, 2H), 6.80, 7.23 (2 m, 4H), 7.36 (s, 1H). (+) LRESIMS m/z 479 [M+H]⁺.

### COMPOUND 12.1.6: 4-{[6,7-DIMETHOXY-2-(THIEN-3-YLMETHYL-1,2,3,4-TETRAHYDROISOQUINOLIN-1-YL]METHOXY}-N,N-DIETHYLBENZAMIDE

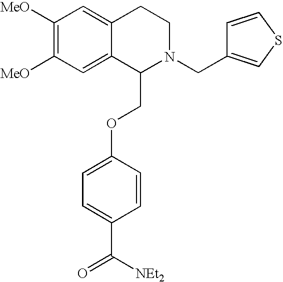

The compound was prepared on an Argonaut Quest 210 synthesiser. To a solution of INTERMEDIATE 5.1.1 (50 mg, 0.125 mmol) in 1,2-dichloroethane (0.5 mL) 3-thiophencarbaldehyde (13 ul, 0.14 mmol) was added. After agitating the solution for 15 min, sodium triacetoxyborohydride (40 mg, 0.19 mmol) was added to each reaction vessel and the agitation continued over night. 1 M aqueous sodium hydroxide solution (1 mL) was added, and after phase separation the organic phase was dried with sodium sulphate. The aqueous layer was extracted another two times with dichloromethane, which was passed through the pad of sodium sulphate, and the combined organic layers were concentrated in vacuo. The crude product was purified by flash chromatography to give the desired product (26 mg, 0.053 mmol, 42%). ¹H NMR (500 MHz, CDCl₃): δ1.08 (brs, 6H), 2.51 (m, 1H), 2.80-2.88 (m, 2H), 3.19 (m, 3H) 3.17 (brs, 4H), 3.76, 3.79 (2 s, 6H), 3.83 (s, 2H), 3.93-4.01 (m, 2H), 4.23 (m, 1H), 6.58, 6.63 (2 s, 2H), 6.80, 7.25 (2 m, 4H), 7.06 (m, 1H), 7.17 (s, 1H), 7.21 (m, 1H). (+) LRESIMS m/z 495 [M+H]⁺.

### COMPOUND 12.1.7: 2-({6,7-DIMETHOXY-2-[(2-PHENYL-1H-IMIDAZOL-5-YL)METHYL]-1,2,3,4-TETRAHYDROISOQUINOLIN-1-YL}METHOXY)-N,N-DIETHYLBENZAMIDE

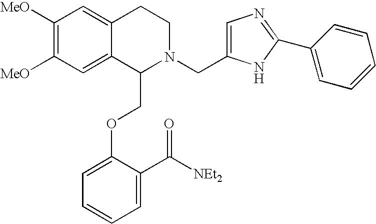

INTERMEDIATE 5.1.3 (50 mg, 0.13 mmol) and 2-phenyl-4(5)-imidazolecarbaldehyde (43 mg, 0.25 mmol) were dissolved in DCE (1.0 mL) and stirred for 15 min at room temperature. Sodium triacetoxyborohydride (80 mg, 0.38 mmol) was added and the reaction mixture was stirred for 18 h at room temperature. 1 M aqueous sodium hydroxide solution (15 mL) and DCM (15 mL) were added, the mixture stirred for 30 min and passed through a Whatman 1PS silicon-treated filter paper. The organic layer was evaporated in vacuo and the crude product was purified by flash chromatography to give the product (34 mg, 0.06 mmol 49%). ¹H NMR (500 MHz, CDCl₃, the spectrum consists of very broad signals: δ 0.95, 1.11 (2 brs, 6H), 2.61 (m, 1H), 2.79-3.63 (m, 7H), 3.48-4.18 (m, 1H), 6.59, 6.73 (2 s, 2H), 6.92-7.43 (m, 8H), 8.14 (brs, 2H). (+) LRESIMS m/z 555 [M+H]⁺.

### COMPOUND 12.1.8: 4-({6,7-DIMETHOXY-2-[(2-PHENYL-1H-IMIDAZOL-5-YL)METHYL]-1,2,3,4-TETRAHYDROISOQUINOLIN-1-YL}METHYL)-N,N-DIETHYLBENZAMIDE

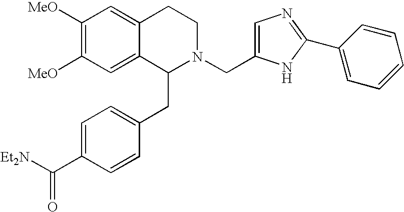

INTERMEDIATE 5.1.5 (15 mg, 0.04 mmol) and 2-phenyl-4(5)-imidazolecarbaldehyde (20 mg, 0.12 mmol) were dissolved in DCE (1.0 mL) and stirred for 15 min at room temperature. Sodium triacetoxyborohydride (34 mg, 0.16 mmol) was added and the reaction mixture was stirred for 18 h at room temperature. 1 M aqueous sodium hydroxide solution (15 mL) and DCM (15 mL) were added, the mixture stirred for 30 min and passed through a Whatman 1PS silicon-treated filter paper. The organic layer was evaporated in vacuo and the crude product was purified by flash chromatography to give the product (12.5 mg, 0.025 mmol, 63%). ¹H NMR (500 MHz, CDCl₃): δ 1.10, 1.27 (2 brs, 6H), 2.92-3.12, 3.42-3.64, 3.85-3.90 (3 m, 10H), 3.24 (brs, 2H, 3.73, 3.88 (2 s, 6H), 3.95 (m, 1H), 6.28 (brs, 1H), 6.63 (s, 1H), 7.12, 7.24 (m, 4H), 7.29 (s, 1H), 7.34 (d, J 7.0 Hz, 1H), 7.43 (dd, J 7.0 Hz, 2H), 7.84 (d, J 7 Hz, 2H). ¹³C NMR (125 MHz, CDCl₃): δ 13.1, 14.4, 23.3, 39.6, 42.3, 43.5, 56.1, 61.6, 111.4, 111.8, 125.3, 126.5, 129.0, 129.9, 128.7, 130.1, 130.3, 135.4, 2×147.2, 147.9, 171.6. (+) LRESIMS m/z 539 [M+H]⁺.

### COMPOUND 12.1.9: 4-{6,7-DIMETHOXY-2-[(2-PHENYL-1H-IMIDAZOL-5-YL)METHYL]-1,2,3,4-TETRAHYDROISOQUINOLIN-1-YL}-N,N-DIETHYLBENZAMIDE

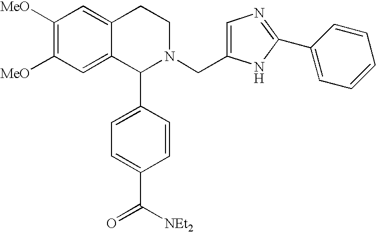

INTERMEDIATE 5.1.7 (250 mg, 0.68 mmol) and 2-phenyl-4(5)-imidazolecarbaldehyde (234 mg, 1.36 mmol) were dissolved in DCE (6 mL) and NMP (0.2 mL). After stirring for 10 min at room temperature, sodium triacetoxyborohydride (431 mg, 2.04 mmol) was added and the mixture was stirred for 18 h at room temperature. Tosylhydrazine scavenger resin (0.48 g, 2.8 mmol/g) was added and the mixture was stirred for another 2 h. DCM and 1 M sodium hydroxide solution were added and the mixture was passed through a Whatman 1PS silicon-treated filter paper. The organic phase was evaporated and the crude product was purified by flash chromatography to yield the product (363 mg, 0.69 mmol, quant); spectra contain signals for NMP. ¹H NMR (500 MHz, CDCl₃): δ 1.15, 1.23 (2 brs, 6H), 2.74, 2.82, 2.96, 3.18 (4 m, 4H), 3.25 (brs, 2H), 3.56-3.85 (m, 3H), 3.62, 3.83 (2 s, 6H), 3.72 (d, J 9.5 Hz, 1H), 4.78 (s, 1H), 6.19 (s, 1H), 6.62 (s, 1H), 6.96 (s, 1H), 7.30-7.41 (m, 7H), 7.90 (d, J 3.5 Hz, 2H). ¹³C NMR (125 MHz, CDCl₃): δ 12.9, 14.5, 27.8, 39.9, 43.9, 46.6, 50.0, 2×56.1, 66.5, 111.3, 2×111.9, 125.5, 126.6, 126.7, 128.7, 129.4, 126.3, 130.0, 136.2, 146.7, 2×147.6, 2×148.1, 171.5. (+) LRESIMS m/z 525 [M+H]⁺.

### COMPOUND 12.1.10: N,N-DIETHYL-4-{6-METHOXY-2-[(2-PHENYL-1H-IMIDAZOL-5-YL)METHYL]-1,2,3,4-TETRAHYDROISOQUINOLIN-1-YL}BENZAMIDE

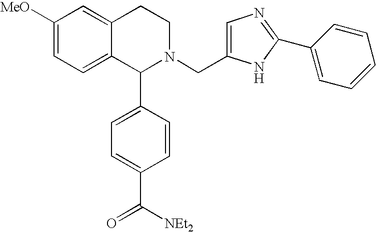

INTERMEDIATE 5.1.7 (15 mg, 0.04 mmol) and 2-phenyl-4(5)-imidazolecarbaldehyde (20 mg, 0.12 mmol) were dissolved in DCE (2 mL). After stirring for 10 min at room temperature, sodium triacetoxyborohydride (34 mg, 0.16 mmol) was added and the mixture was stirred for 18 h at room temperature. DCM and 1 M sodium hydroxide solution were added and the mixture was passed through a Whatman 1PS silicon-treated filter paper. The organic phase was evaporated and the crude product was purified by flash chromatography to yield the product (80 mg, 0.016 mmol, 77%). ¹H NMR (500 MHz, CDCl₃): 1.05, 1.15 (2 brs, 6H), 3.10-3.35 (m, 4H), 3.40-3.65 (m, 4H), 3.68 (s, 3H), 3.82 (m, 2H), 5.82 (s, 1H), 6.55-6.82 (m, 4H), 7.30-7.77 (m, 6H), 7.98 (d, J 9 Hz, 1H), 8.32 (d, J 2.5 Hz, 2H). (+) LRESIMS m/z 493 [M+H]⁺.

### COMPOUND 12.1.11: N,N-DIETHYL-4-{7-METHOXY-2-[(2-PHENYL-1H-IMIDAZOL-5-YL)METHYL]-1,2,3,4-TETRAHYDROISOQUINOLIN-1-YL}BENZAMIDE

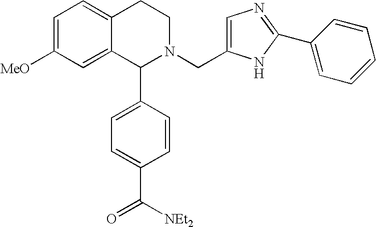

INTERMEDIATE 4.2.2 (24 mg, 0.07 mmol) and 2-phenylimidazole-4(5)-carbaldehyde (48 mg, 0.28 mmol) were dissolved in DCE (2.0 mL) and NMP (0.2 mL). After stirring for 10 min at room temperature, sodium triacetoxyborohydride (59 mg, 0.28 mmol) was added and the mixture was stirred for 18 h at room temperature. DCM and 1 M sodium hydroxide solution were added and the mixture was passed through a Whatman 1PS silicon-treated filter paper. The organic phase was evaporated and the crude product was purified by flash chromatography to yield the product (13 mg, 0.026 mmol, 37%). ¹H NMR (500 MHz, CDCl₃): δ 1.10, 1.24 (2 brs, 6H), 2.73, 2.82, 3.00, 3.17 (4 m, 4H), 3.25, 3.54 (2 brs, 4H), 3.62 (s, 3H), 3.68 (m, 2H), 4.89 (s, 1H), 6.21 (s, 1H), 6.73 (d, J 8.5 Hz, 1H), 6.90 (s, 1H), 7.05 (d, J 8.5 Hz, 1H), 7.27-7.38 (m, 7H), 7.90 (m, 2H). ¹³C NMR (125 MHz, CDCl₃): δ 12.8, 14.2, 26.4, 39.5, 43.4, 46.6, 49.1, 55.2, 66.9, 113.2, 113.8, 125.9, 126.5, 129.0, 129.7, 129.9, 157.9, 171.1. (+) LRESIMS m/z 495 [M+H]⁺.

### COMPOUND 12.1.12: N,N-DIETHYL-4-{2-[(2-PHENYL-1H-IMIDAZOL-5-YL)METHYL]-1,2,3,4-TETRAHYDROISOQUINOLIN-1-YL}BENZAMIDE

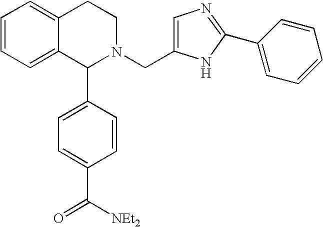

N,N-Diethyl-4-(1,2,3,4-tetrahydroisoquinolin-1-yl)benzamide (28 mg, 0.09 mmol) and 2-phenylimidazole-4(5)-carbaldehyde (63 mg, 0.36 mmol) were dissolved in DCE (2.0 mL) and NMP (0.2 mL). After stirring for 10 min at room temperature, sodium triacetoxyborohydride (76 mg, 0.36 mmol) was added and the mixture was stirred for 18 h at room temperature. Tosylhydrazine scavenger resin (0.45 g, 2.4 mmol/g) was added and the mixture was stirred for another 2 h. DCM and 1 M sodium hydroxide solution were added and the mixture was passed through a Whatman 1PS silicon-treated filter paper. The organic phase was evaporated and the crude product was purified by flash chromatography to yield the product (27 mg, 0.07 mmol, 67%). ¹H NMR (500 MHz, CDCl₃): δ 1.22, 1.26 (2 brs, 6H), 2.69, 2.84, 3.04, 3.17 (4 m, 4H), 3.28 (brs, 2), 3.58 (m, 4H), 4.77 (s, 1H), 6.67, 7.04, 7.13 (3 m, 4H), 6.90 (s, 1H), 7.33-7.40 (m, 7H), 7.90 (dt, J 2, 8 Hz, 2H). ¹³C NMR (125 MHz, CDCl₃): δ 12.9, 14.2, 28.5, 39.5, 43.4, 46.8, 49.4, 67.1, 125.4, 126.0, 126.3, 128.6, 128.8, 129.7, 129.4, 134.3, 135.9, 137.0, 144.4, 146.2, 171.3. (+) LRESIMS m/z 465 [M+H]⁺.

### COMPOUND 12.1.13: 4-{2-[(2-BUTYL-1H-IMIDAZOL-5-YL)METHYL]-6,7-DIMETHOXY-1,2,3,4-TETRAHYDROISOQUINOLIN-1-YL}-N,N-DIETHYLBENZAMIDE

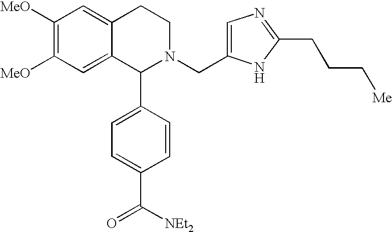

INTERMEDIATE 5.1.7 (30 mg, 0.08 mmol) and 2-n-butylimidazole-4(5)-carbaldehyde (37 mg, 0.24 mmol) were dissolved in DCE (2.0 mL). After stirring for 10 min at room temperature, sodium triacetoxyborohydride (68 mg, 0.32 mmol) was added and the mixture was stirred for 18 h at room temperature. Tosylhydrazine scavenger resin (0.17 g, 2.4 mmol/g) was added and the mixture was stirred for another 2 h. DCM and 1 M sodium hydroxide solution were added and the mixture was passed through a Whatman 1PS silicon-treated filter paper. The organic phase was evaporated and the crude product was purified by flash chromatography to yield the product (33 mg, 0.066 mmol, 83%). ¹H NMR (500 MHz, CDCl₃): δ 0.89 (m, 3H), 1.15, 1.24 (2 brs, 6H), 1.34 (m, 2H), 1.67 (m, 2H), 2.05, 2.60-2.82, 2.88-3.01, 3.17, 3.45 (5 m, 8H), 3.28, 3.58 (2 brs, 4H), 3.61, 3.85 (2 s, 6H), 4.62 (s, 1H), 6.16, 6.62, 6.75 (3 s, 3H), 7.30 (m, 4H). ¹³C NMR (125 MHz, CDCl₃): δ 12.8, 14.2, 13.7, 22.3, 27.8, 27.9, 30.4, 39.5, 43.4, 46.5, 49.8, 2×55.8, 66.5, 111.0, 111.7, 126.3, 129.5, 126.3, 128.9, 135.8, 145.0, 2×147.2, 147.7, 148.5, 171.1. (+) LRESIMS m/z 527 [M+Na]⁺.

### COMPOUND 12.1.14: 4-{2-[(2-BUTYL-4-CHLORO-1H-IMIDAZOL-5-YL)METHYL]-6,7-DIMETHOXY-1,2,3,4-TETRAHYDROISOQUINOLIN-1-YL}-N,N-DIETHYLBENZAMIDE

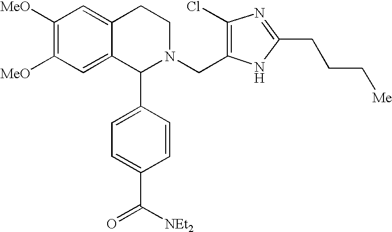

INTERMEDIATE 5.1.7 (30 mg, 0.08 mmol) and 4-chloro-2-n-butylimidazole-5-carbaldehyde (45 mg, 0.24 mmol) were dissolved in DCE (2.0 mL). After stirring for 10 min at room temperature, sodium triacetoxyborohydride (76 mg, 0.36 mmol) was added and the mixture was stirred for 18 h at room temperature. Tosylhydrazine scavenger resin (0.20 g, 2.4 mmol/g) was added and the mixture was stirred for another 2 h. DCM and 1 M sodium hydroxide solution were added and the mixture was passed through a Whatman 1PS silicon-treated filter paper. The organic phase was evaporated and the crude product was purified by flash chromatography to yield the product (26 mg, 0.048 mmol, 60%). ¹H NMR (500 MHz, CDCl₃): δ 0.91 (m, 3H), 1.16, 1.24 (2 brs, 6H), 1.39 (m, 2H), 1.64 (m, 2H), 2.59-2.70, 2.94, 3.09, 3.50-3.62 (4 m, 10H), 3.28 (brs, 2H), 3.63, 3.87 (2 s, 6H), 4.62 (s, 1H), 6.19, 6.62 (2 s, 2H), 7.30 (m, 4H), 9.65 (brs, 1H). (+) LRESIMS m/z 561, 563 [M+Na]⁺.

### COMPOUND 12.1.15: 4-{6,7-DIMETHOXY-2-[(2-METHYL-1H-IMIDAZOL-5-YL)METHYL]-1,2,3,4-TETRAHYDROISOQUINOLIN-1-YL}-N,N-DIETHYLBENZAMIDE

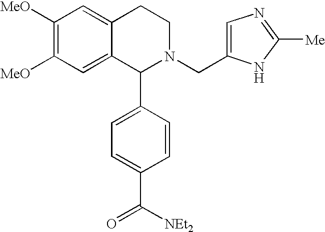

INTERMEDIATE 5.1.7 (30 mg, 0.08 mmol) and 2-methylimidazole-4(5)-carbaldehyde (26 mg, 0.24 mmol) were dissolved in DCE (2.0 mL). After stirring for 10 min at room temperature, sodium triacetoxyborohydride (76 mg, 0.36 mmol) was added and the mixture was stirred for 18 h at room temperature. Tosylhydrazine scavenger resin (0.20 g, 2.4 mmol/g) was added and the mixture was stirred for another 2 h. DCM and 1 M sodium hydroxide solution were added and the mixture was passed through a Whatman 1PS silicon-treated filter paper. The organic phase was evaporated and the crude product was purified by flash chromatography to yield the product (22 mg, 0.047 mmol, 59%). ¹H NMR (500 MHz, CDCl₃): δ 1.11, 1.24 (2 brs, 6H), 2.33 (m, 3H), 2.62, 2.69, 2.92, 3.13, 3.60 (5 m, 8H), 3.28 (brs, 2H), 3.61, 3.85 (2 s, 6H), 4.60 (s, 1H), 6.19, 6.62, 6.73 (3 s, 3H), 7.30 (m, 4H). ¹³C NMR (125 MHz, CDCl₃): δ 12.9, 14.2, 13.7, 27.9, 39.4, 43.4, 46.6, 49.9, 2×55.8, 66.5, 111.0, 111.7, 126.3, 129.5, 120.5, 126.3, 128.9, 131.5, 135.9, 144.2, 145.1, 147.2, 147.6, 171.3. (+) LRESIMS m/z 463 [M+H]⁺.

### COMPOUND 12.1.16: 4-{6,7-DIMETHOXY-2-[(3-PHENYL-1H-PYRAZOL-4-YL)METHYL]-1,2,3,4-TETRAHYDROISOQUINOLIN-1-YL}-N,N-DIETHYLBENZAMIDE

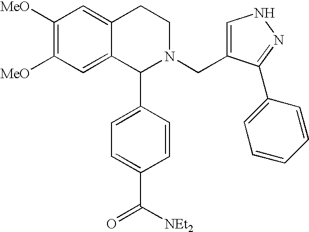

INTERMEDIATE 5.1.7 (50 mg, 0.14 mmol) and 3-phenyl-4-pyrrazolecarbaldehyde (36 mg, 0.21 mmol) were dissolved in DCE (3.0 mL). After stirring for 10 min at room temperature, sodium triacetoxyborohydride (87 mg, 0.41 mmol) was added and the mixture was stirred for 18 h at room temperature. Tosylhydrazine scavenger resin (0.05 g, 2.4 mmol/g) was added and the mixture was stirred for another 2 h. DCM and 1 M sodium hydroxide solution were added and the mixture was passed through a Whatman 1PS silicon-treated filter paper. The organic phase was evaporated and the crude product was purified by flash chromatography to yield the product (63 mg, 0.12 mmol, 86%). ¹H NMR (500 MHz, CDCl₃): δ 1.00, 1.09 (2 brs, 61), 2.55-3.00, 3.65-3.80 (2 brm, 6H), 3.18, 3.45 (2 brs, 4H), 3.55, 3.80 (2 s, 61), 4.60 (s, 1H), 6.16, 6.50, 6.59 (3 s, 3), 7.18-7.75 (m, 9H). (+) LRESIMS m/z 525 [M+H]⁺.

### COMPOUND 12.1.17: 4-(6,7-DIMETHOXY-2-{[1-(PHENYLSULFONYL)-1H-PYRROL-2-YL]METHYL}-1,2,3,4-TETRAHYDROISOQUINOLIN-1-YL)-N,N-DIETHYLBENZAMIDE

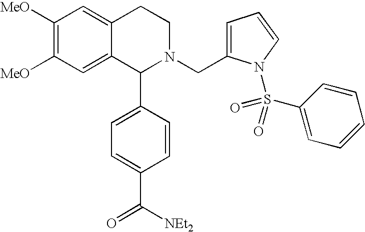

INTERMEDIATE 5.1.7 (30 mg, 0.08 mmol) and 1-phenylsulfonyl-2-pyrrolcarbaldehyde (56 mg, 0.24 mmol) were dissolved in DCE (2.0 mL). After stirring for 10 min at room temperature, sodium triacetoxyborohydride (68 mg, 0.32 mmol) was added and the mixture was stirred for 18 h at room temperature. Tosylhydrazine scavenger resin (0.20 g, 2.4 mmol/g) was added and the mixture was stirred for another 2 h. DCM and 1 M sodium hydroxide solution were added and the mixture was passed through a Whatman 1PS silicon-treated filter paper. The organic phase was evaporated and the crude product was purified by flash chromatography to yield the product (30 mg, 0.05 mmol, 64%) of the desired product. ¹H NMR (500 MHz, CDCl₃): δ 1.03, 1.17 (2 brs, 6H), 2.39, 2.56, 2.73 (3 m, 4H), 3.19 (brs, 2H), 3.40-3.52 (m, 3H), 3.59 (s, 3H), 3.79-3.84 (m, 4H), 4.68 (s, 1H), 6.11-6.15 (m, 3H), 6.49 (s, 1H), 7.09 (d, J 7.5 Hz, 2H), 7.14-7.24 (m, 5H), 7.38 (t, J 7.5 Hz, 1H), 7.62 (d, 7.5 Hz, 2H). ¹³C NMR (125 MHz, CDCl₃): δ 12.9, 14.1, 27.0, 39.2, 43.3, 44.1, 50.3, 2×55.8, 66.4, 111.0, 111.5, 114.9, 123.2, 133.3, 132.7, 136.0, 139.4, 143.3, 147.2, 147.7, 171.1. (+) LRESIMS m/z 495 [M+H]⁺.

### COMPOUND 12.1.18: N,N-DIETHYL-4-{2-[(2-ETHYL-4-METHYL-1H-IMIDAZOL-5-YL)METHYL]-6,7-DIMETHOXY-1,2,3,4-TETRAHYDROISOQUINOLIN-1-YL}BENZAMIDE

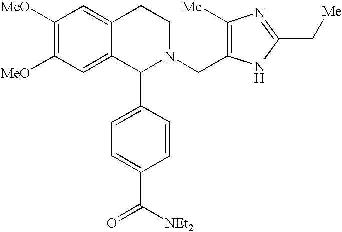

INTERMEDIATE 5.1.7 (50 mg, 0.14 mmol) and 2-ethyl-4-methyl-5-imidazolecarbaldehyde (38 mg, 0.27 mmol) were dissolved in DCE (2.0 mL). After stirring for 10 min at room temperature, sodium triacetoxyborohydride (86 mg, 0.41 mmol) was added and the mixture was stirred for 18 h at room temperature. DCM and 1 M sodium hydroxide solution were added and the mixture was passed through a Whatman 1PS silicon-treated filter paper. The organic phase was evaporated and the crude product was purified by flash chromatography to yield the product (30 mg, 0.05 mmol, 64%) of the desired product (60 mg, 0.12 mmol, 90%). ¹H NMR (500 MHz, CDCl₃): 1.08 (brs, 3H), 1.23 (m, 6H), 2.03 (s, 3H), 2.50, 2.72, 2.93, 3.06 (4 m, 4H), 3.08 (mc, 2H) 3.25, 3.60 (brs, 3H), 3.39, 3.59 (2 d, J 13 Hz, 2H), 3.60, 3.40 (2 s, 6H), 4.53 (s, 1H), 6.16, 6.60 (2 s, 2H), 7.27 (m, 4H). ¹³C NMR (125 MHz, CDCl₃): 11.14, 12.89, 13.43, 14.03, 21.83, 28.00, 39.66, 43.60, 46.67, 49.48, 56.06, 67.43, 111.2, 112.0, 126.5, 127.2, 127.2, 127.3, 129.3, 130.2, 136.1, 136.2, 145.5, 147.4, 148.1, 171.5. (+) LRESIMS m/z 488 [M+H]⁺.

### COMPOUND 12.1.19: 4-{6,7-DIMETHOXY-2-[(4-METHYL-IMIDAZOL-5-YL)METHYL]-1,2,3,4-TETRAHYDROISOQUINOLIN-1-YL}-N,N-DIETHYLBENZAMIDE

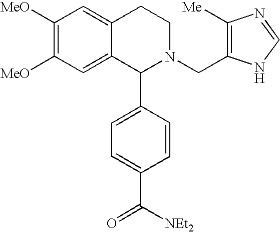

INTERMEDIATE 5.1.7 (50 mg, 0.14 mmol) and 4-methyl-5-imidazolecarbaldehyde (30 mg, 0.27 mmol) were dissolved in DCE (3.0 mL). After stirring for 10 min at room temperature, sodium triacetoxyborohydride (86 mg, 0.41 mmol) was added and the mixture was stirred for 18 h at room temperature. DCM and 1 M sodium hydroxide solution were added and the mixture was passed through a Whatman 1PS silicon-treated filter paper. The organic phase was evaporated and the crude product was purified by flash chromatography to yield the product (57 mg, 0.12 mmol, 91%). ¹H NMR (500 MHz, CDCl₃): 1.08, 1.22 (2 brs, 6H), 2.07 (s, 3H), 2.54, 2.72, 2.90, 3.06 (4 m, 4H), 3.25, 3.54 (2 brs, 4H), 3.39, 3.59 (2 d, J 13 Hz, 2H), 4.56 (s, 1H), 6.16, 6.59 (2 s, 2H), 7.29 (s, 4H), 8.38 (brs, 1H). ¹³C NMR (125 MHz, CDCl₃): 11.14, 13.43, 14.03, 28.07, 39.69, 43.67, 46.67, 49.48, 56.04, 56.06, 67.48, 111.2, 112.0, 126.5, 129.9, 127.2, 127.3, 129.3, 130.2, 136.1, 133.3, 145.9, 147.6, 147.7, 171.8. (+) LRESIMS m/z 369 (100), 463 (35).

### COMPOUND 12.1.20: 4-{5,8-DIMETHOXY-2-[(4-METHYL-1H-IMIDAZOL-5-YL)METHYL]-1,2,3,4-TETRAHYDROISOQUINOLIN-1-YL}-N,N-DIETHYLBENZAMIDE

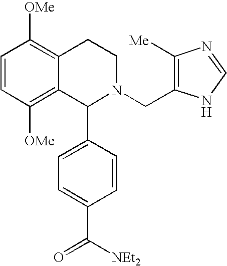

INTERMEDIATE 5.1.10 (115 mg, 0.31 mmol) and 4-methyl-5-imidazolecarbaldehyde (108 mg, 0.62 mmol) were dissolved in DCE (5.0 mL). After stirring for 10 min at room temperature, sodium triacetoxyborohydride (197 mg, 0.93 mmol) was added and the mixture was stirred for 18 h at room temperature. DCM and 1 M sodium hydroxide solution were added and the mixture was passed through a Whatman 1PS silicon-treated filter paper. The organic phase was evaporated and the crude product was purified by flash chromatography to yield the product (103 mg, 0.20 mmol, 65%). ¹H NMR (500 MHz, CDCl₃): δ 0.99, 1.18 (2 brs, 6H), 2.58-2.74, 3.17-3.50 (2 m, 8H), 3.32, 3.35 (2 s, 6H), 3.44, 3.65 (2 d, J 12.0 Hz, 2H), 5.05 (s, 1H), 6.51, 6.63 (2 d, J 9.0 Hz, 2H), 6.87 (s, 1H), 7.00, 7.14 (m, 4H), 7.21 (m, 5H), 7.80 (d, J 3.5 Hz, 2H).

### COMPOUND 12.1.21: N,N-DIETHYL-4-[1,2,3,4-TETRAHYDRO-6-METHOXY-2-[(4-METHYL-1H-IMIDAZOL-5-YL)METHYL]-1-ISOQUINOLINYL]-BENZAMIDE

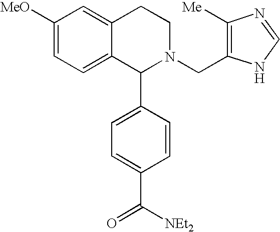

INTERMEDIATE 5.1.8 (1.1 g, 3.25 mmol) was dissolved in 1,2-dichloroethane (70 mL), 4-methyl-imidazole-5-carboxaldehyde (716 mg, 6.5 mmol) was added and the reaction mixture stirred at room temperature for 10 min. Sodium triacetoxyborohydride (2.1 g, 9.8 mmol) was added and the reaction mixture stirred for a further 16 h. MeOH (5 mL) was added and the reaction mixture concentrated to dryness. The resulting residue was partitioned between EtOAc (50 mL) and NaOH (1N, 30 mL), the aqueous phase washed with EtOAc (2×50 mL), dried (MgSO₄), filtered and concentrated to dryness. The resulting residue was purified by flash chromatography on silica gel (10:1, DCM:MeOH) to afford COMPOUND 12.1.21 (736 mg, 54%) as a colourless oil. ¹H NMR (500 MHz, CDCl₃): δ 1.13, 1.24 (br s, 6H), 2.09 (s), 2.53 (td, J 4, 13 Hz), 2.76 (br d, J 17 Hz, 1H), 2.98 (m, 1H), 3.09 (m, 1H), 3.28 (br s, 2H), 3.55 (br s, 2H), 3.33 (d, J 2 Hz, 1H), 3.60 (d, J 2 Hz, 1H), 3.76 (s, 3H), 4.56 (s, 1H), 6.58 (s, 1H), 6.64 (br s, 1H), 7.29-7.30 (m, 5H), 8.30 (s, 1H). ¹³C NMR (125 MHz, CDCl₃): δ 11.2, 13.1, 14.5, 29.2, 39.6, 43.7, 47.0, 49.6, 55.4, 68.0, 112.6, 113.0, 126.5, 127.0, 129.9, 130.0, 130.1, 133.2, 136.1, 136.2, 145.9, 158.1, 171.6. (+) LRESIMS m/z 433 [M+H]⁺.

### COMPOUND 12.1.22: N,N-DIETHYL-4-[2-(1H-IMIDAZOL-5-YLMETHYL)-6-METHOXY-1,2,3,4-TETRAHYDROISOQUINOLIN-1-YL]BENZAMIDE

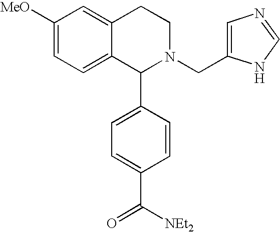

INTERMEDIATE 5.1.8 (80 mg, 0.236 mmol) was dissolved in 1,2-dichloroethane (3 mL) and imidazole-5-carboxaldehyde (45.4 mg, 0.473 mmol) was added under stirring. The reaction mixture was stirred at room temperature for 10 min. Sodium triacetoxyborohydride (250.5 mg, 1.182 mmol) was added and the reaction mixture stirred for a further 16 h. 1 N NaOH (2 mL, 2 mmol) was added and the resulting mixture taken up in EtOAc (10 mL), washed with NaHCO₃ (5 mL) and brine (5 mL) and concentrated to dryness. The residue was purified by flash chromatography on SiO₂ column (DCM:MeOH 10:1) to afford COMPOUND 12.1.22 (58 mg, 64%) as an oil. ¹H NMR (500 MHz, CDCl₃): δ 1.20 (br s, 3H), 1.32 (br s, 3H), 2.68 (br t, J 10 Hz, 1H), 2.86 (br d, J 16.4 Hz, 1H), 3.09 (m, 1H), 3.21 (m, 1H), 3.35 (br s, 2H), 3.53 (m, 1H), 3.61 (br s, 2H), 3.73 (d, J 14 Hz, 1H), 3.83 (s, 3H), 4.68 (s, 1H), 6.65 (s, 2H), 6.71 (s, 1H), 6.93 (s, 1H), 7.38 (m, 4H), 7.51 (s, 1H); ¹³C NMR (125 MHz, CDCl₃): δ 13.01, 14.35, 29.15, 39.55, 43.59, 47.19, 50.02, 55.33, 67.11, 112.57, 112.95, 121.47, 126.57, 129.81, 130.05, 130.05, 131.79, 134.97, 135.98, 136.12, 145.48, 158.03, 171.56; (+) LRESIMS m/z 419.10 [M+H]⁺.

### COMPOUND 12.1.23: N,N-DIETHYL-4-[2-(1H-IMIDAZOL-5-YLMETHYL)-6,7-DIMETHOXY-1,2,3,4-TETRAHYDROISOQUINOLIN-1-YL]BENZAMIDE

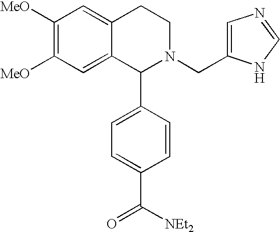

INTERMEDIATE 5.1.7 (80 mg, 0.216 mmol) was dissolved in 1,2-dichloroethane (3 mL) and imidazole-5-carboxaldehyde (41.7 mg, 0.434 mmol) added under stirring. The reaction mixture was stirred at room temperature for 10 min. Sodium triacetoxyborohydride (230 mg, 1.085 mmol) was added and the reaction mixture stirred for a further 16 h. 1 N NaOH (2 mL, 2 mmol) was added and the resulting mixture taken up in EtOAc (10 mL), washed with NaHCO₃ (5 mL) and brine (5 mL) and concentrated to dryness. The residue was purified by flash chromatography on SiO₂ column (DCM:MeOH 10:1) to afford COMPOUND 12.1.23 (66.4 mg, 66%) as an oil. ¹H NMR (500 MHz, CDCl₃) δ 1.28 (br s, 3H), 1.41 (br s, 3H), 2.82 (br s, 1H), 2.94 (br d, J 16.2 Hz, 1H), 3.09 (m, 1H), 3.28 (m, 1H), 3.43 (br s, 2H), 3.63-3.87 (m, 4H), 3.78 (s, 3H), 4.02 (s, 3H), 4.82 (s, 1H), 6.33 (s, 1H), 6.78 (s, 1H), 7.07 (s, 1H), 7.48 (m, 4H), 7.74 (s, 1H); ¹³C NMR (125 MHz, CDCl₃): δ 13.00, 14.34, 27.66, 39.62, 43.55, 46.59, 49.52, 56.00, 66.56, 111.19, 111.81, 121.45, 126.57, 126.76, 128.53, 129.87, 131.12, 135.06, 136.25, 144.71, 147.51, 148.09, 171.51; (+) LRESIMS m/z 449.1 [M+H]⁺.

### COMPOUND 12.1.24: 4-{6,7-DIMETHOXY-2-[(5-PHENYL-2-FURYL)METHYL]-1,2,3,4-TETRAHYDROISOQUINOLIN-1-YL}-N,N-DIETHYLBENZAMIDE

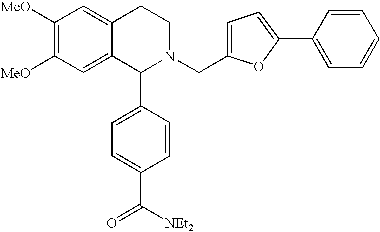

INTERMEDIATE 5.1.7 (80 mg, 0.216 mmol) was dissolved in 1,2-dichloroethane (3 mL) and 5-phenyl-2-furaldehyde (74.8 mg, 0.434 mmol) was added under stirring. The reaction mixture was stirred at room temperature for 10 min. Sodium triacetoxyborohydride (230 mg, 1.085 mmol) was added and the reaction mixture stirred for a further 16 h. 1 N NaOH (2 mL, 2 mmol) was added and the resulting mixture taken up in EtOAc (10 mL), washed with NaHCO₃ (5 mL) and brine (5 mL) and concentrated to dryness. The residue was purified by flash chromatography on SiO₂ column (DCM:MeOH 20:1) to afford COMPOUND 12.1.24 (63.5 mg, 56%) as an oil. ¹H NMR (500 MHz, CDCl₃): δ 1.28 (br s, 3H), 1.41 (br s, 3H), 2.95 (d, J 15.4 Hz, 1H), 3.03 (m, 1H), 3.26 (br s, 1H), 3.43 (br s, 2H), 3.73-3.96 (m, 5H), 3.76 (s, 3H), 4.01 (s, 3H), 4.85 (s, 1H), 6.33 (s, 1H), 6.43 (s, 1H), 6.77 (s, 2H), 7.42 (m, 2H), 7.55-7.58 (m, 5H), 7.83 (m, 2H); ¹³C NMR (125 MHz, CDCl₃): δ 13.71, 14.99, 29.42, 40.07, 44.03, 48.81, 51.63, 56.64, 66.55, 106.43, 111.85, 112.54, 124.44, 127.37, 127.60, 127.99, 129.51, 130.35, 131.81, 137.04, 146.15, 148.03, 148.39, 152.68, 154.23, 172.08; (+) LRESIMS m/z 525.0 [M+H]⁺, 546.9 [M+Na]⁺.

### COMPOUND 12.1.25: N,N-DIETHYL-4-{6-METHOXY-2-[(5-PHENYL-2-FURYL)METHYL]-1,2,3,4-TETRAHYDROISOQUINOLIN-1-YL}BENZAMIDE

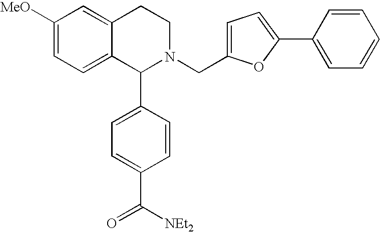

INTERMEDIATE 5.1.8 (80 mg, 0.236 mmol) was dissolved in 1,2-dichloroethane (3 mL) and 5-phenyl-2-furaldehyde (81.4 mg, 0.473 mmol) was added under stirring. The reaction mixture was stirred at room temperature for 10 min. Sodium triacetoxyborohydride (250.5 mg, 1.182 mmol) was added and the reaction mixture stirred for a further 16 h. 1 N NaOH (2 mL, 2 mmol) was added and the resulting mixture taken up in EtOAc (10 mL), washed with NaHCO₃ (5 mL) and brine (5 mL) and concentrated to dryness. The residue was purified by flash chromatography on SiO₂ column (hexane:EtOAc:MeOH 70:29:1) to afford COMPOUND 12.1.25 (61 mg, 57%) as an oil. ¹H NMR (500 MHz, CDCl₃): δ 1.30 (br s, 3H), 1.41 (br s, 3H), 2.99 (m, 2H), 3.35 (m, 1H), 3.45 (m, 2H), 3.60 (s, 3H), 3.72 (br s, 2H), 3.78-3.95 (m, 3H), 3.91 (s, 3H), 4.86 (s, 1H), 6.42 (s, 1H), 6.75 (m, 2H), 6.81 (s, 1H), 7.41 (m, 1H), 7.52-7.60 (m, 6H), 7.82 (m, 2H); ¹³C NMR (125 MHz, CDCl₃): δ 12.98, 14.31, 29.74, 39.45, 43.51, 48.36, 50.97, 55.29, 66.13, 105.71, 111.23, 112.41, 112.92, 123.72, 126.66, 127.30, 128.80, 129.78, 130.06, 130.33, 131.09, 135.86, 136.23, 145.65, 151.94, 153.56, 157.86, 171.50; (+) LRESIMS m/z 495.0 [M+H]⁺.

### COMPOUND 12.1.26: N,N-DIETHYL-4-{7-HYDROXY-6-METHOXY-2-[(5-METHYL-1H-IMIDAZOL-4-YL)METHYL]-1,2,3,4-TETRAHYDROISOQUINOLIN-1-YL}BENZAMIDE

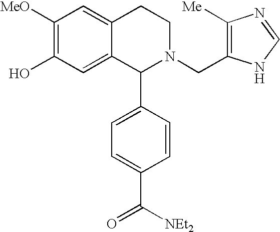

To a solution of INTERMEDIATE 5.1.12 (56 mg, 0.158 mmol) in anhydrous dichloromethane (3.5 mL) was added 4-methyl-1H-imidazole-5-carboxaldehyde (2.5 eq, 43 mg, 0.395 mmol) and followed by sodium triacetoxyborohydride (4 eq, 134 mg, 0.623 mmol). The reaction mixture was stirred at room temperature for 20 hr then quenched with saturated aqueous sodium bicarbonate and extracted with ethyl acetate (2×20 mL), dried over MgSO₄. Solvent was removed and the residue purified by preparative column chromatography to give 60 mg (0.1339 mmol, 84%) of COMPOUND 12.1.26 as a white solid. ¹H NMR (500 MHz, CDCl₃): δ 1.15 (br s, 3H), 1.30 (br s, 3H), 2.08 (s, 3H), 2.61 (m, 1H), 2.78 (m, 1H), 2.98 (m, 1H), 3.18 (m, 1H), 3.36 (br s, 2H), 3.40 (d, J 10 Hz, 1H), 3.58 (br s, 2H), 3.68 (d, J 10 Hz, 1H), 3.80 (s, 3H), 4.58 (s, 1H), 6.15 (s, 1H), 6.70 (s, 1H), 7.35 (d, J 8 Hz, 2H), 7.40 (d, J 8 Hz, 2H), 7.75 (s, 1H). ¹³C NMR (125 MHz, CDCl₃): δ 9.10, 12.00, 13.05, 27.75, 39.80, 43.80, 49.05, 55.25, 68.02, 111.15, 114.97, 125.66, 126.13, 127.44, 128.00, 129.42, 129.89, 133.19, 135.99, 144.60, 146.00, 146.82, 172.50. (+) LRESIMS m/z 449 (M+H)⁺.

### COMPOUND 12.1.27: N,N-DIETHYL-4-{7-HYDROXY-6-METHOXY-2-[(2-PHENYL-1H-IMIDAZOL-4-YL)METHYL]-1,2,3,4-TETRAHYDROISOQUINOLIN-1-YL}BENZAMIDE

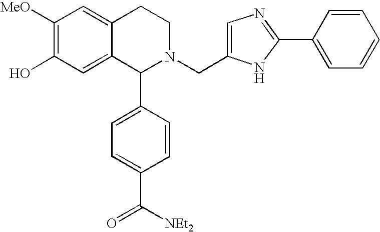

To a solution of INTERMEDIATE 5.1.12 (100 mg, 0.28 mmol) and 2-phenyl-1H-imidazole-5-carboxaldehyde (120 mg, 0.7 mmol) in anhydrous 1,2-dichloroethane (6 mL) was added sodium triacetoxyborohydride (238 mg, 1.126 mmol). The reaction mixture was stirred at room temperature for 20 hr then quenched with saturated aqueous sodium bicarbonate and extracted with ethyl acetate (2×20 mL), dried over MgSO₄. Solvent was removed and the residue was purified by preparative column chromatography to give 49 mg (0.096 mmol, 48%) of COMPOUND 12.1.27 as white solid. ¹H NMR (500 MHz, DMSO-d₆): δ 1.15 (br s, 6H), 2.58 (m, 1H), 2.70 (m, 1H), 2.90 (m, 1H), 3.15 (m, 1H), 3.20-3.60 (m, 6H), 3.71 (s, 3H), 4.68 (s, 1H), 6.10 (s, 1H), 6.61 (s, 1H), 7.00 (s, 1H), 7.25-8.00 (m, 9H), 8.60 (br s, 1H). ¹³C NMR (125 MHz, DMSO-d₆): δ 14.00, 15.00, 29.06, 41.80, 48.02, 52.10, 54.00, 66.03, 112.50, 116.03, 125.00, 127.01, 128.90, 131.08, 131.10, 131.80, 136.80, 139.80, 144.10, 171.05. (+) LRESIMS m/z 511 (M+H)⁺.

### COMPOUND 12.1.28: 4-{2-[(1-BENZYL-1H-IMIDAZOL-5-YL)METHYL]-6,7-DIMETHOXY-1,2,3,4-TETRAHYDROISOQUINOLIN-1-YL}-N,N-DIETHYLBENZAMIDE

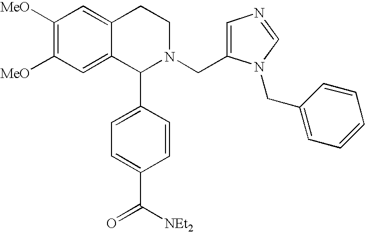

INTERMEDIATE 5.1.7 (50 mg, 0.14 mmol) and 1-benzylimidazole-5-carbaldehyde (51 mg, 2 eq, 0.27 mmol) were dissolved in 1,2-dichloroethane (2 mL) and sodium triacetoxyborohydride (86 mg, 3 eq, 0.41 μmol) was added. The reaction mixture was stirred at room temperature for 18 h. Resin-bound tosylhydrazine (73 mg, 2.4 mmol/g) was added and the mixture was agitated for another 2 h. Dichloromethane and 2 M aqueous sodium hydroxide solution were added, the mixture was shaken and passed through a Whatman 1PS filter paper. The organic phase was evaporated to dryness and purified by flash chromatography (10 g, 100% dichloromethane e 5% methanol in dichloromethane) to yield the product (73 mg, 0.135 mmol, 99%). ¹H NMR (500 MHz, CDCl₃): 1.09, 1.28 (2 brs, 6H), 3.00-3.60 (m, 8H), 3.62, 3.83 (2 s, 6H), 4.12 (m, 2H), 5.12-5.39 (m, 3H), 6.13, 6.64 (2 s, 2H), 7.00 (d, J 7.0 Hz, 2H), 7.35-7.42 (m, 7H), 7.77, (s, 1H), 8.48 (s, 1H). (+) LRESIMS m/z 539 (100).

### COMPOUND 12.1.29: 4-{6,7-DIMETHOXY-2-[(1-METHYL-1H-IMIDAZOL-5-YL)METHYL]-1,2,3,4-TETRAHYDROISOQUINOLIN-1-YL}-N,N-DIETHYLBENZAMIDE

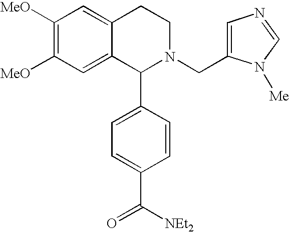

INTERMEDIATE 5.1.7 (50 mg, 0.14 mmol) and 1-methyl-5-imidazolecarbaldehyde (30 mg, 0.27 mmol) were dissolved in DCE (2 mL). After stirring for 10 min at room temperature, sodium triacetoxyborohydride (86 mg, 0.41 mmol) was added and the mixture was stirred for 18 h at room temperature. DCM and 1 M sodium hydroxide solution were added and the mixture was passed through a Whatman 1PS silicon-treated filter paper. The organic phase was evaporated and the crude product was purified by flash chromatography to yield the product (29 mg, 0.06 mmol, 46%). ¹H NMR (500 MHz, CDCl₃): 1.05, 1.28 (2 brs, 6H), 2.54, 2.72, 2.90, 3.06 (4 m, 4H), 3.25 (brs, 2H), 3.39-3.65 (m, 4H), 3.53 (s, 3), 3.62, 3.89 (2 s, 6H), 4.52 (s, 1H), 6.13, 6.64 (2 s, 2H), 7.31 (m, 5H), 8.19 (s, 1H). ¹³C NMR (125 MHz, CDCl₃): 13.12, 14.44, 23.42, 21.00, 28.07, 33.61, 39.70, 43.57, 46.70, 48.12, 53.63, 56.06, 68.38, 111.2, 111.9, 123.2, 126.7, 126.9, 127.1, 127.3, 128.7, 129.9, 136.8, 137.0, 138.3, 144.5, 147.6, 147.7, 148.2, 171.0. (+) LRESIMS m/z 369 (100), 463 (35).

### COMPOUND 12.1.30: 4-{6,7-DIMETHOXY-2-[(1-METHYL-1H-IMIDAZOL-4-YL)METHYL]-1,2,3,4-TETRAHYDROISOQUINOLIN-1-YL}-N,N-DIETHYLBENZAMIDE

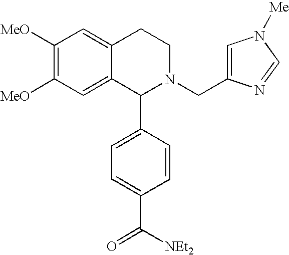

INTERMEDIATE 5.1.7 (50 mg, 0.14 mmol) and 1-methyl-4-imidazolecarbaldehyde (30 mg, 0.27 mmol) were dissolved in DCE (2 mL). After stirring for 10 min at room temperature, sodium triacetoxyborohydride (86 mg, 0.41 mmol) was added and the mixture was stirred for 18 h at room temperature. Resin-bound tosylhydrazine (73 mg, 2.4 mmol/g) was added and the mixture was agitated for another 2 h. DCM and 1 M sodium hydroxide solution were added and the mixture was passed through a Whatman 1PS silicon-treated filter paper. The organic phase was evaporated and the crude product was purified by flash chromatography to yield the product (23 mg, 0.05 mmol, 37%). The ¹H NMR shows very broad signals. ¹H NMR (500 MHz, CDCl₃): 1.05, 1.28 (2 brs, 6H), 2.70-2.83, 3.01, 3.29 (3 m, 6H), 3.50-3.70 (m, 4H), 3.66 (s, 3H), 3.69, 3.86 (2 s, 6H), 4.73 (s, 1H), 6.12, 6.61 (2 s, 2H), 6.78 (s, 1H), 7.34-7.40 (m, 5H). (+) LRESIMS m/z 369 (100), 463 (35).

### COMPOUND 12.1.31: 4-({6,7-DIMETHOXY-2-[(4-METHYL-1H-IMIDAZOL-5-YL)METHYL]-1,2,3,4-TETRAHYDROISOQUINOLIN-1-YL}METHOXY)-N,N-DIETHYLBENZAMIDE

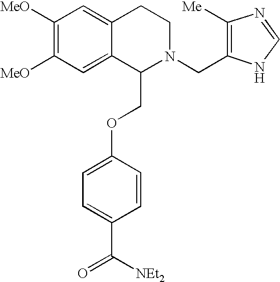

To a solution of INTERMEDIATE 5.1.1 (0.2 mg, 0.5 mmole) and 4-methyl-5-imidazolecarboxaldehyde (0.06 g, 0.6 mmole) in 1,2-dichloromethane (5 mL) was added sodium triacetoxyborohydriden (0.32 g, 1.5 mmole) and the resulting solution was stirred at room temperature for 18 h. Sodium hydroxide (1M, 20 mL) and chloroform (60 mL) was added and the mixture filtered through a Whatman 1PS filter paper. The solvent was removed from the organic phase in vacuo and the residue purified by flash chromatography (2/98 Methanol/Chloroform) to give COMPOUND 12.1.31 (0.06 g, 25%) as a yellow amorphous solid. ¹H NMR (500 MHz, CDCl₃): a 1.17 (br s, 6H), 2.14 (s, 3H), 2.56 (dd, J 4, 14.5 Hz, 1H), 3.83 (m, 2H), 3.84 (s, 3H), 3.86 (s, 3H), 4.03 (app q, J 5 Hz, 2H), 4.27 (app t, J 10 Hz, 1H), 5.95 (br s, 1H), 6.62 (s, 1H), 6.67 (s, 1H), 6.87 (d, J 9 Hz, 2H), 7.31 (d, J 9 Hz, 2H), 7.49 (s, 1). ¹³C NMR (125 MHz, CDCl₃): δ11.28, 24.22, 44.38, 48.34, 55.86, 56.01, 59.20, 71.59, 111.14, 111.56, 114.30, 125.38, 125.64, 127.12, 128.18, 129.78, 130.76, 132.99, 147.53, 148.17, 159.27, 171.15. (+) LRESIMS m/z 493 [M+H]⁺.

### COMPOUND 12.1.32: 4-({6,7-DIMETHOXY-2-[(4-METHYL-1H-IMIDAZOL-5-YL)METHYL]-1,2,3,4-TETRAHYDROISOQUINOLIN-1-YL}METHYL)-N,N-DIETHYLBENZAMIDE

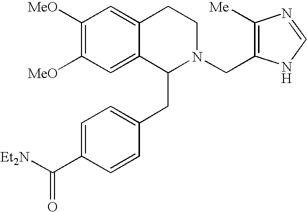

INTERMEDIATE 5.1.5 (25 mg, 0.05 mmol) and 1-methyl-4-imidazolecarbaldehyde (12 mg, 0.10 mmol) were dissolved in DCE (5 mL). After stirring for 10 min at room temperature, sodium triacetoxyborohydride (33 mg, 0.16 mmol) was added and the mixture was stirred for 18 h at room temperature. Resin-bound tosylhydrazine (100 mg, 1.5 mmol/g) was added and the mixture was agitated for another 2 h. DCM and 1 M sodium hydroxide solution were added and the mixture was passed through a Whatman 1PS silicon-treated filter paper. The organic phase was evaporated and the crude product was purified by preparative LCMS to yield the desired product (10 mg, 0.02, 39%). ¹H NMR (500 MHz, CDCl₃): 1.14, 1.28 (2 brs, 6H), 2.17 (s, 3H), 3.10-3.60, 3.80 (2 m, 10H), 4.35 (brs, 2H), 4.41 (s, 1H), 6.14, 6.59 (2 s, 2H), 7.29 (m, 4H), 8.50 (brs, 1H). (+) LRESIMS m/z 369 (100), 463 (35).

### COMPOUND 12.1.33: 1-{4-[(DIETHYLAMINO)CARBONYL]PHENYL}-2-[(4-METHYL-1H-IMIDAZOL-5-YL)METHYL]-1,2,3,4-TETRAHYDROISOQUINOLIN-6-YL METHANESULFONATE

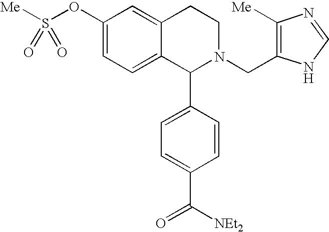

INTERMEDIATE 6.4.1 (27 mg, 0.067 mmol) and 5-methyl-4-imidazolecarbaldehyde (15 mg, 0.134 mmol) were dissolved in 1,2-dichloroethane (3.0 mL) and sodium triacetoxyboronhydride (43 mg, 0.20 mmol) was added. The mixture was stirred for 18 h after which ethyl acetate and 1 M sodium hydroxide solution were added. After phase separation the aqueous phase was extracted with ethyl acetate and the combined organic extracts were washed with water and brine. Tosylhydrazine resin (100 mg, 1.5 mmol/g) was added and the mixture was stirred for 2 h. After filtration and washing of the resin the filtrate was evaporated and the residue was purified by flash chromatography, which yielded the product (7 mg, 0.014 mmol, 21%). ¹H NMR (500 MHz, CDCl₃): 1.12, 1.28 (2 brs, 6H), 2.12 (s, 3H), 2.59, 2.83, 3.05, 3.13 (4 m, 4H), 3.15 (s, 3H), 3.29, 3.56 (2 brs, 4H), 3.37, 3.62 (2 d, J 12.0 Hz, 2H), 4.63 (s, 1H), 6.74 (d, 8.5 Hz, 1H), 6.95 (dd, J 8.5, 2.0 Hz, 1H), 7.09 (d, J 2.0 Hz, 1H), 7.34 (m, 4H), 7.39 (s, 1H). (+) LRESIMS m/z 497 (100) [M+H]⁺.

### COMPOUND 12.1.34: 1-{4-[(DIETHYLAMINO)CARBONYL]PHENYL}-2-[(2-PHENYL-1-IMIDAZOL-5-YL)METHYL]-1,2,3,4-TETRAHYDROISOQUINOLIN-6-YL METHANESULFONATE

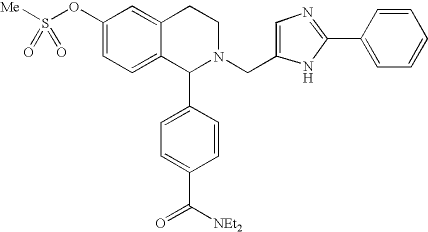

INTERMEDIATE 6.4.1 (27 mg, 0.067 mmol) and 2-phenyl-4(5)-imidazolecarbaldehyde (23 mg, 0.134 mmol) were dissolved in 1,2-dichloroethane (3.0 mL) and sodium triacetoxyboronhydride (43 mg, 0.20 mmol) was added. The mixture was stirred for 18 h after which ethyl acetate and 1 M sodium hydroxide solution were added. After phase separation the aqueous phase was extracted with more ethyl acetate and the combined organic phases were washed with water and brine. Tosylhydrazine resin (100 mg, 1.5 mmol/g) was added and the mixture was stirred for 2 h. After filtration and thorough washing of the resin the filtrate was evaporated and the residue was purified by flash chromatography the product (36 mg (0.064 mmol, 96%). ¹H NMR (500 MHz, CDCl₃): 1.13, 1.27 (2 brs, 6H), 2.64, 2.82, 3.10, 3.12 (4 m, 4H), 3.11 (s, 3H), 3.28, 3.56 (2 brs, 411, 3.45, 3.56 (2 d, J 12.0 Hz, 2H), 4.68 (s, 1H), 6.68 (d, 8.5 Hz, 1H), 6.86 (s, 1H), 6.90 (dd, J 8.5, 2.0 Hz, 1H), 7.05 (d, J 2.0 Hz, 1H), 7.34 (m, 7H), 7.89 (d, J 7.5 Hz, 2H). (+) LRESIMS m/z 559 (100) [M+H]⁺.

### COMPOUND 12.1.35: 1-{4-[(DIETHYLAMINO)CARBONYL]PHENYL}-2-[(4-METHYL-1H-IMIDAZOL-5-YL)METHYL]-1,2,3,4-TETRAHYDROISOQUINOLIN-6-YL DIMETHYLSULFAMATE

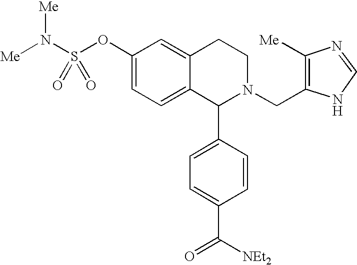

INTERMEDIATE 6.4.2 (12 mg, 0.028 mmol) and 5-methyl-4-imidazolecarbaldehyde (6 mg, 0.056 mmol) were dissolved in 1,2-dichloroethane (3.0 mL) and sodium triacetoxyboronhydride (18 mg, 0.084 mmol) was added. The mixture was stirred for 18 h after which ethyl acetate and 1 M sodium hydroxide solution were added. The aqueous phase was extracted with more ethyl acetate and the combined organic phases were washed with water and brine. Polymer-bound tosylhydrazine (147 mg, 1.5 mmol/g) was added and the mixture was stirred for 2 h. After filtration and washing of the resin the filtrate was evaporated and the residue was purified by preparative LCMS to yield the product (5 mg, 0.01 mmol, 32%). ¹H NMR (500 MHz, CDCl₃): 1.19 (brs, 6H), 2.10 (s, 3H), 2.51 (s, 6H), 2.63, 2.90-3.20 (2 m, 4H), 4.81 (s, 1H), 6.80 (d, J 8.5 Hz, 1H), 7.28 (m, 4H), 8.85 (s, 1H). (+) LRESIMS m/z 526 (100) [M+H]⁺.

### COMPOUND 12.1.36: 1-{4-[(DIETHYLAMINO)CARBONYL]PHENYL}-2-[(2-PHENYL-1H-IMIDAZOL-5-YL)METHYL]-1,2,3,4-TETRAHYDROISOQUINOLIN-6-YL DIMETHYLSULFAMATE

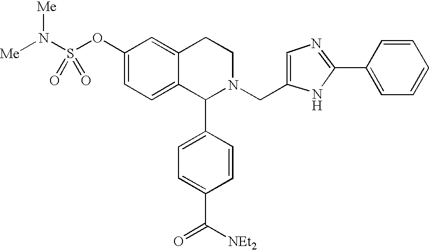

INTERMEDIATE 6.4.2 (12 mg, 0.028 mmol) and 2-phenyl-4(5)-imidazolecarbaldehyde (10 mg, 0.056 mmol) were dissolved in 1,2-dichloroethane (3 mL) and sodium triacetoxyboronhydride (18 mg, 0.084 mmol) was added. The mixture was stirred for 18 h after which ethyl acetate and 1 M aqueous sodium hydroxide solution were added. After phase separation the aqueous phase was extracted with more ethyl acetate and the combined organic phases were washed with water and brine. Resin-bound tosylhydrazine (147 mg, 1.5 mmol/g) was added and the mixture was stirred for 2 h. After filtration and thorough washing of the resin, the filtrate was evaporated and the residue was purified by flash chromatography to yield the product (11 mg, 0.019 mmol, 67%). ¹H NMR (500 MHz, CDCl₃): 1.14, 1.27 (2 brs, 6H), 2.67, 2.86, 3.05, 3.17 (4 m, 4H), 2.97 (s, 6H), 3.18, 3.60 (2 brs, 4H), 3.51, 3.60 (2 d, J 12.0 Hz, 2H), 4.72 (s, 1H), 6.69 (d, 8.5 Hz, 1H), 6.92 (s, 1H), 6.94 (dd, J 8.5, 2.0 Hz, 1H), 7.08 (d, J 2.0 Hz, 1H), 7.34 (m, 7H), 7.86 (d, J 7.5 Hz, 2H). (+) LRESIMS m/z 559 (100) [M+H]⁺.

### COMPOUND 12.1.37: 4-{2-[(2,5-DIMETHYL-1,3-THIAZOL-4-YL)METHYL]-6,7-DIMETHOXY-1,2,3-TETRAHYDROISOQUINOLIN-1-YL}-N,N-DIETHYLBENZAMIDE

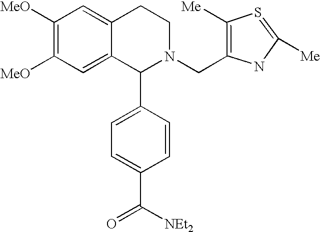

INTERMEDIATE 5.1.7 (50 mg, 0.14 mmol) and 2,4-dimethyl-5-thiazolecarbaldehyde (38 mg, 0.27 mmol) were dissolved in 1,2-dichloroethane (2 mL) and sodium triacetoxyborohydride (115 mg, 0.54 mmol) was added. The reaction mixture was stirred at room temperature for 18 h. Resin-bound tosylhydrazine (0.27 g, 1.5 mmol/g) was added and the mixture was agitated for another 2 h. Dichloromethane and 2 M aqueous sodium hydroxide solution were added, the mixture was shaken and passed through a Whatman 1PS filter paper. The organic phase was evaporated to dryness and purified by flash chromatography to yield the product (70 mg, 0.141 mmol, quant). ¹H NMR (500 MHz, CDCl₃): 1.02, 1.15 (2 brs, 6H), 2.13 (s, 3H), 2.53, 2.68, 2.85, 3.04 (4 m, 7H), 3.19, 3.45 (2 brs, 4H), 3.41, 3.65 (2 d, J 14 Hz, 2H), 3.53, 3.77 (2 s, 6H), 4.53 (s, 1H), 6.11, 6.54 (2 s, 2H), 7.27 (s, 4H). (+) LRESIMS m/z 494 (100) [M+H]⁺.

### COMPOUND 12.1.38: 4-{6,7-DIMETHOXY-2-[(2-PHENYL-1,3-THIAZOL-5-YL)METHYL]-1,2,3,4-TETRAHYDROISOQUINOLIN-1-YL}-N,N-DIETHYLBENZAMIDE

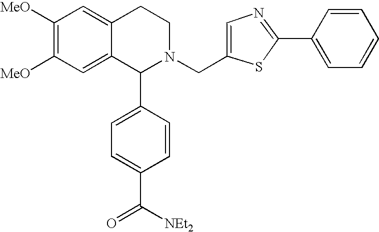

INTERMEDIATE 5.1.7 (50 mg, 0.14 mmol) and 2-phenyl-4-thiazolecarbaldehyde (51 mg, 0.27 mmol) were dissolved in 1,2-dichloroethane (2 mL) and sodium triacetoxyborohydride (115 mg, 0.54 mmol) was added. The reaction mixture was stirred at room temperature for 18 h. Resin-bound tosylhydrazine (0.27 g, 1.5 mmol/g) was added and the mixture was agitated for another 2 h. Dichloromethane and 2 M aqueous sodium hydroxide solution were added, the mixture was shaken and passed through a Whatman 1PS filter paper. The organic phase was evaporated to dryness and purified by flash chromatography to yield the product (74 mg, 0.136 mmol, quant.). ¹H NMR (500 MHz, CDCl₃): 1.09, 1.23 (2 brs, 6H), 2.80, 3.04, 3.28 (3 m, 6H), 3.52 (brs, 2H), 3.82, 3.90 (2 d, J 14 Hz, 2H), 3.61, 3.85 (2 s, 6H), 4.78 (s, 1H), 6.20, 6.63 (2 s, 2H), 7.35-7.43 (m, 7H), 7.94 (d, J 7.5 Hz, 2H). (+) LRESIMS m/z 542 (100) [M+H]⁺.

### COMPOUND 12.1.39: N,N-DIETHYL-4-{7-ISOPROPOXY-6-METHOXY-2-[(5-METHYL-1H-IMIDAZOL-4-YL)METHYL]-1,2,3,4-TETRAHYDROISOQUINOLIN-1-YL}BENZAMIDE

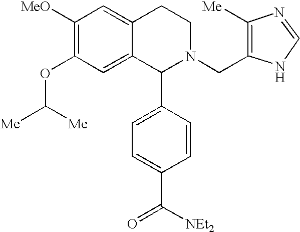

To a solution of INTERMEDIATE 7.1.2 (23.8 mg, 0.0601 mmol) in anhydrous 1,2-dichloroethane (1 mL) was added 4-methyl-1H-imidazole-5-carbaldehyde (9.9 mg, 0.0902 mmol, 1.5 eq). After stirring for 5 min, sodium triacetoxyborohydride (37.9 mg, 0.180 mmol, 3 eq) was added in one lot. The reaction mixture was stirred at RT for 48 hr, then quenched with saturated aqueous sodium bicarbonate (0.8 mL) and extracted with dichloromethane (2×10 mL). The excess aldehyde was removed by stirring the extracted dichloromethane with polymer supported hydrazine for 2 hr. The polymer was filtered off and the filtrate was concentrated and dried under vacuum. The product was purified by flash chromatography, using SiO₂ column with MeOH/DCM (10:90) to give 23.3 mg (0.0475 mmol, 79%) of COMPOUND 12.1.39 as light yellow oil. ¹H NMR (500 MHz, CDCl₃) δ 1.12 (br s, 3H), 1.13 (d, J 7 Hz, 3H), 1.20 (d, J 7 Hz, 3H), 1.21 (br s, 3H), 2.12 (s, 3H), 2.50 (m, 1H), 2.70 (m, 1H), 2.80 (m, 1H), 3.10 (m, 1H), 3.25 (br s, 2H), 3.40 (m, 1H), 3.55 (br s, 2H), 3.60 (m, 1H), 3.82 (s, 3H), 4.20 (q, J 7 Hz, 1H), 4.54 (s, 1H), 6.19 (s, 1H), 6.61 (s, 1H), 7.30 (m, 4H). ¹³C NMR (125 MHz, CDCl₃) δ11.13, 13.10, 14.20, 21.91, 22.19, 28.25, 39.50, 43.80, 47.04, 49.30, 56.11, 67.72, 71.75, 111.28, 117.27, 126.48, 127.83, 129.32, 129.87, 133.60, 136.29, 145.46, 145.75, 149.47, 171.58. (+) LRESIMS m/z 491 (M+H)⁺.

### COMPOUND 12.1.40: N,N-DIETHYL-[6-METHOXY-2-[(5-METHYL-1H-IMIDAZOL-4-YL)METHYL]-7-(2-MORPHOLIN-4-YLETHOXY)-1,2,3,4-TETRAHYDROISOQUINOLIN-1-YL]BENZAMIDE

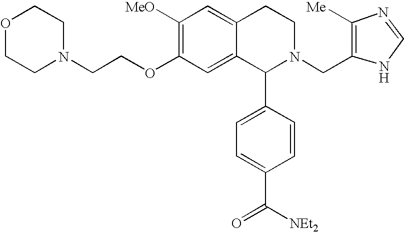

To a solution of INTERMEDIATE 7.1.3 (22 mg, 0.0471 mmol) in anhydrous 1,2-dichloroethane (1 mL) was added 4-methyl-1H-imidazole-5-carbaldehyde (10 mg, 0.0909 mmol, 1.9 eq). After stirring for 5 min, sodium triacetoxyborohydride (29.8 mg, 0.141 mmol, 3 eq) was added in one lot. The reaction mixture was stirred at RT for 48 hr, then quenched with saturated aqueous sodium bicarbonate (0.8 mL) and extracted with dichloromethane (2×10 mL). Excess aldehyde was removed by stirring the extracted dichloromethane with polymer supported hydrazine for 2 hr. The polymer was filtered off and the filtrate was concentrated and dried under vacuum. Product was purified by flash chromatography, using SiO₂ column with MeOH/DCM (10:90) gave 24.6 mg (0.0438 mmol, 93%) of COMPOUND 12.1.40 as oil. ¹H NMR (500 MHz, CDCl₃): δ 1.15 (br s, 3H), 1.28 (br s, 3H), 2.13 (s, 3H), 2.51 (m, 4H), 2.58 (m, 1H), 2.69 (m, 2H), 2.75 (m, 1H), 2.90 (m, 1H), 3.08 (m, 1H), 3.30 (br s, 2H), 3.40 (1H), 3.55 (br s, 2H), 3.60 (m, 1H), 3.69 (m, 4H), 3.70 (s, 3H), 3.85-3.95 (m, 2H), 4.58 (s, 1H), 6.23 (s, 1H), 6.62 (s, 1H), 7.28-7.34 (m, 4H). ¹³C NMR (125 MHz, CDCl₃): δ 10.96, 13.10, 14.10, 27.96, 39.69, 43.66, 46.56, 49.03, 50.84, 54.26, 56.13, 67.08, 67.35, 111.81, 114.92, 126.59, 127.94, 128.96, 129.92, 132.83, 136.37, 145.20, 146.72, 148.79, 171.47. (+) LRESIMS m/z 562 (M+H)⁺.

### COMPOUND 12.1.41: 4-{7-ETHOXY-6-METHOXY-2-[(2-PHENYL-1H-IMIDAZOL-4-YL)METHYL]-1,2,3,4-TETRAHYDROISOQUINOLIN-1-YL}-N,N-DIETHYLBENZAMIDE

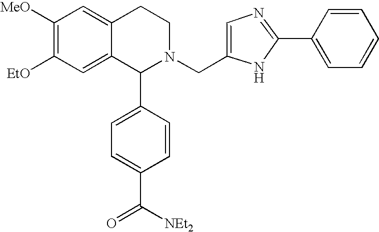

To a solution of INTERMEDIATE 7.1.1 (8 mg, 0.021 mmol) in anhydrous 1,2-dichloroethane (1 mL) was added 2-phenyl-1H-imidazole-5-carbaldehyde (7.2 mg, 0.0419 mmol, 2 eq). After stirring for 5 min, sodium triacetoxyborohydride (26 mg, 0.126 mmol, 3 eq) was added in one lot. The reaction mixture was stirred at RT for 60 hr, then quenched with saturated aqueous sodium bicarbonate (0.8 mL) and extracted with dichloromethane (2×10 mL). Excess aldehyde was removed by stirring the extracted dichloromethane with polymer supported hydrazine for 2 hr. The polymer was filtered off and the filtrate was concentrated and dried under vacuum. Product was purified by flash chromatography, using SiO₂ column with MeOH/DCM (10:90) afford 5.2 mg (0.00966 mmol, 46%) of COMPOUND 12.1.41 as light yellow oil. ¹H NMR (500 MHz, CDCl₃): δ 1.15 br s, 3H), 1.23 (br s, 3H), 1.40 (m, 3H), 2.65-3.15 (m, 4H), 3.30 (br s, 2H), 3.55 (br s, 2H), 3.64 (m, 2H), 3.89 (s, 3H), 3.90 (m, 2H), 3.98 (m, 1H), 6.50 (m, 1H), 6.75 (m, 1H), 6.80 (br s, 1H), 7.20-7.40 (m, 9H), 8.20 (br s, 1H). (+) LRESIMS m/z 539 (M+H)⁺.

### COMPOUND 12.1.42: N,N-DIETHYL-4-{7-ISOPROPOXY-6-METHOXY-2-[(2-PHENYL-1H-IMIDAZOL-4-YL)METHYL]-1,2,3,4-TETRAHYDROISOQUINOLIN-1-YL}BENZAMIDE

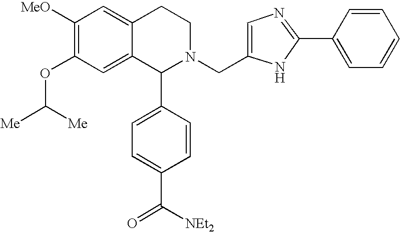

To a solution of INTERMEDIATE 7.1.2 (26 mg, 0.0656 mmol) in anhydrous 1,2-dichloroethane (1.5 mL) was added 2-phenyl-1H-imidazole-5-carbaldehyde (22.5 mg, 0.131 mmol, 2 eq). After stirring for 5 min, sodium triacetoxyborohydride (41.7 mg, 0.196 mmol, 3 eq) was added in one lot. The reaction mixture was stirred at RT for 60 hr, then quenched with saturated aqueous sodium bicarbonate (0.8 mL) and extracted with dichloromethane (2×10 mL). Excess aldehyde was removed by stirring the extracted dichloromethane with polymer supported hydrazine for 2 hr. The polymer was filtered off and the filtrate was concentrated and dried under vacuum. Product was purified by flash chromatography, using SiO₂ column with MeOH/DCM (10:90) afford 11.6 mg (0.021 mmol, 32%) of COMPOUND 12.1.42 as light yellow oil. ¹H NMR (500 MHz, CDCl₃): δ 1.11 (br s, 3H), 1.13 (m, 3H), 1.21 (m, 3H), 1.28 (br s, 3H), 2.78-3.05-3.20 (m, 4H), 3.25 (br s, 2H), 3.56 (br s, 2H), 3.78 (m, 2H), 3.83 (s, 3H), 4.22 (m, 1H), 4.80 (br s, 1H), 6.19 (s, 1H), 6.34 (s, 1H), 6.93 (s, 1H), 7.28-7.49 (m, 9H). ¹³C NMR (125 MHz, CDCl₃): δ 13.00, 14.50, 21.19, 28.00, 39.64, 43.60, 46.74, 49.89, 56.15, 66.62, 71.81, 111.85, 116.97, 125.68 (2C), 126.67 (2C), 126.70, 128.10 (2C), 129.87 (2C), 129.50, 129.70, 130.07, 136.10, 145.00, 146.00, 150.00, 171.42. (+) LRESIMS m/z 553 (M+H)⁺.

### COMPOUND 12.1.43: N,N-DIETHYL-4-{6-METHOXY-7-(2-MORPHOLIN-4-YLETHOXY)-2-[(2-PHENYL-1H-IMIDAZOL-4-YL)METHYL]-1,2,3,4-TETRAHYDROISOQUINOLIN-1-YL}BENZAMIDE

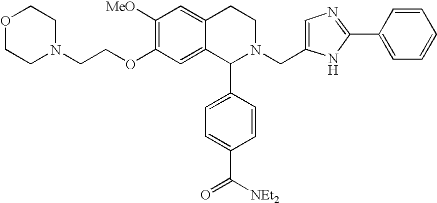

To a solution of INTERMEDIATE 7.1.3 (22 mg, 0.047 mmol) in anhydrous 1,2-dichloroethane (2 mL) was added 2-phenyl-1H-imidazole-5-carbaldehyde (16 mg, 0.094 mmol, 2 eq). After stirring for 5 min, sodium triacetoxyborohydride (30 mg, 0.141 mmol, 3 eq) was added in one lot. The reaction mixture was stirred at RT for 60 hr, then quenched with saturated aqueous sodium bicarbonate (0.8 mL) and extracted with dichloromethane (2×10 mL). Excess aldehyde was removed by stirring the extracted dichloromethane with polymer supported hydrazine for 2 hr. The polymer was filtered off and the filtrate was concentrated and dried under vacuum. Product was purified by flash chromatography, using SiO₂ column with MeOH/DCM (10:90) afford 16.4 mg (0.0263 mmol, 56%) of COMPOUND 12.1.43 as light yellow oil. ¹H NMR (500 MHz, CDCl₃): δ 1.13 (br s, 3H), 1.26 (br s, 3H), 2.54 (br s, 4H), 2.73 (br m, 2H), 2.80-3.18 (m, 4H), 3.35 (br s, 2H), 3.56 (br s, 2H), 3.70 (br s, 4H), 3.86 (s, 3H), 3.95 (br m, 4H), 4.85 (s, 1H), 6.23 (s, 1H), 6.64 (s, 1H), 6.96 (s, 1H), 7.30-7.93 (m, 9H). ¹³C NMR (125 MHz, CDCl₃): δ 13.10, 14.20, 27.50, 39.80, 43.10, 46.00, 49.50, 54.10, 54.30, 56.30, 66.20, 66.50, 66.80, 112.10, 115.50, 125.80, 127.00, 128.80, 128.85, 129.50, 129.60, 129.80, 130.00, 137.00, 147.50, 149.10, 171.54. (+) LRESIMS m/z 624 (M+H)⁺.

### COMPOUND 12.1.44: N,N-DIETHYL-4-{7-METHOXY-2-[(4-METHYL-1H-IMIDAZOL-5-YL)METHYL]-1,2,3,4-TETRAHYDROISOQUINOLIN-1-YL}BENZAMIDE

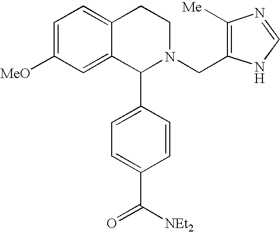

A solution of INTERMEDIATE 4.2.2 (0.25 g, 0.7 mmole), 4-methyl-5-imidazolecarboxaldehyde (0.09 g, 0.8 mmole) and sodium triacetoxyborohydride (0.47 g, 2.2 mmole) in 1,2-dichloroethane (5 mL) was stirred at room temperature for 18 h. 1 M sodium hydroxide (20 mL) was added and the mixture extracted with ethyl acetate (3×20 mL), the combined organics dried (MgSO₄), filtered and the solvent removed in vacuo. The residue was purified by flash chromatography (methanol/chloroform, 2/98) to give COMPOUND 12.1.44 (0.24 g, 76%) as a yellow solid. ¹H NMR (500 MHz, CDCl₃): δ1.12, 1.24 (2 br s, 6H), 2.08 (s, 3H), 2.53 (m, 1H), 2.71 (m, 1H), 2.90 (m, 1), 3.06 (m, 1H), 3.28 (br s, 2H), 3.35 (d, J 14 Hz, 1H), 3.52 (br s, 2H), 3.60 (d, J 14 Hz, 1H), 3.63 (s, 3H), 4.59 (s, 1H), 6.25 (d, J 2 Hz, 1H), 6.71 (dd, J 2, 8 Hz, 1H), 7.04 (d, J 8 Hz, 1H), 7.28 (m, 5H), 8.20 (br s, 1H). (+) LRESIMS m/z 433 [M+H]⁺.

### COMPOUND 12.1.45: METHYL 5-{[1-{4-[(DIETHYLAMINO)CARBONYL]PHENYL}-6,7-DIMETHOXY-3,4-DIHYDROISOQUINOLIN-2(1H)-YL]METHYL}-1H-IMIDAZOLE-4-CARBOXYLATE

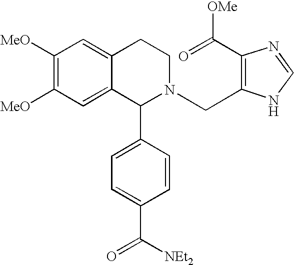

INTERMEDIATE 5.1.7 (63 mg, 0.17 mmol) and methyl 5-formyl-1H-imidazole-4-carboxylate (50 mg, 0.32 mmol) were dissolved in 1,2-dichloroethane (10 mL) and sodium triacetoxyborohydride (0.15 g, 0.70 mmol) was added. The reaction mixture was stirred at room temperature for 18 h. Dichloromethane and 2 M aqueous sodium hydroxide solution were added, the mixture was shaken and passed through a Whatman 1PS filter paper. The organic phase was evaporated to dryness and purified by flash chromatography to yield the product (84 mg, 0.17 mmol, 98%). ¹H NMR (500 MHz, CDCl₃): 1.10, 1.23 (2 brs, 6H), 2.67, 2.80, 3.00, 3.09 (4 m, 4H), 3.28 (brs, 2H), 3.58 (brs, 2H), 3.62 (s, 3H), 3.82 (s, 3H), 3.84 (s, 3H), 3.93 (d, J 12.5 Hz, 1H), 4.19 (d, J 12.5 Hz, 1H), 4.78 (s, 1H), 6.18 (s, 1H), 6.42 (s, 1H), 7.35-7.38 (m, 5H).

### COMPOUND 12.1.46: 1-{4-[(DIETHYLAMINO)CARBONYL]PHENYL}-6-METHOXY-2-[(4-METHYL-1H-IMIDAZOL-5-YL)METHYL]-1,2,3,4-TETRAHYDROISOQUINOLIN-7-YL METHANESULFONATE

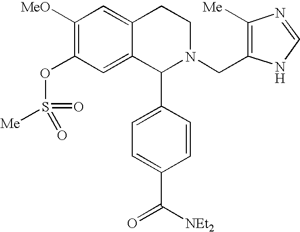

INTERMEDIATE 11.1.1 (37 mg, 0.086 mmol) and 5-methylimidazole-4-carbaldehyde (24 mg, 0.21 mmol) were dissolved in 1,2-dichloroethane (2 mL) and stirred for 10 min at room temperature. Sodium triacetoxyborohydride (73 mg, 0.34 mmol) was added and the mixture was stirred for 18 h. Tosylhydrazine resin (280 mg, 1.5 mmol/g) and DCM (5 mL) were added and the mixture was stirred for another 2 h. The resin was filtered off and washed twice with DCM. The filtrate was washed with 1 M aqueous sodium hydroxide solution, water, and brine, dried, and evaporated. Flash chromatography of the residue yielded the desired product (32 mg, 0.061 mmol, 71%). ¹H NMR (500 MHz, CDCl₃): 1.10, 1.25 (2 brs, 6H), 2.10 (s, 3H), 2.57, 2.88, 3.00, 3.19 (4 m, 4H), 3.09 (s, 3H), 3.29, 3.58 (2 brs, 4H), 3.38, 3.60 (2 d, J 12.5 Hz, 2H), 3.86 (s, 3H), 4.54 (s, 1H), 6.60, 6.73 (2 s, 2H), 7.28 (s, 1H), 7.32 (s, 4H). (+) LRESIMS m/z 527 (100) [M+H]⁺.

### COMPOUND 12.1.47: N,N-DIETHYL-4-{6-[(4-METHYL-1H-IMIDAZOL-5-YL)METHYL]-5,6,7,8-TETRAHYDRO[1,3]DIOXOLO[4,5-G]ISOQUINOLIN-5-YL}BENZAMIDE

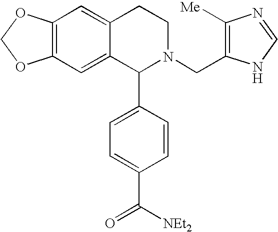

INTERMEDIATE 5.1.13 (50 mg, 0.14 mmol) and 5-methylimidazole-4-carbaldehyde (39 mg, 0.35 mmol) were dissolved in 1,2-dichloroethane (5 mL) and stirred for 10 min at room temperature. Sodium triacetoxyborohydride (118 mg, 0.56 mmol) was added and the mixture was stirred for 18 h. Tosylhydrazine resin (450 mg, 1.5 mmol/g) and ethyl acetate (5 mL) were added and the mixture was stirred for another 2 h. The resin was filtered off and washed twice with ethyl acetate. The filtrate was washed with 1 M aqueous sodium hydroxide solution, water, and brine, dried, and evaporated. Flash chromatography of the residue yielded the desired product (43 mg, 0.10 mmol, 69%). ¹H NMR (500 MHz, CDCl₃): 1.12, 1.25 (2 brs, 6H), 2.09 (s, 3H), 2.53, 2.71, 2.84, 3.09 (4 m, 4H), 3.30, 3.57 (2 brs, 4H), 3.33, 3.58 (2 d, J 14 Hz, 2H), 4.52 (s, 1H), 5.84 (d, J 5 Hz, 2H), 6.16, 6.57 (2 s, 2H), 7.28 (s, 1H), 7.31 (s, 4H). (+) LRESIMS m/z 447 (100) [M+H]⁺.

### COMPOUND 12.1.48: N,N-DIETHYL-4-{6-[(2-PHENYL-1H-IMIDAZOL-5-YL)METHYL]-5,6,7,8-TETRAHYDRO[1,3]DIOXOLO[4,5-G]ISOQUINOLIN-5-YL}BENZAMIDE

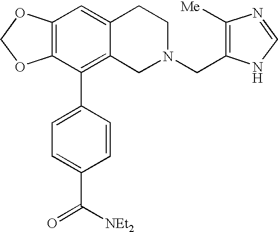

INTERMEDIATE 5.1.13 (50 mg, 0.14 mmol) and 2-phenylimidazole-4-carbaldehyde (74 mg, 0.35 mmol) were dissolved in 1,2-dichloroethane (5 mL) and stirred for 10 min at room temperature. Sodium triacetoxyborohydride (118 mg, 0.56 mmol) was added and the mixture was stirred for 18 h. Tosylhydrazine resin (450 mg, 1.5 mmol/g) and DCM (5 mL) were added and the mixture was stirred for another 2 h. The resin was filtered off and washed twice with DCM. The filtrate was washed with 1 M aqueous sodium hydroxide solution, water, and brine, dried, and evaporated. Flash chromatography of the residue yielded the desired product (43 mg, 0.11 mmol, 79%). ¹H NMR (500 MHz, CDCl₃): 1.04, 1.17 (2 brs, 6H), 2.52, 2.63, 2.77 2.99 (4 m, 4H), 3.19 (brs, 2H), 3.36-3.47 (m, 4H), 4.51 (s, 1H), 5.74 (d, J 3 Hz, 2H), 6.05, 6.47 (2 s, 2H), 6.78 (brs, 1H), 7.17-7.25 (m, 7H), 7.78 (d, J 7.5 Hz, 2H). (+) LRESIMS m/z 509 (100) [+H]⁺.

### COMPOUND 12.1.49: 4-{6-BROMO-7-METHOXY-2-[(4-METHYL-1H-IMIDAZOL-5-YL)METHYL]-1,2,3,4-TETRAHYDROISOQUINOLIN-1-YL}-N,N-DIETHYLBENZAMIDE

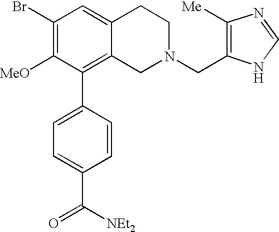

INTERMEDIATE 4.2.3 (25 mg, 0.06 mmol) and 5-methylimidazole-4-carbaldehyde (20 mg, 0.18 mmol) were dissolved in 1,2-dichloroethane (2 mL) and stirred for 10 min at room temperature. Sodium triacetoxyborohydride (64 mg, 0.30 mmol) was added and the mixture was stirred for 18 h. Tosylhydrazine resin (0.24 g, 1.5 mmol/g) and DCM (5 mL) were added and the mixture was stirred for another 2 h. The resin was filtered off and washed twice with DCM. The filtrate was washed with 1 M aqueous sodium hydroxide solution, water, and brine, dried, and evaporated. Flash chromatography of the residue yielded the desired product (13 mg, 0.026 mmol, 43%). ¹H NMR (500 MHz, CDCl₃): 1.12, 1.27 (2 brs, 6H), 2.18 (s, 3H), 2.61, 2.78, 2.90, 3.02 (4 m, 4H), 3.24, 3.57 (2 brs, 4H), 3.51, 3.65 (2 d, J 12.5 Hz, 2H), 3.64 (s, 3H), 4.64 (s, 1H), 6.23, 7.28, 7.53 (3 s, 3H), 7.37 (m, 4H). (+) LRESIMS m/z 512 (100) [M+H]⁺.

### COMPOUND 12.1.50: 4-{6-BROMO-7-METHOXY-2-[(2-PHENYL-1H-IMIDAZOL-5-YL)METHYL]-1,2,3,4-TETRAHYDROISOQUINOLIN-1-YL}-N,N-DIETHYLBENZAMIDE

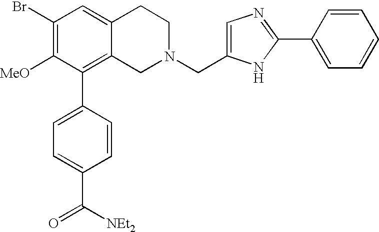

INTERMEDIATE 4.2.3 (25 mg, 0.06 mmol) and 2-phenylimidazole-4-carbaldehyde (31 mg, 0.18 mmol) were dissolved in 1,2-dichloroethane (2 mL) and stirred for 10 min at room temperature. Sodium triacetoxyborohydride (64 mg, 0.30 mmol) was added and the mixture was stirred for 18 h. Tosylhydrazine resin (0.24 g, 1.5 mmol/g) and DCM (5 mL) were added and the mixture was stirred for another 2 h. The resin was filtered off and washed twice with DCM. The filtrate was washed with 1 M aqueous sodium hydroxide solution, water, and brine, dried, and evaporated. Flash chromatography of the residue yielded the desired product (34 mg, 0.06 mmol, quant.). ¹H NMR (500 MHz, CDCl₃): 1.11, 1.27 (2 brs, 6H), 2.61, 2.75, 2.90, 3.10 (4 m, 4H), 3.15 (brs, 2H), 3.50-3.62 (m, 7H), 4.69 (s, 1H), 6.19, 6.90 (2 s, 2H), 7.25-7.37 (m, 8H), 7.91 (d, J 7.5 Hz, 2H). (+) LRESIMS m/z 574 (100) [M+H]⁺.

### COMPOUND 12.1.51: 4-{6,7-DIMETHOXY-3-METHYL-2-[(4-METHYL-1H-IMIDAZOL-5-YL)METHYL]-1,2,3,4-TETRAHYDROISOQUINOLIN-1-YL}-N,N-DIETHYLBENZAMIDE

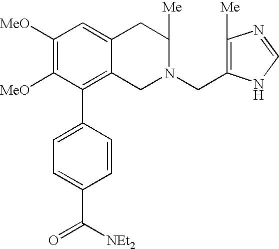

Methyl-imidazole-5-carboxyaldehyde (55.9 mg, 0.51 mmol) was added to a solution of INTERMEDIATE 5.1.14 (97 mg, 0.25 mmol) in 1,2-dichloroethane (4 mL) and the reaction mixture stirred at room temperature for 10 min. Sodium triacetoxyborohydride (378 mg, 1.78 mmol) was added followed by N-methyl-2-pyrrolidinone (320 μL) and the reaction mixture stirred at RT for 22 h. 1 N NaOH (2.5 mL) was added and the organic solvent removed in vacuo. The residue was extracted with DCM (3×10 mL) and the organic layer washed with water (10 mL). The organic layer was concentrated in vacuo and the residue purified by repeated flash chromatography On SiO₂ column (EtOAc:MeOH 95:5) to afford COMPOUND 12.1.51 as an oil (85.9 mg, 71%). ¹H NMR (500 MHz, CDCl₃): δ 1.10 (br s, 3H), 1.24 (br s, 3H), 1.45 (d, J 6.5 Hz, 3H), 2.30 (s, 3H), 3.15-3.30 (m, 4), 3.53 (m, 2H), 3.64 (s, 3H), 3.90 (s, 1H), 3.91 (s, 3H), 4.49 (d, J 14.5 Hz, 1H), 4.62 (d, J 14.5 Hz, 1H), 5.59 (s, 1H), 6.26 (s, 1H), 6.74 (s, 1H), 7.21-7.31 (m, 4H), 8.24 (s, 1H); ¹³C NMR (125 MHz, CDCl₃): δ 9.28, 12.82, 14.11, 19.11, 32.83, 40.13, 43.83, 46.93, 56.19, 56.25, 59.95, 67.61, 111.10, 111.24, 119.36, 121.66, 123.61, 127.29, 129.60, 132.72, 134.35, 137.08, 137.66, 149.15, 150.07, 170.73; (+) LRESIMS m/z 477.39 [M+H]⁺.

### COMPOUND 12.1.52: N,N-DIETHYL-4-[2-(1H-IMIDAZOL-5-YLMETHYL)-6,7-DIMETHOXY-3-METHYL-1,2,3,4-TETRAHYDROISOQUINOLIN-[1-YL]BENZAMIDE

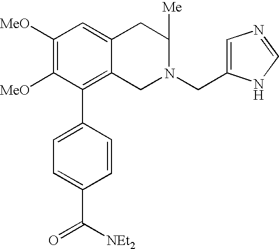

Imidazole-2-carboxyaldehyde (48.7 mg, 0.51 mmol) was added to a solution of INTERMEDIATE 5.1.14 (97 mg, 0.25 mmol) in 1,2-dichloroethane (4 mL) and the reaction mixture stirred at room temperature for 10 min. Sodium triacetoxyborohydride (378 mg, 1.78 mmol) was added followed by N-methyl-2-pyrrolidinone (320 μL) and the reaction mixture stirred at RT for 22 h. 1 N NaOH (2.5 mL) was added and the organic solvent removed in vacuo. The residue was extracted with DCM (3×10 mL) and the organic layer washed with water (10 mL). The organic layer was concentrated in vacuo and the residue purified by repeated flash chromatography on SiO₂ column (EtOAc:MeOH 95:5) to afford COMPOUND 12.1.52 as an oil (56.7 mg, 48%). ¹H NMR (500 MHz, CDCl₃): δ 1.09 (br s, 3H), 1.25 (br s, 3H), 1.35 (d, J 6 Hz, 3H), 2.65 (m, 1H), 2.76 (m, 1H), 3.07 (m, 1H), 3.24 (br s, 2H), 3.54 (br s, 2H), 3.56 (s, 3H), 3.64 (m, 1H), 3.82 (s, 3H), 3.94 (d, J 16 Hz, 1H), 4.84 (br s, 1H), 6.16 (s, 1H), 6.56 (s, 1H), 6.81 (s, 1H), 7.29-7.42 (m, 5H); ¹³C NMR (125 MHz, CDCl₃): δ 13.12, 14.41, 21.71, 38.39, 39.71, 43.66, 46.18, 52.54, 56.05, 65.64, 110.82, 111.83, 119.74, 126.60, 127.52, 129.31, 130.32, 133.33, 134.91, 135.80, 147.31, 147.54, 147.65, 171.80; (+) LRESIMS m/z 463.37 [M+H]⁺.

### COMPOUND 12.1.53: N,N-DIETHYL-4-{6-METHOXY-2-[(4-METHYL-1H-IMIDAZOL-5-YL)METHYL]-7-NITRO-1,2,3,4-TETRAHYDROISOQUINOLIN-1-YL}BENZAMIDE

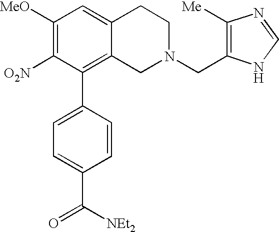

To a solution of INTERMEDIATE 9.2.2 (1.16 g, 3.00 mmole) and 4-methyl-5-imidazolecarboxaldehyde (0.31 g, 2.8 mmole) in 1,2-dichloromethane (25 mL) was added sodium triacetoxyborohydride (1.65 g, 7.8 mmole) and the resulting solution was stirred at room temperature for 18 h. Sodium hydroxide (1M, 100 mL) was added and the mixture extracted with ethyl acetate (3×100 mL). The organic extracts were dried (MgSO₄), filtered and the solvent removed in vacuo. The residue was purified by flash chromatography (ethyl acetate/10% methanol in chloroform, 2/8) to give COMPOUND 12.1.53 (1.12 g, 78%) as a yellow amorphous solid. ¹H NMR (500 MHz, CDCl₃): δ 1.12, 1.23 (2 br s, 6H), 2.05 (s, 3H), 2.55 (m, 1H), 2.82 (dd, J 3.5, 13 Hz, 1H), 3.02 (m, 1H), 3.12 (m, 1H), 3.26 (br s, 2H), 3.30 (d, J 13.5 Hz, 1H), 3.54 (br s, 2H), 3.56 (d, J 13.5 Hz, 1H), 3.91 (s, 3H), 4.56 (s, 1H), 6.73 (br s, 2H), 6.80 (s, 1H), 7.20 (s, 1H), 7.30 (m, 5H); ¹³C NMR (125 MHz, CDCl₃): δ 10.76, 12.82, 14.13, 29.35, 39.39, 43.45, 46.11, 49.33, 56.46, 67.12, 113.00, 126.13, 126.59, 127.03, 129.45, 129.50, 130.33, 133.05, 136.52, 137.67, 142.46, 144.01, 151.23, 171.07; (+) LRESIMS m/z 478 [M+H]⁺.

### COMPOUND 12.1.54: N,N-DIETHYL-4-{6-METHOXY-2-[(4-METHYL-1H-IMIDAZOL-5-YL)METHYL]-5-NITRO-1,2,3,4-TETRAHYDROISOQUINOLIN-1-YL}BENZAMIDE

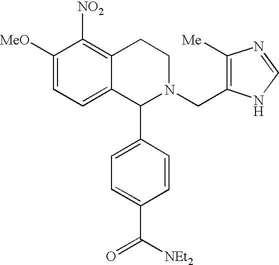

To a solution of Intermediate 9.2.1 (244 mg, 0.64 mmole) and 4-methyl-5-imidazolecarboxaldehyde (77 mg, 0.70 mmole) in 1,2-dichloromethane (10 mL) was added sodium triacetoxyborohydride (407 mg, 1.92 mmole) and the resulting solution was stirred at room temperature for 18 h. Sodium hydroxide (1M, 50 mL) was added and the mixture extracted with ethyl acetate (3×50 mL). The organic extracts were dried (MgSO₄), filtered and the solvent removed in vacuo. The residue was purified by flash chromatography (methanol/chloroform, 2/98) to give COMPOUND 12.1.54 (200 mg, 66%) as a yellow amorphous solid. ¹H NMR (500 MHz, CDCl₃): δ 1.12, 1.23 (2 br s, 6H), 2.04 (s, 3H), 2.48 (m, 1H), 2.65 (dd, J 4, 13 Hz, 1H), 2.88 (m, 1H), 3.09 (m, 1H), 3.28 (br s, 2H), 3.29 (d, J 14 Hz, 1H), 3.50 (br s, 2H), 3.57 (d, J 14 Hz, 1H), 3.80 (s, 3H), 4.57 (s, 1H), 6.73 (app t, J 10 Hz, 2H), 7.28 (m, 5H), 9.10 (br s, 1H); ¹³C NMR (125 MHz, CDCl₃): δ 10.69, 12.74, 14.11, 23.90, 39.33, 43.34, 45.59, 49.30, 56.24, 67.11, 110.35, 126.38, 127.19, 128.13, 129.16, 129.54, 131.06, 131.47, 133.10, 136.29, 140.50, 144.38, 148.89, 171.04; (+) LRESIMS m/z 478 [M+H]⁺.

### COMPOUND 12.1.55: N,N-DIETHYL-4-{7-[(4-METHYL-1H-IMIDAZOL-5-YL)METHYL]-6,7,8,9-TETRAHYDRO[1,3]DIOXOLO[4,5-F]ISOQUINOLIN-6-YL}BENZAMIDE

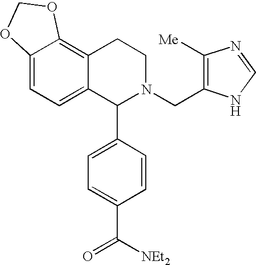

4-Methyl-imidazole-5-carboxyaldehyde (76.4 mg, 0.69 mmol) was added to a solution of INTERMEDIATE 5.1.15 (122.6 mg, 0.35 mmol) in 1,2-dichloroethane (5 mL) and the reaction mixture stirred at room temperature for 10 min. Sodium triacetoxyborohydride (516 mg, 2.43 mmol) was added followed by N-methyl-2-pyrrolidinone (400 μL) and the reaction mixture stirred at RT for 18 h. 1 N NaOH (5 mL) was added and the organic solvent removed in vacuo. The residue was extracted with EtOAc (3×20 mL) and the organic layer washed with water (20 mL). The organic layer was concentrated in vacuo and the residue purified by flash chromatography on SiO₂ column (EtOAc:MeOH 95:5) to afford COMPOUND 12.1.55 as an oil (35.6 mg, 23%). ¹H NMR (500 MHz, CDCl₃): δ 1.13 (br s, 3H), 1.25 (br s, 3H), 2.09 (s, 3H), 2.55 (m, 1H), 2.78 (m, 1H), 3.09 (m, 1H), 3.28 (br s, 2H), 3.34-3.40 (m, 2H), 3.55 (br s, 2H), 3.59 (d, J 13.5 Hz, 1H), 4.58 (s, 1H), 5.96 (s, 1H, 6.21 (d, J 8 Hz, 1H), 6.55 (d, J 8 Hz, 1H), 7.28-7.32 (m, 4H), 7.36 (s, 1H); ¹³C NMR (125 MHz, CDCl₃): δ 11.37, 13.11, 14.42, 22.28, 39.61, 43.64, 45.72, 49.40, 67.41, 101.30, 106.56, 117.66, 122.17, 126.56, 129.73, 130.71, 131.73, 133.45, 136.22, 144.81, 145.28, 145.54, 171.54; (+) LRESIMS m/z 447.20 [M+H]⁺.

### COMPOUND 12.1.56: N,N-DIETHYL-4-{7-[(2-PHENYL-1H-IMIDAZOL-5-YL)METHYL]-6,7,8,9-TETRAHYDRO[1,3]DIOXOLO[4,5-F]ISOQUINOLIN-6-YL}BENZAMIDE

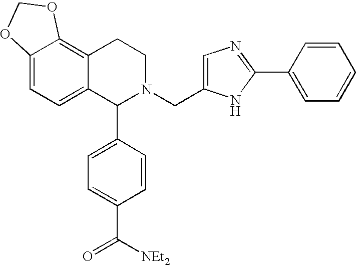

2-Phenyl-imidazole-4-carboxyaldehyde (109.4 mg, 0.63 mmol) was added to a solution of INTERMEDIATE 5.1.15 (111.2 mg, 0.32 mmol) in 1,2-dichloroethane (5 mL) and the reaction mixture stirred at room temperature for 10 min. Sodium triacetoxyborohydride (468 mg, 2.21 mmol) was added followed by N-methyl-2-pyrrolidinone (400 μL) and the reaction mixture stirred at RT for 18 h. 1 N NaOH (5 mL) was added and the organic solvent removed in vacuo. The residue was extracted with EtOAc (3×20 mL) and the organic layer washed with water (20 mL). The organic layer was concentrated in vacuo and the residue purified by flash chromatography on SiO₂ column (EtOAc:MeOH 95:5) to afford COMPOUND 12.1.56 as an oil (53.7 mg, 35%). ¹H NMR (500 MHz, CDCl₃): δ 1.12 (br s, 3H), 1.26 (br s, 3H), 2.63 (m, 1H), 2.78 (m, 2H), 3.10 (m, 1H), 3.27 (br s, 2H), 3.38 (t, J 7 Hz, 1H), 3.47-3.59 (m, 4H), 4.59 (s, 1H), 5.94 (s, 2H), 6.16 (d, J 8 Hz, 1H), 6.53 (d, J 8 Hz, 1H), 6.89 (s, 1H), 7.22-7.34 (m, 7H), 7.87 (d, J 8.5 Hz, 2H); ¹³C NMR (125 MHz, CDCl₃): δ 13.10, 14.42, 22.27, 39.71, 43.73, 45.53, 49.70, 66.53, 101.28, 106.55, 117.58, 122.10, 125.44, 125.56, 126.47, 128.51, 128.90, 129.79, 130.69, 131.63, 135.91, 144.85, 145.03, 145.27, 146.84, 147.13, 171.77; (+) LRESIMS m/z 509.21 [M+H]⁺.

### COMPOUND 12.1.57: N,N-DIETHYL-4-{5,6,7-TRIMETHOXY-2-[(4-METHYL-1H-IMIDAZOL-5-YL)METHYL]-1,2,3,4-TETRAHYDROISOQUINOLIN-1-YL}BENZAMIDE

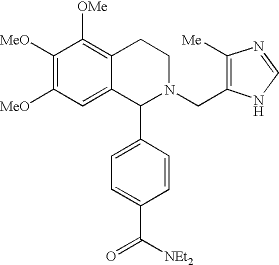

4-Methyl-imidazole-5-carboxyaldehyde (65.2 mg, 0.59 mmol) was added to a solution of INTERMEDIATE 5.1.16 (117.9 mg, 0.30 mmol) in 1,2-dichloroethane (5 mL) and the reaction mixture stirred at room temperature for 10 min. Sodium triacetoxyborohydride (439 mg, 2.07 mmol) was added followed by N-methyl-2-pyrrolidinone (350 mL) and the reaction mixture stirred at RT for 16 h. 1 N NaOH (5 mL) was added and the organic solvent removed in vacuo. The residue was extracted with EtOAc (3×20 mL) and the organic layer washed with water (20 mL). The organic layer was concentrated in vacuo and the residue purified by flash chromatography on SiO₂ column (EtOAc:MeOH 95:5) to afford COMPOUND 12.1.57 as an oil (32.2 mg, 22%). ¹H NMR (500 MHz, CDCl₃): δ 1.11 (br s, 3H), 1.25 (br s, 3H), 2.09 (s, 3H), 2.52 (m, 1H), 2.70 (dt, J 6.5, 17 Hz, 1H), 2.81 (dt, J 4.5, 17 Hz, 1H), 3.05 (m, 1H), 3.27 (br s, 2H), 3.39 (m, 2H), 3.55 (br s, 1H), 3.59 (m, 1H), 3.60 (s, 3H), 3.85 (s, 3H), 3.89 (s, 3H), 4.56 (s, 1H), 6.00 (s, 1H), 7.28-7.32 (m, 4H), 7.39 (s, 1H); ¹³C NMR (125 MHz, CDCl₃): δ 11.12, 13.11, 14.41, 22.43, 39.69, 43.65, 46.30, 49.22, 56.12, 60.71, 61.03, 67.59, 107.92, 121.56, 126.55, 129.87, 130.31, 132.67, 133.31, 136.29, 140.75, 145.12, 150.85, 151.79, 171.51; (+) LRESIMS m/z 493.24 [M+H]⁺.

### COMPOUND 12.1.58: N,N-DIETHYL-4-{5,6,7-TRIMETHOXY-2-[(2-PHENYL-1H-IMIDAZOL-5-YL)METHYL]-1,2,3,4-TETRAHYDROISOQUINOLIN-1-YL}BENZAMIDE

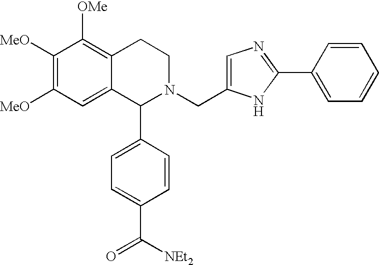

2-Phenyl-imidazole-4-carboxyaldehyde (92.6 mg, 0.54 mmol) was added to a solution of INTERMEDIATE 5.1.16 (107.7 mg, 0.27 mmol) in 1,2-dichloroethane (5 mL) and the reaction mixture stirred at room temperature for 10 min. Sodium triacetoxyborohydride (400 mg, 1.89 mmol) was added followed by N-methyl-2-pyrrolidinone (350 μL) and the reaction mixture stirred at RT for 16 h. 1 N NaOH (5 mL) was added and the organic solvent removed in vacuo. The residue was extracted with EtOAc (3×20 mL) and the organic layer washed with water (20 mL). The organic layer was concentrated in vacuo and the residue purified by flash chromatography on SiO₂ column (EtOAc:MeOH 95:5) to afford COMPOUND 12.1.58 as an oil (33 mg, 22%). ¹H NMR (500 MHz, CDCl₃): δ 1.10 (br s, 3H), 1.24 (br s, 3H), 2.63 (m, 1H), 2.76 (m, 2H), 3.10 (m, 1H), 3.26 (br s, 2H), 3.57 (s, 3H), 3.51-3.62 (m, 4H), 3.84 (s, 3H), 3.88 (s, 3H), 4.65 (s, 1H), 5.98 (s, 1H), 6.91 (s, 1H), 7.25-7.35 (m, 7H, 7.87 (d, J 8.5 Hz, 2H); ¹³C NMR (125 MHz, CDCl₃): δ 13.12, 14.45, 22.51, 39.70, 43.67, 46.18, 49.68, 56.09, 60.70, 61.03, 66.58, 107.94, 121.56, 125.43, 126.50, 128.46, 128.90, 129.91, 130.76, 132.78, 136.08, 140.72, 144.76, 146.76, 147.07, 150.89, 151.73, 171.64; (+) LRESIMS m/z 555.25 [+H]⁺.

### COMPOUND 12.1.59: 4-{7-(CYCLOBUTYLOXY)-6-METHOXY-2-[(5-METHYL-1H-IMIDAZOL-4-YL)METHYL]-1,2,3,4-TETRAHYDROISOQUINOLIN-1-YL}N,N-DIETHYLBENZAMIDE

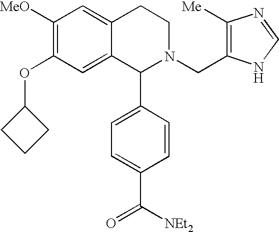

To a solution of INTERMEDIATE 7.1.5 (18 mg, 0.044 mmol), 4-methyl-1H-imidazole-5-carboxaldehyde (5.8 mg, 0.052 mmol, 1.2 eq) in 1,2-dichloroethane (2 mL) was added sodium triacetoxyborohydride (11.1 mg, 0.052 mmol). The reaction mixture was stirred at room temperature for overnight, then diluted with dichloromethane (5 mL), quenched with saturated aqueous sodium bicarbonate (0.5 mL) and separated. The organic phase was washed with brine (1×2 mL), dried (MgSO₄) and filtered. To the filtrate ps-scavenger was added, stirred for 2 hr and filtered The filtrate was concentrated and flash chromatography to give compound COMPOUND 12.1.59 (22 mg, 0.04 mmol, 99%) as colorless oil. ¹H NMR (500 MHz, CD₂Cl₂): δ 1.10 (br s, 3H), 1.24 (br s, 3H), 1.52 (m, 1H), 1.71 (m, 1H), 1.82 (m, 1H), 1.91 (br s, 2H), 2.05 (m, 1H), 2.15 (m, 1H), 2.60 (s, 3H), 2.74 (m, 1H), 2.94 (m, 2H), 3.25 (br s, 2H), 3.40 (m, 1H), 3.53 (br s, 2H), 3.55 (br s, 2H), 3.60 (m, 1H), 6.00 (s, 1H), 6.64 (s, 1H), 7.33-7.55 (m, 4H), 8.20 (s, 1H). (+). LRESIMS m/z 503 [M+H]⁺.

### COMPOUND 12.1.60: N,N-DIETHYL-4-[6-METHOXY-2-[(5-METHYL-1H-IMIDAZOL-4-YL)METHYL]-7-(NEOPENTYLOXY)-1,2,3,4-TETRAHYDROISOQUINOLIN-1-YL]BENZAMIDE

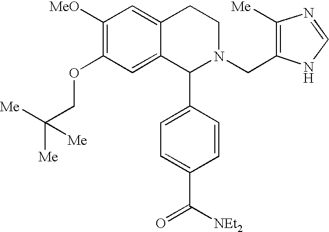

To a solution of Intermediate 7.1.4 (18 mg, 0.0424 mmol), 4-methyl-1H-imidazole-5-carboxaldehyde (5.8 mg, 0.052 mmol, 1.2 eq) in 1,2-dichloroethane (2 mL) was added sodium triacetoxyborohydride (11.1 mg, 0.052 mmol). The reaction mixture was stirred at room temperature for overnight, then diluted with dichloromethane (5 mL), quenched with saturated aqueous sodium bicarbonate (0.5 mL) and separated. The organic phase was washed with brine (1×2 mL), dried MgSO₄) and filtered. To the filtrate ps-scavenger was added, stirred for 2 hr and filtered. The filtrate was concentrated and purified by flash chromatography to give compound COMPOUND 12.1.60 (15 mg, 0.0029 mmol, 68%) as colorless oil. ¹H NMR (500 MHz, CD₂Cl₂): δ 0.89 (s, 9H), 1.00 (br s, 3H), 1.24 (br s, 3H), 1.90 (s, 3H), 1.99 (s, 2H), 2.45 (m, 1H), 2.68 (m, 1H), 2.82 (m, 2H), 3.15 (br s, 2H), 3.28 (m, 2H), 3.40 (br s, 21), 3.70 (s, 3H), 4.45 (s, 1H), 6.10 (br s, 1H), 6.51 (s, 1H), 7.29-7.33 (m, 4H), 8.18 (s, 1H). (+) LRESIMS m/z 519 [M+H]⁺.

### COMPOUND 12.1.61: N,N-DIETHYL-4-{6-FLUORO-7-METHOXY-2-[(4-METHYL-1H-IMIDAZOL-5-YL)METHYL]-1,2,3,4-TETRAHYDROISOQUINOLIN-1-YL}BENZAMIDE

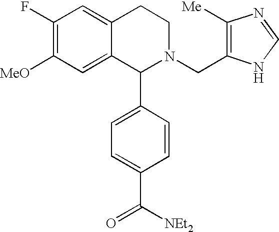

4-Methyl-imidazole-5-carboxyaldehyde (37 mg, 0.34 mmol) was added to a solution of INTERMEDIATE 4.2.1 (60 mg, 0.17 mmol) in 1,2-dichloroethane (3 mL) and the reaction mixture stirred at room temperature for 10 min. Sodium triacetoxyborohydride (250 mg, 1.18 mmol) was added followed by N-methyl-2-pyrrolidinone (200 μL) and the reaction mixture stirred for 24 h. 1 N NaOH (2.5 mL) was added and the organic solvent removed in vacuo. The residue was extracted with EtOAc (3×10 mL) and the organic layer washed with water (10 mL). The organic 0 layer was concentrated in vacuo and the residue purified by flash chromatography on SiO₂ column (CHCl₃:EtOAc:MeOH 63:30:7) to afford COMPOUND 12.1.61 as an oil (57.3 mg, 76%). ¹H NMR (500 MHz, CDCl₃): δ 1.11 (br s, 3H), 1.25 (br s, 3H), 2.08 (s, 3H), 2.52 (m, 1H), 2.70 (dt, J 4.5, 16.5 Hz, 1H), 2.86 (m, 1H), 3.04 (m, 1H), 3.26 (br s, 1H), 3.36 (d, J 13.5 Hz, 2H), 3.54 (br s, 2H), 3.58 (d, J 14 Hz, 2H), 3.62 (s, 3H), 4.57 (s, 1H), 6.26 (d, J 8.5 Hz, 1H), 6.81 (d, J 12 Hz, 1H), 7.27-7.33 (m, 5H), 8.95 (br s, NH); ¹³C NMR (125 MHz, CDCl₃): δ 13.07, 14.40, 27.67, 39.70, 43.63, 46.36, 49.29, 56.55, 67.49, 114.23, 115.73 (d, J 17.6 Hz), 126.60, 127.19, 127.89 (d, J 5.8 Hz), 129.57, 129.84, 133.05, 133.15, 136.39, 145.16, 145.92 (d, J 11.4 Hz), 150.34, 152.30, 171.48; (+) LRESIMS m/z 451.20 [M+H]⁺.

### COMPOUND 12.1.62: N,N-DIETHYL-4-{6-FLUORO-7-METHOXY-2-[(2-PHENYL-1H]IMIDAZOL-5-YL)METHYL-1,2,3,4-TETRAHYDROISOQUINOLIN-1-YL}BENZAMIDE

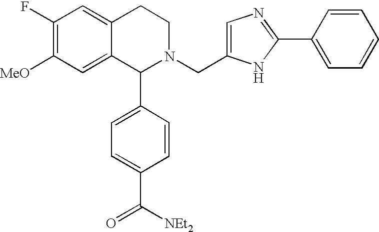

2-Phenyl-imidazole-4-carboxyaldehyde (58 mg, 0.34 mmol) was added to a solution of INTERMEDIATE 4.2.1 (60 mg, 0.17 mmol) in 1,2-dichloroethane (3 mL) and the reaction mixture stirred at room temperature for 10 min. Sodium triacetoxyborohydride (250 mg, 1.18 mmol) was added followed by N-methyl-2-pyrrolidinone (200 μL) and the reaction mixture stirred at RT for 24 h. 1 N NaOH (2.5 mL) was added and the organic solvent removed in vacuo. The residue was extracted with EtOAc (3×10 mL) and the organic layer washed with water (10 mL). The organic layer was concentrated in vacuo and the residue purified by flash chromatography on SiO₂ column (CHCl₃:EtOAc:MeOH 63:30:7) to afford COMPOUND 12.1.62 as an oil (65 mg, 75%). ¹H NMR (500 MHz, CDCl₃): δ 1.10 m (brs, 3H), 1.26 (brs, 3H), 2.62 (m, 1H), 2.71 (dt, J 5.0, 16.5 Hz, 1H), 2.84 (m, 1H), 3.04 (m, 1H), 3.26 (br s, 2H), 3.48-3.55 (m, 4H), 3.57 (s, 3H), 4.65 (s, 1H), 6.22 (d, J 8.5 Hz, 1H), 6.80 (d, J 11.5 Hz, 1H), 6.87 (s, 1H), 7.22-7.32 (m, 7H), 7.87 (d, J 8 Hz, 2H); ¹³C NMR (125 MHz, CDCl₃): δ 13.13, 14.41, 27.78, 39.76, 43.69, 46.19, 50.51, 56.49, 66.34, 114.17, 115.74 (d, J 17.6 Hz), 125.48, 126.54, 127.84 (d, J 5.5 Hz), 128.45, 128.88, 129.84, 130.75, 133.14, 136.11, 144.70, 145.83 (d, J 11.4 Hz), 146.80, 150.30, 152.25, 171.64; (+) LRESIMS m/z 513.20 [M+H]⁺.

### COMPOUND 12.1.63: 1-{4-[(DIETHYLAMINO)CARBONYL]PHENYL}-6-METHOXY-2-[(2-PHENYL-1H-IMIDAZOL-5-YL)METHYL]-1,2,3,4-TETRAHYDROISOQUINOLIN-7-YL DIMETHYLSULFAMATE

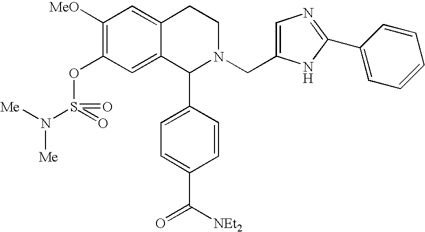

INTERMEDIATE 8.3.1 (53 mg, 0.11 mmol) and 2-phenylimidazole-4-carbaldehyde (48 mg, 0.28 mmol) were dissolved in 1,2-dichloroethane (5 mL) and stirred for 10 min at room temperature. Sodium triacetoxyborohydride (120 mg, 0.55 mmol) was added and the mixture was stirred for 18 h. Tosylhydrazine resin (0.37 g, 1.5 mmol/g) and DCM (5 mL) were added and the mixture was stirred for another 2 h. The resin was filtered off and washed twice with DCM. The filtrate was washed with 1 M aqueous sodium hydroxide solution, water, and brine, dried, and evaporated. Flash chromatography of the residue yielded the desired product (36 mg, 0.06 mmol, 53%). ¹H NMR (500 MHz, DMSO): 1.02, 1.15 (2 brs, 6H), 2.72 (s, 6H), 3.04 (m, 21), 3.18 (m, 3H), 3.40 (m, 3H), 3.82 (s, 3H), 5.38 (s, 1H), 6.42 (s, 1H), 7.18 (s, 1H), 7.40 (m, 4H), 7.58 (m, 4), 7.59 (s, 1H), 7.98 (d, J 8.0 Hz, 2H). (+) LRESIMS m/z 618 [M+H]⁺.

### COMPOUND 13.1.1: N,N-DIETHYL-4-[6-METHOXY-2-[(5-METHYL-1H-IMIDAZOL-4-YL)-METHYL]-7-(TETRAHYDRO-2H-PYRAN-4-YLOXY)-1,2,3,4-(TETRAHYDROISOQUINOLIN-1-YL]BENZAMIDE

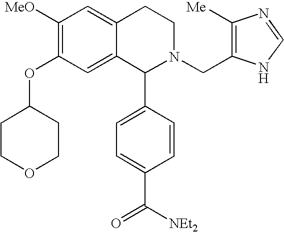

A solution of INTERMEDIATE 5.1.12 (44 mg, 0.124 mmol) and tetrahydro-4H-pyran-4-ol (0.248 mmol, 25.3 mg, 23.6 uL) in anhydrous dichloromethane (1 mL) was added to a freshly prepared betaine [diisopropylazodicarboxylate (50 mg, 0.247 mmol, 49 uL) was added to a solution of triphenylphosphine (65 mg, 0.248 mmol) in anhydrous dichloromethane (1.5 mL) at 0° C.]. The reaction mixture was stirred at 0° C. for 1 hr and followed at RT for further 20 hr then quenched with water (2 mL) and extracted to dichloromethane (2×10 mL), dried over MgSO₄ and concentrated. Crude product was purified by flash chromatography, concentrated and dried under vacuum before using for next step.

A purified compound (70%, 50 mg, 0.079 mmol) was dissolved in anhydrous dichloroethane (2 mL). To this solution was added 4-methyl-1H-imidazole-5-carbaldehyde (12 mg, 0.10 mmol) and followed after 5 min was sodium triacetoxyborohydride (68 mg, 0.327 mmol). The reaction mixture was stirred at RT for 20 hr then quenched with saturated aqueous sodium bicarbonate (2 mL) and extracted with dichloromethane (3×10 mL), dried over MgSO₄ and concentrated. The product was purified by flash chromatography to give 7 mg (0.0131 mmol, 17%) of COMPOUND 13.1.1 as colourless oil. ¹H NMR (500 MHz, CDCl₃): δ 1.15 (br s, 3H), 1.28 (br s, 3H), 1.55 (m, 1H), 1.78 (m, 1H), 2.05 (m, 1H), 2.55 (m, 1H), 2.75 (m, 1H), 2.96 (m, 1H), 3.18 (m, 1H), 3.32 (br s, 2H), 3.35 (m, 2H), 3.45 (m, 2H), 3.58 (br s, 2H), 3.63 (m, 2H), 3.80 (s, 3H), 3.88 (s, 1H), 4.52 (s, 1H), 6.18 (s, 1H), 6.70 (s, 1H), 7.35-7.46 (m, 4H), 7.50 (s, 1H). ¹³C NMR (125 MHz, CDCl₃): δ 9.54, 11.90, 13.23, 27.90, 31.78, 31.89, 39.70, 43.80, 47.22, 49.39, 55.28, 64.89, 64.93, 68.09, 73.80, 111.15, 115.01, 126.03, 129.15, 129.81, 129.87, 133.31, 135.80, 144.38, 146.30, 146.68, 172.46. (+) LRESIMS m/z 533 (M+H)⁺.

### COMPOUND 14.1.1: N,N-DIETHYL-4-{6-METHOXY-7-PHENOXY-2-[(2-PHENYL-1H-IMIDAZOL-4-YL)METHYL]-1,2,3,4-TETRAHYDROISOQUINOLIN-1-YL}BENZAMIDE

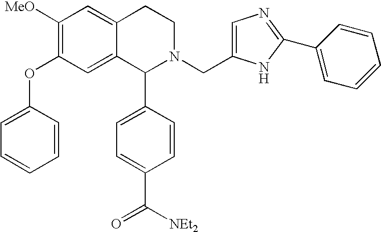

To a solution of INTERMEDIATE 10.2.1 (60 mg, 0.1132 mmol) was added a solution of hydrochloric acid in 1,4-dioxane (4N, 1 mL) at room temperature. The reaction mixture was stirred for an hour then concentrated by a stream of nitrogen and dried under vacuum. The residue was re-dissolved in 1,2-dichloroethane (3 mL) and transferred 1.5 mL of this solution to another flash. To this solution was added 2-phenyl-1H-imidazole-5-carbaldehyde (−2 eq, 0.12 mmol, 20 mg) and followed by sodium triacetoxyborohydride (4 eq, 0.24 mmol, 50 mg). The reaction mixture was stirred for 20 hr then quenched with saturated aqueous sodium bicarbonate and extracted to dichloromethane (15 mL×2). Excess aldehyde was removed by stirring the extracted dichloromethane with polymer supported hydrazine for 2 hr. The polymer was filtered off and the filtrate was concentrated and dried under vacuum. Product was purified by flash chromatography, using SiO₂ column with MeOH/DCM (10:90) afford 19.3 mg (0.0329 mmol, 58% overall yield) of COMPOUND 14.1.1. ¹H NMR (500 MS, CDCl₃): δ 1.08 (br s, 3H), 1.28 (br s, 3H), 2.68-3.16 (br m, 4H), 3.22 (br s, 2H), 3.48 (m, 2H), 3.56 (br s, 2H), 3.78 (s, 3H), 4.62 (s, 1H), 6.34 (s, 1H), 6.73 (s, 1H), 6.78 (s, 1H), 6.85-7.36 (m, 12H), 7.86 (m, 2H). ¹³C NMR (125 MHz, CDCl₃): δ 13.13, 14.44, 28.81, 39.69, 43.71, 46.99, 50.26, 56.10, 66.70, 110.84, 112.67, 114.77, 116.46, 122.12, 125.57, 126.51, 128.78, 128.97, 129.53, 129.78, 130.23, 131.63, 136.21, 142.65, 144.32, 146.47, 150.37, 158.47, 171.58. (+) LRESIMS m/z 587 (M+H)⁺.

### COMPOUND 14.1.2: N,N-DIETHYL-4-{6-METHOXY-2-[(5-METHYL-1H-IMIDAZOL-4-YL)METHYL]-7-PHENOXY-1,2,3,4-TETRAHYDROISOQUINOLIN-1-YL}BENZAMIDE

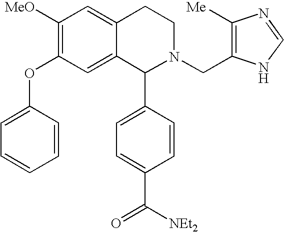

To INTERMEDIATE 10.2.1 (30 mg, 0.06 mmol) was added a solution of hydrochloric acid in 1,4-dioxane (4N, 0.5 mL) at room temperature. The reaction mixture was stirred for an hour then concentrated by a stream of nitrogen and dried under vacuum. The residue was re-dissolved in 1,2-dichloroethane (1.5 mL) and to this solution was added 4-methyl-1H-imidazole-5-carbaldehyde (−2 eq, 0.12 mmol, 12 mg) and followed by sodium triacetoxyborohydride (4 eq, 0.24 mmol, 50 mg). The reaction mixture was stirred for 20 hr then quenched with saturated aqueous sodium bicarbonate and extracted to dichloromethane (15 mL×2). Excess aldehyde was removed by stirring the extracted dichloromethane with polymer supported hydrazine for 2 hr. The polymer was filtered off and the filtrate was concentrated and dried under vacuum. Product was purified by flash chromatography, using SiO₂ column with MeOH/DCM (10:90) afford 17.9 mg (0.034 mmol, 60%) of COMPOUND 14.1.2 as light yellow oil. ¹H NMR (500 MHz, CDCl₃): δ 1.05 (br s, 3H), 1.18 (br s, 3H), 2.00 (s, 3H), 2.50-2.98 (br m, 4H), 3.10 (br s, 2H), 3.28 (nm 1H), 3.49 (br s, 2H), 3.52 (m, 1H), 3.72 (s, 3H), 4.45 (s, 1H), 6.28 (s, 1H), 6.65 (s, 1H), 6.68-7.20 (m, 9H), 7.26 (m, 1H). ¹³C NMR (125 MHz, CDCl₃): δ 10.97, 13.09, 14.42, 28.70, 39.65, 43.68, 46.96, 49.24, 56.20, 67.66, 106.00, 112.63, 115.47, 116.47, 122.02, 126.57, 129.91, 130.26, 131.76, 132.93, 136.34, 142.69, 145.32, 150.37, 150.37, 171.51. (+) LRESIMS m/z 525 (+H)⁺.

### COMPOUND 14.1.3: N,N-DIETHYL-4-{7-(4 FLUOROPHENOXY)-6-METHOXY-2-[(2-PHENYL-1H-IMIDAZOL-4-YL)METHYL]-1,2,3,4-TETRAHYDROISOQUINOLIN-1-YL}BENZAMIDE

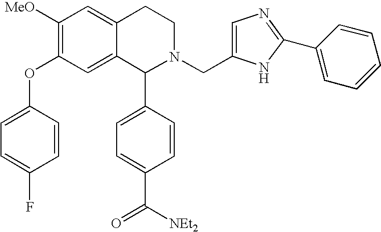

To INTERMEDIATE 10.2.2 (50 mg, 0.111 mmol) was added a solution of hydrogen chloride (4M) in 1,4-dioxane (1 mL) at RT and the mixture was stirred for 1 hr. Solvent was removed by a stream of nitrogen and followed by under vacuum. The residue was de-dissolved in anhydrous 1,2-dichloroethane (1.5 mL) and 2-phenyl-1H-imidazole-5-carboxaldehyde (1.2 eq, 0.1332 mmol, 19 mg), sodium triacetoxyborohydride (3 eq, 0.333 mmol, 70 mg). The reaction mixture was stirred at RT for further 20 hr, quenched with saturated aqueous sodium bicarbonate (0.5 mL), diluted with dichloromethane (10 mL), phase separated and washed the organic layer with brine (1×2 mL), dried over MgSO₄, filtered. The filtrate was stirred with PS-hydrazine to scavenge to remove excess aldehyde for 2 hr then filtered and the filtrate was concentrated, flash purification to give 28 mg (0.046 mmol, 41%) of COMPOUND 14.1.3 as colourless oil. ¹H NMR (500 MHz, CDCl₃): δ 1.08 (br s, 3H), 1.26 (br s, 3H), 1.70-3.05 (m, 4H), 3.25 (br m, 3H), 3.58 (br m, 4H), 3.79 (s, 3H), 4.67 (s, 1H), 6.28 (s, 1H), 6.72 (s, 1H), 6.72-7.92 (m, 13H). ¹³C NMR (125 MHz, CDCl₃): δ 13.09, 14.39, 28.58, 39.70, 43.65, 47.06, 50.09, 56.19, 66.70, 112.64, 115.97 (d, J 24 Hz), 117.93 (d, J 8 Hz), 121.44, 125.77, 126.62, 129.09, 129.69, 131.28, 132.00, 136.41, 143.29, 143.40, 146.36, 150.22, 154.24, 158 (d, J 240 Hz), 171.18. (+) LRESIMS m/z 605 [M+H]⁺.

### COMPOUND 14.1.4: N,N-DIETHYL-4-{7-(4-FLUOROPHENOXY)-6-METHOXY-2-[(5-METHYL-1H-IMIDAZOL-4-YL)METHYL]-1,2,3,4-TETRAHYDROISOQUINOLIN-1-YL}BENZAMIDE

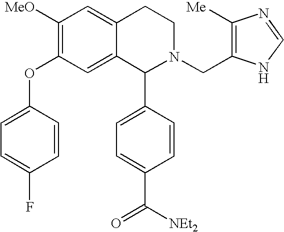

To INTERMEDIATE 10.2.2 (100 mg, 0.223 mmol) was added a solution of hydrogen chloride in 1,4-dioxane (4M, 1 mL) at RT and the mixture was stirred for 1 hr. Solvent was removed by applying a stream of nitrogen and followed by under vacuum. The residue was re-dissolved in anhydrous 1,2-dichloroethane (1.5 mL). To this solution 4-methyl-1H-imidazole-5-carboxaldehyde (1.5 eq, 0.3345 mmol, 37 mg) and sodium triacetoxyborohydride (3 eq, 0.669 mmol, 125 mg) were added. The reaction mixture was stirred at RT for further 20 hr then was quenched with saturated aqueous sodium bicarbonate (0.5 mL), diluted with dichloromethane (10 mL). Organic phase was separated and washed it with brine (1×2 mL), dried over MgSO₄ and filtered. The filtrate was stirred with PS-hydrazine to scavenge excess aldehyde for 2 hr then filtered and the filtrate was concentrated. Product was purified by flash chromatography to afford 42.5 mg (0.078 mmol, 35%) of COMPOUND 14.1.4 as colourless oil. ¹H NMR (500 MHz, CDCl₃): δ 1.08 (br s, 3H), 1.24 (br s, 3H), 2.16 (s, 3H), 2.60 (m, 1H), 2.82 (m, 1H), 3.05 (br m, 2H), 3.22 (br s, 2H), 3.90 (d, J 13 Hz, 1H), 3.54 (br s, 2H), 3.61 (d, J 13 Hz, 1H), 3.80 (s, 3H), 4.52 (s, 1H), 6.30 (s, 1H), 6.72 (s, 1H), 6.74 (m, 2×1H), 6.88 (m, 2×1H), 7.28-7.38 (m, 4H). ¹³C NMR (125 MHz, CDCl₃): δ 10.53, 13.09, 14.38, 28.61, 39.72, 43.67, 47.11, 49.00, 56.19, 67.79, 112.61, 115.96 (d, J 23 Hz), 117.98 (d, J 8 Hz), 121.52, 126.58, 129.91, 131.57, 132.40, 136.42, 143.27, 145.10, 150.19, 154.30, 158.23 (d, J 240 Hz), 171.51. (+) LRESIMS m/z 543 [M+H]⁺.

### COMPOUND 14.1.5: N,N-DIETHYL-4-{6-METHOXY-7-(4-METHOXYPHENOXY)-2-[(5-METHYL-1H-IMIDAZOL-4-YL)METHYL]-1,2,3,4-TETRAHYDROISOQUINOLIN-1-YL}BENZAMIDE

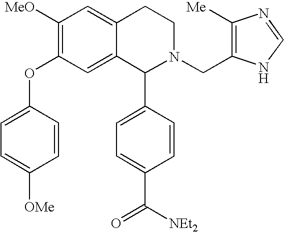

To INTERMEDIATE 10.2.3 (38 mg, 0.068 mmol) was added a solution of hydrogen chloride in 1,4-dioxane (4M, 1 mL) at RT and the mixture was stirred for 1 hr. Solvent was removed by applying a stream of nitrogen and followed by under vacuum. The dried residue was re-dissolved in 1,2-dichloroethane (5 mL). To this solution were added 4-methyl-1H-imidazole-5-carboxaldehyde (9 mg, 0.0816 mmol, 1.2 eq) and sodium triacetoxyborohydride (43 mg, 0.2036 mmol, 3 eq). The reaction mixture was stirred at room temperature for overnight and then quenched with saturated aqueous sodium bicarbonate (2 mL), extracted with ethyl acetate (2×10 mL). The combined extracts were washed with brine (2×5 mL), dried over MgSO₄ and concentrated. Product was purified by flash chromatography to afford COMPOUND 14.1.5 (11 mg, 0.020 mmol, 29%). ¹H NMR (CDCl₃, 500 MHz): δ 1.10 (br s, 3H), 1.25 (br s, 3H), 2.11 (s, 3H), 2.58 (br m, 1H), 2.78 (br m, 1H), 2.96 (m, 1H), 3.07 (br m, 1H), 3.22 (br s, 2H), 3.36 (d, J 13 Hz, 1H), 3.54 (br s, 2H), 3.59 (d, J 13 Hz, 1H), 3.75 (s, 3H0, 3.82 (s, 3H), 4.50 (s, 1H), 6.27 (s, 1H), 6.58 (s, 1H), 6.73 (s, 3H), 7.22-7.35 (m, 5H). ¹³C NMR (125 MHz, CDCl₃): δ 10.94, 13.12, 14.44, 28.46, 39.67, 43.67, 46.88, 49.13, 55.87, 56.24, 67.53, 112.49, 114.74, 118.26, 120.71, 126.52, 129.79, 130.79, 132.91, 136.30, 144.17, 145.25, 149.95, 151.76, 155.89, 171.52. (+) LRESIMS m/z 555 [M+H]⁺.

### COMPOUND 14.1.6: N,N-DIETHYL-4-[6-METHOXY-2-[(5-METHYL-1H-IMIDAZOL-4-YL)METHYL]-7-(PYRIDIN-3-YLOXY)-1,2,3,4-TETRAHYDROISOQUINOLIN-1-YL]BENZAMIDE

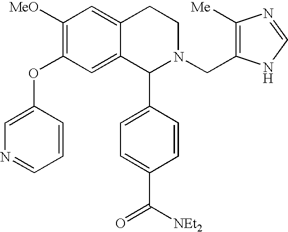

To INTERMEDIATE 10.2.4 (52 mg, 0.097 mmol) was added a solution of hydrogen chloride in 1,4-dioxane (4M, 1 mL) at RT and the mixture was stirred for 1 hr. Solvent was removed by applying a stream of nitrogen and followed by under vacuum. The dried residue was re-dissolved in 1,2-dichloroethane (5 mL). To this solution were added 4-methyl-1H-imidazole-5-carboxaldehyde (13 mg, 0.1176 mmol, 1.2 eq) and sodium triacetoxyborohydride (62 mg, 0.294 mmol, 3 eq) and stirred at room temperature overnight. The reaction mixture was then quenched with saturated aqueous sodium bicarbonate (2 mL), extracted with ethyl acetate (2×10 mL). The extracts were washed with brine (2×5 mL), dried over MgSO₄, concentrated. Product was purified by flash chromatography to afford COMPOUND 14.1.6 (8 mg, 0.015 mmol, 14%). ¹H NMR (500 MHz, CD₃OD): δ 1.25 (br s, 3H), 1.33 (br s, 3H), 1.05 (s, 3H), 2.62 (m, 1H), 2.91 (m, 1H), 3.12 (m, 1H), 3.19 (m, 1H), 3.21 (br s, 2H), 3.35 (s, 3H), 3.36 (d, J 13 Hz, 1H), 3.59 (br s, 2H), 3.67 (d, J 13 Hz, 1H), 3.75 (s, 3H), 4.61 (s, 1H), 6.40 (s, 1H), 6.88 (s, 1H), 7.12-8.00 (m, 8H), 8.18 (s, 1H). ¹³C NMR (125 MHz, CD₃OD): δ 9.42, 11.89, 13.18, 28.47, 39.73, 43.80, 49.42, 53.61, 55.18, 67.96, 112.77, 122.14, 124.43, 126.20, 129.78, 130.98, 133.37, 136.06, 137.59, 141.00, 142.11, 145.98, 150.22, 156.00, 172.33. (+) LRESIMS m/z 526 [M+H]⁺.

### COMPOUND 15.1.1: 4-{7-(BENZYLOXY)-6-METHOXY-2-[(5-METHYL-1H-IMIDAZOL-4-YL)METHYL]-1,2,3,4-TETRAHYDROISOQUINOLIN-1-YL}-N,N-DIETHYLBENZAMIDE

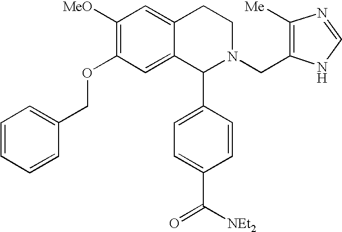

To a solution of triphenylphosphine (147 mg, 0.56 mmol), diethylazodicarboxylate (DIAD, 113 mg, 0.56 mmol) in anhydrous dichloromethane (1 mL) at 0° C. was added a solution of INTERMEDIATE 10.1.1 (85 mg, 0.187 mmol) and benzyl alcohol (120 mg, 0.2072 mmol) in anhydrous dichloromethane (0.5 mL). The reaction mixture was stirred at room temperature for overnight then quenched with water (1 mL), extracted with ethyl acetate, dried MgSO₄ and concentrated. Crude product was purified by flash chromatography to give product which was dried under vacuum, used for the next step.

A dried material (86.4 mg, 0.1588 mmol) was de-protected by hydrochloric acid (4M) in 1,4-dioxane (1 mL) for 1 hr, then the excess reagent and solvent were removed by applying a stream of nitrogen to dryness. The residue was dried flier under vacuum for 1 hr then re-dissolved in 1,2-dichloroethane (5 mL). To this solution were added 4-methyl-1H-imidazole-5-carboxaldehyde (21 mg, 0.1905 mmol, 1.2 eq) and sodium triacetoxyborohydride (1.1 mg, 0.576 mmol, 3 eq). The reaction mixture was stirred at room temperature for overnight, then quenched with saturated aqueous sodium bicarbonate (2 mL), extracted to ethyl acetate (2×10 mL), washed with brine (2×2 mL), dried over MgSO₄. Purification was carried out by using flash chromatography to afford COMPOUND 15.1.1 (16 mg, 0.0297 mmol, 19%) as oil. ¹H NMR (500 MHz, CDCl₃): δ 1.13 (br s, 3H), 1.25 (br s, 3H), 2.09 (s, 3H), 2.56 (br m, 1H), 2.77 (br m, 1H), 2.90 (br m, 1H), 3.03 (br m, 1H), 3.27 (br s, 2H), 3.38 (d, J 13 Hz, 2H), 3.56 (br s, 2H), 3.38 (d, J 13 Hz, 1H), 3.56 (br s, 2H), 3.60 (d, J 13 Hz, 1H), 3.86 (s, 3H), 4.51 (s, 1H), 4.88 (s, 2H), 6.25 (s, 1H), 6.64 (s, 1H), 7.25-7.37 (m, 9H). ¹³C NMR (125 MHz, CDCl₃): δ 10.93, 13.12, 14.44, 27.89, 39.71, 43.68, 46.75, 49.02, 56.19, 67.37, 71.60, 111.84, 115.29, 126.59, 127.66, 127.83, 128.03, 128.66, 128.80, 130.31, 133.05, 136.22, 137.26, 145.32, 146.59, 148.86, 171.54. (+) LRESIMS m/z 539 [M+H]⁺.

### INTERMEDIATE 16.1.1: TERT-BUTYL 5-FORMYL-4-METHYL-1H-IMIDAZOLE-1-CARBOXYLATE

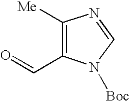

To a solution of 4-methyl-1H-imidazole-5-carbaldehyde (757 mg, 6.88 mmol) in anhydrous methanol (15 mL) at RT were added di-t-butylcarbonate (1.5 g, 6.88 mmol) and triethyl amine (1.12 mL). The reaction mixture was stirred at RT for 3 hr, then quenched with water (10 mL), extracted to EtOAc (100 mL) and washed with 0.1% hydrochloric acid (3×10 mL), water (2×20 mL), dry over MgSO₄ and concentrated to give 550 mg (2.62 mmol, 38%) of INTERMEDIATE 16.1.1 as white solid. ¹H NMR (500 MHz, CDCl₃): δ 1.69 (s, 9H), 2.80 (s, 3H), 8.10 (s, 1H), 10.00 (s, 1H). ¹³C NMR (125 MHz, CDCl₃): δ 11.57, 28.11 (3C), 87.40, 136.54, 137.99, 138.41, 147.32, 187.42. (+) LRESIMS m/z 211 (M+H)⁺.

### INTERMEDIATE 16.2.1: TERT-BUTYL-4-{[1-{4-[(DIETHYLAMINO)CARBONYL]PHENYL}-7-HYDROXY-6-METHOXY-3,4-DIHYDROISOQUINOLIN-2(1H)-YL]METHYL}-5-METHYL-1H-IMIDAZOLE-1-CARBOXYLATE

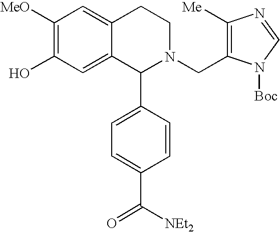

To a solution of INTERMEDIATE 5.1.12 (100 mg, 0.282 mmol) in anhydrous 1,2-dichloroethane (8 mL) were added INTERMEDIATE 16.1.1 (1.1 eq, 0.311 mmol, 65 mg) and triacetoxyborohydride (3 eq, 0.846 mmol, 178 mg). The reaction mixture was m stirred at room temperature for 20 hr then diluted with dichloromethane (10 mL), quenched with saturated aqueous sodium bicarbonate (3 mL) and phase separated. The organic phase was washed with water (2×3 mL), brine (1×3 mL). The excess aldehyde was removed by polymer supported hydrazine resin scavenger for 1 hr. The resin was filtered off and the filtrate was evaporated and purified by flash m chromatography to give 105 mg (0.229 mmol, 81%) of INTERMEDIATE 16.2.1 as light yellow solid. ¹H NMR (500 MHz, CDCl₃): δ 1.12 (br s, 3H), 1.23 (br s, 3H), 1.61 (s, 9H), 2.18 (s, 3H), 2.70-3.70 (brm, 10H), 3.82 (s, 3H), 4.50 (s, 1H), 6.25 (s, 1H), 6.56 (s, 1H), 7.32-7.39 (m, 4H). ¹³C NMR (125 MHz, CDCl₃): δ 10.98, 13.00, 14.10, 28.16, 28.54, 39.10, 43.80, 47.77, 50.80, 53.64, 56.06, 67.83, 85.37, 110.56, 114.83, 126.66, 129.95, 136.50, 137.10, 143.99, 145.62, 148.13, 171.48. (+) LRESIMS m/z 459 (M+H)⁺.

### INTERMEDIATE 16.3.1: TERT-BUTYL 4-{[1-{4-[(DIETHYLAMINO)CARBONYL]PHENYL}-6-METHOXY-7-(3-METHOXYPHENOXY)-3,4-DIHYDROISOQUINOLIN-2(1H)-YL]METHYL}-5-METHYL-1H-IMIDAZOLE-1-CARBOXYLATE

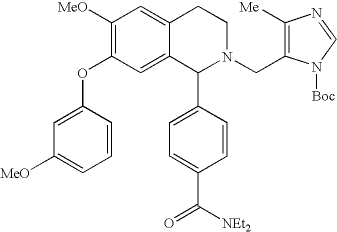

To a solution of INTERMEDIATE 16.2.1 (100 mg, 0.1824 mmol) in anhydrous dichloromethane (2.5 mL) were added copper(II) acetate (66 mg, 0.365 mmol, 2 eq), 3-methoxyphenyl boronic acid (55 mg, 0.365 mmol, 2 eq), molecular sieves (40 mg, 4 A) and triethylamine (36.8 mg, 0.365 mmol, 2 eq). The reaction mixture was stirred at room temperature for overnight and then filtered through a silica layer and wash the silica layer with a solution of methanol/dichloromethane (1:99, 20 mL). After evaporation of solvent, the residue was flash chromatography on silica column to afford INTERMEDIATE 16.3.1 (25 mg, 0.038 mmol, 21%). ¹H NMR (500 MHz, CDCl₃): δ 1.10 (br s, 3H), 1.25 (br, 3H), 2.60 (br s, 9H), 2.50 (br m, 3H), 2.42-2.83 (br m, 4H), 3.25 (br s, 2H), 3.58 (br s, 2H), 3.75 (s, 3H), 3.79-3.82 (m, 2H), 3.81 (s, 3H), 6.32-6.38 (s, 8H), 6.51-7.38 (br m, 8H). (+) LRESIMS m/z 655 [M+H]⁺.

### INTERMEDIATE 16.3.2: TERT-BUTYL 4-{[1-{4-[(DIETHYLAMINO)CARBONYL]PHENYL}-6-METHOXY-7-(4-METHOXYPHENOXY)-3,4-DIHYDROISOQUINOLIN-2(1H)-YL]METHYL}-5-METHYL-1H-IMIDAZOLE-1-CARBOXYLATE

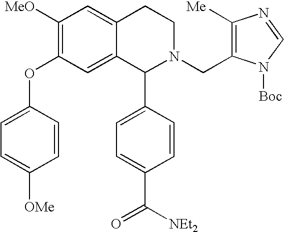

To a solution of INTERMEDIATE 16.2.1 (100 mg, 0.1824 mmol) in anhydrous dichloromethane (2.5 mL) was added copper(II) acetate (66 mg, 0.365 mmol, 2 eq), 4-methoxyphenyl boronic acid (55 mg, 0.365 mmol, 2 eq), molecular sieves (40 mg, 4 A) and triethylamine (36.8 mg, 0.365 mmol, 2 eq). The reaction mixture was stirred at room temperature for overnight and then filtered through a silica layer and wash the silica layer with a solution of methanol/dichloromethane (1:99, 20 mL). After evaporation of solvent, the residue was purified by flash chromatography on silica column to afford INTERMEDIATE 16.3.2 (28 mg, 0.0428 mmol, 23%) as light yellow oil. ¹H NMR (500 MHz, CDCl₃): δ 1.13 (br s, 3H), 1.25 (br, 3H), 1.60 (s, 9H), 2.35 (br s, 3), 2.50-3.06 (br m, 4H), 3.25 (br s, 2H), 3.55 (br m, 2H), 3.73 (s, 3H), 3.80-3.95 (m, 2H), 3.84 (s, 3H), 6.20 (br s, 2H), 6.65-8.00 (m, 8H). (+) LRESIMS m/z 655 [M+H]⁺.

### INTERMEDIATE 16.3.3: 1-{4-[(DIETHYLAMINO)CARBONYL]PHENYL}-6-METHOXY-2-{[5-METHYL-1-(1-NEOPENTYLVINYL)-1H-IMIDAZOL-4-YL]METHYL}-1,2,3,4-TETRAHYDROISOQUINOLIN-7-YL BENZENESULFONATE

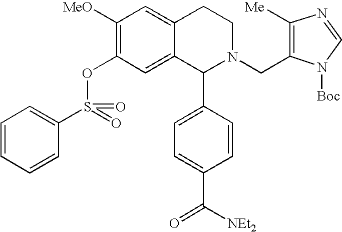

To a mixture of INTERMEDIATE 16.2.1 (50 mg, 0.0912 mmol), benzenesulfonyl chloride (17.6 mg, 0.1 mmol, 1.1 eq) in anhydrous dichloromethane (0.25 mL) at 0° C. was added triethylamine (0.015 mL). The reaction mixture was stirred at room temperature for 20 hr then diluted with dichloromethane (20 mL) and washed with water (1×5 mL), diluted hydrochloric acid solution (0.01M) (1×5 mL), water (1×5 mL), brine (1×5 mL), dried over MgSO₄ and concentrated. Product was purified by flash chromatography to give 15 mg (0.0218 mmol, 24%) of INTERMEDIATE 16.3.3 as colourless oil. ¹H NMR (500 MHz, CDCl₃): δ 1.15 (br s, 3H), 1.28 (br s, 3H), 2.18 (s, 31), 2.40 (m, 2H), 2.78 (m, 2H), 3.30 (br s, 4H), 3.78 (s, 3H), 4.58 (s, 1H), 6.39 (s, 1), 6.58 (s, 1H), 7.48-7.89 (m, 10H). (+) LRESIMS m/z 690 [M+H]⁺.

### COMPOUND 16.4.1: N,N-DIETHYL-4-{6-METHOXY-7-(3-METHOXYPHENOXY-2-[(5-METHYL-1H-IMIDAZOL-4-YL)METHYL]-1,2,3,4-TETRAHYDROISOQUINOLIN-1-YL}BENZAMIDE

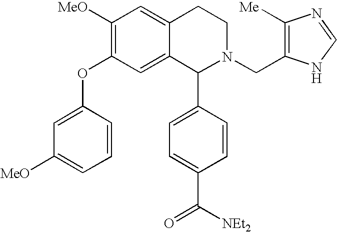

A solution of hydrochloric acid in 1,4-dioxane (4M, 0.5 mL) was slowly added to a compound INTERMEDIATE 16.3.1 (15 mg, 0.023 mmol). The mixture was stirred at room temperature for 1 hr then evaporated solvent by applying a stream of nitrogen to dryness and followed by under vacuum for further 1 hr. The residue was stirred in anhydrous diethyl ether for 5 min (1×1 mL) and filtered. The insoluble compound was washed with fresh anhydrous ethyl ether (1×1 mL) to afford COMPOUND 16.4.1 (3.0 mg, 0.0050 mmol, 22%) as off white solid. ¹H NMR (500 MHz, CD₃OD): δ 1.10 (br s, 3H), 1.26 (br s, 3H), 2.31 (s, 3H), 2.35-2.60 (m, 4H), 3.26 (br s, 2H), 3.33 (s, 2), 3.56 (br s, 2H), 3.67 9 s, 3), 3.81 (s, 3H), 6.28 (s, 1H), 6.58 (s, 1H), 7.10-7.98 (m, 8H), 8.90 (br s, 1H). (+) LRESIMS m/z 555 [M+H]⁺.

### COMPOUND 16.4.2: N,N-DIETHYL-4-{6-METHOXY-7-(4-METHOXYPHENOXY)-2-[(5-METHYL-1H-IMIDAZOL-4-YL)METHYL]-1,2,3,4-TETRAHYDROISOQUINOLIN-1-YL}BENZAMIDE

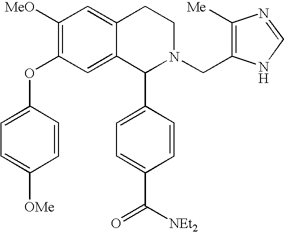

A solution of hydrochloric acid in 1,4-dioxane (4M, 0.5 mL) was slowly added to compound INTERMEDIATE 16.3.2 (15 mg, 0.023 mmol). The mixture was stirred at room temperature for 1 hr then evaporated solvent by applying a stream of nitrogen to dryness and followed by under vacuum for further 1 hr. The residue was stirred in anhydrous diethyl ether for 5 min (1×1 mL) and filtered. The insoluble compound was washed with fresh anhydrous ethyl ether (1×1 mL) to afford COMPOUND 16.4.2 (3.8 mg, 0.0064 mmol, 28%) as off white solid. ¹H NMR (500 MHz, CD₃OD): δ 1.12 (br s, 3H), 1.26 (br s, 3H), 2.29 (s, 3H), 2.28-2.60 (m, 4H), 3.25 (br s, 2H), 3.57 (br s, 2H), 3.58 (m, 1H), 3.74 (s, 3H), 3.81 (m, 1H), 3.87 (s, 3H), 6.72 (s, 1H), 6.78 (s, 1H), 7.30-7.80 (m, 8H), 8.85 (br s, 1H). (+) LRESIMS m/z 555 [M+H]⁺.

### COMPOUND 16.4.3: 1-{4-[(DIETHYLAMINO)CARBONYL]PHENYL}-6-METHOXY-2-[(5-METHYL-1H-IMIDAZOL-4-YL)METHYL]-1,2,3,4-TETRAHYDROISOQUINOLIN-7-YL BENZENESULFONATE

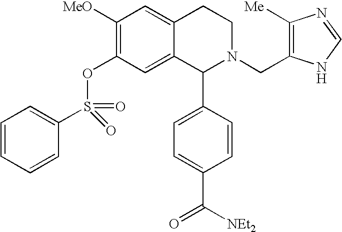

A solution of hydrochloric acid in 1,4-dioxane (4M, 0.5 mL) was slowly added to a compound INTERMEDIATE 16.3.3 (15 mg, 0.0218 mmol). The mixture was stirred at room temperature for 1 hr then evaporated solvent by applying a stream of nitrogen to dryness and followed by under vacuum for further 1 hr. The residue was stirred in anhydrous diethyl ether for 5 min (1×1 mL) and filtered. The insoluble compound was washed with fresh anhydrous ethyl ether (1×1 mL) to afford COMPOUND 16.4.3 (12.36 mg, 0.0197 mmol, 90%) as off white solid. ¹H NMR (500 MHz, CD₃OD): δ 1.16 (br s, 3H), 1.26 (br, 3H), 2.30 (s, 3H), 2.40-3.75 (m, 9H), 3.25 (br s, 2H), 6.50-7.75 (br s, 9H), 8.95 (br s, 1H). (+) LRESIMS m/z 591 [M+H]⁺.

### COMPOUND 17.1.1: 4-{6,7-DIHYDROXY-2-[(2-PHENYL-1H-IMIDAZOL-5-YL)METHYL]-1,2,3,4-TETRAHYDROISOQUINOLIN-1-YL}-N,N-DIETHYLBENZAMIDE

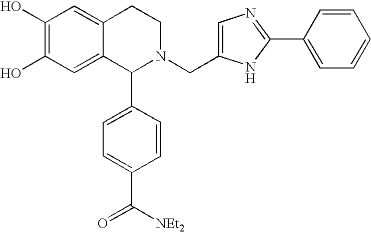

COMPOUND 12.1.9 (75 mg, 0.14 mmol) was dissolved in DCM (10 mL) and boron tribromide (42 ul, 0.43 mmol) was added dropwise as a solution in DCM (1 mL) at −78° C. The reaction was allowed to warm to room temperature and stirred for another 30 min at this temperature after which methanol (1.5 mL) was added at 0° C. After addition of water the aqueous layer was adjusted to pH 7 and extracted with DCM (3×). The combined organic layers were washed with water, brine, dried, and evaporated. Flash chromatography yielded a white foam (49 mg, 0.10 mmol, 71%). ¹H NMR (500 MHz, DMSO): 1.10 (brm, 6H), 2.92 (m, 1H), 4.10 (s, 1H), 5.60 (brs, 1H), 6.08, 6.62 (2 s, 2H), 7.40-7.56 (m, 7H), 8.00 (d, J 7.5 Hz, 2H). ¹³C NMR (125 MHz, CDCl₃): 11.14, 13.43, 14.03, 28.07, 39.69, 43.67, 46.67, 49.48, 56.04, 56.06, 67.48, 111.2, 112.0, 126.5, 129.9, 127.2, 127.3, 129.3, 130.2, 136.1, 133.3, 145.9, 147.6, 147.7, 171.8. (+) LRESIMS m/z 341 (100), 497 (35).

### COMPOUND 17.1.2: N,N-DIETHYL-4-{6-HYDROXY-2-[(2-PHENYL-1H-IMIDAZOL-5-YL)METHYL]-1,2,3,4-TETRAHYDROISOQUINOLIN-1-YL}BENZAMIDE

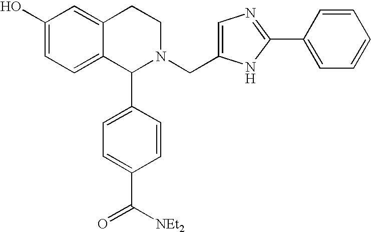

COMPOUND 12.1.10 (0.50 g, 1.01 mmol) was dissolved in DCM (20 mL) and boron tribromide (294 ul, 3.03 mmol) was added dropwise as a solution in DCM (5 mL) at −78° C. The reaction was allowed to warm to room temperature and stirred for another 30 min at this temperature after which methanol (1.5 mL) was added at 0° C. After addition of water the aqueous layer was adjusted to pH 7 and extracted with DCM (3×). The combined organic layers were washed with water, brine, dried, and evaporated. Flash chromatography yielded a white foam (0.33 g, 0.69 mmol, 69%). ¹H NMR (500 MHz, D₆-DMSO): 1.03 (brs, 6H), 2.60-3.60 (m, 10H), 4.79 (s, 1H), 6.42-6.55 (m, 3H), 7.01 (s, 1H), 7.28-7.43 (m, 6H), 7.90 (d, J 3.5 Hz, 2H), 9.20 (brs, 1H). ¹³C NMR (125 MHz, D₆-DMSO): δ 29.0, 43.8, 47.8, 66.1, 108.3, 116.2, 117.2, 125.5, 126.6, 126.7, 128.7, 129.4, 125.6, 129.0, 131.2, 136.1, 136.5, 145.9, 156.2, 171.5. (+) LRESIMS m/z 495 [M+H]⁺.

### COMPOUND 17.1.3: N,N-DIETHYL-4-{7-HYDROXY-2-[(2-PHENYL-1H-IMIDAZOL-5-YL)METHYL]-1,2,3,4-TETRAHYDROISOQUINOLIN-1-YL}BENZAMIDE

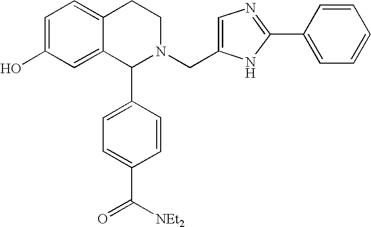

To a solution of COMPOUND 12.1.11 (100 mg, 0.20 mmole) in dichloromethane (10 mL) was added boron tribromide (69 μL, 0.71 mmole) at −78° C. and the resulting solution was allowed to warm to room temperature over 2 h. Saturated sodium hydrogen carbonate (25 mL) was then added and the mixture extracted with ethyl acetate (3×20 mL). The combined organic phase was dried (MgSO₄), filtered and the solvent removed in vacuo. The residue was purified by flash chromatography (methanol/chloroform, 5/95) to give COMPOUND 17.1.3 (70 mg, 72%) as a yellow solid. ¹H NMR (500 MHz, CDCl₃): δ 1.05, 1.26 (2 br s, 6H), 2.56, 2.66, 2.86, 3.07 (m, 4H), 3.19 (br s, 2H), 3.41 (d, J 14 Hz, 1H), 3.55 (br s, 2H), 3.56 (d, J 14 Hz, 1H), 4.47 (s, 1H), 5.88 (s, 1H), 6.64 (d, J 8.5 Hz, 1H), 6.75 (s, 1H), 6.87 (d, J 8.5 Hz, 1H), 7.03 (d, J 8 Hz, 2H), 7.08 (d, J 8 Hz, 21H), 7.27 (m, 3H), 7.75 (m, 2H). (+) LRESIMS m/z/481 [M+H]⁺.

### COMPOUND 17.1.4: N,N-DIETHYL-4-[1,2,3,4-TETRAHYDRO-6-HYDROXY-2-[(4-METHYL-1H-IMIDAZOL-5-YL)METHYL]-1-ISOQUINOLINYL]-BENZAMIDE

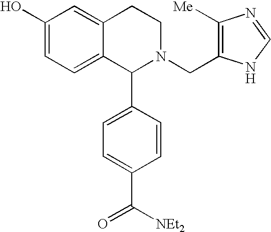

COMPOUND 12.1.21 (0.48 g, 1.11 mmol) was dissolved in dichloromethane (10 mL) and cooled to −78° C., boron tribromide (1.0M in DCM, 5.6 mL, 5.6 mmol) was added and the reaction mixture stirred for 1 h. MeOH (2 mL) was added and the reaction mixture stirred for 5 min. then concentrated to dryness, this process was repeated (×2), the resulting residue partitioned between EtOAc (20 mL) and NaHCO₃ (10 mL), the organics washed with EtOAc (20 mL), dried (MgSO₄), filtered and concentrated. Purification by flash chromatography on silica gel (10:1, CHCl₃:MeOH) gave COMPOUND 17.1.4 (240 mg, 52%) as a pale yellow solid. ¹H NMR (500 MHz, DMSO): δ 1.03 (br s, 6H), 2.05 (s, 3H), 2.62 (m, 1H), 2.73 (m, 1H), 2.89 (m, 1H), 2.99 (m, 1H), 3.30 (br s, 2H), 3.40 (br s, 2H), 3.53 (d, J 14 Hz, 1H), 3.57 (d, J 14 Hz, 1H), 4.70 (s, 1H), 6.47 (s, 1H), 6.54 (br s, 1H), 7.26 (d, J 8 Hz, 2H), 7.31 (d, J 8 Hz, 2H), 7.27 (br s, 1H), 8.62 (s, 1H). ¹³C NMR (125 MHz, DMSO): δ 8.9, 12.8, 14.0, 27.6, 39.6, 43.8, 46.5, 48.6, 66.3, 113.6, 114.2, 126.0, 126.6, 126.9, 129.2, 129.4, 132.9, 135.0, 136.1, 144.1, 155.6, 169.8. (+) LRESIMS m/z 419 [M+H]⁺.

### COMPOUND 17.1.5: N,N-DIETHYL-4-{7-HYDROXY-2-[(4-METHYL-1H-IMIDAZOL-5-YL)METHYL-1,2,3,4-TETRAHYDROISOQUINOLIN-1-YL}BENZAMIDE

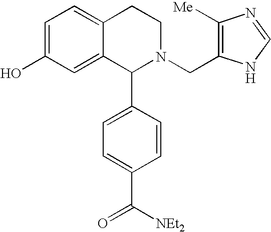

To a solution of COMPOUND 12.1.44 (100 mg, 0.23 mmole) in dichloromethane (10 mL) was added boron tribromide (77 μL, 0.81 mmole) at −78° C. and the resulting solution was allowed to warm to room temperature over 2.5 h. Saturated sodium hydrogen carbonate (20 mL) and dichloromethane (50 mL) was then added and the mixture filtered through a Whatman 1PS filter paper. The solvent was removed from the organic phase in vacuo and the residue purified by flash chromatography (methanol/chloroform, 1/9) to give COMPOUND 17.1.5 (48 mg, 50%) as a yellow solid. ¹H NMR (500 MHz, CDCl₃): δ 1.10 (br s, 6H), 2.02 (s, 3H), 2.55 (m, 1H), 2.69 (m, 1H), 2.84 (m, 1H), 3.03 (m, 1H), 3.19 (br s, 2H), 3.43 (br s, d, J 14.5 Hz, 3H), 3.54 (d, J 14.5 Hz, 1H), 4.66 (s, 1H), 6.10 (d, J 2 Hz, 1H), 6.55 (dd, J 2, 8.5 Hz, 1H), 6.94 (d, J 8.5 Hz, 1H), 7.30 (d, J 8 Hz, 2H), 7.36 (d, J 8 Hz, 2H), 8.27 (s, 1H), 9.04 (br s, 1H). (+) LRESIMS m/z 419 [M+H]⁺.

### COMPOUND 17.1.6: N,N-DIETHYL-4-{6-HYDROXY-7-PHENOXY-2-[(2-PHENYL-1H-IMIDAZOL-4-YL)METHYL]-1,2,3,4-TETRAHYDROISOQUINOLIN-1-YL}BENZAMIDE

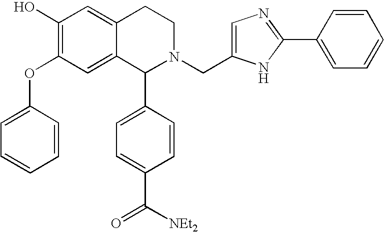

To a solution of COMPOUND 14.1.1 (19.3 mg, 0.0329 mmol) in anhydrous dichloromethane (0.2 mL) at −78° C. was added a solution of boron tribromide (0.2 mL). The reaction mixture was stirred at −78° C. for 1 hr then allowed to warm to room temperature for 2 hr. Solvent was removed by applying a stream of nitrogen. The residue was redissolved in EtOAc (5 mL), washed with aqueous sodium bicarbonate solution (1×2 mL), dried over MgSO₄ and concentrated. Product was purified by flash chromatography to give 3.1 mg (0.0074 mmol) of COMPOUND 17.1.6 as oil. ¹H NMR (500 MHz, CD₃OD): δ 0.96 (br s, 3H), 1.13 (br s, 3H), 2.59 (m, 1H), 2.74 (m, 1H), 2.95 (m, 1H), 3.13 (br s, 2H), 3.18 (m, 1H), 3.43 (br s, 2H), 3.46 (m, 1H), 4.54 (s, 1H), 6.12 (s, 1H), 6.63 (s, 1H), 6.65 (s, 1H), 6.83-7.75 (m, 14H). ¹³C NMR (125 MHz, CD₃OD): δ 11.87, 13.16, 28.08, 40.00, 43.70, 67.19, 116.37, 121.96, 125.27, 126.08, 128.49, 128.72, 129.22, 129.82, 130.37, 132.00, 135.88, 141.76, 146.00, 148.00, 158.00, 172.37. (+) LRESIMS m/z 573 (M+H)⁺.

### COMPOUND 17.1.7: N,N-DIETHYL-4-{6-HYDROXY-2-[(5-METHYL-1H-IMIDAZOL-4-YL)METHYL]-7-PHENOXY-1,2,3,4-TETRAHYDROISOQUINOLIN-1-YL}BENZAMIDE

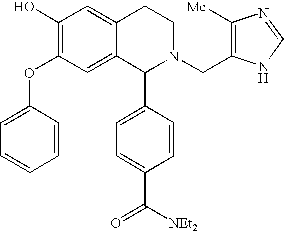

To a solution of COMPOUND 14.1.2 (14.3 mg, 0.0269 mmol) in anhydrous dichloromethane (0.2 mL) at −78° C. was added a solution of boron tribromide (0.2 mL). The reaction mixture was stirred at −78° C. for 1 hr then allowed to warm to room temperature for 2 hr. Solvent was removed by applying a stream of nitrogen. The residue was redissolved in EtOAc (5 mL), washed with aqueous sodium bicarbonate solution (1×2 mL), dry over MgSO₄ and concentrated. Product was purified by flash chromatography to give 3.8 mg (0.0074 mmol) of COMPOUND 17.1.7 as oil. ¹H NMR (500 MHz, CD₃OD): δ 1.07 (br s, 3H), 1.25 (br s, 3H), 2.03 (s, 3H), 2.59 (m, 1H), 2.76 (m, 1H), 2.97 (m, 1H), 3.15 (m, 1H), 3.24 (br s, 2H), 3.32 (m, 1H), 3.54 (br s, 2H), 3.63 (m, 1H), 4.51 (s, 1H), 6.20 (s, 1H), 6.70 (s, 1H), 6.74 (brm, 2H), 6.96 (t, J 8.3 Hz, 1H), 7.19 (t, J 8.3 Hz, 2H), 7.31 (d, J 8.3 Hz, 2H), 7.41 (d, J 8.3 Hz, 2H), 7.53 (s, 1H). ¹³C NMR (125 MHz, CD₃OD): δ 9.43, 11.90, 13.17, 28.04, 39.70, 43.81, 49.34, 68.10, 116.13, 116.35, 121.23, 121.95, 126.06, 129.21, 129.58, 129.75, 131.71, 133.29, 135.89, 141.72, 146.12, 147.57, 158.45, 172.39. (+) LRESIMS m/z 511 (M+H)⁺.

### COMPOUND 17.1.8: N,N-DIETHYL-4-{7-(4-FLUOROPHENOXY)-6-HYDROXY-2-[(2-PHENYL-1H-IMIDAZOL-4-YL)METHYL]-1,2,3,4-TETRAHYDROISOQUINOLIN-1-YL}BENZAMIDE

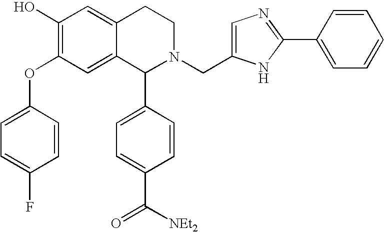

A solution of COMPOUND 14.1.3 (15 mg, 0.0248 mmol) in anhydrous dichloromethane (0.5 mL) was cooled to −78° C. and then to this solution boron tribromide (0.5 mL) was added. The reaction mixture was stirred at −78° C. for 1 hr then was allowed to warm to room temperature for further 2 hr. Excess solvent and reagent were removed from the reaction by a stream of nitrogen. The residue was re-dissolved in dichloromethane (20 mL) and washed the dichloromethane with saturated aqueous sodium bicarbonate (2×5 mL), brine (2×5 mL) then dried over MgSO₄ and concentrated. Product was purified by flash chromatography to give 6.5 mg (0.011 mmol, 44%) of COMPOUND 17.1.8 as colourless oil. ¹H NMR (500 MHz, CD₃OD): δ 1.09 (br s, 3H), 1.25 (br s, 3H), 2.70 (m, 1H), 2.85 (m, 1H), 3.05 (m, 1H), 3.25 (br s, 3H), 3.55 (br s, 4H), 3.70 (br d, 2H), 4.65 (s, 1H), 6.20 (s, 1H), 6.75 (m, 14H). (+) LRESIMS m/z 591 [M+H]⁺.

### COMPOUND 17.1.9: N,N-DIETHYL-1-{7-(4-FLUOROPHENOXY)-6-HYDROXY-2-[(5-METHYL-1H-IMIDAZOL-4-YL)METHYL]-1,2,3,4-TETRAHYDROISOQUINOLIN-1-YL}BENZAMIDE

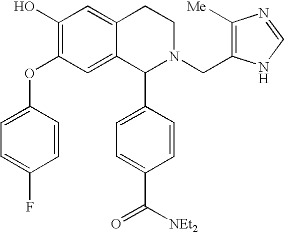

A solution of COMPOUND 14.1.4 (10 mg, 0.0184 mmol) in anhydrous dichloromethane (0.3 mL) was cooled to −78° C. and then to this solution boron tribromide (0.3 mL) was added. The reaction mixture was stirred at −78° C. for 1 hr then was allowed to warm to room temperature for further 2 hr. Excess solvent and reagent were removed from the reaction by a stream of nitrogen. The residue was re-dissolved in dichloromethane (10 mL) and washed with saturated aqueous sodium bicarbonate (2×2 mL), brine (2×2 mL) then dried over MgSO₄ and concentrated. Product was purified by flash chromatography to give 5.87 mg (0.011 mmol, 60%) as colourless oil. ¹H NMR (500 MHz, CD₃OD): δ 0.97 (br s, 3H), 1.40 (br s, 3H), 1.93 (s, 3H), 2.48 (dd, J 9.2 Hz, 8.6 Hz, 1H), 2.66 (d, J 16.2 Hz, 1H), 2.88 (br m, 1H), 3.04 (br m, 1H), 3.14 (br s, 2), 3.22 (d, J 14 Hz, 1H), 3.43 (br s, 2H), 3.51 (d, J 14 hz, 1H), 4.41 (s, 1H), 6.07 (s, 1H), 6.61 (s, 1H), 6.62 (m, 2H), 6.81 (m, 2H), 7.21 (m, 2H), 7.29 (m, 2H), 7.53 (s, 1H). ¹³C NMR (125 MHz, CD₃OD): δ 9.24, 11.89, 13.17, 39.71, 43.78, 49.23, 68.07, 115.52 (d, J 23.6 Hz), 116.21, 117.80, 120.88, 126.10, 127.76, 128.73, 129.49, 129.75, 131.68, 133.29, 135.97, 142.15, 145.90, 147.49, 154.46, 158.24, 172.32. (+) LRESIMS m/z 529 [M+H]⁺.

### COMPOUND 18.1.1: 4-{2-[(1,4-DIMETHYL-1H-IMIDAZOL-5-YL)METHYL]-6,7-DIMETHOXY-1,2,3,4-TETRAHYDROISOQUINOLIN-1-YL}-N,N-DIETHYLBENZAMIDE

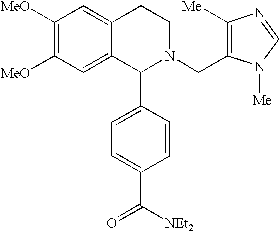

Methyl iodide (8.3 μL, 18.9 mg, 0.133 mmol) was added to a stirring solution of COMPOUND 12.1.19 (56 mg, 0.121 mmol) in anhydrous DMF (4 mL) followed by sodium hydride 60% suspension in oil (7.26 mg, 0.182 mmol). The mixture was stirred at RT for 3 h and the solvent removed in vacuo. The residue was diluted with DCM (10 mL) and washed with brine (5 mL) and water (5 mL). The residue was purified by flash chromatography on SiO₂ column (hexane:DCM:MeOH 60:39:1) to afford a mixture of reioisomers (30 mg, 52%) which was further separated by HPLC on a YMC-Pack Diol (5 μm) Semi-preparative (150×10 mm) HPLC column using isocratic elution (hexane:ethanol:DIEA 60:40:0.1) at 3 mL/min. COMPOUND 18.1.1 eluted pure as a colourless oil (15.9 mg, 53%) at 6.15 min. ¹H NMR (500 MHz, CDCl₃): δ 1.12 (br s, 3H), 1.25 (br s, 3H), 2.16 (s, 3H), 2.45 (m, 1H), 2.72 (dt, J 4.6, 16 Hz, 1H), 2.86 (m, 1H), 3.04 (m, 1H), 3.24 (br s, 2H), 3.28 (4, J 14 Hz, 1H), 3.39 (s, 3H), 3.55 (br s, 2H), 3.61 (m, 1H), 3.63 (s, 3H), 3.87 (s, 3H), 4.48 (s, 1H), 6.16 (s, 1H), 6.63 (s, 1H), 7.31 (m, 3H), 7.35 (m, 2H); ¹³C NMR (125 MHz, CDCl₃): δ 12.32, 12.87, 14.02, 28.10, 31.98, 39.64, 43.56, 46.44, 47.53, 56.06, 56.09, 68.60, 111.17, 111.99, 123.58, 126.58, 127.39, 129.34, 129.86, 136.55, 136.94, 137.64, 145.65, 147.52, 147.98, 171.30; (+) LRESIMS m/z 477.00 [M+H]⁺, 499.00 [M+Na]⁺.

### COMPOUND 18.1.2: 4-[2-[(1,5-DIMETHYL-1H-IMIDAZOL-4-YL)METHYL]-6,7-DIMETHOXY-1,2,3,4-TETRAHYDROISOQUINOLIN-1-YL]-N,N-DIETHYLBENZAMIDE

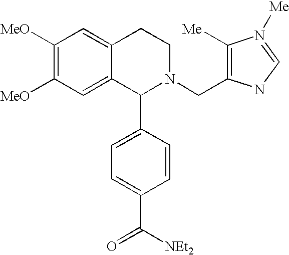

Methyl iodide (8.3 μL, 18.9 mg, 0.133 mmol) was added to a stirring solution of COMPOUND 12.1.19 (56 mg, 0.121 mmol) in anhydrous DMF (4 mL) followed by sodium hydride 60% suspension in oil (7.26 mg, 0.182 mmol). The mixture was stirred at RT for 3 h and the solvent removed in vacuo. The residue was diluted with DCM (10 mL) and washed with brine (5 mL) and water (5 mL). The residue was purified by flash chromatography on SiO₂ column hexane:DCM:MeOH 60:39:1) to afford a mixture of reioisomers (30 mg, 52%) which was further separated by HPLC on a YMC-Pack Diol (5 μm) Semi-preparative (150×10 mm) HPLC column using isocratic elution (hexane:ethanol:DIEA 60:40:0.1) at 3 mL/min. COMPOUND 18.1.2 eluted pure as a colourless oil (6 mg, 20%) at 6.89 min; ¹H NMR (500 MHz, CDCl₃): δ 1.12 (br s, 3H), 1.26 (br s, 3H), 2.04 (s, 3H), 2.75 (m, 1H), 2.76 (m, 1H), 3.13 (m, 1H), 3.27 (m, 2H), 3.32 (m, 1H), 3.49 (m, 1H), 3.52 (s, 3H), 3.57 (m, 2H), 3.60 (s, 3H), 3.75 (m, 1H), 3.86 (s, 3H), 4.84 (br s, 1), 6.17 (s, 1H), 6.62 (s, 1H), 7.36 (d, J 8 Hz, 2H), 7.45 (m, 3H); ¹³C NMR (125 MHz, CDCl₃): δ 8.59, 13.13, 14.48, 28.11, 31.74, 39.90, 43.59, 47.66, 51.13, 56.07, 67.66, 111.16, 111.90, 126.64, 126.95, 130.15, 133.50, 135.79, 136.61, 147.51, 147.98, 171 40; 2 D NMR (600 MHz, CDCl₃; (+) LRESIMS m/z 477.00 [M+H]⁺, 499.00 [M+Na]⁺.

### COMPOUND 19.1.1: 4-{7-ETHOXY-6-METHOXY-2-[(5-METHYL-1H-IMIDAZOL-4-YL)METHYL-1,2,3,4-TETRAHYDROISOQUINOLIN-1-YL}-N,N-DIETHYLBENZAMIDE

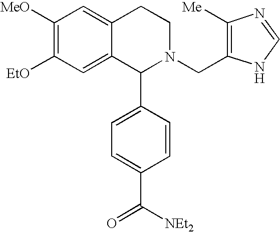

To a solution of triphenylphosphine (3 eq, 0.194 mmol, 50.8 mg) in anhydrous dichloromethane (0.5 mL) was added diisopropylazodicarboxylate (DIAD, 3 eq, 0.194 mmol, 39.2 mg, 38 uL) at 0° C. After 5 min, a solution of absolute ethanol (1.5 eq, 0.097 mmol, 4.5 mg, 5.7 uL) and COMPOUND 12.1.26 (1 eq, 0.0647 mmol, 29 mg) in anhydrous dichloromethane (2 mL) were added. The reaction mixture was clear at the beginning but became cloudy after removal of ice-bath and it was stirred for overnight. The reaction mixture was then quenched with water (2 mL) and extracted to EtOAc (3×10 mL). The combined extracts were washed with water (1×5 mL), brine (1×5 mL), dried (MgSO₄) and concentrated. Product was purified by flash chromatography to give 10 mg (0.021 mmol, 32%) of COMPOUND 19.1.1 as colorless oil. ¹H NMR (500 MHz, CDCl₃): δ 1.08 (br s, 3H), 1.25 (br s, 3H), 1.28 (t, J 7 Hz, 3H), 2.15 (s, 3H), 2.25-3.10 (m, 4H), 3.21 (br s, 2H), 3.42 (s, 3H), 3.48 (br s, 2H), 3.78 (q, J 7 Hz, 2H), 4.95 (br s, 1H), 6.10 (s, 1H), 6.60 (s, 1H), 7.28-7.60 (m, 4H), 7.89 (br s, 1H). ¹³C NMR (125 z, CDCl₃): δ 10.90, 15.00, 16.50, 17.00, 26.30, 39.80, 43.90, 46.50, 47.80, 56.10, 64.80, 66.70, 111.50, 113.80, 126.00, 127.50, 127.80, 130.50, 132.00, 137.80, 146.00, 148.10, 171.10. (+) LRESIMS m/z 477 (M+H)⁺.

### COMPOUND 20.1.1: 4-{(1S)-6,7-DIMETHOXY-2-[(4-METHYL-1H-IMIDAZOL-5-YL)METHYL]-1,2,3,4-TETRAHYDROISOQUINOLIN-1-YL}-N,N-DIETHYLBENZAMIDE

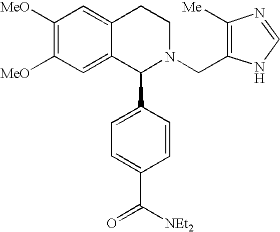

The chiral resolution of COMPOUND 12.1.19 was achieved on a CHIRACEL OD-H analytical (250×4.6 mm) HPLC column using an isocratic elution of hexane/ethanol 90:10 with 0.1% diisopropylamine, with a flow rate of 1.0 mL/min. COMPOUND 20.1.1 eluted pure as a colourless oil at 11.5 min.: (+) LRESIMS m/z 463 [M+H]⁺.

### COMPOUND 20.2.1: 4-{(1R)-6,7-DIMETHOXY-2-[(4-METHYL-1H-IMIDAZOL-5-YL)METHYL]-1,2,3,4-TETRAHYDROISOQUINOLIN-1-YL}-N,N-DIETHYLBENZAMIDE

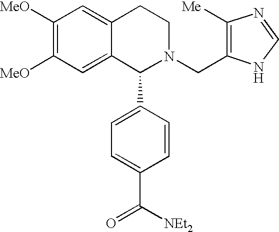

The chiral resolution of COMPOUND 12.1.19 was achieved on a CHIRALCEL OD-H analytical (250×4.6 mm) HPLC column using an isocratic elution of hexane/ethanol 90:10 with 0.1% diisopropylamine, with a flow rate of 1.0 mL/min. COMPOUND 20.2.1 eluted pure as a colourless oil at 15.5 min.: (+) LRESIMS m/z 463 [M+H]⁺.

### COMPOUND 20.1.2: N,N-DIETHYL-4-{(1S)-6-METHOXY-2-[(4-METHYL-1H-IMIDAZOL-5-YL)METHYL]-1,2,3,4-TETRAHYDROISOQUINOLIN-1-YL}BENZAMIDE

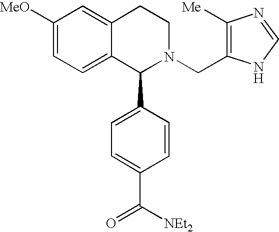

The chiral resolution of COMPOUND 12.1.21 was achieved on a Chiralcel OD-H (250×4.6 mm) analytical HPLC column with isocratic elution (hexane:EtOH:DIEA 90:10:0.1). COMPOUND 20.1.2 eluted pure as a colourless oil at 7.9 min: [alphaD]^{29° C.} +59.51±1.38; (+) LRESIMS m/z 433 [M+H]⁺.

### COMPOUND 20.2.2: N,N-DIETHYL-4-{(1R)-6-METHOXY-2-[(4-METHYL 1H-IMIDAZOL-5-YL)METHYL]-1,2,3,4-TETRAHYDROISOQUINOLIN-1-YL}BENZAMIDE

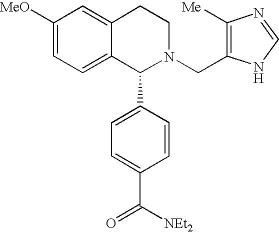

The chiral resolution of COMPOUND 12.1.21 was achieved on a Chiralcel OD-H (250×4.6 mm) analytical HPLC column with isocratic elution (hexane:EtOH:DIEA 90:10:0.1). COMPOUND 20.2.2 eluted pure as a colourless oil at 9.8 min.: [alphaD]^{29° C.} −54.90±0.64; (+) LRESIMS m/z 433 [M+H]⁺.

### COMPOUND 20.1.3: N,N-DIETHYL-4-{(1S)-6-HYDROXY-2-[(2-PHENYL-1H-IMIDAZOL-5-YL)METHYL]-1,2,3,4-TETRAHYDROISOQUINOLIN-1-YL}BENZAMIDE

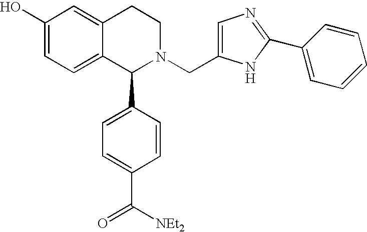

The chiral resolution of COMPOUND 17.1.2 (CJ3.35-3) was achieved on a Chiralcel OD-H (250×4.6 mm) analytical HPLC column with isocratic elution hexane:EtOH:DIEA 90:10:0.1). COMPOUND 20.1.3 eluted pure as a colourless oil at 28 min.: [alphaD]^{29° C.}+83.40±0.97.

### COMPOUND 20.2.3: N,N-DIETHYL-4-{(1R)-6-HYDROXY-2-[(2-PHENYL-1H-IMIDAZOL-5-YL)METHYL]-1,2,3,4-TETRAHYDROISOQUINOLIN-1-YL}BENZAMIDE

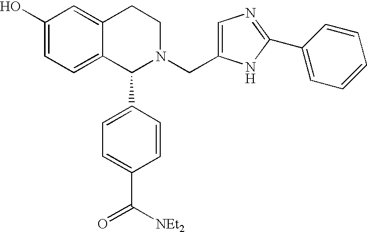

The chiral resolution of COMPOUND 17.1.2 (CJ 3.35-3) was achieved on a Chiralcel OD-H (250×4.6 mm) analytical HPLC column with isocratic elution (hexane:EtOH:DIEA 90:10:0.1). COMPOUND 20.2.3 eluted pure as a colourless oil at 21 min.: [alphaD]^{29° C.} −76.56±0.91.

### COMPOUND 20.1.4: N,N-DIETHYL-4-{(1S)-7-ISOPROPOXY-6-METHOXY-2-[(4-METHYL-1H-IMIDAZOL-5-YL)METHYL]-1,2,3,4-TETRAHYDROISOQUINOLIN-1-YL}BENZAMIDE

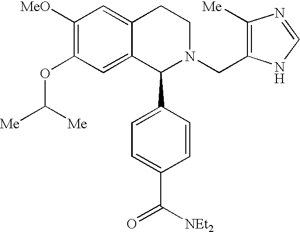

The chiral resolution of COMPOUND 12.1.39 was achieved on a Chiralcel OD-H (250×4.6 mm) analytical HPLC column with isocratic elution (hexane:EtOH:DIEA 90:10:0.1). COMPOUND 20.1.4 eluted pure as a colourless oil at 10 min.: [alphaD]^{28° C.}+ 20.65±1.78; (+) LRESIMS m/z 491.29 [M+H]⁺.

### COMPOUND 20.2.4: N,N-DIETHYL-4{(1R)-7-ISOPROPOXY-6-METHOXY-2-[(4-METHYL-1H-IMIDAZOL-5-YL)METHYL]-1,2,3,4-TETRAHYDROISOQUINOLIN-1-YL}BENZAMIDE

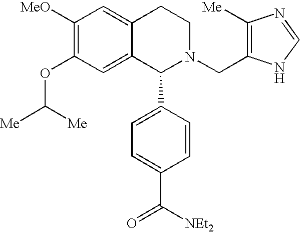

The chiral resolution of COMPOUND 12.1.39 was achieved on a Chiralcel OD-H (250×4.6 mm) analytical HPLC column with isocratic elution (hexane:EtOH:DIEA 90:10:0.1). COMPOUND 20.2.4 eluted pure as a colourless oil at 14 min.: [alphaD]^{28° C.}−15.52±1.07; (+) LRESIMS m/z 491.29 [M+H]⁺.

### COMPOUND 20.1.5: N,N-DIETHYL-4-{(1S)-7-ISOPROPOXY-6-METHOXY-2-[(2-PHENYL-1H-IMIDAZOL-5-YL)METHYL]-1,2,3,4-TETRAHYDROISOQUINOLIN-1-YL}BENZAMIDE

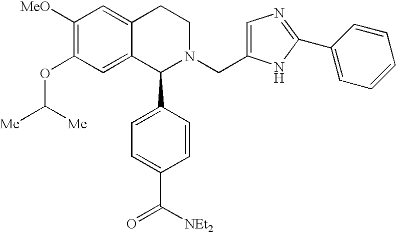

The chiral resolution of COMPOUND 12.1.42 was achieved on a Chiralcel OD-H (250×4.6 mm) analytical HPLC column with isocratic elution (hexane:EtOH:DIEA 90:10:0.1). COMPOUND 20.1.5 eluted pure as a colourless oil at 21.5 min.: [alphaD]^{28° C.} +62.20±1.33; (+) LRESIMS m/z 553.305 [M+H]⁺.

### COMPOUND 20.2.5: N,N-DIETHYL-4-{(1R)-7-ISOPROPOXY-6-METHOXY-2-[(2-PHENYL-1H-IMIDAZOL-5-YL)METHYL]-1,2,3,4-TETRAHYDROISOQUINOLIN-1-YL}BENZAMIDE

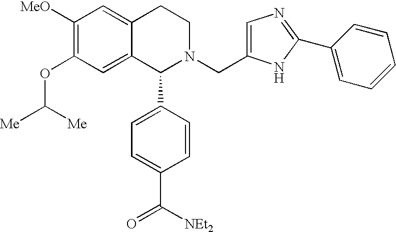

The chiral resolution of COMPOUND 12.1.42 was achieved on a Chiralcel OD-H (250×4.6 mm) analytical HPLC column with isocratic elution (hexane:EtOH:DIEA 90:10:0.1). COMPOUND 20.2.5 eluted pure as a colourless oil at 18 min.: [alphaD]^{28° C.} −47.82±1.35; (+) LRESIMS m/z 553.305 [M+H]⁺.

### COMPOUND 20.1.6: N,N-DIETHYL-4-{(1S)-6-METHOXY-7-(2-MORPHOLIN-4-YLETHOXY)-2-[(2-PHENYL-1H-IMIDAZOL-5-YL)METHYL]-1,2,3,4-TETRAHYDROISOQUINOLIN-1-YL}BENZAMIDE

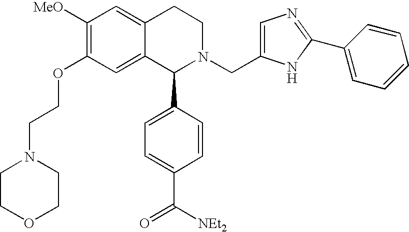

The chiral resolution of COMPOUND 12.1.43 was achieved on a Chiralcel OD-H (250×4.6 mm) analytical HPLC column with isocratic elution (hexane:EtOH:DIEA 90:10:0.1). COMPOUND 20.1.6 eluted as a colourless oil at 26.5 min.: [alphaD]^{28° C.}+ 34.53±1.53.

### COMPOUND 20.2.6: N,N-DIETHYL-4-{(1R)-6-METHOXY-7-(2-MORPHOLIN-4-YLETHOXY)-2-[(2-PHENYL-1H-IMIDAZOL-5-YL)METHYL]-1,2,3,4-TETRAHYDROISOQUINOLIN-1-YL}BENZAMIDE

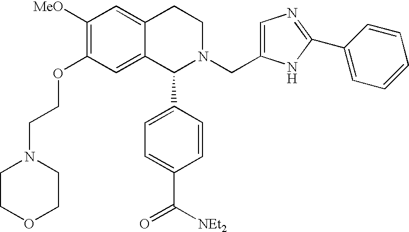

The chiral resolution of COMPOUND 12.1.43 was achieved on a Chiralcel OD-H (250×4.6 mm) analytical HPLC column with isocratic elution (hexane:EtOH:DIEA 90:10:0.1). COMPOUND 20.2.6 eluted as a colourless oil at 22 min.: [alphaD]^{28° C.} −12.58±1.85.

### COMPOUND 20.1.7: N,N-DIETHYL-4-[(1S)-1,2,3,4-TETRAHYDRO-6-METHOXY-2-[(4-METHYL-1H-IMIDAZOL-5-YL)METHYL]-1-ISOQUINOLINYL]-BENZAMIDE

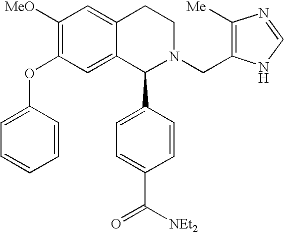

Chiral resolution of COMPOUND 14.1.2 on a CHIRACEL OD-H preparative (250×25 mm) HPLC column using isocratic elution of 90: 10:1 (Hexane:EtOH:Diethylamine), with a flow rate of 10 mL/mini gave COMPOUND 20.1.7 at a retention time of 25.5 min. [alphaD]^{25° C.}+ 18.70±1.86. (+) LRESIMS m/z 497 [M+H]⁺.

### COMPOUND 20.2.7: N,N-DIETHYL-4-[(1R)-1,2,3,4-TETRAHYDRO-6-METHOXY-2-[(4-METHYL-1H-IMIDAZOL-5-YL)METHYL]-1-ISOQUINOLINYL]-BENZAMIDE

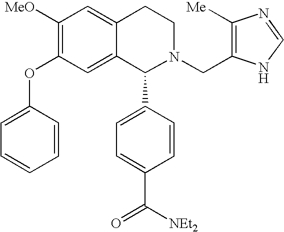

Chiral resolution of COMPOUND 14.1.2 on a CHIRACEL OD-H preparative (250×25 mm) HPLC column using isocratic elution of 90:10:1 (Hexane:EtOH:Diethylamine), with a flow rate of 10 mL/min. gave COMPOUND 20.2.7 at a retention time of 35.2 min. [alphaD]^{25° C.}− 25.56±2.84. (+) LRESIMS m/z 497 [M+H]⁺.

### COMPOUND 20.1.8: N,N-DIETHYL-4-[(1)-1,2,3,4-TETRAHYDRO-6-HYDROXY-2-[(4-METHYL-1H-IMIDAZOL-5-YL)METHYL]-1-ISOQUINOLINYL]-BENZAMIDE

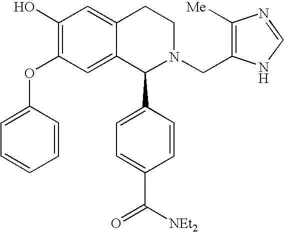

Chiral resolution of COMPOUND 17.1.7 on a CHIRACEL OD-H-preparative (250×25 mm) HPLC column using isocratic elution of 85:15:1 (Hexane:EtOH:Diethylamine), with a flow rate of 10 mL/min. gave COMPOUND 20.1.8 at a retention time of 14.5 min. (+) LRESIMS m/z 511 [+H]⁺.

### COMPOUND 20.2.8: N,N-DIETHYL-4-[(1R)-1,2,3,4-TETRAHYDRO-6-HYDROXY-2-[(4-METHYL-1H-IMIDAZOL-5-YL)METHYL]-1-ISOQUINOLINYL]-BENZAMIDE

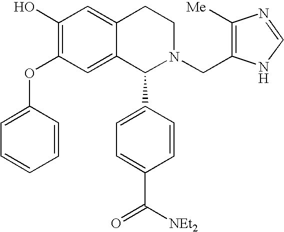

Chiral resolution of COMPOUND 17.1.7 on a CHIRACEL OD-H preparative (250×25 mm) HPLC column using isocratic elution of 85:15:1 (Hexane:EtOH:Diethylamine), with a flow rate of 10 mL/min. gave COMPOUND 20.2.8 at a retention time of 20.4 min. (+) LRESIMS m/z 511 [+H]⁺.

1. A compound of formula I, a pharmaceutically acceptable salt thereof, diastereomers, enantiomers, or mixtures thereof: 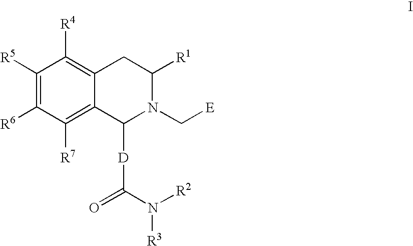
wherein
R¹ is selected from —H and C₁₋₆alkyl;
R² and R³ are independently selected from —H and C₁₋₆alkyl;
R⁴, R⁵, R⁶ and R⁷ are independently selected from —H, —OH, halogen, —NO₂, C₁₋₆alkyl, C₆₋₁₀aryl, C₁₋₆alkoxy, C₃₋₆cycloalkoxy, C₃₋₆heterocyclyl-oxy, C₃₋₆heterocyclyl-C₁₋₄alkoxy, C₆₋₁₀aryl-oxy, C₆₋₁₀aryl-C₁₋₄alkoxy, C₁₋₆alkyl-S(═O)₂—O—, C₆₋₁₀aryl-S(═O)₂—O—, C₁₋₆alkyl-NH—S(═O)₂—O—, and (C₁₋₆alkyl)₂N—S(═O)₂—O—; or any two adjacent groups selected from R⁴, R⁵, R⁶ and R⁷ form a portion of a 5 or 6-membered ring that fused with the benzene ring of formula I, wherein said C₁₋₆alkyl, C₆₋₁₀aryl, C₁₋₆alkoxy, C₃₋₆cycloalkoxy, C₃₋₆heterocyclyl-oxy, C₃₋₆heterocyclyl-C₁₋₄alkoxy, C₆₋₁₀aryl-oxy, C₆₋₁₀aryl-C₁₋₄alkoxy, C₁₋₆alkyl-S(═O)₂—O—, C₆₋₁₀aryl-S(═O)₂—O—, C₁₋₆alkyl-NH—S(═O)₂—O—, and (C₁₋₆alkyl)₂N—S(═O)₂—O— are optionally substituted with one or more groups selected from halogen, C₁₋₃alkoxy, —OH, —NO₂, C₁₋₃alkyl, —NH₂, and —CO₂—C₁₋₃alkyl;
E is a 5-membered heterocyclyl optionally substituted with one or more groups selected from halogen, C₁₋₆alkyl, —C(═O)—O—C₁₋₆alkyl, C₆₋₁₀aryl, C₆₋₁₀aryl-C₁₋₄alkyl, and C₆₋₁₀aryl-S(═O)₂—; and
D is a divalent group comprising a benzene ring.

2. A compound according to claim 1, wherein
R¹ is selected from —H and C₁₋₃alkyl;
R² and R³ are independently C₁₋₃alkyl;
R⁴, R⁵, R⁶ and R⁷ are independently selected from —H, —OH, halogen, —NO₂, C₁₋₆alkyl, phenyl, C₁₋₆alkoxy, C₃₋₆cycloalkoxy, tetrahydropyranyloxy, pyridinyloxy, morpholinyloxy, tetrahydropyranyl-C₁₋₄alkoxy, pyridinyl-C₁₋₄alkoxy, morpholinyl-C₁₋₄alkoxy, phenoxy, benzyloxy, C₁₋₆alkyl-S(═O)₂—O—, phenyl-S(═O)₂—O—, C₁₋₃alkyl-NH—S(═O)₂—O—, and (C₁₋₃alkyl)₂N—S(═O)₂—O—; or any two adjacent groups selected from R⁴, R⁵, R⁶ and R⁷ form a divalent group selected from —O—CH₂—O— and —O—CH₂—CH₂—O—, wherein said C₁₋₆alkyl, phenyl, C₁₋₆alkoxy, C₃₋₆cycloalkoxy, tetrahydropyranyloxy, pyridinyloxy, morpholinyloxy, tetrahydropyranyl-C₁₋₄alkoxy, pyridinyl-C₁₋₄alkoxy, morpholinyl-C₁₋₄alkoxy, phenoxy, benzyloxy, C₁₋₆alkyl-S(═O)₂—O—, phenyl-S(═O)₂—O—, C₁₋₃alkyl-NH—S(═O)₂—O—, and (C₁₋₃alkyl)₂N—S(═O)₂—O— are optionally substituted with one or more groups selected from halogen, methoxy, —OH, —NO₂, and C₁₋₃alkyl;
E is selected from furyl, thienyl, imidazolyl, pyrazolyl, and thiazolyl, wherein said furyl, thienyl, imidazolyl, pyrazolyl, and thiazolyl are optionally substituted with one or more groups selected from halogen, C₁₋₄alkyl, —C(═O)—O—C₁₋₃alkyl, phenyl, benzyl, and benzenesulfonyl; and
D is selected from phenylene, pyridylene,

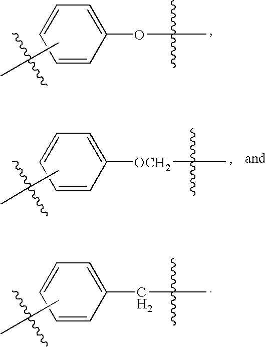

3. A compound according to claim 1, wherein
R¹ is selected from —H and methyl;
R² and R³ are selected from ethyl and isopropyl;
R⁴, R⁵ and R⁵ are independently selected from —H, —OH, halogen, —NO₂, C₁₋₆alkyl, phenyl, C₁₋₆alkoxy, C₃₋₆cycloalkoxy, tetrahydropyranyloxy, pyridinyloxy, morpholinyloxy, tetrahydropyranyl-C₁₋₄alkoxy, pyridinyl-C₁₋₄alkoxy, morpholinyl-C₁₋₄alkoxy, phenoxy, benzyloxy, C₁₋₆alkyl-S(═O)₂—O—, phenyl-S(═O)₂—O—, C₁₋₃alkyl-NH—S(═O)₂—O—, and (C₁₋₃alkyl)₂N—S(═O)₂—O—; or any two adjacent groups selected from R⁴, R⁵ and R⁶ form —O—CH₂—O—, wherein said phenoxy, benzyloxy, and phenyl-S(═O)₂—O— are optionally substituted with one or more groups selected from halogen and methoxy;
R⁷ is selected from —H and C₁₋₃alkoxy;
E is selected from furyl, thienyl, imidazolyl, pyrazolyl, and thiazolyl, wherein said furyl, thienyl, imidazolyl, pyrazolyl, and thiazolyl are optionally substituted with one or more groups selected from halogen, C₁₋₄alkyl, —C(═O)—O—C₁₋₃alkyl, phenyl, benzyl, and benzenesulfonyl; and
D is selected from para-phenylene, para-benzylene,

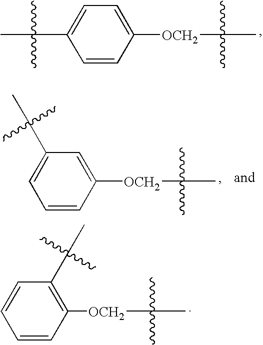

4. A compound according to claim 1, wherein
R¹ is selected from —H and methyl;
R² and R³ are ethyl;
R⁴ is selected from —H, NO₂ and methoxy, R⁵ is selected from —H, —Br, —F, —OH, methoxy, methylsulfonyloxy, N,N-dimethylsulfamyloxy, and R⁶ is selected from —H, —OH, —NO₂, methoxy, ethoxy, isopropyloxy, neopentyloxy, cyclobutyloxy, 4-tetrahydro-2H-pyranyloxy, 2-(4-morpholino)ethoxy, benzyloxy, phenoxy, 4-fluorophenoxy, 3-methoxyphenoxy, 4-methoxyphenoxy, 3-pyridinyloxy, methanesulfonyloxy, benzenesulfonyloxy, dimethylsulfamyloxy; or any two adjacent groups selected from R⁴, R⁵ and R⁶ form —O—CH₂—O—;
R⁷ is selected from —H and methoxy;
E is 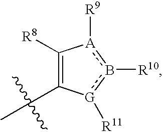
wherein A and B are independently selected from C, N and S, and G is selected from C, N, O and S with a proviso that at least one of A, B and G is C, at most one of A, B and G is S and one of the bonds between A and B, and between B and G is a double bond;
wherein R⁸ is selected from —H, —Cl, methyl, —CO₂Me and phenyl; R⁹ is selected from —H and methyl; R¹⁰ is selected from —H, methyl, n-butyl and phenyl; R¹¹ is selected from —H, methyl, benzyl and benzenesulfonyl.
D is selected from para-phenylene, para-benzylene,

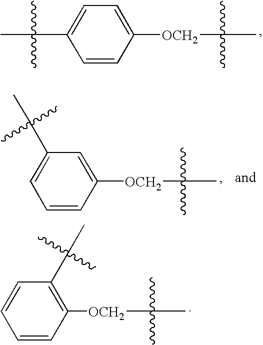

5. A compound selected from:
COMPOUND 12.1.1: N,N-Diethyl-2-{[2-(2-furylmethyl)-6,7-dimethoxy-1,2,3,4-tetrahydroisoquinolin-1-yl]methoxy}benzamide
COMPOUND 12.1.2: 2-{[6,7-Dimethoxy-2-(thien-3-ylmethyl)-1,2,3,4-tetrahydroisoquinolin-1-yl]methoxy}-N,N-diethylbenzamide
COMPOUND 12.1.3: N,N-Diethyl-3-{[2-(2-furylmethyl)-6,7-dimethoxy-1,2,3,4-tetrahydroisoquinolin-1-yl]methoxy}benzamide
COMPOUND 12.1.4: 3-{[6,7-Dimethoxy-2-(thien-3-ylmethyl)-1,2,3,4-tetrahydroisoquinolin-1-yl]methoxy}-N,N-diethylbenzamide
COMPOUND 12.1.5: N,N-Diethyl-4-{[2-(2-furylmethyl)-6,7-dimethoxy-1,2,3,4-tetrahydroisoquinolin-1-yl]methoxy}benzamide
COMPOUND 12.1.6: 4-{[6,7-Dimethoxy-2-(thien-3-ylmethyl)-1,2,3,4-tetrahydroisoquinolin-1-yl]methoxy}-N,N-diethylbenzamide
COMPOUND 12.1.7: 2-({6,7-Dimethoxy-2-[(2-phenyl-1H-imidazol-5-yl)methyl]-1,2,3,4-tetrahydroisoquinolin-1-yl}methoxy)-N,N-diethylbenzamide
COMPOUND 12.1.8: 4-({6,7-Dimethoxy-2-[(2-phenyl-1H-imidazol-5-yl)methyl]-1,2,3,4-tetrahydroisoquinolin-1-yl}methyl)-N,N-diethylbenzamide
COMPOUND 12.1.9: 4-{6,7-Dimethoxy-2-[(2-phenyl-1H-imidazol-5-yl)methyl]-1,2,3,4-tetrahydroisoquinolin-1-yl}-N,N-diethylbenzamide
COMPOUND 12.1.10: N,N-Diethyl-4-{6-methoxy-2-[(2-phenyl-1H-imidazol-5-yl)methyl]-1,2,3,4-tetrahydroisoquinolin-1-yl}benzamide
COMPOUND 12.1.11: N,N-Diethyl-4-{7-methoxy-2-[(2-phenyl-1H-imidazol-5-yl)methyl]-1,2,3,4-tetrahydroisoquinolin-1-yl}benzamide
COMPOUND 12.1.12: N,N-Diethyl-4-{2-[(2-phenyl-1H-imidazol-5-yl)methyl]-1,2,3,4-tetrahydroisoquinolin-1-yl}benzamide
COMPOUND 12.1.13: 4-{2-[(2-Butyl-1H-imidazol-5-yl)methyl]-6,7-dimethoxy-1,2,3,4-tetrahydroisoquinolin-1-yl}-N,N-diethylbenzamide
COMPOUND 12.1.14: 4-{2-[(2-Butyl-4-chloro-1H-imidazol-5-yl)methyl]-6,7-dimethoxy-1,2,3,4-tetrahydroisoquinolin-1-yl}-N,N-diethylbenzamide
COMPOUND 12.1.15: 4-{6,7-Dimethoxy-2-[(2-methyl-1H-imidazol-5-yl)methyl]-1,2,3,4-tetrahydroisoquinolin-1-yl}-N,N-diethylbenzamide
COMPOUND 12.1.16: 4-{6,7-Dimethoxy-2-[(3-phenyl-1H-pyrazol-4-yl)methyl]-1,2,3,4-tetrahydroisoquinolin-1-yl}-N,N-diethylbenzamide
COMPOUND 12.1.17: 4-(6,7-Dimethoxy-2-{[1-(phenylsulfonyl)-1H-pyrrol-2-yl]methyl}-1,2,3,4-tetrahydroisoquinolin-1-yl)-N,N-diethylbenzamide
COMPOUND 12.1.18: N,N-Diethyl-4-{2-[(2-ethyl-4-methyl-1H-imidazol-5-yl)methyl]-6,7-dimethoxy-1,2,3,4-tetrahydroisoquinolin-1-yl}benzamide
COMPOUND 12.1.19: 4-{6,7-Dimethoxy-2-[(4-methyl-1H-imidazol-5-yl)methyl]-1,2,3,4-tetrahydroisoquinolin-1-yl}-N,N-diethylbenzamide
COMPOUND 12.1.20: 4-{5,8-Dimethoxy-2-[(4-methyl-1H-imidazol-5-yl)methyl]-1,2,3,4-tetrahydroisoquinolin-1-yl}-N,N-diethylbenzamide
COMPOUND 12.1.21: N,N-Diethyl-4-[1,2,3,4-tetrahydro-6-methoxy-2-[(4-methyl-1H-imidazol-5-yl)methyl]-1-isoquinolinyl]-benzamide
COMPOUND 12.1.22: N,N-Diethyl-4-[2-(1H-imidazol-5-ylmethyl)-6-methoxy-1,2,3,4-tetrahydroisoquinolin-1-yl]benzamide
COMPOUND 12.1.23: N,N-Diethyl-4-[2-(1H-imidazol-5-ylmethyl)-6,7-dimethoxy-1,2,3,4-tetrahydroisoquinolin-1-yl]benzamide
COMPOUND 12.1.24: 4-{6,7-Dimethoxy-2-[(5-phenyl-2-furyl)methyl]-1,2,3,4-tetrahydroisoquinolin-1-yl}-N,N-diethylbenzamide
COMPOUND 12.1.25: N,N-Diethyl-4-{6-methoxy-2-[(5-phenyl-2-furyl)methyl]-1,2,3,4-tetrahydroisoquinolin-1-yl}benzamide
COMPOUND 12.1.26: N,N-Diethyl-4-{7-hydroxy-6-methoxy-2-[(5-methyl-1H-imidazol-4-yl)methyl]-1,2,3,4-tetrahydroisoquinolin-1-yl}benzamide
COMPOUND 12.1.27: N,N-Diethyl-4-{7-hydroxy-6-methoxy-2-[(2-phenyl-1H-imidazol-4-yl)methyl]-1,2,3,4-tetrahydroisoquinolin-1-yl}benzamide
COMPOUND 12.1.28: 4-{2-[(1-Benzyl-1H-imidazol-5-yl)methyl]-6,7-dimethoxy-1,2,3,4-tetrahydroisoquinolin-1-yl}-N,N-diethylbenzamide
COMPOUND 12.1.29: 4-{6,7-Dimethoxy-2-[(1-methyl-1H-imidazol-5-yl)methyl]-1,2,3,4-tetrahydroisoquinolin-1-yl}-N,N-diethylbenzamide
COMPOUND 12.1.30: 4-{6,7-Dimethoxy-2-[(1-methyl-1H-imidazol-4-yl)methyl]-1,2,3,4-tetrahydroisoquinolin-1-yl}-N,N-diethylbenzamide
COMPOUND 12.1.31: 4-({6,7-Dimethoxy-2-[(4-methyl-1H-imidazol-5-yl)methyl]-1,2,3,4-tetrahydroisoquinolin-1-yl}methoxy)-N,N-diethylbenzamide
COMPOUND 12.1.32: 4-({6,7-Dimethoxy-2-[(4-methyl-1H-imidazol-5-yl)methyl]-1,2,3,4-tetrahydroisoquinolin-1-yl}methyl)-N,N-diethylbenzamide
COMPOUND 12.1.33: 1-{4-[(Diethylamino)carbonyl]phenyl}-2-[(4-methyl-1H-imidazol-5-yl)methyl]-1,2,3,4-tetrahydroisoquinolin-6-yl methanesulfonate
COMPOUND 12.1.34: 1-{4-[(Diethylamino)carbonyl]phenyl}-2-[(2-phenyl-1H-imidazol-5-yl)methyl]-1,2,3,4-tetrahydroisoquinolin-6-yl methanesulfonate
COMPOUND 12.1.35: 1-{4-[(Diethylamino)carbonyl]phenyl}-2-[(4-methyl-1H-imidazol-5-yl)methyl]-1,2,3,4-tetrahydroisoquinolin-6-yl dimethylsulfamate
COMPOUND 12.1.36: 1-{4-[(Diethylamino)carbonyl]phenyl}-2-[(2-phenyl-1H-imidazol-5-yl)methyl]-1,2,3,4-tetrahydroisoquinolin-6-yl dimethylsulfamate
COMPOUND 12.1.37: 4-{2-[(2,5-Dimethyl-1,3-thiazol-4-yl)methyl]-6,7-dimethoxy-1,2,3,4-tetrahydroisoquinolin-1-yl}-N,N-diethylbenzamide
COMPOUND 12.1.38: 4-{6,7-Dimethoxy-2-[(2-phenyl-1,3-thiazol-5-yl)methyl]-1,2,3,4-tetrahydroisoquinolin-1-yl}-N,N-diethylbenzamide
COMPOUND 12.1.39: N,N-Diethyl-4-{7-isopropoxy-6-methoxy-2-[(5-methyl-1H-imidazol-4-yl)methyl]-1,2,3,4-tetrahydroisoquinolin-1-yl}benzamide
COMPOUND 12.1.40: N,N-Diethyl-4-[6-methoxy-2-[(5-methyl-1H-imidazol-4-yl)methyl]-7-(2-morpholin-4-ylethoxy)-1,2,3,4-tetrahydroisoquinolin-1-yl]benzamide
COMPOUND 12.1.41: 4-{7-Ethoxy-6-methoxy-2-[(2-phenyl-1H-imidazol-4-yl)methyl]-1,2,3,4-tetrahydroisoquinolin-1-yl}-N,N-diethylbenzamide
COMPOUND 12.1.42: N,N-Diethyl-4-{7-isopropoxy-6-methoxy-2-[(2-phenyl-1H-imidazol-4-yl)methyl]-1,2,3,4-tetrahydroisoquinolin-1-yl}benzamide
COMPOUND 12.1.43: N,N-Diethyl-4-{6-methoxy-7-(2-morpholin-4-ylethoxy)-2-[(2-phenyl-1H-imidazol-4-yl)methyl]-1,2,3,4-tetrahydroisoquinolin-1-yl}benzamide
COMPOUND 12.1.44: N,N-Diethyl-4-{7-methoxy-2-[(4-methyl-1H-imidazol-5-yl)methyl]-1,2,3,4-tetrahydroisoquinolin-1-yl}benzamide
COMPOUND 12.1.45: Methyl 5-{[1-{4-[(diethylamino)carbonyl]phenyl}-6,7-dimethoxy-3,4-dihydroisoquinolin-2(1H)-yl]methyl}-1H-imidazole-4-carboxylate
COMPOUND 12.1.46: 1-{4-[(Diethylamino)carbonyl]phenyl}-6-methoxy-2-[(4-methyl-1H-imidazol-5-yl)methyl]-1,2,3,4-tetrahydroisoquinolin-7-yl methanesulfonate
COMPOUND 12.1.47: N,N-Diethyl-4-{6-[(4-methyl-1H-imidazol-5-yl)methyl]-5,6,7,8-tetrahydro[1,3]dioxolo[4,5-g]isoquinolin-5-yl}benzamide
COMPOUND 12.1.48: N,N-Diethyl-4-{6-[(2-phenyl-1H-imidazol-5-yl)methyl]-5,6,7,8-tetrahydro[1,3]dioxolo[4,5-g]isoquinolin-5-yl}benzamide
COMPOUND 12.1.49: 4-{6-Bromo-7-methoxy-2-[(4-methyl-1H-imidazol-5-yl)methyl]-1,2,3,4-tetrahydroisoquinolin-1-yl}-N,N-diethylbenzamide
COMPOUND 12.1.50: 4-{6-Bromo-7-methoxy-2-[(2-phenyl-1H-imidazol-5-yl)methyl]-1,2,3,4-tetrahydroisoquinolin-1-yl}-N,N-diethylbenzamide
COMPOUND 12.1.51: 4-{6,7-Dimethoxy-3-methyl-2-[(4-methyl-1H-imidazol-5-yl)methyl]-1,2,3,4-tetrahydroisoquinolin-1-yl}-N,N-diethylbenzamide
COMPOUND 12.1.52: N,N-Diethyl-4-[2-(1H-imidazol-5-ylmethyl)-6,7-dimethoxy-3-methyl-1,2,3,4-tetrahydroisoquinolin-1-yl]benzamide
COMPOUND 12.1.53: N,N-Diethyl-4-{6-methoxy-2-[(4-methyl-1H-imidazol-5-yl)methyl]-7-nitro-1,2,3,4-tetrahydroisoquinolin-1-yl}benzamide
COMPOUND 12.1.54: N,N-Diethyl-4-{6-methoxy-2-[(4-methyl-1H-imidazol-5-yl)methyl]-5-nitro-1,2,3,4-tetrahydroisoquinolin-1-yl}benzamide
COMPOUND 12.1.55: N,N-Diethyl-4-{7-[(4-methyl-1H-imidazol-5-yl)methyl]-6,7,8,9-tetrahydro[1,3]dioxolo[4,5-f]isoquinolin-6-yl}benzamide
COMPOUND 12.1.56: N,N-Diethyl-4-{7-[(2-phenyl-1H-imidazol-5-yl)methyl]-6,7,8,9-tetrahydro[1,3]dioxolo[4,5-f]isoquinolin-6-yl}benzamide
COMPOUND 12.1.57: N,N-Diethyl-4-{5,6,7-trimethoxy-2-[(4-methyl-1H-imidazol-5-yl)methyl]-1,2,3,4-tetrahydroisoquinolin-1-yl}benzamide
COMPOUND 12.1.58: N,N-Diethyl-4-{5,6,7-trimethoxy-2-[(2-phenyl-1H-imidazol-5-yl)methyl]-1,2,3,4-tetrahydroisoquinolin-1-yl}benzamide
COMPOUND 12.1.59: 4-{7-(Cyclobutyloxy)-6-methoxy-2-[(5-methyl-1H-imidazol-4-yl)methyl]-1,2,3,4-tetrahydroisoquinolin-1-yl}-N,N-diethylbenzamide
COMPOUND 12.1.60: N,N-Diethyl-4-[6-methoxy-2-[(5-methyl-1H-imidazol-4-yl)methyl]-7-(neopentyloxy)-1,2,3,4-tetrahydroisoquinolin-1-yl]benzamide
COMPOUND 12.1.61: N,N-Diethyl-4-{6-fluoro-7-methoxy-2-[(4-methyl-1H-imidazol-5-yl)methyl]-1,2,3,4-tetrahydroisoquinolin-1-yl}benzamide
COMPOUND 12.1.62: N,N-Diethyl-4-{6-fluoro-7-methoxy-2-[(2-phenyl-1H-imidazol-5-yl)methyl]-1,2,3,4-tetrahydroisoquinolin-1-yl}benzamide
COMPOUND 12.1.63: 1-{4-[(Diethylamino)carbonyl]phenyl}-6-methoxy-2-[(2-phenyl-1H-imidazol-5-yl)methyl]-1,2,3,4-tetrahydroisoquinolin-7-yl dimethylsulfamate
COMPOUND 13.1.1: N,N-Diethyl-4-[6-methoxy-2-[(5-methyl-1H-imidazol-4-yl)methyl]-7-(tetrahydro-2H-pyran-4-yloxy)-1,2,3,4-tetrahydroisoquinolin-1-yl]benzamide
COMPOUND 14.1.1: N,N-Diethyl-4-{6-methoxy-7-phenoxy-2-[(2-phenyl-1H-imidazol-4-yl)methyl]-1,2,3,4-tetrahydroisoquinolin-1-yl}benzamide
COMPOUND 14.1.2: N,N-Diethyl-4-{6-methoxy-2-[(5-methyl-1H-imidazol-4-yl)methyl]-7-phenoxy-1,2,3,4-tetrahydroisoquinolin-1-yl}benzamide
COMPOUND 14.1.3: N,N-diethyl-4-{7-(4-fluorophenoxy)-6-methoxy-2-[(2-phenyl-1H-imidazol-4-yl)methyl]-1,2,3,4-tetrahydroisoquinolin-1-yl}benzamide
COMPOUND 14.1.4: N,N-Diethyl-4-{7-(4-fluorophenoxy)-6-methoxy-2-[(5-methyl-1H-imidazol-4-yl)methyl]-1,2,3,4-tetrahydroisoquinolin-1-yl}benzamide
COMPOUND 14.1.5: N,N-Diethyl-4-{6-methoxy-7-(4-methoxyphenoxy)-2-[(5-methyl-1H-imidazol-4-yl)methyl]-1,2,3,4-tetrahydroisoquinolin-1-yl}benzamide
COMPOUND 14.1.6: N,N-Diethyl-4-[6-methoxy-2-[(5-methyl-1H-imidazol-4-yl)methyl]-7-(pyridin-3-yloxy)-1,2,3,4-tetrahydroisoquinolin-1-yl]benzamide
COMPOUND 15.1.1: 4-{7-(Benzyloxy)-6-methoxy-2-[(5-methyl-1H-imidazol-4-yl)methyl]-1,2,3,4-tetrahydroisoquinolin-1-yl}-N,N-diethylbenzamide
COMPOUND 16.4.1: N,N-Diethyl-4-{6-methoxy-7-(3-methoxyphenoxy)-2-[(5-methyl-1H-imidazol-4-yl)methyl]-1,2,3,4-tetrahydroisoquinolin-1-yl}benzamide
COMPOUND 16.4.2: N,N-Diethyl-4-{6-methoxy-7-(4-methoxyphenoxy)-2-[(5-methyl-1H-imidazol-4-yl)methyl]-1,2,3,4-tetrahydroisoquinolin-1-yl}benzamide
COMPOUND 16.4.3: 1-{4-[(Diethylamino)carbonyl]phenyl}-6-methoxy-2-[(5-methyl-1H-imidazol-4-yl)methyl]-1,2,3,4-tetrahydroisoquinolin-7-yl benzenesulfonate
COMPOUND 17.1.1: 4-{6,7-Dihydroxy-2-[(2-phenyl-1H-imidazol-5-yl)methyl]-1,2,3,4-tetrahydroisoquinolin-1-yl}-N,N-diethylbenzamide
COMPOUND 17.1.2: N,N-Diethyl-4-{6-hydroxy-2-[(2-phenyl-1H-imidazol-5-yl)methyl]-1,2,3,4-tetrahydroisoquinolin-1-yl}benzamide
COMPOUND 17.1.3: N,N-Diethyl-4-{7-hydroxy-2-[(2-phenyl-1H-imidazol-5-yl)methyl]-1,2,3,4-tetrahydroisoquinolin-1-yl}benzamide
COMPOUND 17.1.4: N,N-Diethyl-4-[1,2,3,4-tetrahydro-6-hydroxy-2-[(4-methyl-1H-imidazol-5-yl)methyl]-1-isoquinolinyl]-benzamide
COMPOUND 17.1.5: N,N-Diethyl-4-{7-hydroxy-2-[(4-methyl-1H-imidazol-5-yl)methyl]-1,2,3,4-tetrahydroisoquinolin-1-yl}benzamide
COMPOUND 17.1.6: N,N-Diethyl-4-{6-hydroxy-7-phenoxy-2-[(2-phenyl-1H-imidazol-4-yl)methyl]-1,2,3,4-tetrahydroisoquinolin-1-yl}benzamide
COMPOUND 17.1.7: N,N-Diethyl-4-{6-hydroxy-2-[(5-methyl-1H-imidazol-4-yl)methyl]-7-phenoxy-1,2,3,4-tetrahydroisoquinolin-1-yl}benzamide
COMPOUND 17.1.8: N,N-Diethyl-4-{7-(4-fluorophenoxy)-6-hydroxy-2-[(2-phenyl-1H-imidazol-4-yl)methyl]-1,2,3,4-tetrahydroisoquinolin-1-yl}benzamide
COMPOUND 17.1.9: N,N-Diethyl-4-{7-(4-fluorophenoxy)-6-hydroxy-2-[(5-methyl-1H-imidazol-4-yl)methyl]-1,2,3,4-tetrahydroisoquinolin-1-yl}benzamide
COMPOUND 18.1.1: 4-{2-[(1,4-Dimethyl-1H-imidazol-5-yl)methyl]-6,7-dimethoxy-1,2,3,4-tetrahydroisoquinolin-1-yl}-N,N-diethylbenzamide
COMPOUND 18.1.2: 4-{2-[(1,5-Dimethyl-1H-imidazol-4-yl)methyl]-6,7-dimethoxy-1,2,3,4-tetrahydroisoquinolin-1-yl}-N,N-diethylbenzamide
COMPOUND 19.1.1: 4-{7-Ethoxy-6-methoxy-2-[(5-methyl-1H-imidazol-4-yl)methyl]-1,2,3,4-tetrahydroisoquinolin-1-yl}-N,N-diethylbenzamide
COMPOUND 20.1.1: 4-{(1S)-6,7-Dimethoxy-2-[(4-methyl-1H-imidazol-5-yl)methyl]-1,2,3,4-tetrahydroisoquinolin-1-yl}-N,N-diethylbenzamide
COMPOUND 20.2.1: 4-{(1R)-6,7-Dimethoxy-2-[(4-methyl-1H-imidazol-5-yl)methyl]-1,2,3,4-tetrahydroisoquinolin-1-yl}-N,N-diethylbenzamide
COMPOUND 20.1.2: N,N-Diethyl-4-{(1S)-6-methoxy-2-[(4-methyl-1H-imidazol-5-yl)methyl]-1,2,3,4-tetrahydroisoquinolin-1-yl}benzamide
COMPOUND 20.2.2: N,N-Diethyl-4-{(1R)-6-methoxy-2-[(4-methyl-1H-imidazol-5-yl)methyl]-1,2,3,4-tetrahydroisoquinolin-1-yl}benzamide
COMPOUND 20.1.3: N,N-Diethyl-4-{(1S)-6-hydroxy-2-[(2-phenyl-1H-imidazol-5-yl)methyl]-1,2,3,4-tetrahydroisoquinolin-1-yl}benzamide
COMPOUND 20.2.3: N,N-Diethyl-4-{(1R)-6-hydroxy-2-[(2-phenyl-1H-imidazol-5-yl)methyl]-1,2,3,4-tetrahydroisoquinolin-1-yl}benzamide
COMPOUND 20.1.4: N,N-Diethyl-4-{(1S)-7-isopropoxy-6-methoxy-2-[(4-methyl-1H-imidazol-5-yl)methyl]-1,2,3,4-tetrahydroisoquinolin-1-yl}benzamide
COMPOUND 20.2.4: N,N-Diethyl-4-{(1R)-7-isopropoxy-6-methoxy-2-[(4-methyl-1H-imidazol-5-yl)methyl]-1,2,3,4-tetrahydroisoquinolin-1-yl}benzamide
COMPOUND 20.1.5: N,N-Diethyl-4-{(1S)-7-isopropoxy-6-methoxy-2-[(2-phenyl-1H-imidazol-5-yl)methyl]-1,2,3,4-tetrahydroisoquinolin-1-yl}benzamide
COMPOUND 20.2.5: N,N-Diethyl-4-{(1R)-7-isopropoxy-6-methoxy-2-[(2-phenyl-1H-imidazol-5-yl)methyl]-1,2,3,4-tetrahydroisoquinolin-1-yl}benzamide
COMPOUND 20.1.6: N,N-Diethyl-4-{(1S)-6-methoxy-7-(2-morpholin-4-ylethoxy)-2-[(2-phenyl-1H-imidazol-5-yl)methyl]-1,2,3,4-tetrahydroisoquinolin-1-yl}benzamide
COMPOUND 20.2.6: N,N-Diethyl-4-{(1R)-6-methoxy-7-(2-morpholin-4-ylethoxy)-2-[(2-phenyl-1H-imidazol-5-yl)methyl]-1,2,3,4-tetrahydroisoquinolin-1-yl}benzamide
COMPOUND 20.1.7: N,N-Diethyl-4-[(1S)-1,2,3,4-tetrahydro-6-methoxy-2-[(4-methyl-1H-imidazol-5-yl)methyl]-1-isoquinolinyl]-benzamide
COMPOUND 20.2.7: N,N-Diethyl-4-[(1R)-1,2,3,4-tetrahydro-6-methoxy-2-[(4-methyl-1H-imidazol-5-yl)methyl]-1-isoquinolinyl]-benzamide
COMPOUND 20.1.8: N,N-Diethyl-4-[(1S)-1,2,3,4-tetrahydro-6-hydroxy-2-[(4-methyl-1H-imidazol-5-yl)methyl]-1-isoquinolinyl]-benzamide
COMPOUND 20.2.8: N,N-Diethyl-4-[(1R)-1,2,3,4-tetrahydro-6-hydroxy-2-[(4-methyl-1H-imidazol-5-yl)methyl]-1-isoquinolinyl]-benzamide;
and pharmaceutically acceptable salts thereof.

6-7. (canceled)

8. A pharmaceutical composition comprising a compound according to claim 1 and a pharmaceutically acceptable carrier.

9. A method for the therapy of pain in a warm-blooded animal, comprising the step of administering to said animal in need of such therapy a therapeutically effective amount of a compound according claim 1.

10. A method for the therapy of functional gastrointestinal disorders in a warm-blooded animal, comprising the step of administering to said animal in need of such therapy a therapeutically effective amount of a compound according to claim 1.

11. A process for preparing a compound of formula II, 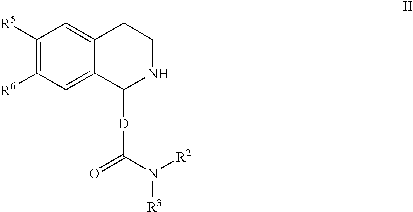
comprising of the step of reacting a compound of formula III with a compound of formula IV in the presence of HNR²R³: 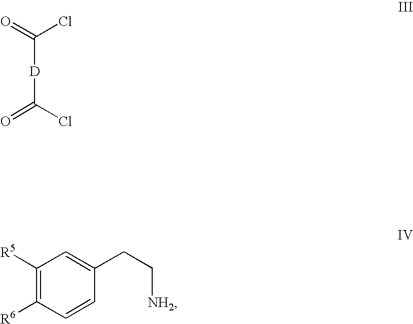
wherein
R² and R³ are independently selected from —H and C₁₋₆alkyl;
R⁵ and R⁶ are independently selected from —H, —OH, halogen, —NO₂, C₁₋₆alkyl, C₆₋₁₀aryl, C₁₋₆alkoxy, C₃₋₆cycloalkoxy, C₃₋₆heterocyclyl-oxy, C₃₋₆heterocyclyl-C₁₋₄alkoxy, C₆₋₁₀aryl-oxy, C₆₋₁₀aryl-C₁₋₄alkoxy, C₁₋₆alkyl-S(═O)₂—O—, C₆₋₁₀aryl-S(═O)₂—O—, C₁₋₆alkyl-NH—S(═O)₂—O—, and (C₁₋₆alkyl)₂N—S(═O)₂—O—; or R⁵ and R⁶ together form a portion of a 5 or 6-membered ring that fused with the benzene ring of formula I, wherein said C₁₋₆alkyl, C₆₋₁₀aryl, C₁₋₆alkoxy, C₃₋₆cycloalkoxy, C₃₋₆heterocyclyl-oxy, C₃₋₆heterocyclyl-C₁₋₄alkoxy, C₆₋₁₀aryl-oxy, C₆₋₁₀aryl-C₁₋₄alkoxy, C₁₋₆alkyl-S(═O)₂—O—, C₆₋₁₀aryl-S(═O)₂—O—, C₁₋₆alkyl-NH—S(═O)₂—O—, and (C₁₋₆alkyl)₂N—S(═O)₂—O— are optionally substituted with one or more groups selected from halogen, C₁₋₃alkoxy, —OH, —NO₂, C₁₋₃alkyl, —NH₂, and —CO₂—C₁₋₃alkyl; and
D is a divalent group comprising a benzene ring.

12. A process for preparing a compound of formula V, 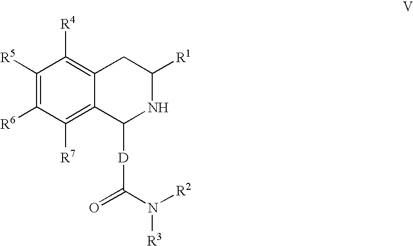
comprising of the step of reacting a compound of formula VI with a compound of formula VII in the presence of an acid catalyst: 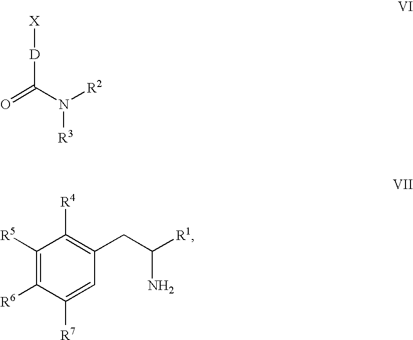
wherein
X is selected from —CH(OEt)₂, ═CHOMe and —CHO;
R¹ is selected from —H and C₁₋₆alkyl;
R² and R³ are independently selected from —H and C₁₋₆alkyl;
R⁴, R⁵, R⁵ and R⁷ are independently selected from —H, —OH, halogen, —NO₂, C₁₋₆alkyl, C₆₋₁₀aryl, C₁₋₆alkoxy, C₃₋₆cycloalkoxy, C₃₋₆heterocyclyl-oxy, C₃₋₆heterocyclyl-C₁₋₄alkoxy, C₆₋₁₀aryl-oxy, C₆₋₁₀aryl-C₁₋₄alkoxy, C₁₋₆alkyl-S(═O)₂—O—, C₆₋₁₀aryl-S(═O)₂—O—, C₁₋₆alkyl-NH—S(═O)₂—O—, and (C₁₋₆alkyl)₂N—S(═O)₂—O—; or any two adjacent groups selected from R⁴, R⁵, R⁶ and R⁷ form a portion of a 5 or 6-membered ring that fused with the benzene ring of formula I, wherein said C₁₋₆alkyl, C₆₋₁₀aryl, C₁₋₆alkoxy, C₃₋₆cycloalkoxy, C₃₋₆heterocyclyl-oxy, C₃₋₆heterocyclyl-C₁₋₄alkoxy, C₆₋₁₀aryl-oxy, C₆₋₁₀aryl-C₁₋₄alkoxy, C₁₋₆alkyl-S(═O)₂—O—, C₆₋₁₀aryl-S(═O)₂—O—, C₁₋₆alkyl-NH—S(═O)₂—O—, and (C₁₋₆alkyl)₂N—S(═O)₂—O— are optionally substituted with one or more groups selected from halogen, C₁₋₃alkoxy, —OH, —NO₂, C₁₋₃alkyl, —NH₂, and —CO₂—C₁₋₃alkyl; and
D is a divalent group comprising a benzene ring.

13. A process for preparing a compound of formula I, 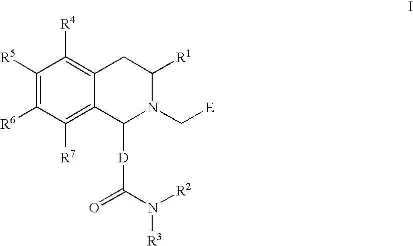
comprising: reacting a compound of formula VIII with E-CHO: 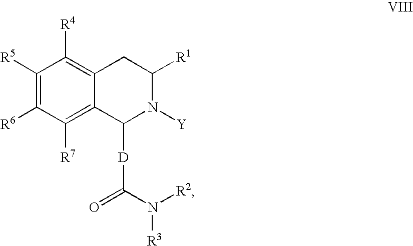
wherein
Y is selected from —H and —C(═O)—O-t-butyl;
R¹ is selected from —H and C₁₋₆alkyl;
R² and R³ are independently selected from —H and C₁₋₆alkyl;
R⁴, R⁵, R⁶ and R⁷ are independently selected from —H, —OH, halogen, —NO₂, C₁₋₆alkyl, C₆₋₁₀aryl, C₁₋₆alkoxy, C₃₋₆cycloalkoxy, C₃₋₆heterocyclyl-oxy, C₃₋₆heterocyclyl-C₁₋₄alkoxy, C₆₋₁₀aryl-oxy, C₆₋₁₀aryl-C₁₋₄alkoxy, C₁₋₆alkyl-S(═O)₂—O—, C₆₋₁₀aryl-S(═O)₂—O—, C₁₋₆alkyl-NH—S(═O)₂—O—, and (C₁₋₆alkyl)₂N—S(═O)₂—O—; or any two adjacent groups selected from R⁴, R⁵, R⁶ and R⁷ form a portion of a 5 or 6-membered ring that fused with the benzene ring of formula I, wherein said C₁₋₆alkyl, C₆₋₁₀aryl, C₁₋₆alkoxy, C₃₋₆cycloalkoxy, C₃₋₆heterocyclyl-oxy, C₃₋₆heterocyclyl-C₁₋₄alkoxy, C₆₋₁₀aryl-oxy, C₆₋₁₀aryl-C₁₋₄alkoxy, C₁₋₆alkyl-S(═O)₂—O—, C₆₋₁₀aryl-S(═O)₂—O—, C₁₋₆alkyl-NH—S(═O)₂—O—, and (C₁₋₆alkyl)₂N—S(═O)₂—O— are optionally substituted with one or more groups selected from halogen, C₁₋₃alkoxy, —OH, —NO₂, C₁₋₃alkyl, —NH₂, and —CO₂—C₁₋₃alkyl;
E is a 5-membered heterocyclyl optionally substituted with one or more groups selected from halogen, C₁₋₆alkyl, —C(═O)—O—C₁₋₆alkyl, C₆₋₁₀aryl, C₆₋₁₀aryl-C₁₋₄alkyl, and C₆₋₁₀aryl-S(═O)₂—; and
D is a divalent group comprising a benzene ring.

14. A process for preparing a compound of formula IX, 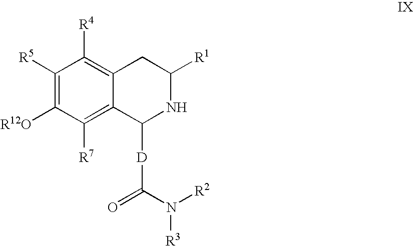
comprising: reacting a compound of formula X with R¹²—OH or R¹²—B(OH)₂: 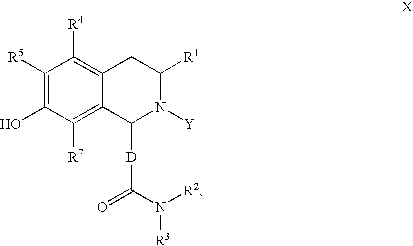
wherein
Y is selected from —H and —C(═O)—O-t-butyl;
R¹² is selected from C₁₋₆alkyl, C₃₋₆cycloalkyl, C₆₋₁₀aryl-C₁₋₄alkyl, C₃₋₆heterocyclyl-C₁₋₄alkyl, C₆₋₁₀aryl, and C₃₋₆heteroaryl, wherein said C₆₋₁₀aryl, C₃₋₆heterocyclyl and C₃₋₆heteroaryl are optionally substituted with one or more groups selected from halogen, C₁₋₃alkoxy, —OH, —NO₂, C₁₋₃alkyl, —NH₂ and —CO₂—C₁₋₃alkyl; and
R¹ is selected from —H and C₁₋₆alkyl;
R² and R³ are independently selected from —H and C₁₋₆alkyl;
R⁴, R⁵, and R⁷ are independently selected from —H, —OH, halogen, —NO₂, C₁₋₆alkyl, C₆₋₁₀aryl, C₁₋₆alkoxy, C₃₋₆cycloalkoxy, C₃₋₆heterocyclyl-oxy, C₃₋₆heterocyclyl-C₁₋₄alkoxy, C₆₋₁₀aryl-oxy, C₆₋₁₀aryl-C₁₋₄alkoxy, C₁₋₆alkyl-S(═O)₂—O—, C₆₋₁₀aryl-S(═O)₂—O—, C₁₋₆alkyl-NH—S(═O)₂—O—, and (C₁₋₆alkyl)₂N—S(═O)₂—O—; or R⁴ and R⁵ together form a portion of a 5 or 6-membered ring that fused with the benzene ring of formula I, wherein said C₁₋₆alkyl, C₆₋₁₀aryl, C₁₋₆alkoxy, C₃₋₆cycloalkoxy, C₃₋₆heterocyclyl-oxy, C₃₋₆heterocyclyl-C₁₋₄alkoxy, C₆₋₁₀aryl-oxy, C₆₋₁₀aryl-C₁₋₄alkoxy, C₁₋₆alkyl-S(═O)₂—O—, C₆₋₁₀aryl-S(═O)₂—O—, C₁₋₆alkyl-NH—S(═O)₂—O—, and (C₁₋₆alkyl)₂N—S(═O)₂—O— are optionally substituted with one or more groups selected from halogen, C₁₋₃alkoxy, —OH, —NO₂, C₁₋₃alkyl, —NH₂, and —CO₂—C₁₋₃alkyl; and
D is a divalent group comprising a benzene ring.

15. A process for preparing a compound of formula XI, 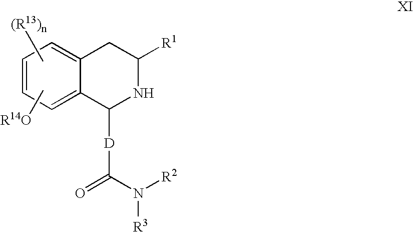
comprising:
reacting a compound of formula XII with NsCl, NsBr, or (CF₃CO)₂O to protect the ═NH group of formula XI;
reacting the protected compound with R¹⁴—Y¹ followed by deprotecting the ═NH group: 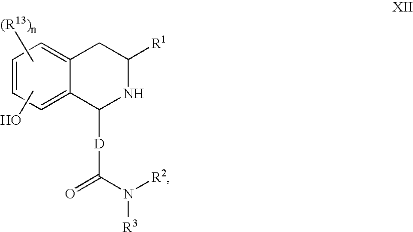
wherein
n is 0, 1, 2 or 3;
each R¹³ is independently selected from —H, —OH, halogen, —NO₂, C₁₋₆alkyl, C₆₋₁₀aryl, C₁₋₆alkoxy, C₃₋₆cycloalkoxy, C₃₋₆heterocyclyl-oxy, C₃₋₆heterocyclyl-C₁₋₄alkoxy, C₆₋₁₀aryl-oxy, C₆₋₁₀aryl-C₁₋₄alkoxy, C₁₋₆alkyl-S(═O)₂—O—, C₆₋₁₀aryl-S(═O)₂—O—, C₁₋₆alkyl-NH—S(═O)₂—O—, and (C₁₋₆alkyl)₂N—S(═O)₂—O—; or any two adjacent R¹³ form a portion of a 5 or 6-membered ring that fused with the benzene ring of formula I, wherein said C₁₋₆alkyl, C₆₋₁₀aryl, C₁₋₆alkoxy, C₃₋₆cycloalkoxy, C₃₋₆heterocyclyl-oxy, C₃₋₆heterocyclyl-C₁₋₄alkoxy, C₆₋₁₀aryl-oxy, C₆₋₁₀aryl-C₁₋₄alkoxy, C₁₋₆alkyl-S(═O)₂—O—, C₆₋₁₀aryl-S(═O)₂—O—, C₁₋₆alkyl-NH—S(═O)₂—O—, and (C₁₋₆alkyl)₂N—S(═O)₂—O— are optionally substituted with one or more groups selected from halogen, C₁₋₃alkoxy, —OH, —NO₂, C₁₋₃alkyl, —NH₂, and —CO₂—C₁₋₃alkyl;
Y¹ is halogen;
R¹⁴ is selected from C₁₋₆alkyl-S(═O)₂—, C₆₋₁₀aryl-S(═O)₂—, C₁₋₆alkyl-NH—S(═O)₂—, and (C₁₋₆alkyl)₂N—S(═O)₂—;
R¹ is selected from —H and C₁₋₆alkyl;
R² and R³ are independently selected from —H and C₁₋₆alkyl; and
D is a divalent group comprising a benzene ring.

16. A method for the therapy of anxiety in a warm-blooded animal, comprising the step of administering to said animal in need of such therapy a therapeutically effective amount of a compound according to claim 1.
